(19) United States
(12) Patent Application Publication
(10) Pub. No.: US 2018/0155788 A1
(43) Pub. Date: Jun. 7, 2018

(54) METHODS OF PREDICTING THE DEVELOPMENT OF COMPLEMENT-MEDIATED DISEASE

(71) Applicant: University of Utah Research Foundation, Salt Lake City, UT (US)

(72) Inventors: GREGORY S. HAGEMAN, Salt Lake City, UT (US); Christian Matthew Pappas, Salt Lake City, UT (US); Eric N. Brown, Overland Park, KS (US); David Hutcheson, Salt Lake City, UT (US)

(73) Assignee: University of Utah Research Foundation, Salt Lake City, UT (US)

(21) Appl. No.: 15/634,892

(22) Filed: Jun. 27, 2017

Related U.S. Application Data
(63) Continuation of application No. 14/114,751, filed on May 12, 2014, now abandoned.
(60) Provisional application No. 61/480,929, filed on Apr. 29, 2011.

(51) Int. Cl. C12Q 1/6883

(52) U.S. Cl. CPC C12Q 1/6883; C12Q 2600/172; C12Q 2600/118

(57) ABSTRACT

Described herein are methods for determining a Caucasian subject's susceptibility to having or developing a complement-mediated disease comprising determining in the Caucasian subject the identity of one or more haplotypes, wherein the presence of one or more of the haplotypes indicates the subject's susceptibility for having or developing a complement-mediated disease.

### CROSS-REFERENCES TO RELATED APPLICATIONS

This application is a continuation of U.S. application Ser. No. 14/114,751 (published as US 2014/0357732 A1), maintaining the international filing date of Apr. 27, 2012, which is a National Phase Application of International Application No. PCT/US2012/035467 (published as WO 2012/149329), filed Apr. 27, 2012, which claims priority to U.S. Patent Application No. 61/480,929, filed Apr. 29, 2011, all of which are incorporated herein by reference in their entirety for all purposes.

### ACKNOWLEDGEMENTS

This invention was made with government support under Grant R24 EY017404 awarded by the NIH. The government has certain rights in the invention.

### FIELD OF THE INVENTION

The invention relates generally to the diagnosis and treatment of complement-mediated disease in a human subject. Specifically, the invention relates to the prediction or diagnosis of age-related macular degeneration through the detection of a collection of polymorphisms and haplotypes comprised of multiple variations in the CFH-to-F13B locus.

### BACKGROUND

Age related macular degeneration (AMD), a complement-mediated disease, is the leading cause of blindness in the elderly, a problem that will grow worse with an aging population. Accurate identification of risk for developing complement-mediated diseases such as AMD is essential for both starting treatment early in disease progression and identifying causal genetic variants to better target therapeutic strategies and development. Previously, loci have been implicated in risk for AMD on Chr1, Chr10, Chr6 (CFB), and Chr19 (C3).

The RCA gene cluster, sometimes referred to below as the “CFH-to-F13B locus,” is located on chromosome 1q32 and includes the genes for complement factor H (CFH), five Factor H-related genes (CFHR1, CFHR2, CFHR3, CFHR4 and CFHR5), and the gene encoding the beta subunit of coagulation factor XIII See US Pat. Pub. US 2007/0020647, incorporated herein by reference). CFH is a significant regulator of complement activity and is thought to be essential to prevent injury to self-tissues by inappropriate C3 activation. Additionally, alterations in CFH activity have been associated with altered binding to membrane bound glycoproteins, other complement inhibitors, and the surface of pathogenic bacteria.

Previously, haplotype studies have focused on CFH and only rarely extended as far as CFHR2. However, the full CFH locus is an area of the genome that contains additional variants that associate with complement-mediated diseases such as AMD. Therefore, what is needed are more effective methods of describing risk for having or developing complement-mediated disease at this locus. Additionally, what is needed are methods to place known risk and protective variants into the broader genetic background in which they exist, that is the entire CHF-to-F13B locus.

### SUMMARY

Described herein are methods for determining a Caucasian subject's susceptibility to having or developing a complement-mediated disease comprising determining in the Caucasian subject the identity of one or more haplotypes, wherein the one or more haplotypes are H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, or a complement thereof, and wherein the presence of one or more of the haplotypes indicates the subject's susceptibility for having or developing a complement-mediated disease.

Also described herein are methods for determining a Caucasian subject's susceptibility to having or developing a complement-mediated disease comprising determining in the Caucasian subject the identity of one or more haplotypes, wherein the one or more haplotypes are H1_51_A, H2_51_A, H3_51_A, H4_51_A, H5_51_A, H6_51_A, H7_51_A, H8_51_A, H9_51_A, H10_51_A, H11_51_A, H12_51_A, H13_51_A, H14_51_A, H15_51_A, H16_51_A, H17_51_A, or a complement thereof, and wherein the presence of one or more of the haplotypes indicates the subject's susceptibility for having or developing a complement-mediated disease.

Also described herein are methods for determining a Caucasian subject's susceptibility to having or developing a complement-mediated disease comprising determining in the Caucasian subject the identity of one or more haplotypes, wherein the one or more haplotypes are H1_51_A, H2_51_A, H3_51_A, H4_51_A, H5_51_A, H6_51_A, H7_51_A, H8_51_A, H9_51_A, H10_51_A, H11_51_A, H12_51_A, H13_51_A, H14_51_A, H15_51_A, H16_51_A, H17_51_A, or a complement thereof, and wherein the presence of one or more of the haplotypes indicates the subject's susceptibility for having or developing a complement-mediated disease.

Also described herein are methods for determining a Caucasian subject's susceptibility to having or developing a complement-mediated disease comprising determining in the Caucasian subject the identity of at least six SNPs in the CFH-to-F13B locus, wherein the SNPs are: (i) rs35928059, rs800292, rs1061170, rs12144939, rs7546940, rs1409153, rs10922153, or rs698859, or (ii) a SNP in linkage disequilibrium with the SNPs of (i), and wherein the presence of at least six of the SNPs indicates the subject's susceptibility for having or developing a complement-mediated disease.

Also described herein are methods for determining a Caucasian subject's susceptibility to having or developing a complement-mediated disease comprising determining in the subject the identity of one or more SNPs in the CFH-to-F13B locus, wherein the SNPs is: (i) rs1061170, rs1410996, rs2274700, rs3753395, rs403846, or rs3753396 or (ii) a SNP in linkage disequilibrium with the SNPs of (i), and wherein the presence of one or more of the SNPs indicates the subject's susceptibility for having or developing a complement-mediated disease.

Also described herein are methods for determining a Caucasian subject's susceptibility to having or developing a complement-mediated disease comprising determining in the subject the identity of a deletion tagging SNP in the CFH-to-F13B locus, wherein the SNP is: (i) rs12144939 or (ii) a SNP in linkage disequilibrium with rs12144939, and wherein the presence of the SNP indicates the subject's susceptibility for having or developing a complement-mediated disease.

Also described herein are methods of identifying a Caucasian subject in need of treatment for a complement-mediated disease comprising determining in the Caucasian subject the identity of one or more haplotypes, wherein the one or more haplotypes are H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, or a complement thereof, and wherein the presence of one or more of the haplotypes indicates whether the subject is in need of treatment for a complement-mediated disease.

Also described herein are methods of identifying a Caucasian subject in need of treatment for a complement-mediated disease comprising determining in the Caucasian subject the identity of one or more haplotypes, wherein the one or more haplotypes are H1_51_A, H2_51_A, H3_51_A, H4_51_A, H5_51_A, H6_51_A, H7_51_A, H8_51_A, H9_51_A, H10_51_A, H11_51_A, H12_51_A, H13_51_A, H14_51_A, H15_51_A, H16_51_A, H17_51_A, or a complement thereof, and wherein the presence of one or more of the haplotypes indicates whether the subject is in need of treatment for a complement-mediated disease.

Also described herein are methods of identifying a Caucasian subject in need of treatment for a complement-mediated disease comprising determining in the Caucasian subject the identity of one or more haplotypes, wherein the one or more haplotypes are H1_51_A, H2_51_A, H3_51_A, H4_51_A, H5_51_A, H6_51_A, H7_51_A, H8_51_A, H9_51_A, H10_51_A, H11_51_A, H12_51_A, H13_51_A, H14_51_A, H15_51_A, H16_51_A, H17_51_A, or a complement thereof, and wherein the presence of one or more of the haplotypes indicates whether the subject is in need of treatment for a complement-mediated disease.

Also described herein are methods of identifying a Caucasian subject in need of treatment for a complement-mediated disease comprising determining in the Caucasian subject the identity of at least six SNPs in the CFH-to-F13B locus, wherein the SNPs are: (i) rs35928059, rs800292, rs1061170, rs12144939, rs7546940, rs1409153, rs10922153, or rs698859, or (ii) a SNP in linkage disequilibrium with the SNPs of (i), and wherein the presence of at least six of the SNPs indicates whether the subject is in need of treatment for a complement-mediated disease.

Also described herein are methods of identifying a Caucasian subject in need of treatment for a complement-mediated disease comprising determining in the subject the identity of one or more SNPs in the CFH-to-F13B locus, wherein the SNPs is: (i) rs1061170, rs1410996, rs2274700, rs3753395, rs403846, or rs3753396 or (ii) a SNP in linkage disequilibrium with the SNPs of (i), and wherein the presence of one or more of the SNPs indicates whether the subject is in need of treatment for a complement-mediated disease.

Also described herein are methods of identifying a Caucasian subject in need of treatment for a complement-mediated disease comprising determining in the subject the identity of a deletion tagging SNP in the CFH-to-F13B locus, wherein the SNPs are: (i) rs12144939 or (ii) a SNP in linkage disequilibrium with rs12144939, and wherein the presence of one or more of the SNPs indicates whether the subject is in need of treatment for a complement-mediated disease.

Also described herein are methods of identifying a Caucasian subject in need of a prophylactic treatment for a complement-mediated disease comprising determining in the Caucasian subject the identity of one or more haplotypes, wherein the one or more haplotypes are H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, or a complement thereof, and wherein the presence of one or more of the haplotypes indicates whether the subject is in need of prophylactic treatment for a complement-mediated disease.

Also described herein are methods of identifying a Caucasian subject in need of a prophylactic treatment for a complement-mediated disease comprising determining in the Caucasian subject the identity of one or more haplotypes, wherein the one or more haplotypes are H1_51_A, H2_51_A, H3_51_A, H4_51_A, H5_51_A, H6_51_A, H7_51_A, H8_51_A, H9_51_A, H10_51_A, H11_51_A, H12_51_A, H13_51_A, H14_51_A, H15_51_A, H16_51_A, H17_51_A, or a complement thereof, and wherein the presence of one or more of the haplotypes indicates whether the subject is in need of prophylactic treatment for a complement-mediated disease.

Also described herein are methods of identifying a Caucasian subject in need of a prophylactic treatment for a complement-mediated disease comprising determining in the Caucasian subject the identity of one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, and wherein the presence of one or more of the haplotypes indicates whether the subject is in need of prophylactic treatment for a complement-mediated disease.

Also described herein are methods of identifying a Caucasian subject in need of a prophylactic treatment for a complement-mediated disease comprising determining in the Caucasian subject the identity of one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, and wherein the presence of one or more of the haplotypes indicates whether the subject is in need of prophylactic treatment for a complement-mediated disease.

Also described herein are methods of identifying a Caucasian subject in need of a prophylactic treatment for a complement-mediated disease comprising determining in the subject the identity of one or more SNPs in the CFH-to-F13B locus, wherein the SNPs is: (i) rs1061170, rs1410996, rs2274700, rs3753395, rs403846, or rs3753396 or (ii) a SNP in linkage disequilibrium with the SNPs of (i), and wherein the presence of one or more of the SNPs indicates whether the subject is in need of prophylactic treatment for a complement-mediated disease.

Also described herein are methods of identifying a Caucasian subject in need of a prophylactic treatment for a complement-mediated disease comprising determining in the subject the identity of a deletion tagging SNP in the CFH-to-F13B locus, wherein the SNPs are: (i) rs12144939 or (ii) a SNP in linkage disequilibrium with rs12144939, and wherein the presence of one or more of the SNPs indicates whether the subject is in need of prophylactic treatment for a complement-mediated disease.

Also described herein are methods of identifying a Caucasian subject appropriate for an a complement-mediated disease clinical trial comprising determining in the Caucasian subject the identity of one or more haplotypes, wherein the one or more haplotypes are H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, or a complement thereof, and wherein the presence of one or more of the haplotypes indicates whether the subject is appropriate for the clinical trial.

Also described herein are methods of of identifying a Caucasian subject appropriate for an a complement-mediated disease clinical trial comprising determining in the Caucasian subject the identity of one or more haplotypes, wherein the one or more haplotypes are H1_51_A, H2_51_A, H3_51_A, H4_51_A, H5_51_A, H6_51_A, H7_51_A, H8_51_A, H9_51_A, H10_51_A, H11_51_A, H12_51_A, H13_51_A, H14_51_A, H15_51_A, H16_51_A, H17_51_A, or a complement thereof, and wherein the presence of one or more of the haplotypes indicates whether the subject is appropriate for the clinical trial.

Also described herein are methods of of identifying a Caucasian subject appropriate for an a complement-mediated disease clinical trial comprising determining in the Caucasian subject the identity of one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, and wherein the presence of one or more of the haplotypes indicates whether the subject is appropriate for the clinical trial.

Also described herein are methods of identifying a Caucasian subject appropriate for an a complement-mediated disease clinical trial comprising determining in the Caucasian subject the identity of at least six SNPs in the CFH-to-F13B locus, wherein the SNPs are: (i) rs35928059, rs800292, rs1061170, rs12144939, rs7546940, rs1409153, rs10922153, or rs698859, or (ii) a SNP in linkage disequilibrium with the SNPs of (i), and wherein the presence of at least six of the SNPs indicates whether the subject is appropriate for the clinical trial.

Also described herein are methods of identifying a Caucasian subject appropriate for an a complement-mediated disease clinical trial comprising determining in the subject the identity of one or more SNPs in the CFH-to-F13B locus, wherein the SNPs is: (i) rs1061170, rs1410996, rs2274700, rs3753395, rs403846, or rs3753396 or (ii) a SNP in linkage disequilibrium with the SNPs of (i), and wherein the presence of one or more of the SNPs indicates whether the subject is appropriate for the clinical trial.

Also described herein are methods of of identifying a Caucasian subject appropriate for an a complement-mediated disease clinical trial comprising determining in the subject the identity of a deletion tagging SNP in the CFH-to-F13B locus, wherein the SNPs are: (i) rs12144939 or (ii) a SNP in linkage disequilibrium with rs12144939, and wherein the presence of one or more of the SNPs indicates whether the subject is appropriate for the clinical trial.

Also described herein are kits kit comprising an assay for detecting one or more haplotypes in a nucleic acid sample of a subject, wherein the one or more haplotypes are H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, H1_51_A, H2_51_A, H3_51_A, H4_51_A, H5_51_A, H6_51_A, H7_51_A, H8_51_A, H9_51_A, H10_51_A, H11_51_A, H12_51_A, H13_51_A, H14_51_A, H15_51_A, H16_51_A, H17_51_A, H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof.

Also described herein are methods for determining a Caucasian subject's susceptibility to having or developing age-related macular degeneration comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, and wherein the presence of one or more of the diplotypes indicates the subject's susceptibility for having or developing age-related macular degeneration.

Also descibed herein are methods of identifying a Caucasian subject in need of treatment for age-related macular degeneration comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, and wherein the presence of one or more of the diplotypes indicates whether the subject is in need of treatment for age-related macular degeneration.

Also described herein are methods of identifying a Caucasian subject in need of a prophylactic treatment for age-related macular degeneration comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, and wherein the presence of one or more of the diplotypes indicates whether the subject is in need of prophylactic treatment for age-related macular degeneration.

Also described herein are methods of identifying a Caucasian subject appropriate for an age-related macular degeneration clinical trial comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, and wherein the presence of one or more of the diplotypes indicates whether the subject is appropriate for the clinical trial.

### BRIEF DESCRIPTION OF THE FIGURES

The accompanying figures, which are incorporated in and constitute a part of this specification, illustrate several aspects and together with the description serve to explain the principles of the invention.

FIG. 1 shows major haplotypes H1_61_A through H15_62_A.

FIG. 2 shows the 63 SNPs utilized in the preliminary haplotype studies.

FIG. 3 shows minor haplotypes H16_62_A through H163_62_A.

FIG. 4 shows the verification of halotypes derived from the GSH 1073 cohort and the GSH 927 cohort.

FIG. 5 shows the single SNP association data for the 62 SNP panel in the GSH cohort.

FIG. 6 shows disease associations that were analyzed for the entire extended haplotypes using Haploview 4.2.

FIG. 7 shows the haplotype tagging SNPs for 62 SNP haplotypes.

FIG. 8 shows the 51_A haplotype tagging SNPS (51_A htSNPs).

FIG. 9 shows the 51_B haplotype tagging SNPS (51_A htSNPs).

FIG. 10 shows the effect of generating haplotypes using fewer and fewer SNPs has on association values for haplotypes.

FIG. 11 shows the qPCR assay for R3-1 deletion, regular PCR assay for homozygous deletion or non-deletion.

FIG. 12 shows the assessment of 51 SNP haplotypes in the Sib-pair cohort.

FIGS. 13A-D shows the 62 single marker (SNPs) and haplotypes association with gender in the GSH case-control cohort the male and female gender association in the '927 cohort.

FIG. 14 shows the haplotype boundaries extended beyond that covered by 63 SNPs. Provided in the figure is a list of all Hapmap SNPs.

FIG. 15 shows the CFH locus and the haplotypes in the 51_B cohort.

FIG. 16 shows a comparison of 51_A and 62_A haplotypes.

FIG. 17 shows the frequencies of the 62_A haplotypes.

FIG. 18 shows the 62_A SNP haplotype schema regrouped with P values.

FIG. 19 shows a comparison of the 51 SNP haplotypes in the 1073 cohort and combined cohorts.

FIG. 20 shows the '927 cohort and the 62_A to 51_A conversion.

FIGS. 21-1 and 21-2 shows the diplotype associations; Controls (0) vs CNV (4B) in the 51_B cohort.

FIG. 22 shows the association of haplotypes in the '927 cohort using a 51 SNP-based haplotype.

FIG. 23 shows the gender associations of the 62_A haplotypes.

FIGS. 24A and 24B show the gender associations of the 51_B haplotypes.

FIG. 25 shows haplotype disease association.

FIG. 26 shows haplotype disease association based on gender.

FIG. 27 shows the SNPs used in a haplotype reconstruction.

FIG. 28 shows Taqman qPCR copy number variation data.

FIG. 29 shows the frequency of subjects with varying number of SNPs screened.

FIG. 30 shows a deletion decision tree made to predict deletion status from three SNPs.

FIG. 31 shows the results of the real-time quantitative PCR (qPCR) used to determine the copy number state of the CFHR3 and CFHR1 genes.

FIG. 32 shows the H1-H19 haplotypes based on 1073 cohort screened for 63 SNPs.

FIG. 33 shows haplotype frequencies calculated on 136 unrelated UGRP individuals, who were mostly first generation grandparents, using Haploview program's phased haplotype input mode, Haps format.

FIG. 34 shows association of 63 SNP-based CFH-to-F13B Haplotypes with AMD.

FIG. 35 shows the diplotype analysis of the overall 51_B cohort.

FIG. 36 shows diplotype analysis for males in the 51_B cohort.

FIG. 37 shows diplotype analysis for females in the 51_B cohort.

FIGS. 38-1 through 38-8 show the combined cohort with all haplotypes, 0vs1b-4, highlighted to match major HapMap haplotypes.

FIG. 39 shows the major CFH-to-F13B HapMap haplotypes with >0.5% frequencies for CEU.

FIGS. 40-1 through 40-4 show the marker-rs number IDs shown in FIG. 39.

FIGS. 41-1 through 41-5 show a comparison of the 51_B-based haplotypes to HapMap haplotypes >0.05% frequencies.

FIGS. 42-1 and 42-2 show single marker associations; Controls (0) vs all AMD in the 51_B cohort.

FIGS. 43-1 and 43-2 show single marker associations; Controls (0) vs ealy AMD (1b-3) in the 51_B cohort.

FIGS. 44-1 and 44-2 show single marker associations; Controls (0) vs geographic atrophy (4A) in the 51B cohort.

FIGS. 45-1 and 45-2 show the single marker associations; Controls (0) vs CNV (4B) in the 51_B cohort.

FIGS. 46-1 through 46-10 show the haplotype associations; Controls (0) vs all AMD, early AMD (1B-3), GA only (4A) & CNV only (4B) in the 51_B cohort.

FIGS. 47-1 and 47-2 show the diplotype associations; Controls (0) vs early AMD (1B-3) in the 51_B cohort.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values described herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data are provided in a number of different formats, and that these data, represent endpoints, starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

“Optional” or “optionally” means that the subsequently described event or circumstance may or may not occur and that the description includes instances where said event or circumstance occurs and instances where it does not.

The word “or” as used herein means any one member of a particular list and also includes any combination of members of that list.

“Complement-Mediated Disease” as used herein refers to a disease caused by or resulting from abnormal complement activation or function of its four major pathways. The complement pathway consists of three stages: recognition, enzymatic activation and membrane attack leading to cell death. “Complement-Mediated Disease” as used herein also refers to a disease caused by or resulting from alterations of the classical, exogenous, mannan-binding lectin (MBL) or alternative pathways. Examples of Complement-Mediated Disease include, but are not limited to, atypical haemolytic uraemic syndrome (aHUS), membranoproliferative glomerulonephritis type II (MPGN II; also known as dense deposit disease), systemic lupus erythematosus (SLE), pyogenic infections, hemolysis and thrombosis, partial lipodystrophy, hereditary angioedema, paroxysmal nocturnal haemoglobinuria (PNH), age-related macular degeneration (AMD), rheumatois arthritis, myocarditis, multiple sclerosis, IgA nephropathy, HenochSchoenlein purpura, glaucoma, post-streptococcal disease, anti-phospholipid antibody syndrome, recurrent pregnancy loss, asthma, antibody-mediated cutaneous disease and anti-nuclear cytoplasmic antigen-associated pauci-immune vasculitis.

“Haplotype” as used herein refers to a DNA sequence or a combination of DNA sequences present at various loci on a chromosome that are transmitted together; a haplotype may be one locus, several loci, or an entire chromosome depending on the number of recombination events that have occurred between a given set of loci.

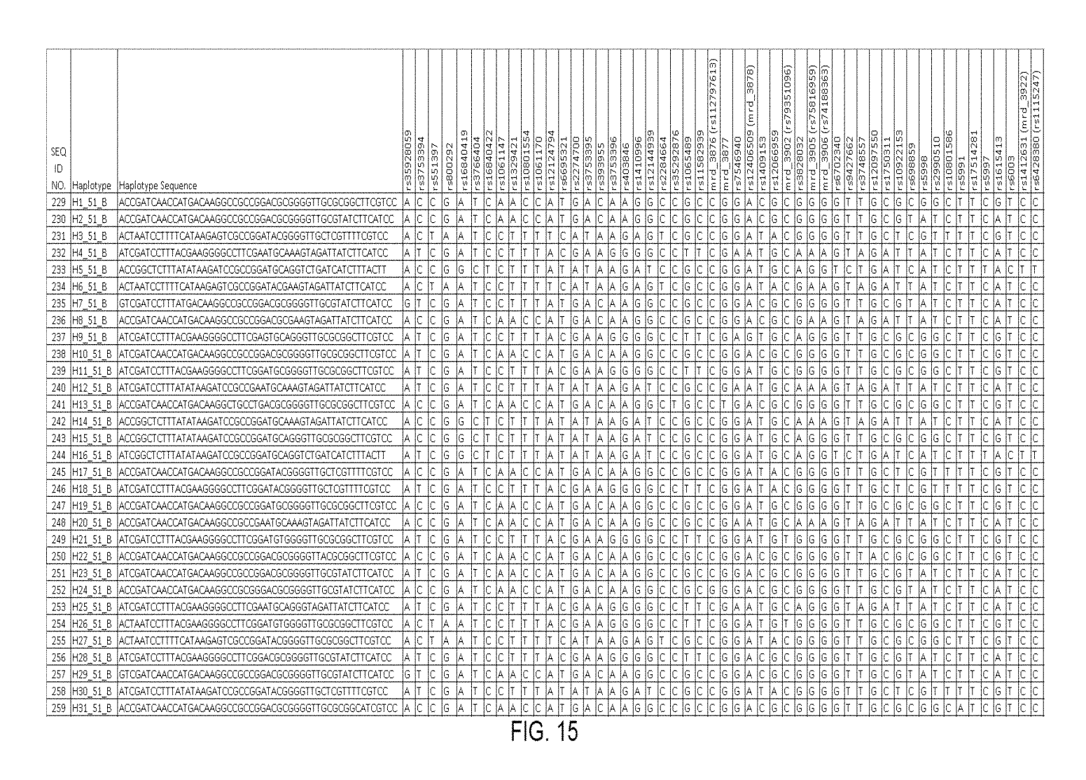

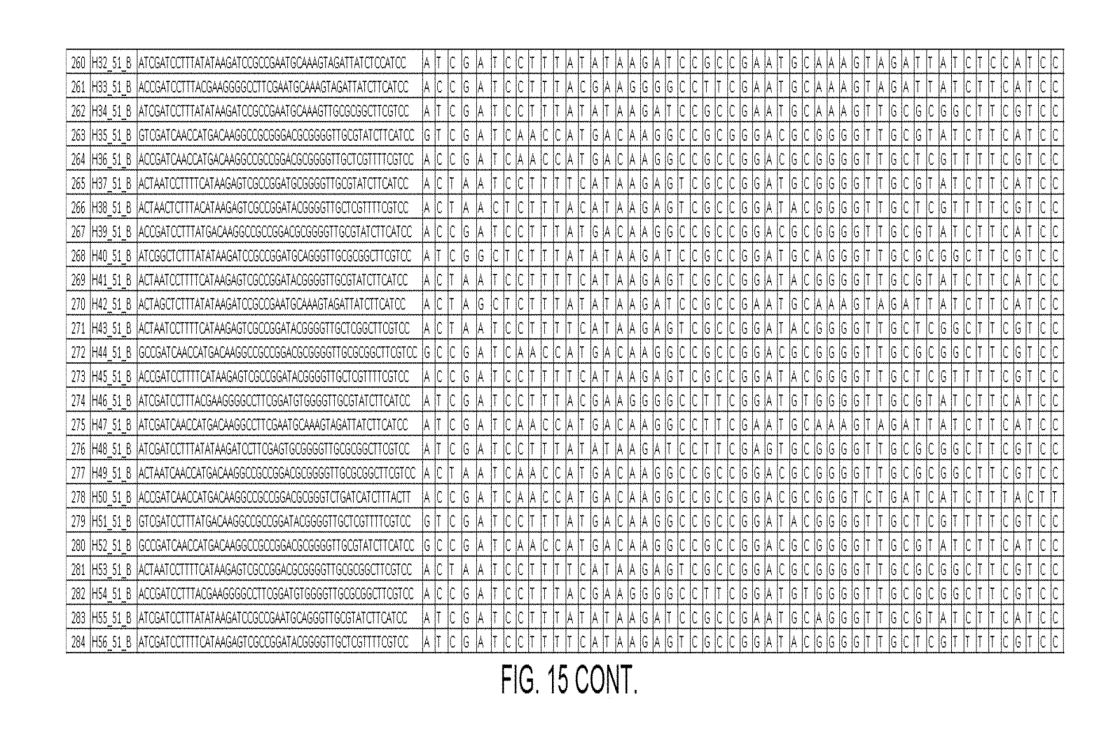

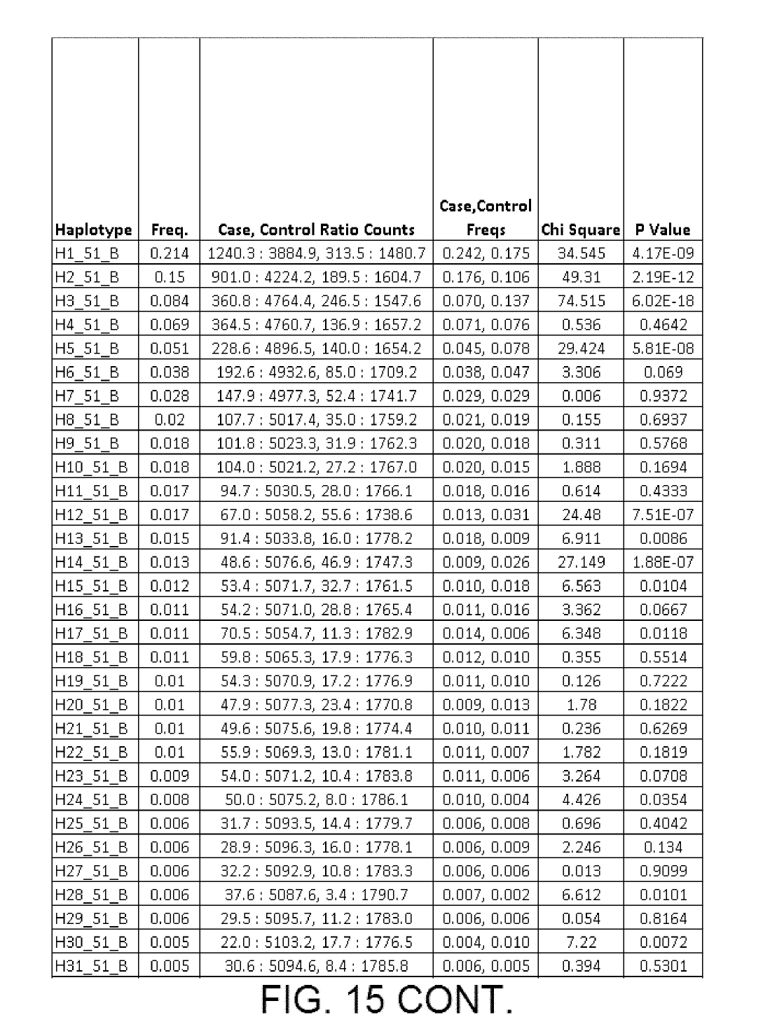

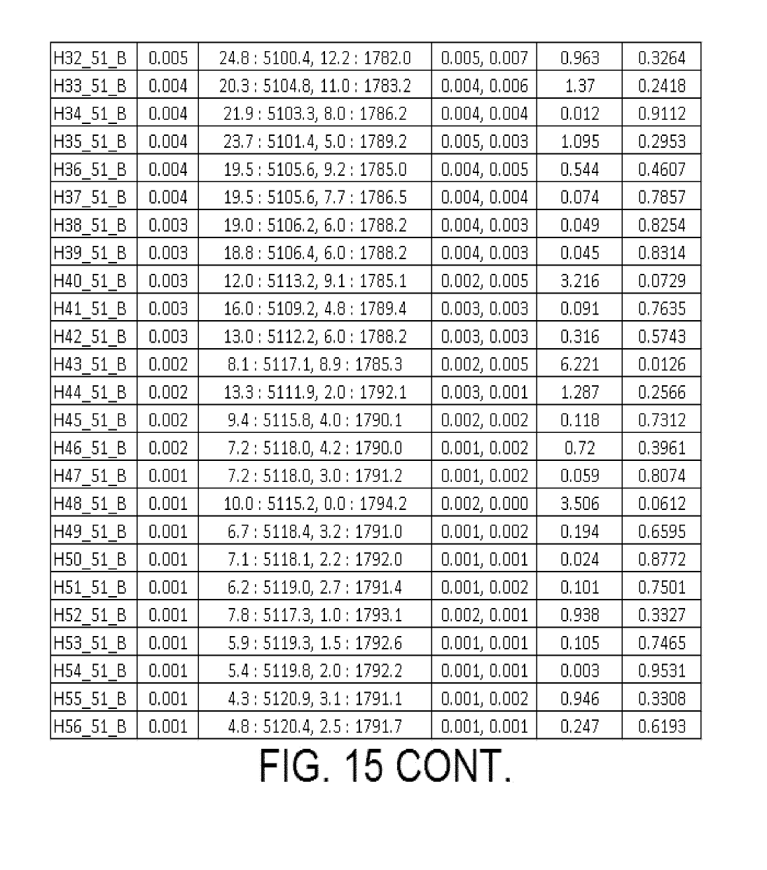

“Risk Haplotype” as used herein refers to a haplotype of a subject wherein the presence of the haplotype indicates the subject's increased risk of developing a complement-mediated disease (e.g. AMD) when compared to a subject without said risk haplotype. A “Risk Haplotype” also refers to a haplotype that occurs more often in subjects with Complement-Mediated Disease (e.g. AMD) (cases) than in subjects without Complement-Mediated Disease (e.g. AMD) (controls) in a given cohort. In other words, the case versus control percentage is different in cases than in controls as illustrated in the tables herein (see FIG. 15). Although not required, the difference between the number of subjects with Complement-Mediated Disease (e.g. AMD) (case) and the number of subjects without Complement-Mediated Disease (e.g. AMD) (control) can be statistically significant. For example, H2_62_A, H2_51_A, and H2_51_B are examples of “Risk Haplotypes” as they relate to AMD.

“Protective Haplotype” as used herein refers to a haplotype of a subject wherein the presence of the haplotype indicates the subject's decreased risk of developing Complement-Mediated Disease (e.g. AMD) when compared to a subject without said protective haplotype. A “Protective Haplotype” also refers to a haplotype that occurs less often in subjects with Complement-Mediated Disease (e.g. AMD) (cases) than in subjects without Complement-Mediated Disease (e.g. AMD) (controls), in other words, the case versus control percentage is lower in cases than in controls as illustrated in the tables herein (See FIG. 15). Although not required, the difference between the number of subjects with Complement-Mediated Disease (e.g. AMD) (case) and the number of subjects without Complement-Mediated Disease (e.g. AMD) (control) can be statistically significant. For example, H5_62_A, H5_51_A, and H5_51_B are examples of “protective haplotypes” as they relate to AMD.

“Neutral Haplotype” as used herein refers to a haplotype of a subject wherein the presence of the haplotype does not indicate the subject's increased or decreased risk of developing Complement-Mediated Disease (e.g. AMD). A “Neutral Haplotype” also refers to a haplotype that occurs about equally in subjects with Complement-Mediated Disease (e.g. AMD) (cases) and in subjects without Complement-Mediated Disease (e.g. AMD) (controls), in other words, the case versus control percentage is about equal in cases and controls as illustrated in the tables herein (See FIG. 15). Although not required, the difference between the number of subjects with Complement-Mediated Disease (e.g. AMD) (case) and the number of subjects without Complement-Mediated Disease (e.g. AMD) (control) can be non-statistically significant. For example, H4_62_A, H4_51_A, and H4_51_B are examples of “neutral haplotypes” as they relate to AMD.

Haplotypes as described herein can be referred to by the generic nomenclature: “Hx_Y_Z”, wherein “Hx” indicates the numbering of the specific haplotype as determined by the frequency of the specific haplotype in a given population or cohort. For example, H2 indicates a specific haplotype that occurs with the second highest frequency in the given data set, population or cohort. “Y”, in the context of this nomenclature, specifies the SNP panel from which the overall haplotype was determined. For example, Hx_62_Z would refer to the 62 SNP Panel as described herein, whereas Hx_51_Z refers to the 51 SNP Panel as described herein. “Z”, in the context of this nomenclature, refers to the specific cohort used in ascertaining the specific haplotype. For example, Hx_Y_A would refer to the ‘927 cohort’ as described herein, whereas Hx_Y_B refers to the combined cohort as described herein.

“Haplotype Tagging Haplotype” refers to haplotypes that can tag one or more of the haplotypes described herein. Such haplotypes can be referred to as “tagging haplotypes”. Such tagging haplotypes can be identified based on HapMap Analysis as described herein. When such tagging haplotypes are referenced herein, the generic nomenclature: “Hx_Y_Z_t” can be used wherein “t” represents a “tagging haplotype” and Hx, Y, and Z represent the same categories as described above for the generic nomenclature “Hx_Y_Z”. For example, H2_62_A_1 would represent a first haplotype that tags the H2_62_A haplotype.

“Major Haplotype” as used herein refers to a haplotype that occurs in greater than 1% of the population, data set or cohort being examined.

“Minor Haplotype” as used herein refers to a haplotype that occurs in 1% or less than 1% of the population, data set or cohort being examined.

“CFH-to-F13B Locus” as used herein refers to a region of chromosome 1q25-q31 (approximately 196,580,000 to 197,053,000; hg19) containing the following genes and pseudogenes: complement factor H (CFH) and its truncated isoform (CFHT), the complement factor H related genes 3, 1, 4, 2 and 5 (CFHR3, CFHR1, CFHR4, CFHR2 and CFHR5), a CFHR pseudogene 0 and factor 13B (F13B). The CFH-to-F13B Locus includes all non-genic DNA, including that extending proximal to CFH and distal to F13B. Blocks of linkage disequilibrium (LD) that initiate within the CFH-to-F13B and extend proximal to CFH and/or distal to F13b are also included.

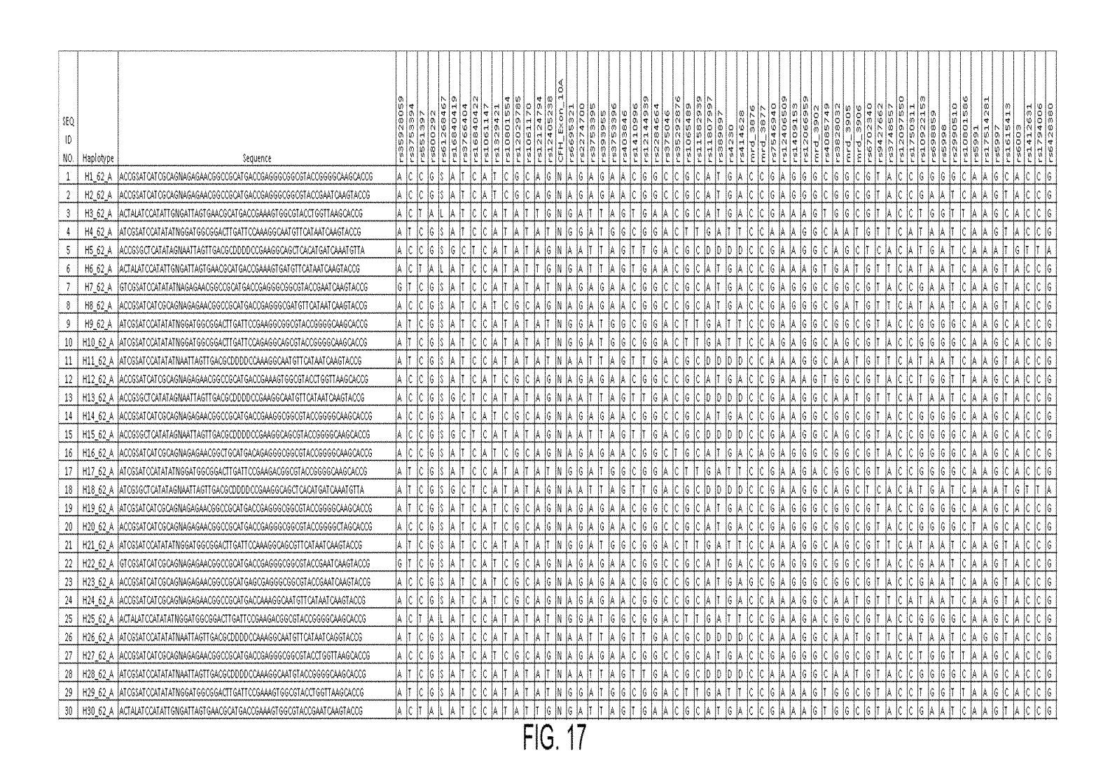

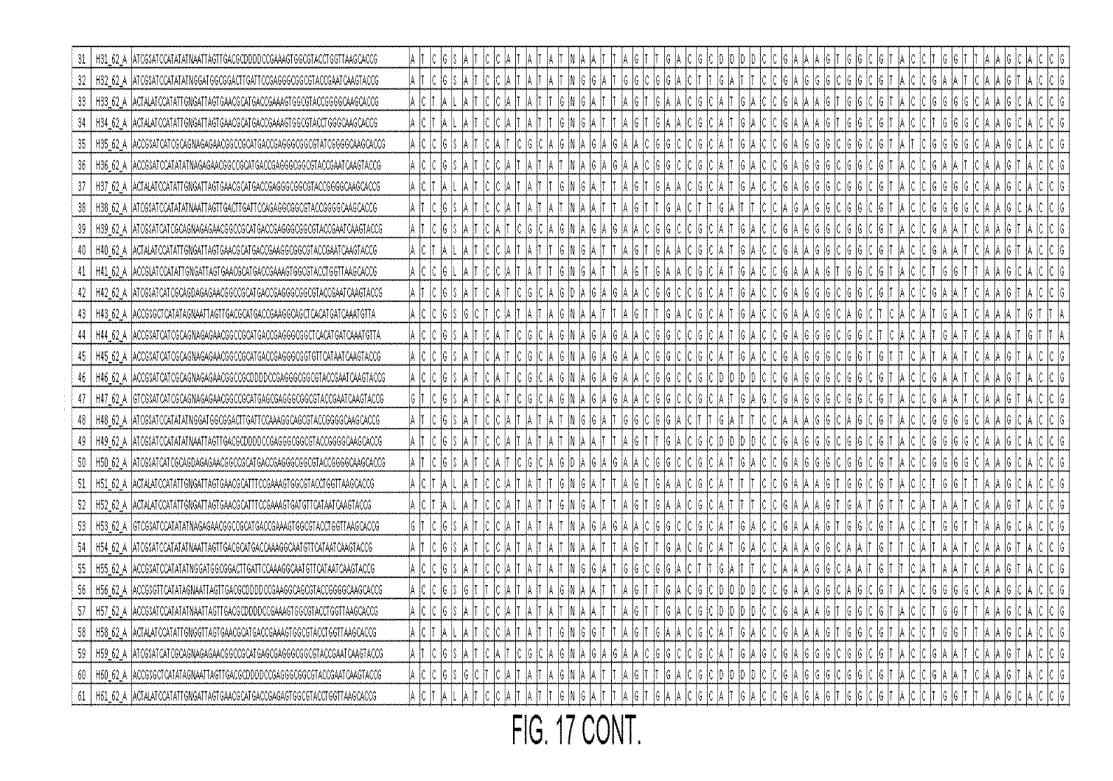

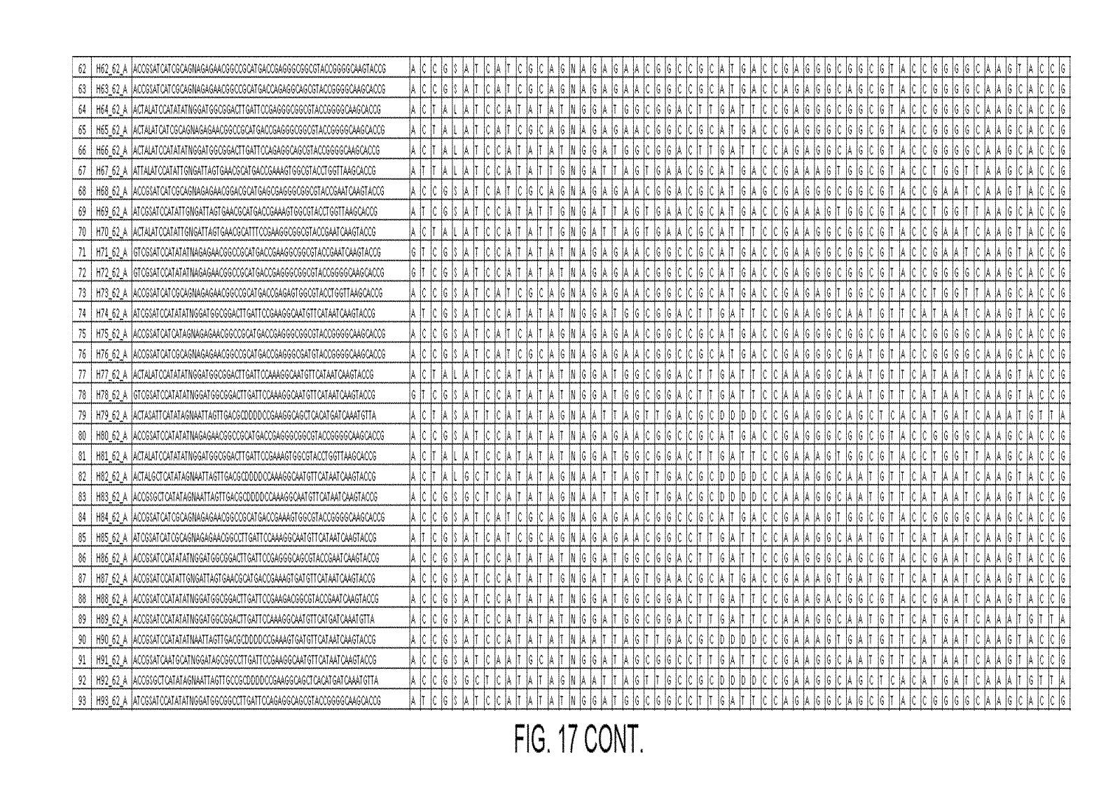

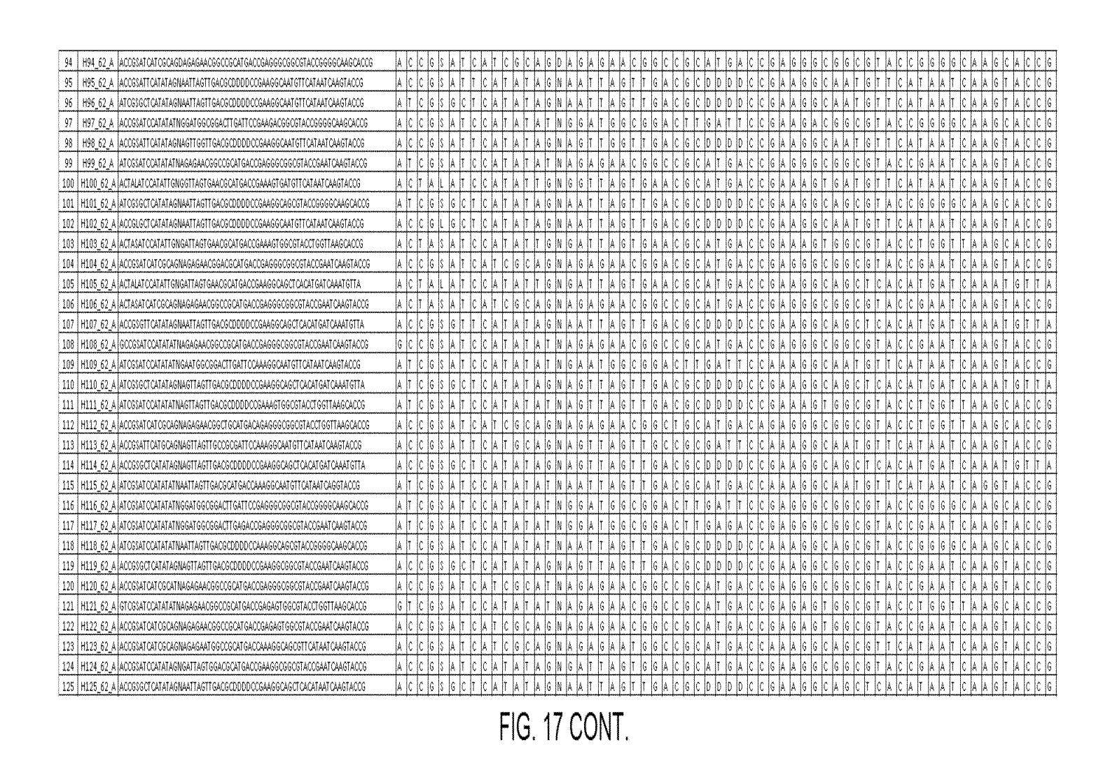

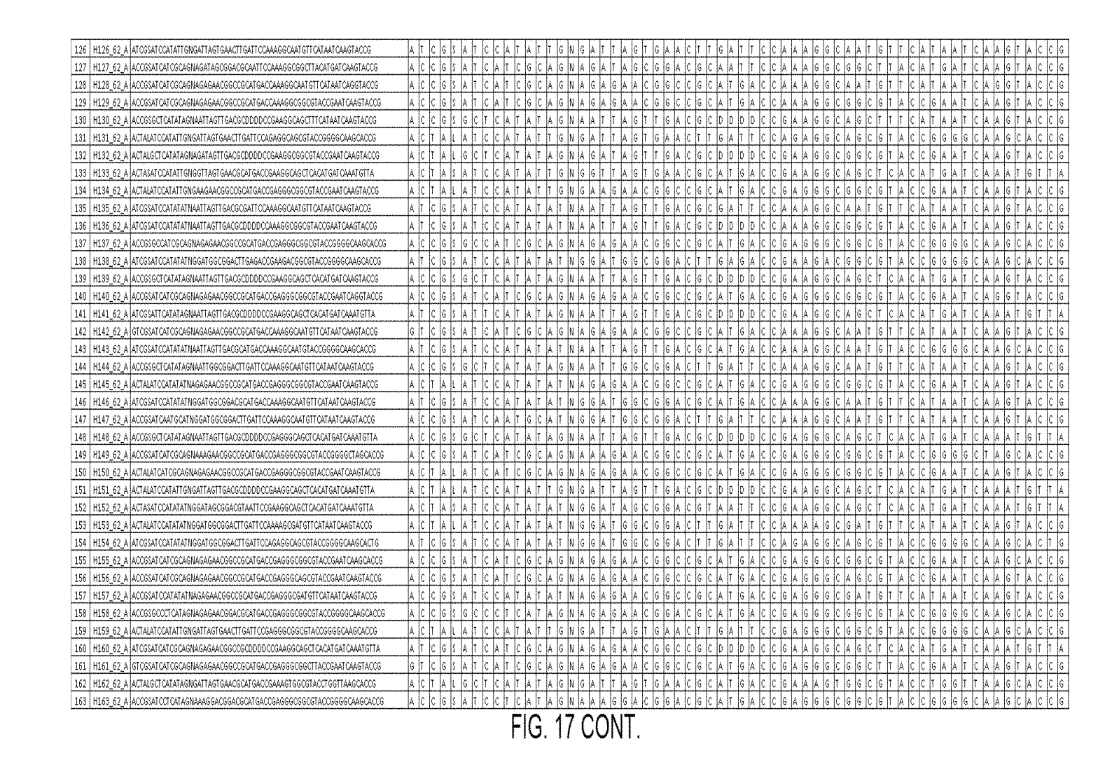

“62 SNP Panel” as used herein refers to 62 SNPs that were selected based on their significance of association as well as locations selected to provide coverage across the entire CFH-to-F13B Locus and its LD blocks. The 62 SNP Panel consists of the SNPs shown in FIG. 17.

“51 SNP Panel” as used herein refers to 51 SNPs of the 62 SNP Panel described herein. The 51 SNP Panel consists of the SNPs shown in FIG. 15.

“HapMap” as used herein refers to a haplotype map of the human genome that is a catalog of common genetic variants that occur in human beings. HapMap describes what these variants are, where they occur in DNA, how they distribute in haplotypes, and their frequencies among people within populations and among populations in different parts of the world. (The International HapMap Consortium. The International HapMap Project. Nature 426, 789-796 (2003)).

“HapMap Analysis” as used herein refers to an analysis that can be conducted using Haploview software (Barrett J C, Fry B, Maller J, Daly M J. Haploview: analysis and visualization of LD and haplotype maps. Bioinformatics. 2005 Jan. 15 [PubMed ID: 15297300]).

“Linkage Disequilibrium” as used herein refers to the non-random association between two or more alleles at two or more loci (not necessarily on the same chromosome) such that certain combinations of alleles are more likely to occur together on a chromosome than other combinations of alleles than would be expected from a random formation of haplotypes from alleles based on their frequencies.

### A. METHODS OF PREDICTING A CAUCASIAN SUBJECT'S RISK FOR HAVING OR DEVELOPING A COMPLEMENT-MEDIATED DISEASE

Described herein are methods for determining a Caucasian subject's susceptibility to having or developing a complement-mediated disease comprising determining in the Caucasian subject the identity of one or more major or minor haplotypes. The haplotypes can include, but are not limited to H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, H1_51_A, H2_51_A, H3_51_A, H4_51_A, H5_51_A, H6_51_A, H7_51_A, H8_51_A, H9_51_A, H10_51_A, H11_51_A, H12_51_A, H13_51_A, H14_51_A, H15_51_A, H16_51_A, H17_51_A, H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof. As used herein, “complement thereof” means a SNP, haplotype, or diplotype that is located on the complement DNA or RNA strand of any SNP, haplotype, or diplotype described herein. For example, and not to be limiting, the haplotype sequence “ATGC” can be converted to its complement thereof “TACG”. Therefore, by way of example, if “ATGC” represents a haplotype indicative of a subject's susceptibility to having or developing a complement-mediated disease, its complement haplotype sequence, “TACG”, also represents a haplotype indicative of a subject's susceptibility to having or developing a complement-mediated disease. As described herein, the presence of one or more of the haplotypes disclosed herein can indicate a subject's susceptibility for having or developing a complement-mediated disease. In one aspect, the complement-mediated disease can be age-related macular degeneration (AMD). In another aspect, the complement-mediated disease can include, but is not limited to atypical haemolytic uraemic syndrome (aHUS), membranoproliferative glomerulonephritis type II (MPGN II; also known as dense deposit disease), systemic lupus erythematosus (SLE), pyogenic infections, hemolysis and thrombosis, partial lipodystrophy, hereditary angioedema, paroxysmal nocturnal haemoglobinuria (PNH), rheumatois arthritis, myocarditis, multiple sclerosis, IgA nephropathy, Henoch Schoenlein purpura, glaucoma, post-streptococcal disease, anti-phospholipid antibody syndrome, recurrent pregnancy loss, asthma, antibody-mediated cutaneous disease, or anti-nuclear cytoplasmic antigen-associated pauci-immune vasculitis.

In one aspect, determining the identity of a certain haplotype or certain haplotypes in a subject can indicate that the subject has an increased risk of having or developing a complement-mediated disease. Such hapltoypes can be risk haplotypes. For example, and not to be limiting, a risk haplotype can be H2_62_A, H2_51_A, H1_51_B, H2_51_B, or a complement thereof. Thus, described herein are methods for determining a Caucasian subject's susceptibility to having or developing a complement-mediated disease comprising determining in the Caucasian subject the identity of one or more haplotypes described herein, wherein a risk haplotype is indicative of the subject's increased risk for having or developing a complement-mediated disease. In one aspect, also disclosed are methods of determining a Caucasian subject's increased risk susceptibility to having or developing complement-mediated disease, wherein the method further comprises administering a therapeutic composition to the subject when an increased risk is determined. In one aspect, also disclosed are methods of determining a Caucasian subject's increased risk susceptibility to having or developing complement-mediated disease, wherein the subject has an increased risk of having or developing complement-mediated disease and further comprises administering a therapeutic composition to the subject when an increased risk is determined.

In another aspect, determining the identity of a certain haplotype or certain haplotypes in a subject can indicate that the subject has a deacreased risk of having or developing a complement-mediated disease. Such haplotypes can be protective haplotypes. For example, and not to be limiting, a protective haplotype can be H3_62_A, H5_62_A, H11_62_A, H3_51_A, H5_51_A, H10_51_A, H3_51_B, H5_51_B, H12_51_B, H14_51_B, or a complement thereof. Thus, described herein are methods for determining a Caucasian subject's susceptibility to having or developing a complement-mediated disease comprising determining in the Caucasian subject the identity of one or more haplotypes described herein, wherein a protective haplotype is indicative of the subject's decreased risk for having or developing a complement-mediated disease.

In yet another aspect, determining the identity of a certain haplotype or certain haplotypes in a subject can indicate that the subject does not have an increased risk or a decreased risk of having or developing a complement-mediated disease. Such haplotypes can be neutral haplotypes. For example, and not to be limiting, a neutral haplotype can be H1_62_A, H4_62_A, H6_62_A, H7_62_A, H8_62_A, H9_62_A, H10_62_A, H12_62_A, H13_62_A, H14_62_A, H15_62_A, H1_51_A, H4_51_A, H6_51_A, H7_51_A, H8_51_A, H9_51_A, H11_51_A, H12_51_A, H13_51_A, H14_51_A, H15_51_A, H16_51_A, H17_51_A, H4_51_B, H6_51_B, H7_51_B, H8_51_B, H9_51_B, H10_51_B, H11_51_B, H13_51_B, H15_51_B, H16_51_B, H17_51_B, H18_51_B, H19_51_B, H20_51_B, H21_51_B, H22_51_B, or a complement thereof. Thus, described herein are methods for determining a Caucasian subject's susceptibility to having or developing a complement-mediated disease comprising determining in the Caucasian subject the identity of one or more haplotypes described herein, wherein a neutral haplotype does not indicate that the subject has an increased risk or a decreased risk for having or developing a complement-mediated disease.

As used herein, the term “subject” means an individual. In one aspect, a subject is a mammal such as a human. In another aspect, the subject can be a Caucasian subject. In a further aspect a subject can be a non-human primate. Non-human primates include marmosets, monkeys, chimpanzees, gorillas, orangutans, and gibbons, to name a few. The term “subject” also includes domesticated animals, such as cats, dogs, etc., livestock (for example, cattle (cows), horses, pigs, sheep, goats, etc.), laboratory animals (for example, ferret, chinchilla, mouse, rabbit, rat, gerbil, guinea pig, etc.) and avian species (for example, chickens, turkeys, ducks, pheasants, pigeons, doves, parrots, cockatoos, geese, etc.). Subjects can also include, but are not limited to fish (for example, zebrafish, goldfish, tilapia, salmon, and trout), amphibians and reptiles. As used herein, a “subject” is the same as a “patient,” and the terms can be used interchangeably.

A haplotype can refer to a set of polymorphisms in a single gene, an intergenic sequence, or in larger sequences including both gene and intergenic sequences, e.g., a collection of genes, or of genes and intergenic sequences. For example, a haplotype can refer to a set of polymorphisms in the CFH-to-F13B locus. The CFH-to-F13B locus comprises the chromosome region of chromosome 1q25-q31 (approximately 196,580,000 to 197,053,000; hg19) that contains the following genes and pseudogenes: complement factor H (CFH) and its truncated isoform (CFHT), the complement factor H related genes 3, 1, 4, 2, and 5 (CFHR3, CFHR1, CFHR4, CFHR2 and CFHR5), a CFHR pseudogene, and factor 13B (F13B). The locus can also incude all non-genic DNA, including that extending proximal to CFH and distal to F13B. Blocks of linkage disequilibrium (LD) that initiate within the CFH-to-F13B and extend proximal to CFH and/or distal to F13b are also included in the locus.

As used herein the term “haplotype” can also refer to a set of single nucleotide polymorphisms (SNPs) found to be statistically associated with each other on a single chromosome. A haplotype can also refer to a combination of polymorphisms (e.g., SNPs) and other genetic markers (e.g., an insertion or a deletion) found to be statistically associated with each other on a single chromosome.

The term “polymorphism” refers to the occurrence of one or more genetically determined alternative sequences or alleles in a population. A “polymorphic site” is the locus at which sequence divergence occurs. Polymorphic sites have at least one allele. A diallelic polymorphism has two alleles. A triallelic polymorphism has three alleles. Diploid organisms may be homozygous or heterozygous for allelic forms. A polymorphic site can be as small as one base pair. Examples of polymorphic sites include: restriction fragment length polymorphisms (RFLPs), variable number of tandem repeats (VNTRs), hypervariable regions, minisatellites, dinucleotide repeats, trinucleotide repeats, tetranucleotide repeats, and simple sequence repeats. As used herein, reference to a “polymorphism” can encompass a set of polymorphisms (i.e., a haplotype).

A “single nucleotide polymorphism (SNP)” can occur at a polymorphic site occupied by a single nucleotide, which is the site of variation between allelic sequences. The site can be preceded by and followed by highly conserved sequences of the allele. A SNP can arise due to substitution of one nucleotide for another at the polymorphic site. Replacement of one purine by another purine or one pyrimidine by another pyrimidine is called a transition. Replacement of a purine by a pyrimidine or vice versa is called a transversion. A synonymous SNP refers to a substitution of one nucleotide for another in the coding region that does not change the amino acid sequence of the encoded polypeptide. A non-synonymous SNP refers to a substitution of one nucleotide for another in the coding region that changes the amino acid sequence of the encoded polypeptide. A SNP may also arise from a deletion or an insertion of a nucleotide or nucleotides relative to a reference allele.

A “set” of polymorphisms means one or more polymorphism, e.g., at least 1, at least 2, at least 3, at least 4, at least 5, at least 6, or more than 6 polymorphisms known, for example, in the CFH-to-F13B locus.

As used herein, a “nucleic acid,” “polynucleotide,” or “oligonucleotide” can be a polymeric form of nucleotides of any length, can be DNA or RNA, and can be single- or double-stranded. Nucleic acids can include promoters or other regulatory sequences. Oligonucleotides can be prepared by synthetic means. Nucleic acids include segments of DNA, or their complements spanning or flanking any one of the polymorphic sites known in the CFH-to-F13B locus. The segments can be between 5 and 100 contiguous bases and can range from a lower limit of 5, 10, 15, 20, or 25 nucleotides to an upper limit of 10, 15, 20, 25, 30, 50, or 100 nucleotides (where the upper limit is greater than the lower limit). Nucleic acids between 5-10, 5-20, 10-20, 12-30, 15-30, 10-50, 20-50, or 20-100 bases are common. The polymorphic site can occur within any position of the segment. A reference to the sequence of one strand of a double-stranded nucleic acid defines the complementary sequence and except where otherwise clear from context, a reference to one strand of a nucleic acid also refers to its complement.

“Nucleotide” as described herein refers to molecules that, when joined, make up the individual structural units of the nucleic acids RNA and DNA. A nucleotide is composed of a nucleobase (nitrogenous base), a five-carbon sugar (either ribose or 2-deoxyribose), and one phosphate group. “Nucleic acids” are polymeric macromolecules made from nucleotide monomers. In DNA, the purine bases are adenine (A) and guanine (G), while the pyrimidines are thymine (T) and cytosine (C). RNA uses uracil (U) in place of thymine (T). As used in the figures, “S” represents short; “L” represents “long, and “D” represents deleted in the context of the SNP analysis. For purposes for the sequence listing “S”, “L” and “D” are represented as “N” which refers to any specific nucleotide in the posotion identified.

Exemplary polymorphic sites in the CFH-to-F13B locus are described herein as examples and are not intended to be limiting. These polymorphic sites, SNPs, or haplotypes can also be used in carrying out methods described herein. Moreover, it will be appreciated that these CFH-to-F13B locus polymorphisms are useful for linkage and association studies, genotyping clinical populations, correlation of genotype information to phenotype information, loss of heterozygosity analysis, identification of the source of a cell sample, and can also be useful to target potential therapeutics to cells.

It will be appreciated that additional polymorphic sites in the CFH-to-F13B locus, which are not explicitly described herein, may further refine this analysis. A SNP analysis using non-synonymous polymorphisms in the CFH-to-F13B locus can be useful to identify variant polypeptides. Other SNPs associated with risk may encode a protein with the same sequence as a protein encoded by a neutral or protective SNP but contain an allele in a promoter or intron, for example, changes the level or site of gene expression. It will also be appreciated that a polymorphism in the CFH-to-F13B locus may be linked to a variation in a neighboring gene. The variation in the neighboring gene may result in a change in expression or form of an encoded protein and have detrimental or protective effects in the carrier.

As used herein, the term “variant” can also refer to a nucleotide sequence in which the sequence differs from the sequence most prevalent in a population, for example by one nucleotide, in the case of the SNPs described herein. For example, some variations or substitutions in the nucleotide sequence of the CFH-to-F13B locus alter a codon so that a different amino acid is encoded resulting in a variant polypeptide. The term “variant,” can also refer to a polypeptide in which the sequence differs from the sequence most prevalent in a population at a position that does not change the amino acid sequence of the encoded polypeptide (i.e., a conserved change). Variant polypeptides can be encoded by a risk haplotype, encoded by a protective haplotype, or can be encoded by a neutral haplotype. Variant polypeptides can be associated with risk, associated with protection, or can be neutral.

The methods and materials described herein can be used to determine whether the nucleic acid of a subject (e.g., human) contains a polymorphism, such as a single nucleotide polymorphism (SNP). For example, methods and materials provided herein can be used to determine whether a subject has a variant SNP. Any method can be used to detect a polymorphism in the CFH-to-F13B locus. For example, polymorphisms can be detected by sequencing exons, introns, or untranslated sequences, denaturing high performance liquid chromatography (DHPLC), allele-specific hybridization, allele-specific restriction digests, mutation specific polymerase chain reactions, single-stranded conformational polymorphism detection, and combinations of such methods.

In one aspect, polymorphisms can be detected in a target nucleic acid isolated from a subject. Typically genomic DNA is analyzed. For assay of genomic DNA, virtually any biological sample containing genomic DNA or RNA, e.g., nucleated cells, is suitable. For example, in the experiments described in the Examples section herein, genomic DNA was obtained from peripheral blood leukocytes collected from case and control subjects (QIAamp DNA Blood Maxi kit, Qiagen, Valencia, Calif.). Other suitable samples include, but are not limited to, saliva, cheek scrapings, biopsies of retina, kidney or liver or other organs or tissues; skin biopsies; amniotic fluid or CNS samples; and the like. In one aspect RNA or cDNA can be assayed. In one aspect, the assay can detect variant proteins encoded by one or more genes present in the CFH-to-F13B. Methods for purification or partial purification of nucleic acids or proteins from patient samples for use in diagnostic or other assays are known in the art.

By “isolated nucleic acid” or “purified nucleic acid” is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

The identity of bases occupying the polymorphic sites in CFH-to-F13B locus can be determined in a subject, e.g., in a patient being analyzed, using any of several methods known in the art. For example, and not to be limiting use of allele-specific probes, use of allele-specific primers, direct sequence analysis, denaturing gradient gel electrophoresis (DGGE) analysis, single-strand conformation polymorphism (SSCP) analysis, and denaturing high performance liquid chromatography (DHPLC) analysis. Other well known methods to detect polymorphisms in DNA include use of: Molecular Beacons technology (see, e.g., Piatek et al., 1998; Nat. Biotechnol. 16:359-63; Tyagi, and Kramer, 1996, Nat. Biotechnology 14:303-308; and Tyagi, et al., 1998, Nat. Biotechnol. 16:49-53), Invader technology (see, e.g., Neri et al., 2000, Advances in Nucleic Acid and Protein Analysis 3826:117-125 and U.S. Pat. No. 6,706,471), nucleic acid sequence based amplification (Nasba) (Compton, 1991), Scorpion technology (Thelwell et al., 2000, Nuc. Acids Res, 28:3752-3761 and Solinas et al., 2001, “Duplex Scorpion primers in SNP analysis and FRET applications” Nuc. Acids Res, 29:20.), restriction fragment length polymorphism (RFLP) analysis, and the like. Additional methods will be apparent to one of skill in the art.

The term “primer” refers to a single-stranded oligonucleotide capable of acting as a point of initiation of template-directed DNA synthesis under appropriate conditions, in an appropriate buffer and at a suitable temperature. The appropriate length of a primer depends on the intended use of the primer but typically ranges from 15 to 30 nucleotides. A primer sequence need not be exactly complementary to a template but must be sufficiently complementary to hybridize with a template. The term “primer site” refers to the area of the target DNA to which a primer hybridizes. The term “primer pair” means a set of primers including a 5′ upstream primer, which hybridizes to the 5′ end of the DNA sequence to be amplified and a 3′ downstream primer, which hybridizes to the complement of the 3′ end of the sequence to be amplified.

Exemplary hybridization conditions for short probes and primers is about 5 to 12 degrees C., below the calculated Tm. Formulas for calculating Tm are known and include: Tm=4° C.×(number of G's and C's in the primer)+2° C.×(number of A's and T's in the primer) for oligos <14 bases and assumes a reaction is carried out in the presence of 50 mM monovalent cations. For longer oligos, the following formula can be used: Tm=64.9° C.+41° C.×(number of G's and C's in the primer-16.4)/N, where N is the length of the primer. Another commonly used formula takes into account the salt concentration of the reaction (Rychlik, supra, Sambrook, supra, Mueller, supra.): Tm=81.5° C.+16.6° C.×(log 10[Na+]+[K+])+0.41° C.×(% GC)−675/N, where N is the number of nucleotides in the oligo. The aforementioned formulas provide a starting point for certain applications; however, the design of particular probes and primers may take into account additional or different factors. Methods for design of probes and primers for use in the methods of the invention are well known in the art.

The design and use of allele-specific probes for analyzing polymorphisms are described by e.g., Saiki et al., 1986; Dattagupta, EP 235,726, Saiki, WO 89/11548. Briefly, allele-specific probes are designed to hybridize to a segment of target DNA from one individual but not to the corresponding segment from another individual, if the two segments represent different polymorphic forms. Hybridization conditions are chosen that are sufficiently stringent so that a given probe essentially hybridizes to only one of two alleles. Typically, allele-specific probes can be designed to hybridize to a segment of target DNA such that the polymorphic site aligns with a central position of the probe.

As used herein, “probes” are nucleic acids capable of binding in a base-specific manner to a complementary strand of nucleic acid. Such probes include nucleic acids and peptide nucleic acids (Nielsen et al., 1991). Hybridization may be performed under stringent conditions which are known in the art. For example, see Berger and Kimmel (1987) Methods In Enzymology, Vol. 152: Guide To Molecular Cloning Techniques, San Diego: Academic Press, Inc.; Sambrook et al. (1989) Molecular Cloning: A Laboratory Manual, 2nd Ed., Vols. 1-3, Cold Spring Harbor Laboratory; Sambook (2001) 3rd Edition; Rychlik, W. and Rhoads, R. E., 1989, Nucl. Acids Res. 17, 8543; Mueller, P. R. et al. (1993) In: Current Protocols in Molecular Biology 15.5, Greene Publishing Associates, Inc. and John Wiley and Sons, New York; and Anderson and Young, Quantitative Filter Hybridization in Nucleic Acid Hybridization (1985)). As used herein, the term “probe” includes primers. Probes and primers are sometimes referred to as “oligonucleotides.”

Allele-specific probes can be used in pairs, one member of a pair designed to hybridize to the reference allele of a target sequence and the other member designed to hybridize to the variant allele. Several pairs of probes can be immobilized on the same support for simultaneous analysis of multiple polymorphisms within the same target gene sequence.

The design and use of allele-specific primers for analyzing polymorphisms are described by, e.g., WO 93/22456 and Gibbs, 1989. Briefly, allele-specific primers are designed to hybridize to a site on target DNA overlapping a polymorphism and to prime DNA amplification according to standard PCR protocols only when the primer exhibits perfect complementarity to the particular allelic form. A single-base mismatch prevents DNA amplification and no detectable PCR product is formed. The method works best when the polymorphic site is at the extreme 3′-end of the primer, because this position is most destabilizing to elongation from the primer.

In some embodiments, genomic DNA can be used to detect polymorphisms in the CFH-to-F13B locus. Genomic DNA can be extracted from a sample, such as a peripheral blood sample or a tissue sample. Standard methods can be used to extract genomic DNA from a sample, such as phenol extraction. In some cases, genomic DNA can be extracted using a commercially available kit (e.g., from Qiagen, Chatsworth, Calif.; Promega, Madison, Wis.; or Gentra Systems, Minneapolis, Minn.).

Other methods for detecting polymorphisms can involve amplifying a nucleic acid from a sample obtained from a subject (e.g., amplifying the segments of nucleic acids from the CFH-to-F13B locus using CFH-to-F13B locus-specific primers) and analyzing the amplified nucleic acids. This can be accomplished by standard polymerase chain reaction (PCR, qPCT, & RT-PCR) protocols or other methods known in the art. The amplifying can result in the generation of CFH/F13B allele-specific oligonucleotides, which span the single nucleotide polymorphic sites in the CFH-to-F13B locus. The CFH/F13B specific primer sequences and CFH/F13B allele-specific oligonucleotides can be derived from the coding (exons) or non-coding (promoter, 5′ untranslated, introns or 3′ untranslated) regions of the CFH-to-F13B locus. In one aspect Genomic DNA from all subjects can be isolated from peripheral blood leukocytes with QIAamp DNA Blood Maxi kits (Qiagen, Valencia, Calif.). DNA samples can be screened for SNPs in the genes contained within the CFH-to-F13B locus. Genotyping can be performed byTaqMan assays (Applied Biosystems, Foster City, Calif.) using 10 ng of template DNA in a 5 uL reaction. The thermal cycling conditions in the 384-well thermocycler (PTC-225, MJ Research) can consist of an initial hold at 95° C. for 10 minutes, followed by 40 cycles of a 15-second 95° C. denaturation step and a 1-minute 60° C. annealing and extension step. Plates can be read in the 7900HT Fast Real-Time PCR System (Applied Biosystems).

Amplification products generated using PCR can be analyzed by the use of denaturing gradient gel electrophoresis (DGGE). Different alleles can be identified based on sequence-dependent melting properties and electrophoretic migration in solution. See Erlich, ed., PCR Technology, Principles and Applications for DNA Amplification, Chapter 7 (W.H. Freeman and Co, New York, 1992).

Alleles of target sequences can be differentiated using single-strand conformation polymorphism (SSCP) analysis. Different alleles can be identified based on sequence- and structure-dependent electrophoretic migration of single stranded PCR products (Orita et al., 1989). Amplified PCR products can be generated according to standard protocols and heated or otherwise denatured to form single stranded products, which may refold or form secondary structures that are partially dependent on base sequence.

Alleles of target sequences can be differentiated using denaturing high performance liquid chromatography (DHPLC) analysis. Different alleles can be identified based on base differences by alteration in chromatographic migration of single stranded PCR products (Frueh and Noyer-Weidner, 2003). Amplified PCR products can be generated according to standard protocols and heated or otherwise denatured to form single stranded products, which may refold or form secondary structures that are partially dependent on the base sequence.

Direct sequence analysis of polymorphisms can be accomplished using DNA sequencing procedures that are well-known in the art. See Sambrook et al., Molecular Cloning, A Laboratory Manual (2nd Ed., CSHP, New York 1989) and Zyskind et al., Recombinant DNA Laboratory Manual (Acad. Press, 1988).

A wide variety of other methods are known in the art for detecting polymorphisms in a biological sample. See, e.g., Ullman et al. “Methods for single nucleotide polymorphism detection” U.S. Pat. No. 6,632,606; Shi, 2002, “Technologies for individual genotyping: detection of genetic polymorphisms in drug targets and disease genes” Am J Pharmacogenomics 2:197-205; Kwok et al., 2003, “Detection of single nucleotide polymorphisms” Curr Issues Biol. 5:43-60).

Also described herein are methods for determining a female Caucasian subject's susceptibility to having or developing a complement-mediated disease comprising determining in the female Caucasian subject the identity of one or more major or minor haplotypes. The haplotypes can include, but are not limited to H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, or a complement thereof.

In one aspect, determining the identity of a certain haplotype or certain haplotypes in a female subject can indicate that the female subject has an increased risk of having or developing a complement-mediated disease. Such hapltoypes can be risk haplotypes. For example, and not to be limiting, a risk haplotype can be H1_62_A, H2_62_A, H1_51_B H2_51_B or a complement thereof. Thus, described herein are methods for determining a female Caucasian subject's susceptibility to having or developing a complement-mediated disease comprising determining in the female Caucasian subject the identity of one or more haplotypes described herein, wherein a risk haplotype is indicative of the female subject's increased risk for having or developing a complement-mediated disease.

In another aspect, determining the identity of a certain haplotype or certain haplotypes in a female subject can indicate that the female subject has a deacreased risk of having or developing a complement-mediated disease. Such haplotypes can be protective haplotypes. For example, and not to be limiting, a protective haplotype can be H3_62_A, H5_62_A, H11_62_A, H3_51_B, H5_51_B, H12_51_B, H14_51_B, or a complement thereof. Thus, described herein are methods for determining a female Caucasian subject's susceptibility to having or developing a complement-mediated disease comprising determining in the female Caucasian subject the identity of one or more haplotypes described herein, wherein a protective haplotype is indicative of the female subject's decreased risk for having or developing a complement-mediated disease.

In yet another aspect, determining the identity of a certain haplotype or certain haplotypes in a female subject can indicate that the female subject does not have an increased risk or a decreased risk of having or developing a complement-mediated disease. Such haplotypes can be neutral haplotypes. For example, and not to be limiting, a neutral haplotype can be H4_62_A, H6_62_A, H7_62_A, H8_62_A, H9_62_A, H10_62_A, H12_62_A, H13_62_A, H14_62_A, H15_62_A, H4_51_B, H6_51_B, H7_51_B, H8_51_B, H9_51_B, H10_51_B, H11_51_B, H13_51_B, H15_51_B, H16_51_B, H17_51_B, H18_51_B, H19_51_B, H20_51_B, or a complement thereof. Thus, described herein are methods for determining a female Caucasian subject's susceptibility to having or developing a complement-mediated disease comprising determining in the female Caucasian subject the identity of one or more haplotypes described herein, wherein a neutral haplotype does not indicate that the female subject has an increased risk or a decreased risk for having or developing a complement-mediated disease.

Also described herein are methods for determining a male Caucasian subject's susceptibility to having or developing a complement-mediated disease comprising determining in the male Caucasian subject the identity of one or more major or minor haplotypes. The haplotypes can include, but are not limited to H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B H15_51_B H16_51_B H17_51_B H18_51_B H19_51_B or a complement thereof.

In one aspect, determining the identity of a certain haplotype or certain haplotypes in a male subject can indicate that the male subject has a increased risk of having or developing a complement-mediated disease. Such hapltoypes can be risk haplotypes. For example, and not to be limiting, a risk haplotype can be H2_62_A, H1_51_B, H2_51_B, or a complement thereof. Thus, described herein are methods for determining a male Caucasian subject's susceptibility to having or developing a complement-mediated disease comprising determining in the male Caucasian subject the identity of one or more haplotypes described herein, wherein a risk haplotype is indicative of the male subject's increased risk for having or developing a complement-mediated disease.

In another aspect, determining the identity of a certain haplotype or certain haplotypes in a male subject can indicate that the male subject has a deacreased risk of having or developing a complement-mediated disease. Such haplotypes can be protective haplotypes. For example, and not to be limiting, a protective haplotype can be H11_62_A H3_51_B H14_51_B or a complement thereof. Thus, described herein are methods for determining a male Caucasian subject's susceptibility to having or developing a complement-mediated disease comprising determining in the male Caucasian subject the identity of one or more haplotypes described herein, wherein a protective haplotype is indicative of the male subject's decreased risk for having or developing a complement-mediated disease.

In yet another aspect, determining the identity of a certain haplotype or certain haplotypes in a male subject can indicate that the male subject does not have an increased risk or a decreased risk of having or developing a complement-mediated disease. Such haplotypes can be neutral haplotypes. For example, and not to be limiting, a neutral haplotype can be H1_62_A, H3_62_A, H4_62_A, H5_62_A, H6_62_A, H7_62_A, H8_62_A, H9_62_A, H10_62_A, H12_62_A, H13_62_A, H14_62_A, H15_62_A H4_51_B, H5_51_B, H6_51_B, H7_51_B, H8_51_B, H9_51_B, H10_51_B, H11_51_B, H12_51_B, H13_51_B, H15_51_B, H16_51_B, H17_51_B, H18_51_B, H19_51_B, H20_51_B, H21_51_B, H22_51_B, or a complement thereof. Thus, described herein are methods for determining a male Caucasian subject's susceptibility to having or developing a complement-mediated disease comprising determining in the male Caucasian subject the identity of one or more haplotypes described herein, wherein a neutral haplotype does not indicate that the male subject has an increased risk or a decreased risk for having or developing a complement-mediated disease.

In one aspect, the SNPs, haplotypes, or diplotypes described herein can be determined from a sample obtained from the subject. In another aspect, the subject's SNPs, haplotype, or diplotype can be determined by amplifying or sequencing a nucleic acid sample obtained from the subject. In yet a further aspect, the methods descrined herein can further comprise administering a therapeutic composition to the subject or treating the subject with an effective amount of a therapeutic composition.

As used herein, “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

The terms “administering” and “administration” refer to any method of providing a pharmaceutical preparation to a subject. Such methods are well known to those skilled in the art and include, but are not limited to, oral administration, sublingual administration, trans-buccal mucosa administration, transdermal administration, administration by inhalation, nasal administration, topical administration, intravaginal administration, ophthalmic administration, intraaural administration, intracerebral administration, intrathecal administration, rectal administration, intraperitoneal administration, and parenteral administration, including injectable such as intravenous administration, intra-arterial administration, intramuscular administration, intradermal administration, and subcutaneous administration. Ophthalmic administration can include topical administration, subconjunctival administration, sub-Tenon's administration, epibulbar administration, retrobulbar administration, intra-orbital administration, and intraocular administration, which includes intra-vitreal administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “therapeutically effective amount” refers to an amount that is sufficient to achieve the desired result or to have an effect on an undesired condition.

In yet another aspect, the methods for determining a Caucasian subject's susceptibility to having or developing a complement-mediated disease described herein can further comprise examining the subject with an ophthalmological procedure.

Various ophthalmological procedures known to persons of ordinary skill in the art can be performed to examine the subject including, but not limited to, autofluorescent imaging techniques, infrared imaging techniques, optical coherence tomography (OCT), Stratus optical coherence tomography (Stratus OCT), Fourier-domain optical coherence tomography (Fd-OCT), two-photon-excited fluorescence (TPEF) imaging, adaptive optics scanning laser ophthalmoscopy (AOSLO), scanning laser ophthalmoscopy, near-infrared imaging combined with spectral domain optical coherence tomography (SD-OCT), color fundus photography, fundus autofluorescence imaging, red-free imaging, fluorescein angiography, indocyanin green angiography, multifocal electroretinography (ERG) recording, microperimetry, color Doppler optical coherence tomography (CDOCT), and visual field assessment. Additionally, the subject can be examined by using the Heidelberg Spectralis, the Zeiss Cirrus, the Topcon 3D OCT 2000, the Optivue RTVue SD-OCT, the Opko OCT SLO, the NIDEK F-10, or the Optopol SOCT Copernicus HR.

### 1. Predicting Progression of Disease

Also described herein are methods for predicting progression of a complement-mediated disease in a Caucasian subject. The complement-mediated disease can be, but is not limited to, age-related macular degeneration. In one aspect the methods comprise determining in the Caucasian subject the identity of one or more major or minor haplotypes. The haplotypes can include, but are not limited to H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, H1_51_A, H2_51_A, H3_51_A, H4_51_A, H5_51_A, H6_51_A, H7_51_A, H8_51_A, H9_51_A, H10_51_A, H11_51_A, H12_51_A, H13_51_A, H14_51_A, H15_51_A, H16_51_A, H17_51_A, H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof. As described herein, the presence of one or more of the haplotypes disclosed herein can indicate a subject's susceptibility for having or developing late-stage age-related macular degeneration.

In one aspect, determining the identity of a certain haplotype or certain haplotypes in a subject can indicate that the subject has a increased risk of having or developing late-stage age-related macular degeneration. Such hapltoypes can be risk haplotypes. For example, and not to be limiting, a risk haplotype can be H2_62_A, H2_51_A H1_51_B H2_51_B or a complement thereof. Thus, described herein are methods for determining a Caucasian subject's susceptibility to having or developing late-stage age-related macular degeneration comprising determining in the Caucasian subject the identity of one or more haplotypes described herein, wherein a risk haplotype is indicative of the subject's increased risk for having or developing late-stage age-related macular degeneration.

In another aspect, determining the identity of a certain haplotype or certain haplotypes in a subject can indicate that the subject has a deacreased risk of having or developing late-stage age-related macular degeneration. Such haplotypes can be protective haplotypes. For example, and not to be limiting, a protective haplotype can be H3_62_A, H5_62_A, H11_62_A, H3_51_A, H5_51_A, H10_51_A, H3_51_B, H5_51_B, H12_51_B, H14_51_B, or a complement thereof. Thus, described herein are methods for determining a Caucasian subject's susceptibility to having or developing late-stage age-related macular degeneration comprising determining in the Caucasian subject the identity of one or more haplotypes described herein, wherein a protective haplotype is indicative of the subject's decreased risk for having or developing late-stage age-related macular degeneration.

In yet another aspect, determining the identity of a certain haplotype or certain haplotypes in a subject can indicate that the subject does not have an increased risk or a decreased risk of having or developing late-stage age-related macular degeneration. Such haplotypes can be neutral haplotypes. For example, and not to be limiting, a neutral haplotype can be H1_62_A, H4_62_A, H6_62_A, H7_62_A, H8_62_A, H9_62_A, H10_62_A, H12_62_A, H13_62_A, H14_62_A, H15_62_A, H1_51_A, H4_51_A, H6_51_A, H7_51_A, H8_51_A, H9_51_A, H11_51_A, H12_51_A, H13_51_A, H14_51_A, H15_51_A, H16_51_A, H17_51_A, H4_51_B, H6_51_B, H7_51_B, H8_51_B, H9_51_B, H10_51_B, H11_51_B, H13_51_B, H15_51_B, H16_51_B, H17_51_B, H18_51_B, H19_51_B, H20_51_B, H21_51_B, H22_51_B, or a complement thereof. Thus, described herein are methods for determining a Caucasian subject's susceptibility to having or developing late-stage age-related macular degeneration comprising determining in the Caucasian subject the identity of one or more haplotypes described herein, wherein a neutral haplotype does not indicate that the subject has an increased risk or a decreased risk for having or developing late-stage age-related macular degeneration.

Also described herein are methods for determining a female Caucasian subject's susceptibility to having or developing late-stage age-related macular degeneration comprising determining in the female Caucasian subject the identity of one or more major or minor haplotypes. The haplotypes can include, but are not limited to H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, or a complement thereof.

In one aspect, determining the identity of a certain haplotype or certain haplotypes in a female subject can indicate that the female subject has a increased risk of having or developing late-stage age-related macular degeneration. Such hapltoypes can be risk haplotypes. For example, and not to be limiting, a risk haplotype can be H1_62_A, H2_62_A, H1_51_B, H2_51_B, or a complement thereof. Thus, described herein are methods for determining a female Caucasian subject's susceptibility to having or developing late-stage age-related macular degeneration comprising determining in the female Caucasian subject the identity of one or more haplotypes described herein, wherein a risk haplotype is indicative of the female subject's increased risk for having or developing late-stage age-related macular degeneration.

In another aspect, determining the identity of a certain haplotype or certain haplotypes in a female subject can indicate that the female subject has a deacreased risk of having or developing late-stage age-related macular degeneration. Such haplotypes can be protective haplotypes. For example, and not to be limiting, a protective haplotype can be H3_62_A, H5_62_A, H11_62_A, H3_51_B, H5_51_B, H12_51_B, H14_51_B, or a complement thereof. Thus, described herein are methods for determining a female Caucasian subject's susceptibility to having or developing late-stage age-related macular degeneration comprising determining in the female Caucasian subject the identity of one or more haplotypes described herein, wherein a protective haplotype is indicative of the female subject's decreased risk for having or developing late-stage age-related macular degeneration.

In yet another aspect, determining the identity of a certain haplotype or certain haplotypes in a female subject can indicate that the female subject does not have an increased risk or a decreased risk of having or developing late-stage age-related macular degeneration. Such haplotypes can be neutral haplotypes. For example, and not to be limiting, a neutral haplotype can be H4_62_A, H6_62_A, H7_62_A, H8_62_A, H9_62_A, H10_62_A, H12_62_A, H13_62_A, H14_62_A, H15_62_A, H4_51_B, H6_51_B, H7_51_B, H8_51_B, H9_51_B, H10_51_B, H11_51_B, H13_51_B, H15_51_B, H16_51_B, H17_51_B, H18_51_B, H19_51_B, H20_51_B, or a complement thereof. Thus, described herein are methods for determining a female Caucasian subject's susceptibility to having or developing late-stage age-related macular degeneration comprising determining in the female Caucasian subject the identity of one or more haplotypes described herein, wherein a neutral haplotype does not indicate that the female subject has an increased risk or a decreased risk for having or developing late-stage age-related macular degeneration.

Also described herein are methods for determining a male Caucasian subject's susceptibility to having or developing late-stage age-related macular degeneration comprising determining in the male Caucasian subject the identity of one or more major or minor haplotypes. The haplotypes can include, but are not limited to H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, or a complement thereof.

In one aspect, determining the identity of a certain haplotype or certain haplotypes in a male subject can indicate that the male subject has a increased risk of having or developing late-stage age-related macular degeneration. Such hapltoypes can be risk haplotypes. For example, and not to be limiting, a risk haplotype can be H2_62_A H1_51_B H2_51_B or a complement thereof. Thus, described herein are methods for determining a male Caucasian subject's susceptibility to having or late-stage age-related macular degeneration comprising determining in the male Caucasian subject the identity of one or more haplotypes described herein, wherein a risk haplotype is indicative of the male subject's increased risk for having or developing late-stage age-related macular degeneration.

In another aspect, determining the identity of a certain haplotype or certain haplotypes in a male subject can indicate that the male subject has a deacreased risk of having or developing late-stage age-related macular degeneration. Such haplotypes can be protective haplotypes. For example, and not to be limiting, a protective haplotype can be H11_62_A, H3_51_B, H14_51_B, or a complement thereof. Thus, described herein are methods for determining a male Caucasian subject's susceptibility to having or developing late-stage age-related macular degeneration comprising determining in the male Caucasian subject the identity of one or more haplotypes described herein, wherein a protective haplotype is indicative of the male subject's decreased risk for having or developing late-stage age-related macular degeneration.

In yet another aspect, determining the identity of a certain haplotype or certain haplotypes in a male subject can indicate that the male subject does not have an increased risk or a decreased risk of having or developing late-stage age-related macular degeneration. Such haplotypes can be neutral haplotypes. For example, and not to be limiting, a neutral haplotype can be H1_62_A, H3_62_A, H4_62_A, H5_62_A, H6_62_A, H7_62_A, H8_62_A, H9_62_A, H10_62_A, H12_62_A, H13_62_A, H14_62_A, H15_62_A H4_51_B, H5_51_B, H6_51_B, H7_51_B, H8_51_B, H9_51_B, H10_51_B, H11_51_B, H12_51_B, H13_51_B, H15_51_B, H16_51_B, H17_51_B, H18_51_B, H19_51_B, H20_51_B, H21_51_B, H22_51_B, or a complement thereof. Thus, described herein are methods for determining a male Caucasian subject's susceptibility to having or developing late-stage age-related macular degeneration comprising determining in the male Caucasian subject the identity of one or more haplotypes described herein, wherein a neutral haplotype does not indicate that the male subject has an increased risk or a decreased risk for having or developing late-stage age-related macular degeneration.

### 2. Diplotypes

Also described herein are methods for determining a Caucasian subject's susceptibility to having or developing a complement-mediated disease comprising determining in the Caucasian subject the identity of one or more diplotypes described herein. Human autosomal chromosomes normally come in pairs, and the combination in one individual of these two haplotypes is referred to herein as a “Diplotype”. As such, “based on” when used in the context of a diplotype “based on” a haplotype, can refer to identifying the combination in one individual of these two haplotypes.

Any haplotype or haplotypes described herein can be used to create a diplotype. For example, a diplotype can comprise a protective and a risk haplotype, two protective haplotypes, two risk haplotypes, a neutral and a risk haplotype, a neutral and a protective haplotype, or two neutral haplotypes. In an aspect, one haplotype may be dominant or one haplotype may be recessive.

For example, and not to be limiting, a single H1 haplotype when combined with another H1 haplotype can be an H1_Y_Z:H1_Y_Z diplotype; a single H1 haplotype when combined a H2, H8, or H13 haplotype can be an H1_Y_Z:H2 Y_Z diplotype, an H1_Y_Z:H8 Y_Z diplotype, or an H1_Y_Z:H13 Y_Z diplotype. In one aspect, the diplotypes described herein can be associated with increased risk for developing AMD. In another aspect, the diplotypes described herein can be associated with a decreased risk for developing AMD.

Described here is are methods for determining a Caucasian subject's susceptibility to having or developing age-related macular degeneration comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, and wherein the presence of one or more of the diplotypes indicates the subject's susceptibility for having or developing age-related macular degeneration. In some aspects, the subject's haplotype can be determined from a sample obtained from the subject. In some aspects the subject's haplotype can be determined by amplifying or sequencing a nucleic acid sample obtained from the subject.

Described here is are methods for determining a Caucasian subject's susceptibility to having or developing age-related macular degeneration comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, and wherein the presence of one or more of the diplotypes indicates the subject's susceptibility for having or developing age-related macular degeneration,

Described here is are methods for determining a Caucasian subject's susceptibility to having or developing age-related macular degeneration comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, wherein the diplotype H1_51_B: H1_51_B, H1_51_B:H2_51_B, H1_51_B:H8_51_B, H1_51_B:H13_51_B, H2_51_B:H2_51_B, H2_51_B:H10_51_B, H1_51_B:any minor 51_B haplotype, H2_51_B:any minor 51_B haplotype, or a complement thereof, is indicative of the subject's increased risk for having or developing age-related macular degeneration.

In some aspects wherein an increased risk is identified, the method can further comprise administering a therapeutic composition to the subject.

Described here is are methods for determining a Caucasian subject's susceptibility to having or developing age-related macular degeneration comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, wherein diplotype H1_51_B: H3_51_B, H1_51_B:H5_51_B, H1_51_B:H14_51_B, H3_51_B: H3_51_B, H3_51_B: H4_51_B, H3_51_B: H5_51_B, H3_51_B: H6_51_B, H3_51_B: any 51_B minor haplotype, H4_51_B: H5_51_B, H5_51_B: H5_51_B, H5_51_B: any 51_B minor haplotype, H6_51_B: H6_51_B, H6_51_B: any 51_B minor haplotype, or a complement thereof, is indicative of the subject's decreased risk for having or developing age-related macular degeneration.

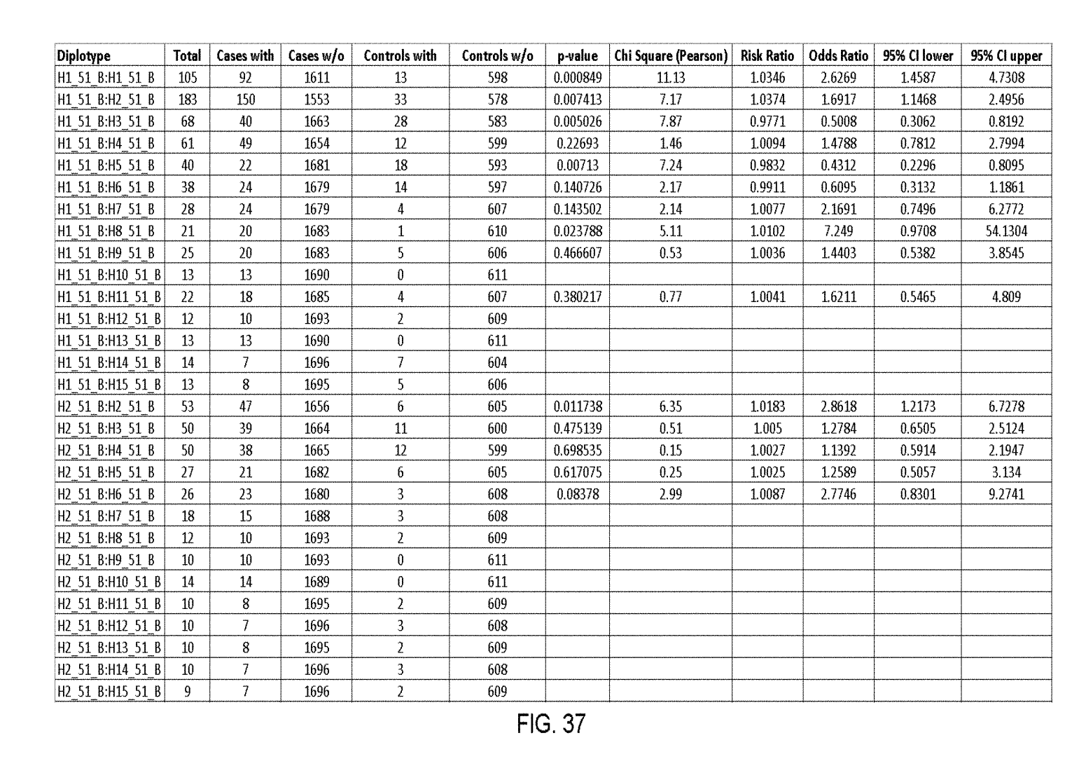

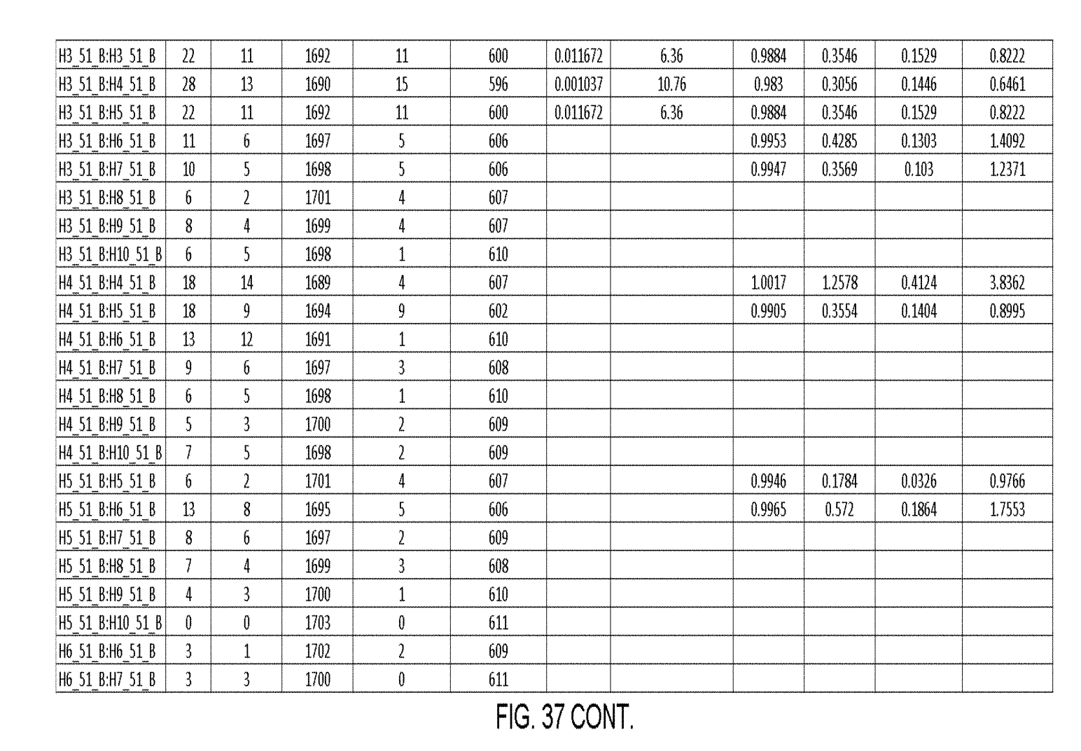

In some aspects the subject can be female. In some aspects wherein the subject is female, one or more of the diplotypes identified in FIG. 37 can be used to indicate the subject's risk for having or developing age-related macular degeneration.

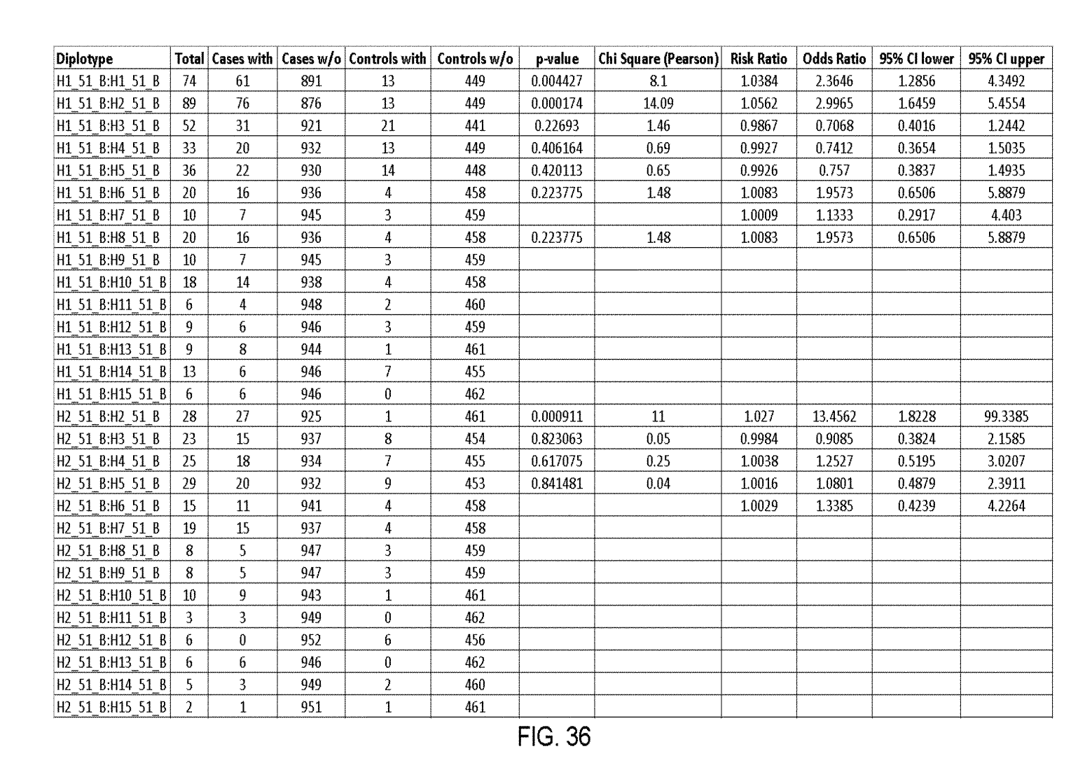

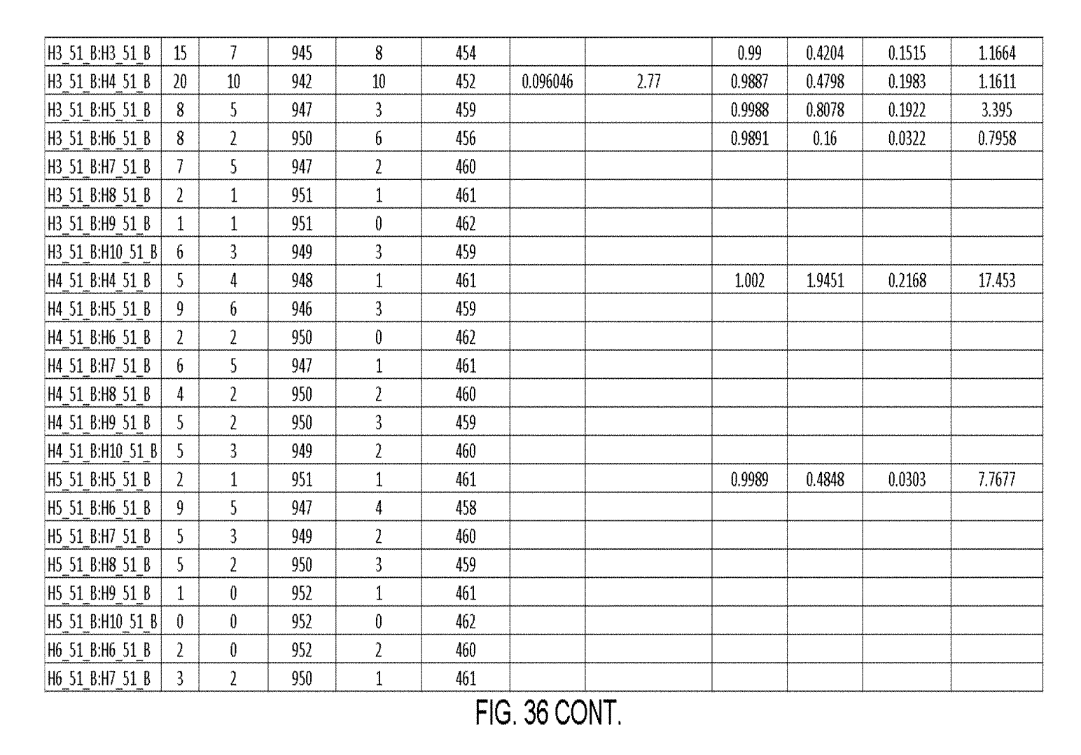

In some aspects the subject can be male. In some aspects wherein the subject is male, one or more of the diplotypes identified in FIG. 36 can be used to indicate the subject's risk for having or developing age-related macular degeneration.

Described herein are methods of identifying a Caucasian subject in need of treatment for age-related macular degeneration comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, and wherein the presence of one or more of the diplotypes indicates whether the subject is in need of treatment for age-related macular degeneration.

In some aspects, the subject's haplotype can be determined from a sample obtained from the subject. In some aspects the subject's haplotype can be determined by amplifying or sequencing a nucleic acid sample obtained from the subject.

Described herein are methods of identifying a Caucasian subject in need of treatment for age-related macular degeneration comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, wherein diplotype H1_51_B: H1_51_B, H1_51_B:H2_51_B, H1_51_B:H8_51_B, H1_51_B:H13_51_B, H2_51_B:H2_51_B, H2_51_B:H10_51_B, H1_51_B:any minor 51_B haplotype, H2_51_B:any minor 51_B haplotype, or a complement thereof, indicates the subject is in need of treatment for age-related macular degeneration.

Described herein are methods of identifying a Caucasian subject in need of treatment for age-related macular degeneration comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, wherein diplotype H1_51_B: H3_51_B, H1_51_B:H5_51_B, H1_51_B:H14_51_B, H3_51_B: H3_51_B, H3_51_B: H4_51_B, H3_51_B: H5_51_B, H3_51_B: H6_51_B, H3_51_B: any 51_B minor haplotype, H4_51_B: H5_51_B, H5_51_B: H5_51_B, H5_51_B: any 51_B minor haplotype, H6_51_B: H6_51_B, H6_51_B: any 51_B minor haplotype, or a complement thereof, indicates the subject is not in need of treatment for age-related macular degeneration.

Described herein are methods of identifying a Caucasian subject in need of treatment for age-related macular degeneration comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, wherein the presence of one or more of the diplotypes identified in FIG. 36 can be used to determine whether, or a complement thereof, can indicates the subject may be in need of treatment for age-related macular degeneration. In some aspects the subject can be female. In some aspects wherein the subject is female, one or more of the diplotypes identified in FIG. 37 can be used to deterime whether the subject is in need of treatment for age-related macular degeneration. In some aspects the subject can be male. In some aspects wherein the subject is male, one or more of the diplotypes identified in FIG. 36 can be used to determine whether the subject is in need of treatment for age-related macular degeneration.

Described herein are methods of identifying a Caucasian subject in need of a prophylactic treatment for age-related macular degeneration comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, and wherein the presence of one or more of the diplotypes indicates whether the subject is in need of prophylactic treatment for age-related macular degeneration.

Described herein are methods of identifying a Caucasian subject in need of a prophylactic treatment for age-related macular degeneration comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, wherein diplotype H1_51_B: H1_51_B, H1_51_B:H2_51_B, H1_51_B:H8_51_B, H1_51_B:H13_51_B, H2_51_B:H2_51_B, H2_51_B:H10_51_B, H1_51_B:any minor 51_B haplotype, H2_51_B:any minor 51_B haplotype, or a complement thereof, indicates the subject is in need of prophylactic treatment for age-related macular degeneration.

Described herein are methods of identifying a Caucasian subject in need of a prophylactic treatment for age-related macular degeneration comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, wherein diplotype H1_51_B: H3_51_B, H1_51_B:H5_51_B, H1_51_B:H14_51_B, H3_51_B: H3_51_B, H3_51_B: H4_51_B, H3_51_B: H5_51_B, H3_51_B: H6_51_B, H3_51_B: any 51_B minor haplotype, H4_51_B: H5_51_B, H5_51_B: H5_51_B, H5_51_B: any 51_B minor haplotype, H6_51_B: H6_51_B, H6_51_B: any 51_B minor haplotype, or a complement thereof, indicates the subject is not in need of prophylactic treatment for age-related macular degeneration.

Described herein are methods of identifying a Caucasian subject in need of a prophylactic treatment for age-related macular degeneration comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, wherein the presence of one or more of the diplotypes identified in FIG. 36, or a complement thereof, can be used to determine whether, the subject may be in need of prophylactic treatment for age-related macular degeneration.

Described herein are methods of identifying a Caucasian subject appropriate for an age-related macular degeneration clinical trial comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, and wherein the presence of one or more of the diplotypes indicates whether the subject is appropriate for the clinical trial.

Described herein are methods of identifying a Caucasian subject appropriate for an age-related macular degeneration clinical trial comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, wherein diplotype H1_51_B: H1_51_B, H1_51_B:H2_51_B, H1_51_B:H8_51_B, H1_51_B:H13_51_B, H2_51_B:H2_51_B, H2_51_B:H10_51_B, H1_51_B:any minor 51_B haplotype, H2_51_B:any minor 51_B haplotype, or a complement thereof, indicates the subject is appropriate for the clinical trial.

Described herein are methods of identifying a Caucasian subject appropriate for an age-related macular degeneration clinical trial comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, wherein diplotype H1_51_B: H3_51_B, H1_51_B:H5_51_B, H1_51_B:H14_51_B, H3_51_B: H3_51_B, H3_51_B: H4_51_B, H3_51_B: H5_51_B, H3_51_B: H6_51_B, H3_51_B: any 51_B minor haplotype, H4_51_B: H5_51_B, H5_51_B: H5_51_B, H5_51_B: any 51_B minor haplotype, H6_51_B: H6_51_B, H6_51_B: any 51_B minor haplotype, or a complement thereof, indicates the subject is not appropriate for the clinical trial.

Described herein are methods of identifying a Caucasian subject appropriate for an age-related macular degeneration clinical trial comprising determining in the Caucasian subject the identity of one or more diplotypes based on one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, wherein the presence of one or more of the diplotypes identified in FIG. 36, or a complement thereof, indicates the subject may be appropriate for the clinical trial.

### 3. Haplotype Tagging Snps

Described herein are methods for determining a Caucasian subject's susceptibility to having or developing a complement-mediated disease comprising determining in the Caucasian subject the identity of at least six SNPs in the CFH-to-F13B locus. The SNPs can be, but are not limited to: (i) rs35928059, rs800292, rs1061170, rs12144939, rs7546940, rs1409153, rs10922153, or rs698859, or (ii) a SNP in linkage disequilibrium with the SNPs of (i). In one aspect, the presence of at least six of the SNPs can indicate the subject's susceptibility for having or developing age-related macular degeneration. In another aspect, the SNPs can be haplotype tagging SNPs (htSNPs). As used herein, haplotype tagging SNP means a SNP or multiple SNPs that can identify or represent a haplotype or multiple haplotypes described herein. A tagging SNP or multiple tagging SNPs can be used to identify or depict a haplotype or multiple haplotypes in a subject, and, therefore, can be used to determine a subject's susceptibility to having or developing a complement-mediated disease.

In one aspect, an A at rs35928059, a G at rs800292, a C at rs1061170, a Gat rs12144939, a G at rs7546940, a G at rs1409153, a G at rs10922153, or an A at rs698859 can indicate the subject's increased risk for having or developing age-related macular degeneration. In another aspect, an A at rs35928059, a G at rs800292, a C at rs1061170, a G at rs12144939, a G at rs7546940, a C at rs1409153, a G at rs10922153, or a T at rs698859 can indicate the subject's increased risk for having or developing age-related macular degeneration. In another aspect, an A at rs35928059, a G at rs800292, a C at rs1061170, a G at rs12144939, a G at rs7546940, a C at rs1409153, a G at rs10922153, or a C at rs698859 can indicate the subject's increased risk for having or developing age-related macular degeneration. In yet another aspect, an A at rs35928059, an A at rs800292, a T at rs1061170, a G at rs12144939, a G at rs7546940, an A at rs1409153, a T at rs10922153, or a G at rs698859 can indicate the subject's decreased risk for having or developing age-related macular degeneration. In still another aspect, an A at rs35928059, a G at rs800292, a T at rs1061170, a T at rs12144939, a G at rs7546940, an A at rs1409153, a T at rs10922153, or a G at rs698859 can indicate the subject's decreased risk for having or developing age-related macular degeneration. In another aspect, an A at rs35928059, a G at rs800292, a T at rs1061170, a T at rs12144939, an A at rs7546940, an A at rs1409153, a T at rs10922153, or an A at rs698859 can indicate the subject's decreased risk for having or developing age-related macular degeneration. In yet another aspect, an A at rs35928059, an A at rs800292, a T at rs1061170, a G at rs12144939, a G at rs7546940, a T at rs1409153, a T at rs10922153, or a C at rs698859 can indicate the subject's decreased risk for having or developing age-related macular degeneration. In still another aspect, an A at rs35928059, a G at rs800292, a T at rs1061170, a T at rs12144939, a G at rs7546940, a T at rs1409153, a T at rs10922153, or a C at rs698859 can indicate the subject's decreased risk for having or developing age-related macular degeneration. In another aspect, an A at rs35928059, a G at rs800292, a T at rs1061170, a T at rs12144939, an A at rs7546940, a T at rs1409153, a T at rs10922153, or a T at rs698859 can indicate the subject's decreased risk for having or developing age-related macular degeneration. In yet another aspect, an A at rs35928059, a G at rs800292, a T at rs1061170, a T at rs12144939, a G at rs7546940, a T at rs1409153, a T at rs10922153, or a T at rs698859 can indicate the subject's decreased risk for having or developing age-related macular degeneration.

Also described herein are methods for determining a female Caucasian subject's susceptibility to having or developing a complement-mediated disease comprising determining in the female Caucasian subject the identity of at least six SNPs in the CFH-to-F13B locus. The SNPs can be, but are not limited to: (i) rs35928059, rs800292, rs1061170, rs12144939, rs7546940, rs1409153, rs10922153, or rs698859, or (ii) a SNP in linkage disequilibrium with the SNPs of (i). In one aspect, the presence of at least six of the SNPs can indicate the subject's susceptibility for having or developing a complement-mediated disease.

In one aspect, an A at rs35928059, a G at rs800292, a C at rs1061170, a Gat rs12144939, a G at rs7546940, a G at rs1409153, a G at rs10922153, or an G at rs698859 can indicate the subject's increased risk for having or developing age-related macular degeneration. In another aspect, an A at rs35928059, a G at rs800292, a C at rs1061170, a G at rs12144939, a G at rs7546940, a G at rs1409153, a G at rs10922153, or an A at rs698859 can indicate the subject's increased risk for having or developing age-related macular degeneration. In yet another aspect, an A at rs35928059, a G at rs800292, a C at rs1061170, a G at rs12144939, a G at rs7546940, a C at rs1409153, a G at rs10922153, or a C at rs698859 can indicate the subject's increased risk for having or developing age-related macular degeneration. In still another aspect, an A at rs35928059, a G at rs800292, a C at rs1061170, a G at rs12144939, a G at rs7546940, a C at rs1409153, a G at rs10922153, or a T at rs698859 can indicate the subject's increased risk for having or developing age-related macular degeneration. In another aspect, an A at rs35928059, an A at rs800292, a T at rs1061170, a G at rs12144939, a G at rs7546940, an A at rs1409153, a T at rs10922153, or a G at rs698859 can indicate the subject's decreased risk for having or developing age-related macular degeneration. In yet another aspect, an A at rs35928059, a G at rs800292, a T at rs1061170, a T at rs12144939, a G at rs7546940, an A at rs1409153, a T at rs10922153, or a G at rs698859 can indicate the subject's decreased risk for having or developing age-related macular degeneration. In still another aspect, an A at rs35928059, a G at rs800292, a T at rs1061170, a T at rs12144939, an A at rs7546940, an A at rs1409153, a T at rs10922153, or an A at rs698859 can indicate the subject's decreased risk for having or developing age-related macular degeneration. In another aspect, an A at rs35928059, an A at rs800292, a T at rs1061170, a G at rs12144939, a G at rs7546940, a T at rs1409153, a T at rs10922153, or a C at rs698859 can indicate the subject's decreased risk for having or developing age-related macular degeneration. In yet another aspect, an A at rs35928059, a G at rs800292, a T at rs1061170, a T at rs12144939, a G at rs7546940, a T at rs1409153, a T at rs10922153, or a C at rs698859 can indicate the subject's decreased risk for having or developing age-related macular degeneration. In yet another aspect, an A at rs35928059, a G at rs800292, a T at rs1061170, a T at rs12144939, an A at rs7546940, a T at rs1409153, a T at rs10922153, or a T at rs698859 can indicate the subject's decreased risk for having or developing age-related macular degeneration. In sill another aspect, an A at rs35928059, a G at rs800292, a T at rs1061170, a T at rs12144939, a G at rs7546940, a T at rs1409153, a T at rs10922153, or a T at rs698859 can indicate the subject's decreased risk for having or developing age-related macular degeneration.

Also described herein are methods for determining a male Caucasian subject's susceptibility to having or developing a complement-mediated disease comprising determining in the male Caucasian subject the identity of at least six SNPs in the CFH-to-F13B locus. The SNPs can include, but are not limited to: (i) rs35928059, rs800292, rs1061170, rs12144939, rs7546940, rs1409153, rs10922153, or rs698859, or (ii) a SNP in linkage disequilibrium with the SNPs of (i). In one aspect, the presence of at least six of the SNPs indicates the subject's susceptibility for having or developing a complement-mediated disease.

In one aspect, an A at rs35928059, a G at rs800292, a C at rs1061170, a Gat rs12144939, a G at rs7546940, a G at rs1409153, a G at rs10922153, or an A at rs698859 can indicate the subject's increased risk for having or developing age-related macular degeneration. In another aspect, an A at rs35928059, a G at rs800292, a C at rs1061170, a G at rs12144939, a G at rs7546940, a C at rs1409153, a G at rs10922153, or a C at rs698859 can indicate the subject's increased risk for having or developing age-related macular degeneration. In yet another aspect, an A at rs35928059, a G at rs800292, a C at rs1061170, a G at rs12144939, a G at rs7546940, a C at rs1409153, a G at rs10922153, or a T at rs698859 can indicate the subject's increased risk for having or developing age-related macular degeneration. In still another aspect, an A at rs35928059, a G at rs800292, a T at rs1061170, a T at rs12144939, an A at rs7546940, an A at rs1409153, a T at rs10922153, or an A at rs698859 can indicate the subject's decreased risk for having or developing age-related macular degeneration. In another aspect, an A at rs35928059, an A at rs800292, a T at rs1061170, a G at rs12144939, a G at rs7546940, a T at rs1409153, a T at rs10922153, or a C at rs698859 can indicate the subject's decreased risk for having or developing age-related macular degeneration. In yet another aspect, an A at rs35928059, a G at rs800292, a T at rs1061170, a T at rs12144939, a G at rs7546940, a T at rs1409153, a T at rs10922153, or a T at rs698859 can indicate the subject's decreased risk for having or developing age-related macular degeneration.

Also described herein are methods for predicting progression of a complement-mediated disease in a Caucasian subject comprising determining in the Caucasian subject the identity of at least six SNPs in the CFH-to-F13B locus. The SNPs can be, but are not limited to: (i) rs35928059, rs800292, rs1061170, rs12144939, rs7546940, rs1409153, rs10922153, rs698859, or (ii) a SNP in linkage disequilibrium with the SNPs of (i). In one aspect, the presence of at least six of the SNPs can indicate the subject's risk for having or developing late-stage age-related macular degeneration.

In one aspect, an A at rs35928059, a G at rs800292, a C at rs1061170, a Gat rs12144939, a G at rs7546940, a G at rs1409153, a G at rs10922153, or an A at rs698859 can indicate the subject's increased risk for having or developing late-stage age-related macular degeneration. In another aspect, an A at rs35928059, a G at rs800292, a C at rs1061170, a G at rs12144939, a G at rs7546940, a C at rs1409153, a G at rs10922153, or a T at rs698859 can indicate the subject's increased risk for having or developing late-stage age-related macular degeneration. In yet another aspect, an A at rs35928059, a G at rs800292, a C at rs1061170, a G at rs12144939, a G at rs7546940, a C at rs1409153, a G at rs10922153, or a C at rs698859 can indicate the subject's increased risk for having or developing late-stage age-related macular degeneration. In still another aspect, an A at rs35928059, an A at rs800292, a T at rs1061170, a G at rs12144939, a G at rs7546940, an A at rs1409153, a T at rs10922153, or a G at rs698859 can indicate the subject's decreased risk for having or developing late-stage age-related macular degeneration. In another aspect, an A at rs35928059, a G at rs800292, a T at rs1061170, a T at rs12144939, a G at rs7546940, an A at rs1409153, a T at rs10922153, or a G at rs698859 can indicate the subject's decreased risk for having or developing late-stage age-related macular degeneration. In yet another aspect, an A at rs35928059, a G at rs800292, a T at rs1061170, a T at rs12144939, an A at rs7546940, an A at rs1409153, a T at rs10922153, or an A at rs698859 can indicate the subject's decreased risk for having or developing late-stage age-related macular degeneration. In still another aspect, an A at rs35928059, an A at rs800292, a T at rs1061170, a G at rs12144939, a G at rs7546940, a T at rs1409153, a T at rs10922153, or a C at rs698859 can indicate the subject's decreased risk for having or developing late-stage age-related macular degeneration. In another aspect, an A at rs35928059, a G at rs800292, a T at rs1061170, a T at rs12144939, a G at rs7546940, a T at rs1409153, a T at rs10922153, or a C at rs698859 can indicate the subject's decreased risk for having or developing late-stage age-related macular degeneration. In yet another aspect, an A at rs35928059, a G at rs800292, a T at rs1061170, a T at rs12144939, an A at rs7546940, a T at rs1409153, a T at rs10922153, or a T at rs698859 can indicate the subject's decreased risk for having or developing late-stage age-related macular degeneration. In still another aspect, an A at rs35928059, a G at rs800292, a T at rs1061170, a T at rs12144939, a G at rs7546940, a T at rs1409153, a T at rs10922153, or a T at rs698859 can indicate the subject's decreased risk for having or developing late-stage age-related macular degeneration.

Also described herein are methods for predicting progression of complement-mediated disease in a female Caucasian subject comprising determining in the female Caucasian subject the identity of at least six SNPs in the CFH-to-F13B locus. The SNPs can be, but are not limited to: (i) rs35928059, rs800292, rs1061170, rs12144939, rs7546940, rs1409153, rs10922153, or rs698859, or (ii) a SNP in linkage disequilibrium with the SNPs of (i). In one aspect, the presence of at least six of the SNPs is indicative of the subject's risk for having or developing late-stage age-related macular degeneration.

In one aspect, an A at rs35928059, a G at rs800292, a C at rs1061170, a Gat rs12144939, a G at rs7546940, a G at rs1409153, a G at rs10922153, or an G at rs698859 can indicate the subject's increased risk for having or developing late-stage age-related macular degeneration. In another aspect, an A at rs35928059, a G at rs800292, a C at rs1061170, a G at rs12144939, a G at rs7546940, a G at rs1409153, a G at rs10922153, or an A at rs698859 can indicate the subject's increased risk for having or developing late-stage age-related macular degeneration. In yet another aspect, an A at rs35928059, a G at rs800292, a C at rs1061170, a G at rs12144939, a G at rs7546940, a C at rs1409153, a G at rs10922153, or a C at rs698859 can indicate the subject's increased risk for having or developing late-stage age-related macular degeneration. In still another aspect, an A at rs35928059, a G at rs800292, a C at rs1061170, a G at rs12144939, a G at rs7546940, a C at rs1409153, a G at rs10922153, or a T at rs698859 can indicate the subject's increased risk for having or developing late-stage age-related macular degeneration. In another aspect, an A at rs35928059, an A at rs800292, a T at rs1061170, a G at rs12144939, a G at rs7546940, an A at rs1409153, a T at rs10922153, or a G at rs698859 can indicate the subject's decreased risk for having or developing late-stage age-related macular degeneration. In yet another aspect, an A at rs35928059, a G at rs800292, a T at rs1061170, a T at rs12144939, a G at rs7546940, an A at rs1409153, a T at rs10922153, or a G at rs698859 can indicate the subject's decreased risk for having or developing late-stage age-related macular degeneration. In still another aspect, an A at rs35928059, a G at rs800292, a T at rs1061170, a T at rs12144939, an A at rs7546940, an A at rs1409153, a T at rs10922153, or an A at rs698859 can indicate the subject's decreased risk for having or developing late-stage age-related macular degeneration. In another aspect, an A at rs35928059, an A at rs800292, a T at rs1061170, a G at rs12144939, a G at rs7546940, a T at rs1409153, a T at rs10922153, or a C at rs698859 can indicate the subject's decreased risk for having or developing late-stage age-related macular degeneration. In yet another aspect, an A at rs35928059, a G at rs800292, a T at rs1061170, a T at rs12144939, a G at rs7546940, a T at rs1409153, a T at rs10922153, or a C at rs698859 can indicate the subject's decreased risk for having or developing late-stage age-related macular degeneration. In still another aspect, an A at rs35928059, a G at rs800292, a T at rs1061170, a T at rs12144939, an A at rs7546940, a T at rs1409153, a T at rs10922153, or a T at rs698859 can indicate the subject's decreased risk for having or developing late-stage age-related macular degeneration. In another aspect, an A at rs35928059, a G at rs800292, a T at rs1061170, a T at rs12144939, a G at rs7546940, a T at rs1409153, a T at rs10922153, or a T at rs698859 can indicate the subject's decreased risk for having or developing late-stage age-related macular degeneration.

Also described herein are methods for predicting progression of a complement-mediated disease in a male Caucasian subject comprising determining in the male Caucasian subject the identity of at least six SNPs in the CFH-to-F13B locus. The SNPs can include, but are not limited to: (i) rs35928059, rs800292, rs1061170, rs12144939, rs7546940, rs1409153, rs10922153, or rs698859, or (ii) a SNP in linkage disequilibrium with the SNPs of (i). In one aspect, the presence of at least six of the SNPs can indicate the subject's risk for having or developing late-stage age-related macular degeneration.

In another aspect, an A at rs35928059, a G at rs800292, a C at rs1061170, a G at rs12144939, a G at rs7546940, a G at rs1409153, a G at rs10922153, or an A at rs698859 can indicate the subject's increased risk for having or developing late-stage age-related macular degeneration. In yet another aspect, an A at rs35928059, a G at rs800292, a C at rs1061170, a G at rs12144939, a G at rs7546940, a C at rs1409153, a G at rs10922153, or a C at rs698859 can indicate the subject's increased risk for having or developing late-stage age-related macular degeneration. In still another aspect, an A at rs35928059, a G at rs800292, a C at rs1061170, a G at rs12144939, a G at rs7546940, a C at rs1409153, a G at rs10922153, or a T at rs698859 can indicate the subject's increased risk for having or developing late-stage age-related macular degeneration. In another aspect, an A at rs35928059, a G at rs800292, a T at rs1061170, a T at rs12144939, an A at rs7546940, an A at rs1409153, a T at rs10922153, or an A at rs698859 can indicate the subject's decreased risk for having or developing late-stage age-related macular degeneration. In yet another aspect, an A at rs35928059, an A at rs800292, a T at rs1061170, a G at rs12144939, a G at rs7546940, a T at rs1409153, a T at rs10922153, or a C at rs698859 can indicate the subject's decreased risk for having or developing late-stage age-related macular degeneration. In still another aspect, an A at rs35928059, a G at rs800292, a T at rs1061170, a T at rs12144939, a G at rs7546940, a T at rs1409153, a T at rs10922153, or a T at rs698859 can indicate the subject's decreased risk for having or developing late-stage age-related macular degeneration.

### 4. Single SNPs

Described herein are a collection of polymorphisms and haplotypes comprised of multiple variations in the CFH-to-F13B locus. One or more of these polymorphisms or haplotypes can be associated with complement-mediated disease. Detection of these and other polymorphisms and sets of polymorphisms (e.g., haplotypes) can be useful in designing and performing diagnostic assays for complement-mediated disease. Polymorphisms and sets of polymorphisms can be detected by analysis of nucleic acids, by analysis of polypeptides encoded the CFH-to-F13B locus coding sequences (including polypeptides encoded by splice variants), by analysis of the CFH-to-F13B locus non-coding sequences, or by other means known in the art. Analysis of such polymorphisms and haplotypes can also be useful in designing prophylactic and therapeutic regimes for complement-mediated disease.

More specifically, described herein are methods for determining a Caucasian subject's susceptibility to having or developing a complement-mediated disease comprising determining in the subject the identity of one or more SNPs in the CFH-to-F13B locus. In one aspect, the the SNPs can include, but are not limited to: (i) rs1061170, rs1410996, rs2274700, rs3753395, rs403846, or rs3753396 or (ii) a SNP in linkage disequilibrium with the SNPs of (i). In another aspect, the presence of one or more of the SNPs described herein can indicate the subject's susceptibility for having or developing age-related macular degeneration.

In one aspect, an A at the rs1061170 SNP can be indicative of the subject's increased risk for having or developing a complement-mediated disease. In another aspect, an A at the rs1410996 SNP, an A at the rs2274700 SNP, a T at the rs3753395 SNP, or a G at the rs403846 SNP can be indicative of the subject's decreased risk for having or developing age-related macular degeneration. In yet another aspect, a G at the rs3753396 SNP does not indicate that the subject has an increased risk or a decreased risk for having or developing age-related macular degeneration.

Also described herein are methods for predicting progression of age-related macular degeneration in a Caucasian subject comprising determining in the subject the identity of one or more SNPs in the CFH-to-F13B locus. In one aspect, the SNPs can include, but are not limited to: (i) rs1061170, rs1410996, rs2274700, rs3753395, rs403846, or rs3753396 or (ii) a SNP in linkage disequilibrium with the SNPs of (i). In another aspect, the presence of one or more of the SNPs described herein can be indicative of the subject's risk for having or developing late-stage age-related macular degeneration.

In one aspect, an A at the rs1061170 SNP can be indicative of the subject's increased risk for having or developing late-stage age-related macular degeneration. In another aspect, an A at the rs1410996 SNP, an A at the rs2274700 SNP, a T at the rs3753395 SNP, or a G at the rs403846 SNP can be indicative of the subject's decreased risk for having or developing late-stage age-related macular degeneration. In yet another aspect, a G at the rs3753396 SNP does not indicate whether a subject has an increased risk or a decreased risk for having or developing late-stage age-related macular degeneration.

Also described herein are methods of screening for polymorphic sites in other genes that are in linkage disequilibrium (LD) with a risk, protective, or otherwise informative SNP or genetic marker described herein, including but not limited to the polymorphic sites in the CFH-to-F13B locus. These methods can involve identifying a polymorphic site in a gene that is in linkage disequilibrium with a polymorphic site in the CFH-to-F13B locus, wherein the polymorphic form of the polymorphic site in the CFH-to-F13B locus is associated with complement-mediated disease, (e.g., increased or decreased risk), and determining haplotypes in a population of individuals to indicate whether the linked polymorphic site has a polymorphic form in linkage disequilibrium with the polymorphic form of the CFH/F13B gene that correlates with the complement-mediated disease phenotype. SNPs in LD with the SNPs described herein include, but are not limited to rs10737680, rs7535263, rs10922106, rs395998, rs1329428 and rs7540032. In another aspect, SNPs in LD with the SNPs described herein can be, but are not limited to rs10489456, rs70620, rs742855, rs11799380, rs1065489, rs11582939, rs385390, rs421820, rs426736, rs370953, rs371075.

As used herein, “linkage disequilibrium” is the non-random association of alleles at two or more loci, not necessarily on the same chromosome. It is not the same as linkage, which describes the association of two or more loci on a chromosome with limited recombination between them. Linkage disequilibrium describes a situation in which some combinations of alleles or genetic markers occur more or less frequently in a population than would be expected from a random formation of haplotypes from alleles based on their frequencies. Non-random associations between polymorphisms at different loci are measured by the degree of linkage disequilibrium (LD). The level of linkage disequilibrium can be influenced by a number of factors including genetic linkage, the rate of recombination, the rate of mutation, random drift, non-random mating, and population structure. “Linkage disequilibrium” or “allelic association” thus means the non-random association of a particular allele or genetic marker with another specific allele or genetic marker more frequently than expected by chance for any particular allele frequency in the population. A marker in linkage disequilibrium with an informative marker, such as one of the CFH/F13B SNPs or haplotypes described herein can be useful in detecting susceptibility to complement-mediated disease. A SNP that is in linkage disequilibrium with a risk, protective, or otherwise informative SNP, haplotype, or genetic marker described herein can be referred to as a “proxy” or “surrogate” SNP. A proxy SNP may be in at least 50%, 60%, or 70% in linkage disequilibrium with risk, protective, or otherwise informative SNP or genetic marker described herein, and in one aspect is at least about 80%, 90%, and in another aspect 91%, 92%, 93%, 94%, 95%, 96%, 97%, 98%, 99%, or about 100% in LD with a risk, protective, or otherwise informative SNP, haplotype, or genetic marker described herein.

Publicly available databases such as the HapMap database (http://ftp.hapmap.org/ld_data/latest/) and Haploview (Barrett, J. C. et al., Bioinformatics 21, 263 (2005)) may be used to calculate linkage disequilibrium between two SNPs. The frequency of identified alleles in disease versus control populations can be determined using the methods described herein. Statistical analyses can be employed to determine the significance of a non-random association between the two SNPs (e.g., Hardy-Weinberg Equilibrium, Genotype likelihood ratio (genotype p value), Chi Square analysis, Fishers Exact test). A statistically significant non-random association between the two SNPs indicates that they are in linkage disequilibrium and that one SNP can serve as a proxy for the second SNP.

In another aspect, polymorphisms in the CFH-to-F13B locus, such as those described herein, can be used to establish physical linkage between a genetic locus associated with a trait of interest and polymorphic markers that are not associated with the trait, but are in physical proximity with the genetic locus responsible for the trait and co-segregate with it. Mapping a genetic locus associated with a trait of interest facilitates cloning the gene(s) responsible for the trait following procedures that are well-known in the art.

As used herein, “physical linkage” describes the tendency of genes, alleles, loci or genetic markers to be inherited together as a result of their location on the same chromosome. Linkage can be measured by percent recombination between the two genes, alleles, loci or genetic markers. Typically, loci occurring within a 50 centimorgan (cM) distance of each other are linked. Linked markers may occur within the same gene or gene cluster.

### 5. Deletion Tagging SNPs

As disclosed herein, are SNPs that tag the deletion status of the CFHR-3/CFHR-1 genes. Therefore, also described herein are methods for determining a Caucasian subject's susceptibility to having or developing a complement-mediated disease comprising determining in the subject the identity of a deletion tagging SNP in the CFH-to-F13B locus. The SNP can be, but is not limited to: (i) rs12144939 or (ii) a SNP in linkage disequilibrium with rs12144939. In one aspect, the presence of the SNP can indicate the subject's susceptibility for having or developing age-related macular degeneration. In another aspect, a T at the rs12144939 SNP can indicate the subject's decreased risk for having or developing age-related macular degeneration. In another aspect, also described herein are methods determining for determining a Caucasian subject's susceptibility to having or developing a complement-mediated disease comprising determining in the subject the identity of at lease one SNP that tags the deletion tagging SNPs described herein. The tagging SNPs can be, but are not limited to, rs6689009, rs35253683, rs731557, and rs60642321. In another aspect, also described herein are methods determining for determining a Caucasian subject's susceptibility to having or developing a complement-mediated disease comprising determining in the subject the identity of at lease one SNP in LD with the deletion tagging SNPs described herein. The SNPs can be, but are not limited to, rs6677604, rs16840522 and rs2019727.

Also described herein are methods for predicting progression of a complement-mediated disease in a Caucasian subject comprising determining in the subject the identity of a deletion tagging SNP in the CFH-to-F13B locus. The SNP can include, but is not limited to: (i) rs12144939 or (ii) a SNP in linkage disequilibrium with rs12144939. In one aspect, the presence of the deletion tagging SNP can indicate the subject's risk for having or developing late-stage age-related macular degeneration. In another aspect, a T at the rs12144939 SNP can indicate the subject's decreased risk for having or developing late-stage age-related macular degeneration.

### 6. Haplotype Tagging Haplotypes

In one aspect, the haplotypes described herein can be compared to haplotypes generated using a genetic variation database, for example, HapMap, in order to further refine haplotype association with complement-mediated diseases, such as AMD. The International HapMap Project, available at http://hapmap.ncbi.nlm.nih.gov/, contains over 500 SNPs in the region encompassing the CFH-to-F13B locus (Hg18 chr1: 194869137-195303491). Thus, in one aspect, genotypes from Caucasian individuals (referred to as the CEU population) can be downloaded for each of these 500 plus SNPs. Phased genotype data from the CEU set can then be downloaded, including the most informative SNPs (i.e. those that segregate in the CEU population) in this region of interest. In one aspect, Haploview (www.broadinstitute.org) can be used to construct haplotypes for the CEU dataset and their frequencies can be calculated. The overlapping SNPs obtiained from that analysis can be used as ‘tagging’ SNPs to match the case/control haplotypes described herein to their equivalent HapMap CEU haplotypes, thereby allowing the disease associations described herein to be extended to the equivalent HapMap haplotypes.

In one aspect, the methods can identify HapMap SNPs that were not included in the 51 SNP-based haplotypes described herein. These newly identified SNPs can further refine the discriminative value of the 51 SNP-based haplotypes described herein, and the association of those haplotypes with complement-mediated disease.

Described herein are methods for identifying a single nucleotide polymorphism or haplotype that tags one or more haplotypes in the CFH-to-F13B locus. The one or more haplotypes can be any haplotype described herein as being associated with having or developing a complement-mediated disease. In one aspect, the methods can comprise entering the SNP sequence of one or more CFH/F13B haplotypes into a genetic variation database and determining the identity of one or more tagging SNPs or haplotypes. In one aspect, the one or more CFH/F13B haplotypes can be H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, H1_51_A, H2_51_A, H3_51_A, H4_51_A, H5_51_A, H6_51_A, H7_51_A, H8_51_A, H9_51_A, H10_51_A, H11_51_A, H12_51_A, H13_51_A, H14_51_A, H15_51_A, H16_51_A, H17_51_A, H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof.

In another aspect, the genetic variation database can be HapMap. In yet another aspect, the one or more CFH/F13B haplotypes can be risk haplotypes, including, but not limited to, H2_62_A, H2_51_A, H1_51_B, H2_51_B, or a complement thereof. In still another aspect, the one or more CFH/F13B haplotypes can be protective haplotypes, including, but not limited to, H3_62_A, H5_62_A, H11_62_A, H3_51_A, H5_51_A, H10_51_A, H3_51_B, H5_51_B, H12_51_B, H14_51_B, or a complement thereof.

### B. METHODS OF IDENTIFYING A CAUCASIAN SUBJECT IN NEED OF TREATMENT FOR AGE-RELATED MACULAR DEGENERATION

Described herein are methods of identifying a Caucasian subject in need of treatment for complement-mediated disease comprising determining in the Caucasian subject the identity of one or more major or minor haplotypes. The haplotypes can include, but are not limited to H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, H1_51_A, H2_51_A, H3_51_A, H4_51_A, H5_51_A, H6_51_A, H7_51_A, H8_51_A, H9_51_A, H10_51_A, H11_51_A, H12_51_A, H13_51_A, H14_51_A, H15_51_A, H16_51_A, H17_51_A, H1_51_B, H2_51_B, H3_51_B, H4_51_B, H5_51_B H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof. As described herein, the presence of one or more of the haplotypes disclosed herein can indicate whether the subject is in need of treatment for a complement-mediated disease. As used herein, “treatment” can also mean prophylactic, or preventative treatment. In one aspect, the complement-mediated disease can be age-related macular degeneration (AMD). Thus, the methods described herein can be used to identify a Caucasian subject in need of treatment for age-related macular degeneration.

In one aspect, determining the identity of a certain haplotype or certain haplotypes in a subject can indicate that the subject is in need of treatment for a complement-mediated disease. Such hapltoypes can be risk haplotypes. For example, and not to be limiting, a risk haplotype can be H2_62_A, H2_51_A, H1_51_B, H2_51_B, or a complement thereof. Thus, described herein are methods for identifying a Caucasian subject in need of treatment for a complement-mediated disease comprising determining in the Caucasian subject the identity of one or more haplotypes described herein, wherein a risk haplotype can indicate that the subject is in need of treatment. In one aspect, the method of identifying a subject in need of treatment can further comprise administering a therapeutic composition to the subject identified as being in need of treatment. In another aspect, the methods described herein can be used to identify a Caucasian subject in need of more frequent screening for complement-mediated diseases, including, but not limited to, age-related macular degeneration. For example, identifying a subject with a risk haplotype can indicate that the subject needs to be more closely and frequently monitored for the development of a complement-mediated disease such as AMD.

In another aspect, determining the identity of a certain haplotype or certain haplotypes in a subject can indicate that the subject is not in need of treatment for a complement-mediated disease. Such haplotypes can be protective haplotypes. For example, and not to be limiting, a protective haplotype can be H3_62_A, H5_62_A, H11_62_A, H3_51_A, H5_51_A, H10_51_A, H3_51_B, H5_51_B, H12_51_B, H14_51_B, or a complement thereof. Thus, described herein are methods for identifying a Caucasian subject in need of treatment for a complement-mediated disease comprising determining in the Caucasian subject the identity of one or more haplotypes described herein, wherein a protective haplotype can indicate that the subject is not in need of treatment for a complement-mediated disease.

In yet another aspect, determining the identity of a certain haplotype or certain haplotypes in a subject can indicate that the subject is in need of continued screening for the development of a complement-mediated disease. Such haplotypes can be neutral haplotypes. For example, and not to be limiting, a neutral haplotype can be H1_62_A, H4_62_A, H6_62_A, H7_62_A, H8_62_A, H9_62_A, H10_62_A, H12_62_A, H13_62_A, H14_62_A, H15_62_A, H1_51_A, H4_51_A, H6_51_A, H7_51_A, H8_51_A, H9_51_A, H11_51_A, H12_51_A, H13_51_A, H14_51_A, H15_51_A, H16_51_A, H17_51_A, H4_51_B, H6_51_B, H7_51_B, H8_51_B, H9_51_B, H10_51_B, H11_51_B, H13_51_B, H15_51_B, H16_51_B, H17_51_B, H18_51_B, H19_51_B, H20_51_B, H21_51_B, H22_51_B, or a complement thereof. Thus, described herein are methods identifying a Caucasian subject in need of screening for the development of a complement-mediated disease comprising determining in the Caucasian subject the identity of one or more haplotypes described herein, wherein a neutral haplotype can indicate a subject in need of screening for the development of a complement-mediated disease. For example, and not to be limiting, identifying a subject with a neutral haplotype can indicate that the subject should be screened for the development of a complement-mediated disease, such as AMD, at an earlier age, and more often than would typically be done in the general population.

Also described herein are methods for identifying a female Caucasian subject in need of treatment for complement-mediated disease comprising determining in the female Caucasian subject the identity of one or more major or minor haplotypes. The haplotypes can include, but are not limited to H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, or a complement thereof.

In one aspect, determining the identity of a certain haplotype or certain haplotypes in a female subject can indicate that the female subject is in need of treatment for a complement-mediated disease. Such hapltoypes can be risk haplotypes. For example, and not to be limiting, a risk haplotype can be H1_62_A, H2_62_A, H1_51_B, H2_51_B, or a complement thereof. Thus, described herein are methods of identifying a female Caucasian subject in need of treatment for a complement-mediated disease comprising determining in the female Caucasian subject the identity of one or more haplotypes described herein, wherein a risk haplotype can indicate that the subject is in need of treatment. In one aspect, the method of identifying a female subject in need of treatment can further comprise administering a therapeutic composition to the female subject identified as being in need of treatment. In another aspect, the methods described herein can be used to identify a female Caucasian subject in need of more frequent screening for complement-mediated diseases, including, but not limited to, age-related macular degeneration. For example, identifying a female subject with a risk haplotype can indicate that the female subject needs to be more closely and frequently monitored for the development of a complement-mediated disease such as AMD.

In another aspect, determining the identity of a certain haplotype or certain haplotypes in a female subject can indicate that the female subject is not in need of treatment for complement-mediated disease. Such haplotypes can be protective haplotypes. For example, and not to be limiting, a protective haplotype can be H3_62_A, H5_62_A, H11_62_A, H3_51_B, H5_51_B, H12_51_B, H14_51_B, or a complement thereof. Thus, described herein are methods for identifying a female Caucasian subject in need of treatment for a complement-mediated disease comprising determining in the female Caucasian subject the identity of one or more haplotypes described herein, wherein a protective haplotype can indicate that the female subject is not in need of treatment for a complement-mediated disease.

In yet another aspect, determining the identity of a certain haplotype or certain haplotypes in a female subject can indicate that the female subject is in need of continued screening for the development of a complement-mediated disease. Such haplotypes can be neutral haplotypes. For example, and not to be limiting, a neutral haplotype can be H4_62_A, H6_62_A, H7_62_A, H8_62_A, H9_62_A, H10_62_A, H12_62_A, H13_62_A, H14_62_A, H15_62_A, H4_51_B, H6_51_B, H7_51_B, H8_51_B, H9_51_B, H10_51_B, H11_51_B, H13_51_B, H15_51_B, H16_51_B, H17_51_B, H18_51_B, H19_51_B, H20_51_B, or a complement thereof. Thus, described herein are methods identifying a female Caucasian subject in need of screening for the development of a complement-mediated disease comprising determining in the female Caucasian subject the identity of one or more haplotypes described herein, wherein a neutral haplotype can indicate a female subject in need of screening for the development of a complement-mediated disease. For example, and not to be limiting, identifying a female subject with a neutral haplotype can indicate that the female subject should be screened for the development of a complement-mediated disease, such as AMD, at an earlier age, and more often than would typically be done in the general population.

Also described herein are methods identifying a male Caucasian subject in need of treatment for complement-mediated disease comprising determining in the male Caucasian subject the identity of one or more major or minor haplotypes. The haplotypes can include, but are not limited to H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, or a complement thereof.

In one aspect, determining the identity of a certain haplotype or certain haplotypes in a male subject can indicate that the male subject is in need of treatment for a complement-mediated disease. Such hapltoypes can be risk haplotypes. For example, and not to be limiting, a risk haplotype can be H2_62_A, H1_51_B, H2_51_B, or a complement thereof. Thus, described herein are methods of identifying a male Caucasian subject in need of treatment for a complement-mediated disease comprising determining in the male Caucasian subject the identity of one or more haplotypes described herein, wherein a risk haplotype can indicate that the male subject is in need of treatment. In one aspect, the method of identifying a male subject in need of treatment can further comprise administering a therapeutic composition to the male subject identified as being in need of treatment. In another aspect, the methods described herein can be used to identify a male Caucasian subject in need of more frequent screening for complement-mediated diseases, including, but not limited to, age-related macular degeneration. For example, identifying a male subject with risk haplotype can indicate that the male subject needs to be more closely and frequently monitored for the development of a complement-mediated disease such as AMD.

In another aspect, determining the identity of a certain haplotype or certain haplotypes in a male subject can indicate that the male subject is not in need of treatment for complement-mediated disease. Such haplotypes can be protective haplotypes. For example, and not to be limiting, a protective haplotype can be H11_62_A, H3_51_B, H14_51_B, or a complement thereof. Thus, described herein are methods for identifying a male Caucasian subject in need of treatment for a complement-mediated disease comprising determining in the male Caucasian subject the identity of one or more haplotypes described herein, wherein a protective haplotype can indicate that the male subject is not in need of treatment for a complement-mediated disease.

In yet another aspect, determining the identity of a certain haplotype or certain haplotypes in a male subject can indicate that the male subject is in need of continued screening for the development of a complement-mediated disease. Such haplotypes can be neutral haplotypes. For example, and not to be limiting, a neutral haplotype can be H1_62_A, H3_62_A, H4_62_A, H5_62_A, H6_62_A, H7_62_A, H8_62_A, H9_62_A, H10_62_A, H12_62_A, H13_62_A, H14_62_A, H15_62_A H4_51_B, H5_51_B, H6_51_B, H7_51_B, H8_51_B, H9_51_B, H10_51_B, H11_51_B, H12_51_B, H13_51_B, H15_51_B, H16_51_B, H17_51_B, H18_51_B, H19_51_B, H20_51_B, H21_51_B, H22_51_B, or a complement thereof. Thus, described herein are methods for identifying a male Caucasian subject in need of screening for the development of a complement-mediated disease comprising determining in the male Caucasian subject the identity of one or more haplotypes described herein, wherein a neutral haplotype can indicate a male subject in need of screening for the development of a complement-mediated disease. For example, and not to be limiting, identifying a male subject with a neutral haplotype can indicate that the male subject should be screened for the development of a complement-mediated disease, such as AMD, at an earlier age, and more often than would typically be done in the general population.

Also described herein are methods of identifying a Caucasian subject in need of treatment for a complement-mediated disease comprising determining in the subject the identity of one or more SNPs in the CFH-to-F13B locus. The SNPs can include, but are not limited to: (i) rs1061170, rs1410996, rs2274700, rs3753395, rs403846, or rs3753396 or (ii) a SNP in linkage disequilibrium with the SNPs of (i). In one aspect, the presence of one or more of the SNPs can indicate whether the subject is in need of treatment for a complement-mediated disease. In one aspect, an A at the rs1061170 SNP can indicate the subject is in need of treatment for a complement-mediated disease. In another aspect, an A at the rs1410996 SNP, an A at the rs2274700 SNP, a T at the rs3753395 SNP, or a G at the rs403846 SNP can indicate the subject is not in need of treatment for a complement-mediated disease. In yet another aspect, a G at the rs3753396 SNP can indicate the subject may be in need of treatment for a complement-mediated disease.

Also described herein are methods of identifying a Caucasian subject in need of treatment for a complement-mediated disease comprising determining in the subject the identity of a deletion tagging SNP in the CFH-to-F13B locus. The SNP can include, but is not limited to: (i) rs12144939 or (ii) a SNP in linkage disequilibrium with rs12144939. In one aspect, the presence of one or more of the SNPs can indicate whether the subject is in need of treatment for age-related macular degeneration. In another aspect, a T at the rs12144939 SNP can indicate the subject is not in need of treatment for age-related macular degeneration.

Also described herein are methods for identifying a Caucasian subject in need of treatment for age-related macular degeneration comprising determining in the Caucasian subject the identity of at least six SNPs in the CFH-to-F13B locus. The SNPs can include, but are not limited to: (i) rs35928059, rs800292, rs1061170, rs12144939, rs7546940, rs1409153, rs10922153, or rs698859, or (ii) a SNP in linkage disequilibrium with the SNPs of (i). In one aspect, the presence of at least six of the SNPs can indicate whether the subject is in need of treatment for age-related macular degeneration.

In one aspect, an A at rs35928059, a G at rs800292, a C at rs1061170, a G at rs12144939, a G at rs7546940, a G at rs1409153, a G at rs10922153, or an A at rs698859 can indicate the subject is in need of treatment for age-related macular degeneration. In another aspect, an A at rs35928059, a G at rs800292, a C at rs1061170, a G at rs12144939, a G at rs7546940, a C at rs1409153, a G at rs10922153, or a T at rs698859 can indicate the subject is in need of treatment for age-related macular degeneration. In yet another aspect, an A at rs35928059, a G at rs800292, a C at rs1061170, a G at rs12144939, a G at rs7546940, a C at rs1409153, a G at rs10922153, or a C at rs698859 can indicate the subject is in need of treatment for age-related macular degeneration. In still another aspect, an A at rs35928059, an A at rs800292, a T at rs1061170, a G at rs12144939, a G at rs7546940, an A at rs1409153, a T at rs10922153, or a G at rs698859 can indicate the subject is not in need of treatment for age-related macular degeneration. In another aspect, an A at rs35928059, a G at rs800292, a T at rs1061170, a T at rs12144939, a G at rs7546940, an A at rs1409153, a T at rs10922153, or a G at rs698859 can indicate the subject is not in need of treatment for age-related macular degeneration. In yet another aspect, an A at rs35928059, a G at rs800292, a T at rs1061170, a T at rs12144939, an A at rs7546940, an A at rs1409153, a T at rs10922153, or an A at rs698859 can indicate the subject is not in need of treatment for age-related macular degeneration. In still another aspect, an A at rs35928059, an A at rs800292, a T at rs1061170, a G at rs12144939, a G at rs7546940, a T at rs1409153, a T at rs10922153, or a C at rs698859 can indicate the subject is not in need of treatment for age-related macular degeneration. In another aspect, an A at rs35928059, a G at rs800292, a T at rs1061170, a T at rs12144939, a G at rs7546940, a T at rs1409153, a T at rs10922153, or a C at rs698859 can indicate the subject is not in need of treatment for age-related macular degeneration. In yet another aspect, an A at rs35928059, a G at rs800292, a T at rs1061170, a T at rs12144939, an A at rs7546940, a T at rs1409153, a T at rs10922153, or a T at rs698859 can indicate the subject is not in need of treatment for age-related macular degeneration. In still another aspect, an A at rs35928059, a G at rs800292, a T at rs1061170, a T at rs12144939, a G at rs7546940, a T at rs1409153, a T at rs10922153, or a T at rs698859 can indicate the subject is not in need of treatment for age-related macular degeneration.

Also described herein are methods of identifying a female Caucasian subject in need of treatment for a complement-mediated disease comprising determining in the female Caucasian subject the identity of at least six SNPs in the CFH-to-F13B locus. The SNPs can be, but are not limited to: (i) rs35928059, rs800292, rs1061170, rs12144939, rs7546940, rs1409153, rs10922153, or rs698859, or (ii) a SNP in linkage disequilibrium with the SNPs of (i). In one aspect, the presence of at least six of the SNPs can indicate whether the subject is in need of treatment for age-related macular degeneration.

In one aspect, an A at rs35928059, a G at rs800292, a C at rs1061170, a G at rs12144939, a G at rs7546940, a G at rs1409153, a G at rs10922153, or an G at rs698859 can indicate the subject is in need of treatment for age-related macular degeneration. In another aspect, an A at rs35928059, a G at rs800292, a C at rs1061170, a G at rs12144939, a G at rs7546940, a G at rs1409153, a G at rs10922153, or an A at rs698859 can indicate the subject is in need of treatment for age-related macular degeneration. In yet another aspect, an A at rs35928059, a G at rs800292, a C at rs1061170, a G at rs12144939, a G at rs7546940, a C at rs1409153, a G at rs10922153, or a C at rs698859 can indicate the subject is in need of treatment for age-related macular degeneration. In still another aspect, an A at rs35928059, a G at rs800292, a C at rs1061170, a G at rs12144939, a G at rs7546940, a C at rs1409153, a G at rs10922153, or a T at rs698859 can indicate the subject is in need of treatment for age-related macular degeneration. In another aspect, an A at rs35928059, an A at rs800292, a T at rs1061170, a G at rs12144939, a G at rs7546940, an A at rs1409153, a T at rs10922153, or a G at rs698859 can indicate the subject is not in need of treatment for age-related macular degeneration. In still another aspect, an A at rs35928059, a G at rs800292, a T at rs1061170, a T at rs12144939, a G at rs7546940, an A at rs1409153, a T at rs10922153, or a G at rs698859 can indicate the subject is not in need of treatment for age-related macular degeneration. In another aspect, an A at rs35928059, a G at rs800292, a T at rs1061170, a T at rs12144939, an A at rs7546940, an A at rs1409153, a T at rs10922153, or an A at rs698859 can indicate the subject is not in need of treatment for age-related macular degeneration. In yet another aspect, an A at rs35928059, an A at rs800292, a T at rs1061170, a G at rs12144939, a G at rs7546940, a T at rs1409153, a T at rs10922153, or a C at rs698859 can indicate the subject is not in need of treatment for age-related macular degeneration. In still another aspect, an A at rs35928059, a G at rs800292, a T at rs1061170, a T at rs12144939, a G at rs7546940, a T at rs1409153, a T at rs10922153, or a C at rs698859 can indicate the subject is not in need of treatment for age-related macular degeneration. In still another aspect, an A at rs35928059, a G at rs800292, a T at rs1061170, a T at rs12144939, an A at rs7546940, a T at rs1409153, a T at rs10922153, or a T at rs698859 can indicate the subject is not in need of treatment for age-related macular degeneration. In another aspect, an A at rs35928059, a G at rs800292, a T at rs1061170, a T at rs12144939, a G at rs7546940, a T at rs1409153, a T at rs10922153, or a T at rs698859 can indicate the subject is not in need of treatment for age-related macular degeneration.

Also described herein are methods of identifying a male Caucasian subject in need of treatment for a complement-mediated disease comprising determining in the male Caucasian subject the identity of at least six SNPs in the CFH-to-F13B locus. The SNPs can be, but are not limited to: (i) rs35928059, rs800292, rs1061170, rs12144939, rs7546940, rs1409153, rs10922153, or rs698859, or (ii) a SNP in linkage disequilibrium with the SNPs of (i). In one aspect, the presence of at least six of the SNPs can indicate whether the subject is in need of treatment for age-related macular degeneration.

In one aspect, an A at rs35928059, a G at rs800292, a C at rs1061170, a G at rs12144939, a G at rs7546940, a G at rs1409153, a G at rs10922153, or an A at rs698859 can indicate the subject is in need of treatment for age-related macular degeneration. In another aspect, an A at rs35928059, a G at rs800292, a C at rs1061170, a G at rs12144939, a G at rs7546940, a C at rs1409153, a G at rs10922153, or a C at rs698859 can indicate the subject is in need of treatment for age-related macular degeneration. In yet another aspect, an A at rs35928059, a G at rs800292, a C at rs1061170, a G at rs12144939, a G at rs7546940, a C at rs1409153, a G at rs10922153, or a T at rs698859 can indicate the subject is in need of treatment for age-related macular degeneration. In still another aspect, an A at rs35928059, a G at rs800292, a T at rs1061170, a T at rs12144939, an A at rs7546940, an A at rs1409153, a T at rs10922153, or an A at rs698859 can indicate the subject is not in need of treatment for age-related macular degeneration. In another aspect, an A at rs35928059, an A at rs800292, a T at rs1061170, a G at rs12144939, a G at rs7546940, a T at rs1409153, a T at rs10922153, or a C at rs698859 can indicate the subject is not in need of treatment for age-related macular degeneration. In yet another aspect, an A at rs35928059, a G at rs800292, a T at rs1061170, a T at rs12144939, a G at rs7546940, a T at rs1409153, a T at rs10922153, or a T at rs698859 can indicate the subject is not in need of treatment for age-related macular degeneration.

### 1. Methods of Identifying Patients for Clinical Trials

Furthermore, described herein are methods of identifying a Caucasian subject that is appropriate for a complement-mediated disease clinical trial comprising determining in the Caucasian subject the identity of one or more major or minor haplotypes. The haplotypes can include, but are not limited to H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, H1_51_A, H2_51_A, H3_51_A, H4_51_A, H5_51_A, H6_51_A, H7_51_A, H8_51_A, H9_51_A, H10_51_A, H11_51_A, H12_51_A, H13_51_A, H14_51_A, H15_51_A, H16_51_A, H17_51_A, H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof. As described herein, the presence of one or more of the haplotypes disclosed herein can indicate whether the subject is appropriate for a complement-mediated disease clinical trial. In one aspect, the complement-mediated disease can be age-related macular degeneration (AMD), and the clinical trial can be an age-related macular degeneration clinical trial. Thus, the methods described herein can be used to identify a Caucasian subject that is appropriate for an age-related macular degeneration clinical trial. As used herein, clinical trial means an experimental study conducted to evaluate the effectiveness and safety of a treatment or medical device by monitoring their effects on a subject or group of subjects. In one aspect, the clinical trial can be conducted to evaluate the effectiveness and safety of medications for a complement-mediated disease, for example, AMD.

In one aspect, determining the identity of a certain haplotype or certain haplotypes in a subject can indicate that the subject is appropriate for a complement-mediated disease clinical trial. Such hapltoypes can be risk haplotypes. For example, and not to be limiting, a risk haplotype can be H2_62_A, H2_51_A, H1_51_B, H2_51_B, or a complement thereof. Thus, described herein are methods for identifying a Caucasian subject that is appropriate for a complement-mediated disease clinical trial comprising determining in the Caucasian subject the identity of one or more haplotypes described herein, wherein a risk haplotype can indicate that the subject is appropriate for the clinical trial. In one aspect, the method of identifying a subject for the clinical trial can further comprise administering a therapeutic composition to the subject identified as being appropriate for the clinical trial.

In another aspect, determining the identity of a certain haplotype or certain haplotypes in a subject can indicate that the subject is not appropriate for a complement-mediated disease clinical trial. Such haplotypes can be protective haplotypes. For example, and not to be limiting, a protective haplotype can be H3_62_A, H5_62_A, H11_62_A, H3_51_A, H5_51_A, H10_51_A, H3_51_B, H5_51_B, H12_51_B, H14_51_B, or a complement thereof. Thus, described herein are methods for identifying a Caucasian subject that is appropriate for a complement-mediated disease clinical trial comprising determining in the Caucasian subject the identity of one or more haplotypes described herein, wherein a protective haplotype can indicate that the subject is not appropriate for a complement-mediated disease clinical trial. For example, a subject identified to have a protective haplotype described herein can be excluded from participation in a clinical trial designed to evaluate a treatment for a complement-mediated disease.

In yet another aspect, determining the identity of a certain haplotype or certain haplotypes in a subject can indicate that the subject may be appropriate for a complement-mediated disease clinical trial. Such haplotypes can be neutral haplotypes. For example, and not to be limiting, a neutral haplotype can be H1_62_A, H4_62_A, H6_62_A, H7_62_A, H8_62_A, H9_62_A, H10_62_A, H12_62_A, H13_62_A, H14_62_A, H15_62_A, H1_51_A, H4_51_A, H6_51_A, H7_51_A, H8_51_A, H9_51_A, H11_51_A, H12_51_A, H13_51_A, H14_51_A, H15_51_A, H16_51_A, H17_51_A, H4_51_B, H6_51_B, H7_51_B, H8_51_B, H9_51_B, H10_51_B, H11_51_B, H13_51_B, H15_51_B, H16_51_B, H17_51_B, H18_51_B, H19_51_B, H20_51_B, H21_51_B, H22_51_B, or a complement thereof. Thus, described herein are methods of identifying a Caucasian subject appropriate for a complement-mediated disease clinical trial comprising determining in the Caucasian subject the identity of one or more haplotypes described herein, wherein a neutral haplotype can indicate a subject that may be appropriate for a complement-mediated disease clinical trial.

Furthermore, described herein are methods of identifying a female Caucasian subject that is appropriate for a complement-mediated disease clinical trial comprising determining in the female Caucasian subject the identity of one or more major or minor haplotypes. The haplotypes can include, but are not limited to H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, or a complement thereof. As described herein, the presence of one or more of the haplotypes disclosed herein can indicate whether the female subject is appropriate for a complement-mediated disease clinical trial. In one aspect, the complement-mediated disease can be age-related macular degeneration (AMD), and the clinical trial can be an age-related macular degeneration clinical trial. Thus, the methods described herein can be used to identify a female Caucasian subject that is appropriate for an age-related macular degeneration clinical trial.

In one aspect, determining the identity of a certain haplotype or certain haplotypes in a female subject can indicate that the female subject is appropriate for a complement-mediated disease clinical trial. Such hapltoypes can be risk haplotypes. For example, and not to be limiting, a risk haplotype can be H1_62_A, H2_62_A, H1_51_B, H2_51_B, or a complement thereof. Thus, described herein are methods for identifying a female Caucasian subject that is appropriate for a complement-mediated disease clinical trial comprising determining in the female Caucasian subject the identity of one or more haplotypes described herein, wherein a risk haplotype can indicate that the female subject is appropriate for the clinical trial. In one aspect, the method of identifying a female subject for the clinical trial can further comprise administering a therapeutic composition to the subject identified as being appropriate for the clinical trial.

In another aspect, determining the identity of a certain haplotype or certain haplotypes in a female subject can indicate that the female subject is not appropriate for a complement-mediated disease clinical trial. Such haplotypes can be protective haplotypes. For example, and not to be limiting, a protective haplotype can be H3_62_A, H5_62_A, H11_62_A, H3_51_B, H5_51_B, H12_51_B, H14_51_B, or a complement thereof. Thus, described herein are methods for identifying a female Caucasian subject that is appropriate for a complement-mediated disease clinical trial comprising determining in the female Caucasian subject the identity of one or more haplotypes described herein, wherein a protective haplotype can indicate that the female subject is not appropriate for a complement-mediated disease clinical trial. For example, a female subject identified to have a protective haplotype described herein can be excluded from participation in a clinical trial designed to evaluate a treatment for a complement-mediated disease.

In yet another aspect, determining the identity of a certain haplotype or certain haplotypes in a female subject can indicate that the subject may be appropriate for a complement-mediated disease clinical trial. Such haplotypes can be neutral haplotypes. For example, and not to be limiting, a neutral haplotype can be H4_62_A, H6_62_A, H7_62_A, H8_62_A, H9_62_A, H10_62_A, H12_62_A, H13_62_A, H14_62_A, H15_62_A, H4_51_B, H6_51_B, H7_51_B, H8_51_B, H9_51_B, H10_51_B, H11_51_B, H13_51_B, H15_51_B, H16_51_B, H17_51_B, H18_51_B, H19_51_B, H20_51_B, or a complement thereof. Thus, described herein are methods identifying a female Caucasian subject appropriate for a complement-mediated disease clinical trial comprising determining in the female Caucasian subject the identity of one or more haplotypes described herein, wherein a neutral haplotype can indicate a female that may be appropriate for a complement-mediated disease clinical trial.

Furthermore, described herein are methods of identifying a male Caucasian subject that is appropriate for a complement-mediated disease clinical trial comprising determining in the male Caucasian subject the identity of one or more major or minor haplotypes. The haplotypes can include, but are not limited to H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, or a complement thereof. As described herein, the presence of one or more of the haplotypes disclosed herein can indicate whether the male subject is appropriate for a complement-mediated disease clinical trial. In one aspect, the complement-mediated disease can be age-related macular degeneration (AMD), and the clinical trial can be an age-related macular degeneration clinical trial. Thus, the methods described herein can be used to identify a male Caucasian subject that is appropriate for an age-related macular degeneration clinical trial.

In one aspect, determining the identity of a certain haplotype or certain haplotypes in a male subject can indicate that the male subject is appropriate for a complement-mediated disease clinical trial. Such hapltoypes can be risk haplotypes. For example, and not to be limiting, a risk haplotype can be H2_62_A, H1_51_B, H2_51_B, or a complement thereof. Thus, described herein are methods for identifying a male Caucasian subject that is appropriate for a complement-mediated disease clinical trial comprising determining in the male Caucasian subject the identity of one or more haplotypes described herein, wherein a risk haplotype can indicate that the male subject is appropriate for the clinical trial. In one aspect, the method of identifying a male subject for the clinical trial can further comprise administering a therapeutic composition to the male subject identified as being appropriate for the clinical trial.

In another aspect, determining the identity of a certain haplotype or certain haplotypes in a male subject can indicate that the male subject is not appropriate for a complement-mediated disease clinical trial. Such haplotypes can be protective haplotypes. For example, and not to be limiting, a protective haplotype can be H11_62_A, H3_51_B, H14_51_B, or a complement thereof. Thus, described herein are methods for identifying a male Caucasian subject that is appropriate for a complement-mediated disease clinical trial comprising determining in the male Caucasian subject the identity of one or more haplotypes described herein, wherein a protective haplotype can indicate that the male subject is not appropriate for a complement-mediated disease clinical trial. For example, a male subject identified to have a protective haplotype described herein can be excluded from participation in a clinical trial designed to evaluate a treatment for a complement-mediated disease.

In yet another aspect, determining the identity of a certain haplotype or certain haplotypes in a male subject can indicate that the subject may be appropriate for a complement-mediated disease clinical trial. Such haplotypes can be neutral haplotypes. For example, and not to be limiting, a neutral haplotype can be H1_62_A, H3_62_A, H4_62_A, H5_62_A, H6_62_A, H7_62_A, H8_62_A, H9_62_A, H10_62_A, H12_62_A, H13_62_A, H14_62_A, H15_62_A H4_51_B, H5_51_B, H6_51_B, H7_51_B, H8_51_B, H9_51_B, H10_51_B, H11_51_B, H12_51_B, H13_51_B, H15_51_B, H16_51_B, H17_51_B, H18_51_B, H19_51_B, H20_51_B, H21_51_B, H22_51_B, or a complement thereof. Thus, described herein are methods of identifying a male Caucasian subject appropriate for a complement-mediated disease clinical trial comprising determining in the male Caucasian subject the identity of one or more haplotypes described herein, wherein a neutral haplotype can indicate a male that may be appropriate for a complement-mediated disease clinical trial.

Also described herein are methods of identifying a Caucasian subject appropriate for complement-mediated disease clinical trial comprising determining in the subject the identity of one or more SNPs in the CFH-to-F13B locus. The SNPs can be, but are not limited to: (i) rs1061170, rs1410996, rs2274700, rs3753395, rs403846, or rs3753396 or (ii) a SNP in linkage disequilibrium with the SNPs of (i). In one aspect, the presence of one or more of the SNPs described herein can indicate whether the subject is appropriate for the clinical trial. In one aspect, an A at the rs1061170 SNP can indicate the subject is appropriate for the clinical trial. In another aspect, an A at the rs1410996 SNP, an A at the rs2274700 SNP, a T at the rs3753395 SNP, or a G at the rs403846 SNP can indicate the subject is not appropriate for the clinical trial. Thus, for example, when it is determined that a Caucasian subject has an A at the rs1410996 SNP, an A at the rs2274700 SNP, a T at the rs3753395 SNP, or a G at the rs403846 SNP, the subject can be excluded from a complement-mediated disease clinical trial. In yet another aspect, a G at the rs3753396 SNP can indicate the subject may be appropriate for the clinical trial.

Also described herein are methods of identifying a Caucasian subject appropriate for an a complement-mediated disease clinical trial comprising determining in the subject the identity of a deletion tagging SNP in the CFH-to-F13B locus. The SNP can include, but is not limited to: (i) rs12144939 or (ii) a SNP in linkage disequilibrium with rs12144939. In one aspect, the presence of one or more of the SNPs can indicate whether the subject is appropriate for the clinical trial. In another aspct, a T at the rs12144939 SNP can indicate the subject is not appropriate for the clinical trial.

Also described herein are methods of identifying a Caucasian subject appropriate for a complement-mediated disease clinical trial comprising determining in the Caucasian subject the identity of at least six SNPs in the CFH-to-F13B locus. The SNPs can be, but are not limited to: (i) rs35928059, rs800292, rs1061170, rs12144939, rs7546940, rs1409153, rs10922153, or rs698859, or (ii) a SNP in linkage disequilibrium with the SNPs of (i). In one aspect, the presence of at least six of the SNPs can indicate whether the subject is appropriate for the clinical trial.

In another aspect, an A at rs35928059, a G at rs800292, a C at rs1061170, a G at rs12144939, a G at rs7546940, a G at rs1409153, a G at rs10922153, or an A at rs698859 can indicate the subject is appropriate for the clinical trial. In yet another aspect, an A at rs35928059, a G at rs800292, a C at rs1061170, a G at rs12144939, a G at rs7546940, a C at rs1409153, a G at rs10922153, or a T at rs698859 can indicate the subject is appropriate for the clinical trial. In still another aspect, an A at rs35928059, a G at rs800292, a C at rs1061170, a G at rs12144939, a G at rs7546940, a C at rs1409153, a G at rs10922153, or a C at rs698859 can indicate the subject is appropriate for the clinical trial. In another aspect, an A at rs35928059, an A at rs800292, a T at rs1061170, a G at rs12144939, a G at rs7546940, an A at rs1409153, a T at rs10922153, or a G at rs698859 can indicate the subject is not appropriate for the clinical trial. In yet another aspect, an A at rs35928059, a G at rs800292, a T at rs1061170, a Tat rs12144939, a G at rs7546940, an A at rs1409153, a Tat rs10922153, or a G at rs698859 can indicate the subject is not appropriate for the clinical trial. In yet another aspect, an A at rs35928059, a G at rs800292, a T at rs1061170, a T at rs12144939, an A at rs7546940, an A at rs1409153, a T at rs10922153, or an A at rs698859 can indicate the subject is not appropriate for the clinical trial. In still another aspect, an A at rs35928059, an A at rs800292, a T at rs1061170, a G at rs12144939, a G at rs7546940, a T at rs1409153, a T at rs10922153, or a C at rs698859 can indicate the subject is not appropriate for the clinical trial. In another aspect, an A at rs35928059, a G at rs800292, a T at rs1061170, a T at rs12144939, a G at rs7546940, a T at rs1409153, a T at rs10922153, or a C at rs698859 can indicate the subject is not appropriate for the clinical trial. In yet another aspect, an A at rs35928059, a G at rs800292, a T at rs1061170, a T at rs12144939, an A at rs7546940, a T at rs1409153, a T at rs10922153, or a T at rs698859 can indicate the subject is not appropriate for the clinical trial. In still another aspect, an A at rs35928059, a G at rs800292, a T at rs1061170, a T at rs12144939, a G at rs7546940, a T at rs1409153, a T at rs10922153, or a T at rs698859 can indicate the subject is not appropriate for the clinical trial.

Also described herein are methods of identifying a female Caucasian subject appropriate for a complement-mediated disease clinical trial comprising determining in the female Caucasian subject the identity of at least six SNPs in the CFH-to-F13B locus. The SNPs can include, but are not limited to: (i) rs35928059, rs800292, rs1061170, rs12144939, rs7546940, rs1409153, rs10922153, or rs698859, or (ii) a SNP in linkage disequilibrium with the SNPs of (i). In one aspect, the presence of at least six of the SNPs can indicate whether the subject is appropriate for the clinical trial.

In another aspect, an A at rs35928059, a G at rs800292, a C at rs1061170, a G at rs12144939, a G at rs7546940, a G at rs1409153, a G at rs10922153, or an G at rs698859 can indicate the subject is appropriate for the clinical trial. In yet another aspect, an A at rs35928059, a G at rs800292, a C at rs1061170, a G at rs12144939, a G at rs7546940, a G at rs1409153, a G at rs10922153, or an A at rs698859 can indicate the subject is appropriate for the clinical trial. In still another aspect, an A at rs35928059, a G at rs800292, a C at rs1061170, a G at rs12144939, a G at rs7546940, a C at rs1409153, a G at rs10922153, or a C at rs698859 can indicate the subject is appropriate for the clinical trial. In another aspect, an A at rs35928059, a G at rs800292, a C at rs1061170, a G at rs12144939, a G at rs7546940, a C at rs1409153, a G at rs10922153, or a T at rs698859 can indicate the subject is appropriate for the clinical trial. In yet another aspect, an A at rs35928059, an A at rs800292, a T at rs1061170, a G at rs12144939, a G at rs7546940, an A at rs1409153, a T at rs10922153, or a G at rs698859 can indicate the subject is not appropriate for the clinical trial. In still another aspect, an A at rs35928059, a G at rs800292, a T at rs1061170, a T at rs12144939, a G at rs7546940, an A at rs1409153, a T at rs10922153, or a G at rs698859 can indicate the subject is not appropriate for the clinical trial. In another aspect, an A at rs35928059, a G at rs800292, a T at rs1061170, a T at rs12144939, an A at rs7546940, an A at rs1409153, a T at rs10922153, or an A at rs698859 can indicate the subject is not appropriate for the clinical trial. In yet another aspect, an A at rs35928059, an A at rs800292, a T at rs1061170, a G at rs12144939, a G at rs7546940, a T at rs1409153, a T at rs10922153, or a C at rs698859 can indicate the subject is not appropriate for the clinical trial. In still another aspect, an A at rs35928059, a G at rs800292, a T at rs1061170, a T at rs12144939, a G at rs7546940, a T at rs1409153, a T at rs10922153, or a C at rs698859 can indicate the subject is not appropriate for the clinical trial. In another aspect, an A at rs35928059, a G at rs800292, a T at rs1061170, a T at rs12144939, an A at rs7546940, a T at rs1409153, a T at rs10922153, or a T at rs698859 can indicate the subject is not appropriate for the clinical trial. In yet another aspect, an A at rs35928059, a G at rs800292, a T at rs1061170, a T at rs12144939, a G at rs7546940, a T at rs1409153, a T at rs10922153, or a T at rs698859 can indicate the subject is not appropriate for the clinical trial.

Also described herein are methods of identifying a male Caucasian subject appropriate for a complement-mediated disease clinical trial comprising determining in the male Caucasian subject the identity of at least six SNPs in the CFH-to-F13B locus. The SNPs can be, but are not limited to: (i) rs35928059, rs800292, rs1061170, rs12144939, rs7546940, rs1409153, rs10922153, or rs698859, or (ii) a SNP in linkage disequilibrium with the SNPs of (i). In one aspect, the presence of at least six of the SNPs can indicate whether the subject is appropriate for the clinical trial.

In another aspect, an A at rs35928059, a G at rs800292, a C at rs1061170, a G at rs12144939, a G at rs7546940, a G at rs1409153, a G at rs10922153, or an A at rs698859 can indicate the subject is appropriate for the clinical trial. In another aspect, an A at rs35928059, a G at rs800292, a C at rs1061170, a G at rs12144939, a G at rs7546940, a C at rs1409153, a G at rs10922153, or a C at rs698859 can indicate the subject is appropriate for the clinical trial. In still another aspect, an A at rs35928059, a G at rs800292, a C at rs1061170, a G at rs12144939, a G at rs7546940, a C at rs1409153, a G at rs10922153, or a T at rs698859 can indicate the subject is appropriate for the clinical trial. In another aspect, an A at rs35928059, a G at rs800292, a T at rs1061170, a T at rs12144939, an A at rs7546940, an A at rs1409153, a T at rs10922153, or an A at rs698859 can indicate the subject is not appropriate for the clinical trial. In yet another aspect, an A at rs35928059, an A at rs800292, a T at rs1061170, a G at rs12144939, a G at rs7546940, a T at rs1409153, a T at rs10922153, or a C at rs698859 can indicate the subject is not appropriate for the clinical trial. In still another aspect, an A at rs35928059, a G at rs800292, a T at rs1061170, a T at rs12144939, a G at rs7546940, a T at rs1409153, a T at rs10922153, or a T at rs698859 can indicate the subject is not appropriate for the clinical trial.

Also described herein are methods of enhancing clinical trials comprising choosing appropriate patient populations for those clinical trials. In one aspect, the methods can be used to ensure patients are identified to participate in clinical trials based upon whether or not they are responsive to the experimental therapeutic agent or agents being studied. The methods can comprise monitoring the condition of subjects receiving treatment for a complement-mediated disease such as AMD. A successful treatment outcome can be indicated by return of complement pathway activity, such as expression level, biochemical activity (e.g., enzymatic activity of a complement component), or serum auto antibodies against complement pathway associated molecules, from abnormal levels to or toward normal levels. In one aspect, the methods can comprise measuring an initial value for the level of abnormal activity (e.g., abnormal presence of an autoantibody, or abnormal levels of complement pathway molecules) before the subject has received treatment. Repeat measurements can then be made over a period of time. For example, and not to be limiting, that period of time can be about 1 day, 2 days, 5 days, 1 week, 2 weeks, 3 weeks, 1 month, 2 months, 3 months, 4 months, 6 months, 9 months, 1 year, or greater than 1 year. If the initial level is elevated relative to the mean level in a control population, a significant reduction in level in subsequent measurements can indicate a positive treatment outcome. Likewise, if the initial level of a measure marker is reduced relative to the mean in a control population, a significant increase in measured levels relative to the initial level can signal a positive treatment outcome. Subsequently measured levels are considered to have changed significantly relative to initial levels if a subsequent measured level differs by more than one standard deviation from the mean of repeat measurements of the initial level. If monitoring reveals a positive treatment outcome, that indicates a patient that can be chosen to participate in a clinical trial for that particular therapeutic agent or agents. If monitoring reveals a negative treatment outcome, that indicates a patient that should not be chosen to participate in a clinical trial for that particular therapeutic agent or agents.

### C. KITS

Also described herein are kits for utilizing the methods described herein. The kits described herein can comprise an assay or assays for detecting one or more haplotypes in a nucleic acid sample of a subject, wherein the one or more haplotypes are the haplotypes described herein. For example, and not to be limiting, the one or more haplotypes can be H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, H1_51_A, H2_51_A, H3_51_A, H4_51_A, H5_51_A, H6_51_A, H7_51_A, H8_51_A, H9_51_A, H10_51_A, H11_51_A, H12_51_A, H13_51_A, H14_51_A, H15_51_A, H16_51_A, H17_51_A, H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof. In one aspect, the one or more haplotypes can be the haplotype tagging haplotypes described herein as well as the haplotype tagging haplotypes shown in FIG. 41.

The kits described herein can further comprise amplification reagents for amplifying the CHF-to-F13B locus, or any of the genes or nucleic acids described herein.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Patient Cohorts

DNA samples derived from well-characterized cohorts of study patients with and without AMD were used for the various studies described herein (see Abrera-Abeleda et al 2006; Allikmets et al 2009; Baird et al 2006; DeAngelis et al 2008; Gold et al 2006; Hageman et al, 2005, 2006, 2011; Lim et al 2012; Robman et al 2010; Wickremasinghe et al 2011; Zhang 2008). These collections consist of over 5,000 DNA samples from Caucasian patients with and without AMD. All cohorts were case-control cohorts, with the exception of two sib-pair cohorts, one highly discordant and one both discordant and concordant (657 subjects in 284 families). Index patients in the sib-pair cohort were aged 50 years or older and had CNV in at least one eye, as defined by sub-retinal hemorrhage, fibrosis or fluorescein angiographic presence of neovascularization documented at the time or, or prior to, enrolment in the study. The unaffected siblings had normal maculas at an age older than that at which the index patient was first diagnosed with CNV. All individuals in both the case-control and sib-pair cohorts were of European-American descent, over the age of sixty, and matched for age and ethnicity. Patients were examined and photographed by trained ophthalmologists. Urine, serum, plasma, DNA and extensive medical and ophthalmological data were available for the majority of these patients. Stereo fundus photographs were graded according to standardized classification systems as described in the references noted above. A large proportion of the samples in all cohorts were genotyped for variants and SNPs in the CFH to F13B locus, and other genes, as described herein.

DNA samples from several additional cohorts were also available: 1) early onset drusen cases (EOD) (103); 2) Caucasian newborns (1,870); 3) MPGN II (65); atypical hemolytic uremic syndrome (aHUS) (211); 4) glaucoma (330); 5) Alzheimer (1,214); 6) 45 families from the Utah Genetic Reference Project (UGRP); and 7) individuals from the Human Diversity Panels. Moreover, numerous other systemic and ocular diseases were represented in the collective set of samples.

### Example 2: Determination of Deletion Status and Extended Haplotypes Spanning the Chromosome 1q32 CFH-to-F13B Locus; Discovery Phase

2.1 Background

Recent genetic studies that have shown an association between age-related macular degeneration (AMD) and specific haplotypes of the complement factor H gene (CFH), which lies within the “regulators of complement activation” (RCA) gene cluster on chromosome 1q25-q31 (Hageman et al 2005). Although the understanding of nucleotide polymorphisms in loci associated with AMD has increased, characterization of the disease-associated CFH risk and protective haplotypes has not yet been assessed fully. Recent reports suggest that risk variants of the CFH gene in the Japanese and Chinese populations are either not associated with AMD, differ substantially from the Caucasian population and/or that the risk haplotype occurs at a substantially lower frequency (Lau et al 2006; Gotoh et al 2006).

Five CFH-related genes (CFHR1, CFHR2, CFHR3, CFHR4 and CFHR5), one CFHR pseudogene (LOC100289145) and one additional gene containing complement SCR domains (Factor 13B, or F13B) lie within the regulators of complement activation (RCA) locus on chromosome 1q32. A large, common deletion that encompasses both the CFHR1 and CFHR3 genes has been identified on one of the CFH protective haplotypes (Hageman et al 2006).

Due to the strong homology between these seven genes, substantial copy number variation with this 1q32 locus, and the lack of understanding of Complement-Mediated Disease causality within this locus, extended haplotypes encompassing CFH, the five CFHR genes, the CFHR pseudogene (LOC100289145) and Factor 13B (F13B) were determined, and their association with AMD was assessed.

2.2 Methods: Study Patient Samples

DNA samples from study patients, including sib pairs, with and without AMD were used. DNA samples from the 1,073 subjects employed for this study (referred to as ‘1,073 Cohort’, ‘GSH Cohort’ or ‘ParAllele Cohort’) were of European-American decent and selected from a larger cohort (see Example 1 above). The average age of subjects was 76.2 years. Fifty-nine percent were female. 56% of subjects had age related maculopathy (ARM) or age-related macular degeneration (AMD) and 34.5% were controls. 0.6% of the patients in this cohort could not be graded.

2.3 Methods: Overview of Strategy for CFH-to-F13B Haplotype Determination and Validation in the Discovery Cohort

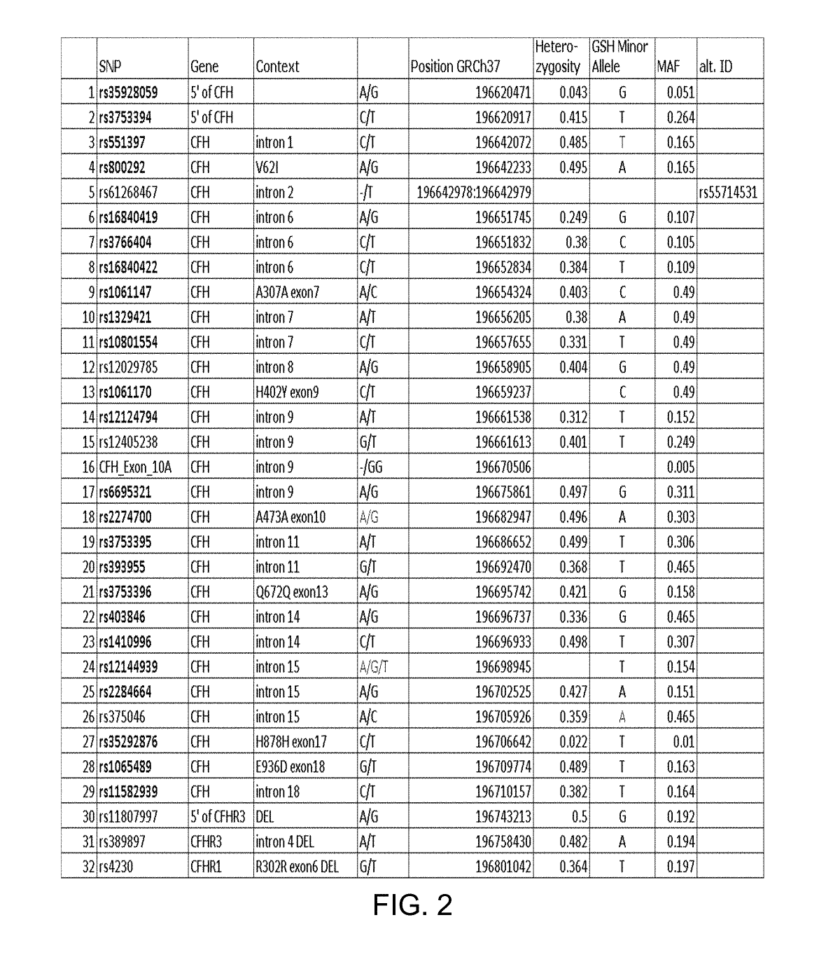

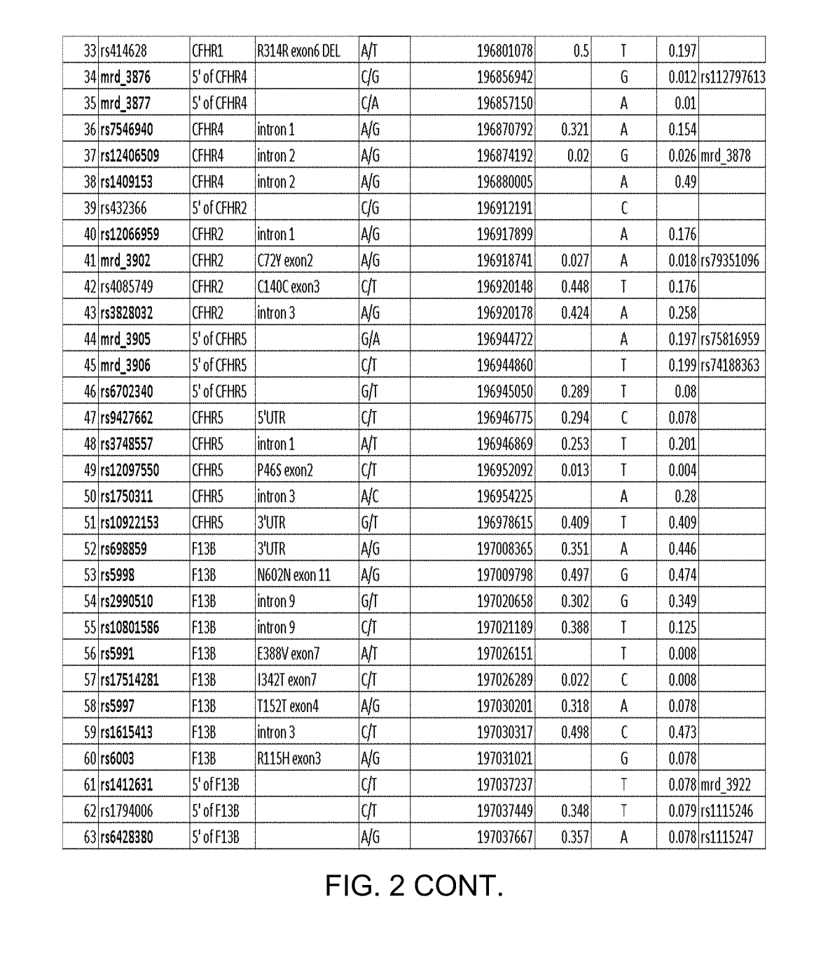

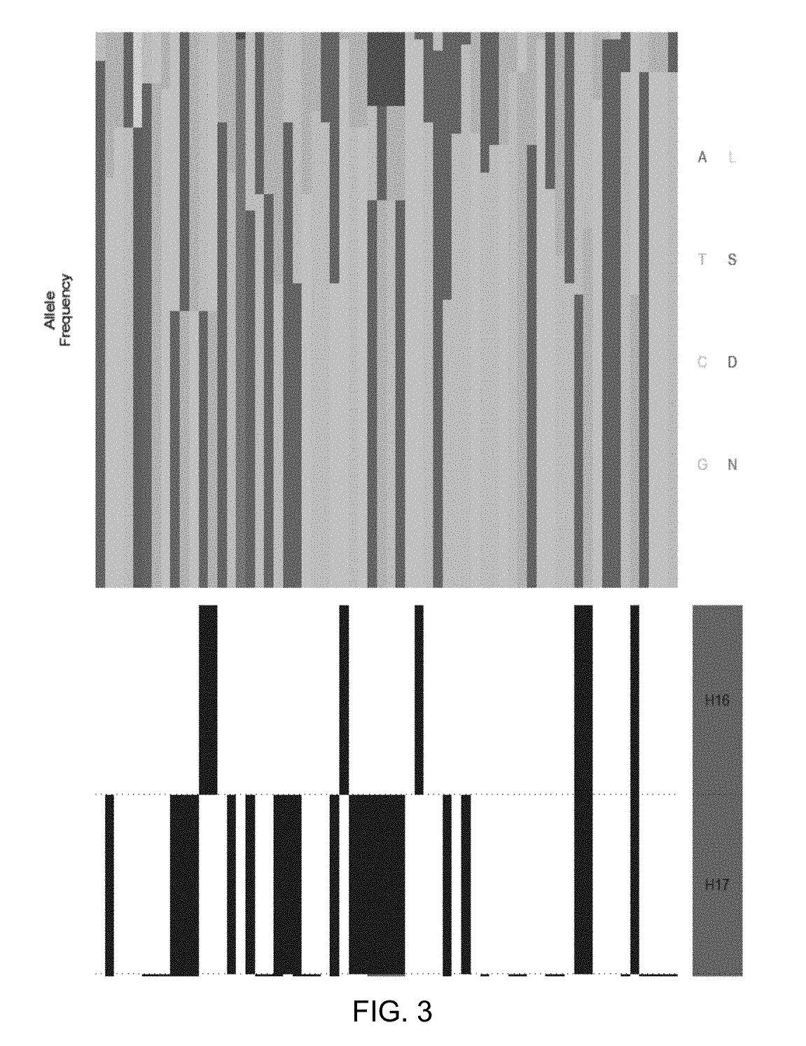

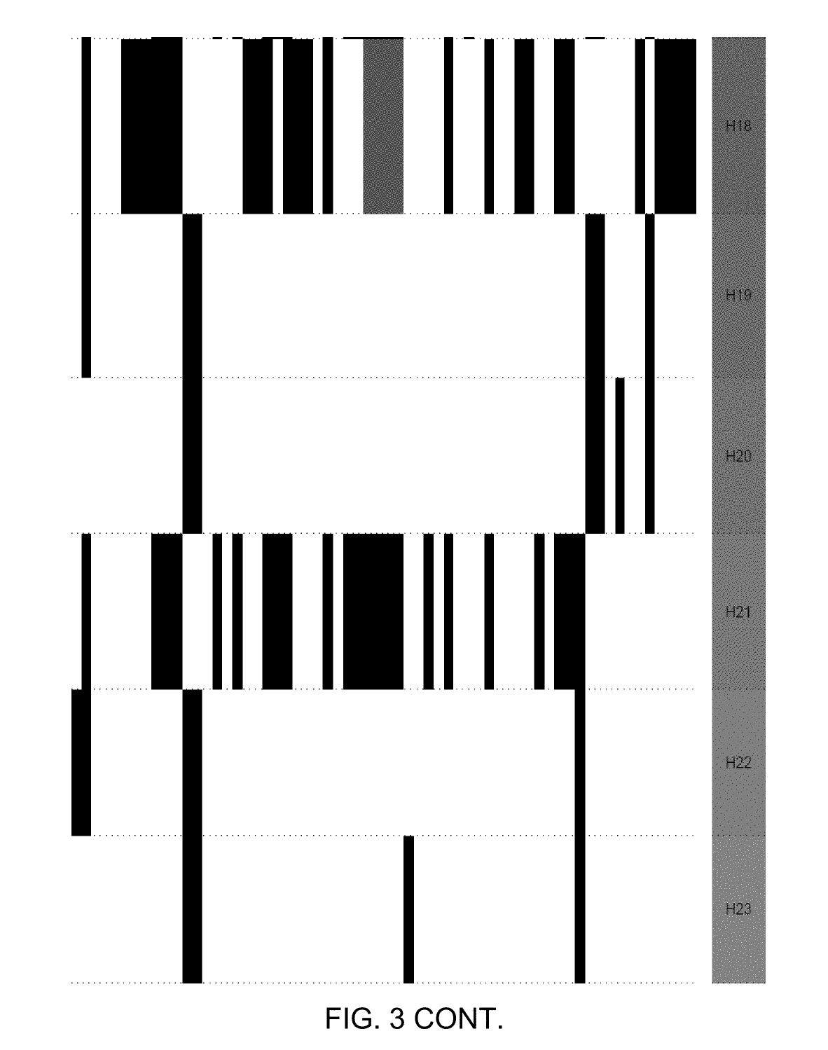

Sixty-three SNPs (FIG. 2) spanning the CFH-to-F13B region were genotyped in 1,073 Caucasian individuals. Missing genotypes were imputed. The deletion status of the CFHR3/CFHR1 genes was assessed initially using single-strand conformation polymorphism (SSCP) and a qPCR assay developed to detect the deletion on single chromosomes. Genotypes were phased five independent times using PHASE v2.1.1, results were combined, and the most likely phased haplotypes for all 1,073 individuals were extracted. Haplotypes for all chromosomes were subsequently constructed (phased). Haplotype validity was confirmed in three-generation families. Haplotype and SNP disease associations were assessed using appropriate statistical assessment methods.

2.4 Methods: Single Nucleotide Polymorphism (SNPs) Genotyping

Sixty-three single nucleotide polymorphisms (SNPs) within the CFH-to-F13B locus, including non-genic regions both proximal and distal to the CFH and F13B genes (FIG. 2), were genotyped using SSCP-based genotyping [5], Taqman based genotyping (ABI), Sanger sequencing, and molecular inversion probe genotyping (ParAllele BioScience) [6] in 1,073 human genomic DNA samples. SNPs were selected based on significance of association in a prior ParAllele Study (a study in which 1,162 DNA samples were genotyped using the Affymetrix 3K ‘Iowa AMD Panel’; the final data yielded successful genotypes for 1,113 samples, with a final number of 3,266 successful assays out 3,308 assays attempted), as well as location to provide relatively dense coverage across the entire CFH-to-F13B locus. Missing values were multiply imputed during haplotype construction using PHASE v. 2.1.1 (Am. J. Hum. Gen. 68:978-989, 2001).

2.5 Methods: Identification of CFHR3/CFHR1 Deletion Chromosomes

The deletion status of the CFHR-3/CFHR-1 genes was assessed using SSCP and a qPCR assay developed to detect the deletion on single chromosomes. SSCP assay was used to identify all individuals homozygous for the CFHR3/CFHR1 deletion. Using a pair of PCR primers that amplify both CFH exon 22 and a homologous region in CFHR1 (see Hageman et al 2006 for primers), one of the resulting SSCP conformations (referred to as ‘shift 4’) was diagnostic of a subject for which both copies of CFHR1 were absent.

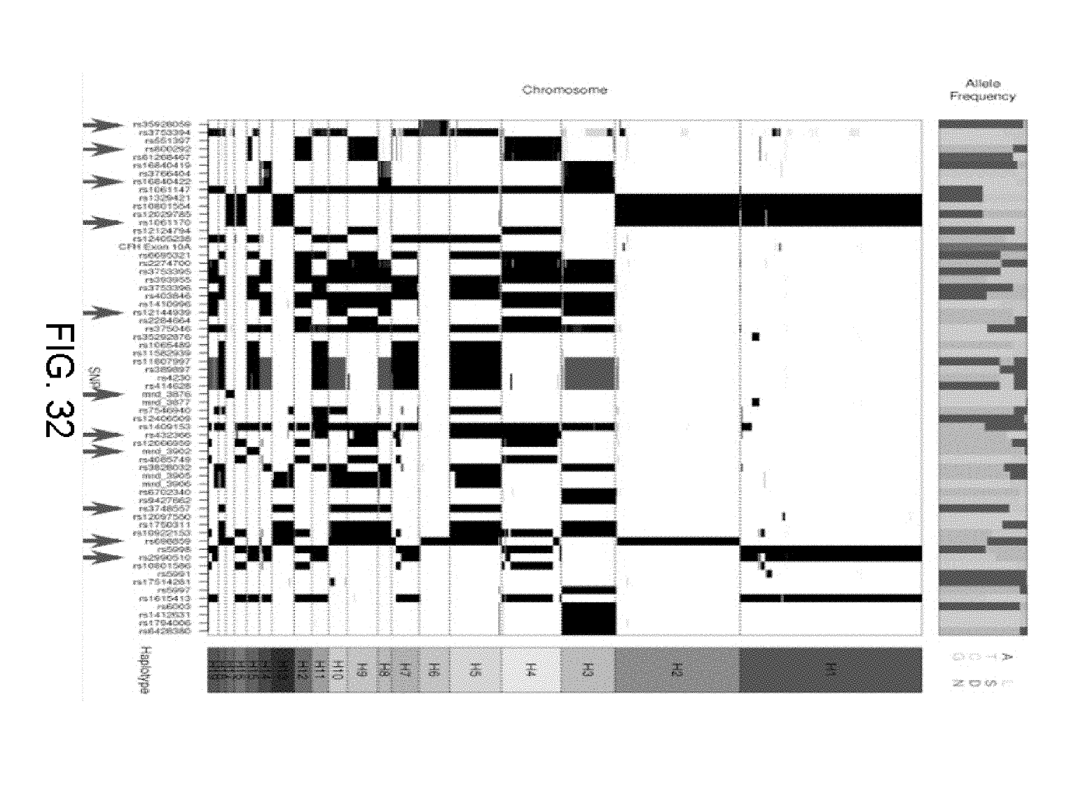

Real-time quantitative PCR (qPCR) was also used to determine the copy number state of the CFHR3 and CFHR1 genes (FIG. 31). A set of five to six independent TaqMan assays measured simultaneously the amount of chromosomal DNA upstream of CFHR3 (the deletion amplicon′) using a 6-FAM fluorescent signal and the endogenous control amplicon of RNase P using a VIC tag (Applied Biosystems RNase P Control Reagent). The sequences of the primers and probe for the deletion assay were designed based on the mrd 3855 amplicon. They were designated mrd_3855-ANYF (TCT GCT CCT GCT CTT CTT TTC C SEQ ID NO. 285), mrd_3855-ANYR (CTC TAT GCT TGC TAC AAG AGA CAC A SEQ ID NO. 286), and mrd_3855-PROBE (6-FAM-AAC AGC ATG GAA TAT C-MGB SEQ ID NO. 287). The reference amplicon was of RNase P, a single copy gene, and reagents were purchased from Applied Biosystems (part number 4316844) with a VIC fluorescent tag attached. Thus, the FAM tag of the deletion amplicon and the VIC tag of the control amplicon could be detected simultaneously in the same rtPCR well. Applied Biosystems Fast Universal PCR Master Mix provided both the polymerase and ROX exogenous non-amplifying background fluorophore. An Applied Biosystems 7900HT Fast Real-Time PCR System was used for all experiments.

Following data acquisition, analysis was performed using a program written in the R statistical software language. The optimal fluorescence threshold for detection of the exponential amplification phase was determined using a set of serial dilutions of control samples containing one copy of CFHR3/CFHR1 per chromosome. Uncertainties were propagated at all stages to obtain a distribution of Ct values for all samples and all replicates. A sample was classified as having no deletion chromosomes (homozygous normal) if most sample replicates had Ct values for both the deletion amplicon and RNase P similar to the normal control. Samples heterozygous for the CFHR3/CFHR1 deletion had Ct values for the deletion amplicon approximately one cycle later than expected based on their RNase P Ct values. Finally, samples homozygous for the deletion had either no or very minor amplification of the deletion amplicon. A serial dilution of a normal control was run on each qPCR plate.

2.6 Methods: Deletion Prediction

Deletion status was modeled as a biallelic marker: deletion or no-deletion (normal) for each chromosome. Real-Time qPCR was capable of identifying individuals homozygous (DD) or heterozygous (ND) for the deletion and those not containing a deletion (NN). Based upon the deletion status and the genotyping call of CFH locus SNPs, a decision tree capable of predicting deletion status from genotyped SNPs was created using the rpart library in the R statistical software package. In addition to this predictive tree, the strategy that any individual who was heterozygous for one of the four SNPs screened in the CFHR3/CFHR1 gene region (rs11807997, rs389897, rs4230, or r5414628) cannot contain a deletion for CFHR3 and CFHR1 was also employed. All of these SNPs are in the central portion of the deletion region and none were present when the CFHR3 and CFHR1 genes were absent.

2.7 Methods: Haplotype Inference

The qPCR deletion assay was used to classify 536 (and 801 in a second assay) individuals as homozygous deletion (DD), heterozygous deletion (ND), or homozygous non-deletion (NN). All remaining individuals were classified according to their genotypes for SNPs in CFHR3 and CFHR1. All individuals containing more than one heterozygous SNP in CFHR3 or CFHR1 were assumed to be not consistent with a deletion on either chromosome (genotype NN). All other individuals were assigned to have one normal chromosome and one unknown chromosome (N?). All heterozygous deletion individuals were assigned one unknown allele for those SNPs between CFH and CFHR4, the deleted region; homozygous SNPs in this region (ex. CC) were replaced with one known and one unknown allele (C?) while heterozygous SNPs, being inconsistent with a heterozygous deletion, were replaced with two unknown alleles (??). All homozygous deletion individuals were assigned two unknown alleles (??) for each SNPs in the same region.

PHASE version 2.1.1 was used to impute missing genotypes and phase genotypes for each individual, care was taken to correctly handle the presence or deletion of CFHR3/1 SNPs as a group. Chromosomal positions for each SNP were obtained from NCBI db SNP build 129 using the reference human genome (build 18). All individuals typed at SNPs in the CFHR3/CFHR1 region and seven distinct four-SNP haplotypes were imputed for the CFHR3/CFHR1 deletion region using the four SNPs in the deletion region spanning from rs11807997 through rs414628. Five independent runs of PHASE were used to confirm haplotype consistency and two haplotypes were assigned to each individual. Seven distinct four-SNP haplotypes were imputed for this region. Although the first two haplotypes were sufficient to phase 99% of the chromosomes, all seven imputed haplotypes were utilized later to avoid over-fitting of the phased genotypes.

Deletion status was subsequently treated as an octa-allelic SNP one for each of the seven imputed CFHR-3/CFHR-1 haplotypes and an eighth allele to signify the deletion. The entire CFH locus was thus modeled as 58 bi-allelic and one octa-alleic SNP. Five independent runs of PHASE were used to impute and phase the genotypes. Five independent runs of PHASE were used to confirm phased genotype consistency. Following genotype phasing, the multi-allelic deletion status was expanded back into four separate tri-allelic SNPs, with a D allele representing the deletion haplotype. In some assessments, all minor haplotypes with frequency less than 1% were iteratively re-assigned to the most similar remaining haplotype.

PHASE provided probability-weighted, imputed, and phased genotypes for all individuals. Each individual run of PHASE produced multiple weighted estimates of each individual's phased genotypes (using the pairs output file from PHASE). The five independent runs of PHASE produced up to five different imputations of missing genotypes, all of which were then equally weighted. To assign a final pair of haplotypes to each individual, the following algorithm was employed: 1) Input was a collection of individuals where each individual had a set of potential diplotypes and its estimated probability (from PHASE pairs output files); 2) The active set was initialized to include all possible haplotypes; 3) Individual diplotypes were assigned based on the individual's diplotype probabilities with the constraint that diplotypes could only use haplotypes in the active set. It was possible for an individual to not be assigned a diplotype if all of the individual's potential diplotypes required haplotypes not in the active set; 4) Step #7 was skipped to if any individual was not assigned a diplotype; 5) The assigned individual diplotypes were saved as the current solution; 6) The least-used haplotype was removed from the active set and step #3 was returned to; 7) The current solution was outputted as the list of individual diplotypes, and thus haplotypes, present in the population.

Phased genotypes were also processed in some analyses using Beagle (version 3.0.1) to obtain two haplotype networks—one merging similar haplotypes in the 5′→3′ direction, the second in the 3′→22 5′ direction. All ten phased genotypes for each individual from the five independent runs of PHASE were used as input to account for uncertainty in the phasing or imputation of missing genotypes. The scale parameter for Beagle's haplotype merging process was set to 4.0. The directed acyclic graph haplotype output by Beagle indicated which SNPs form haplotype blocks and how those blocks were interrelated. Each chromosome was assigned a haplotype based on its path through the two networks leading to the rs4230 SNP. Haplotypes accounting for less than 1% of the chromosomes were not reported.

2.8 Methods: Haplotype Validation

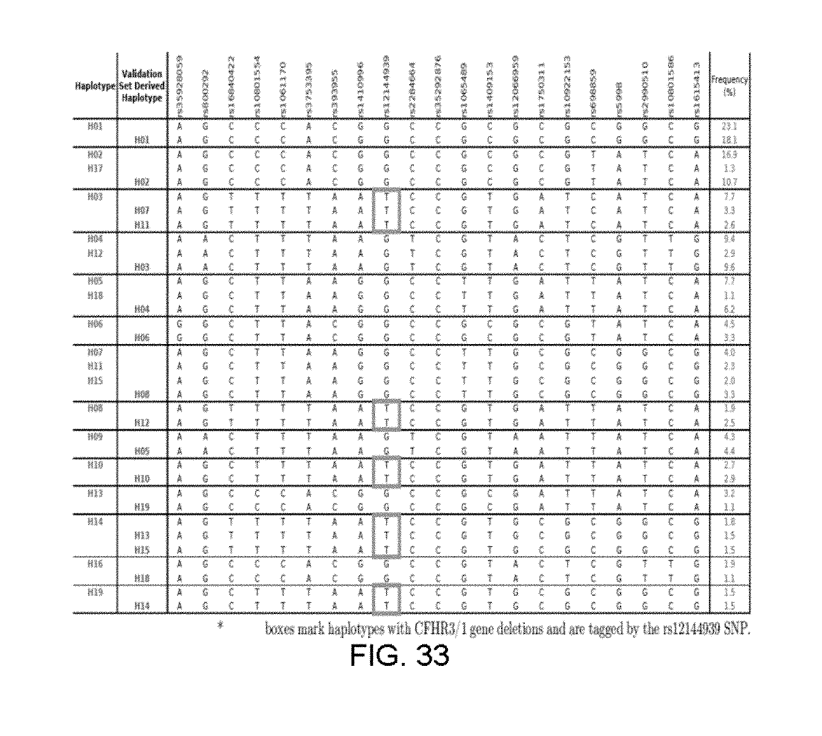

Reference control phased haplotypes were obtained from the members of the Utah Genetic Reference Project families (Ann. Rev. Genomics Hum. Genet. 9:347-358 2008). The UGRP family members consisted of over 600 individuals from 43 three-generation families that were also part of the original Utah CEPH (the Center d'Etude du Polymorphisme Humain) families. Sixty-six SNP genotypes were obtained on blood DNA samples from the entire UGRP members using the Affymetrix Genome-Wide Human SNP array 6.0. Genotype calling was carried out using the CRLMM calling algorithm from the Bioconductor 2.3 Oligo Package. Genotype calls whose confidence was less than 0.95 were replaced with missing values. SNP markers whose call rate was less than 95% were also dropped. Taqman SNP genotyping assays were also carried out to obtain genotypes for 23 additional SNP markers. PedCheck program was used to detect Mendelian inconsistencies and misinherited genotype calls were removed. Phased haplotypes were constructed based on the Mendelian inheritance model in each of the three-generation families. None of the haplotype inference programs were used. Haplotype frequencies were calculated on 136 unrelated UGRP individuals, who were mostly first generation grandparents, using Haploview program's phased haplotype input mode, Haps format (FIG. 33).

2.9 Methods: AMD Disease Association

Association of haplotypes with AMD was assessed by Bayesian logistic regression in individual SNPs, in 536 individuals (median age=79 yrs): 216 individuals without AMD and 320 individuals with AREDS categories 2, 3, geographic atrophy, or neovascular AMD. Complement-Mediated Disease association was determined using a Bayesian logistic regression model with Cauchy priors on the coefficients (Ann. Appl. Stat. 2:1360-1383, 2008). Single SNP associations in this case-control cohort were also assessed.

2.10 Results: Genotyping and Identification of Novel CFH Locus SNPs

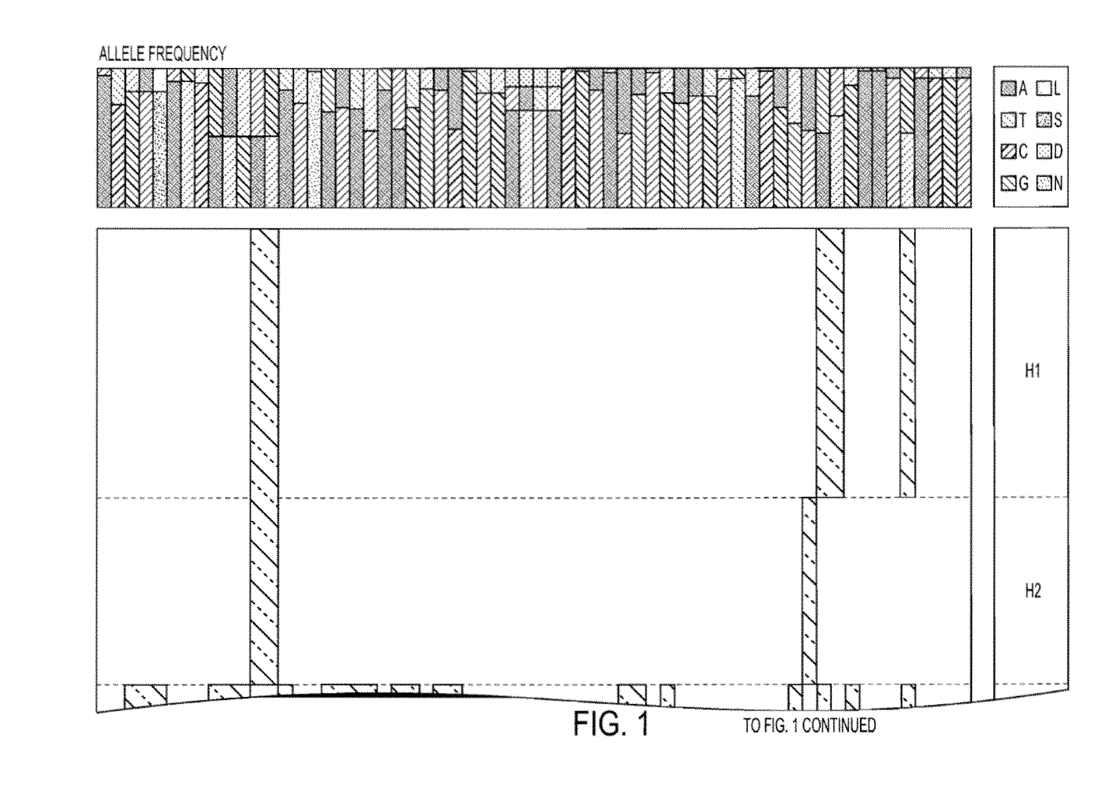

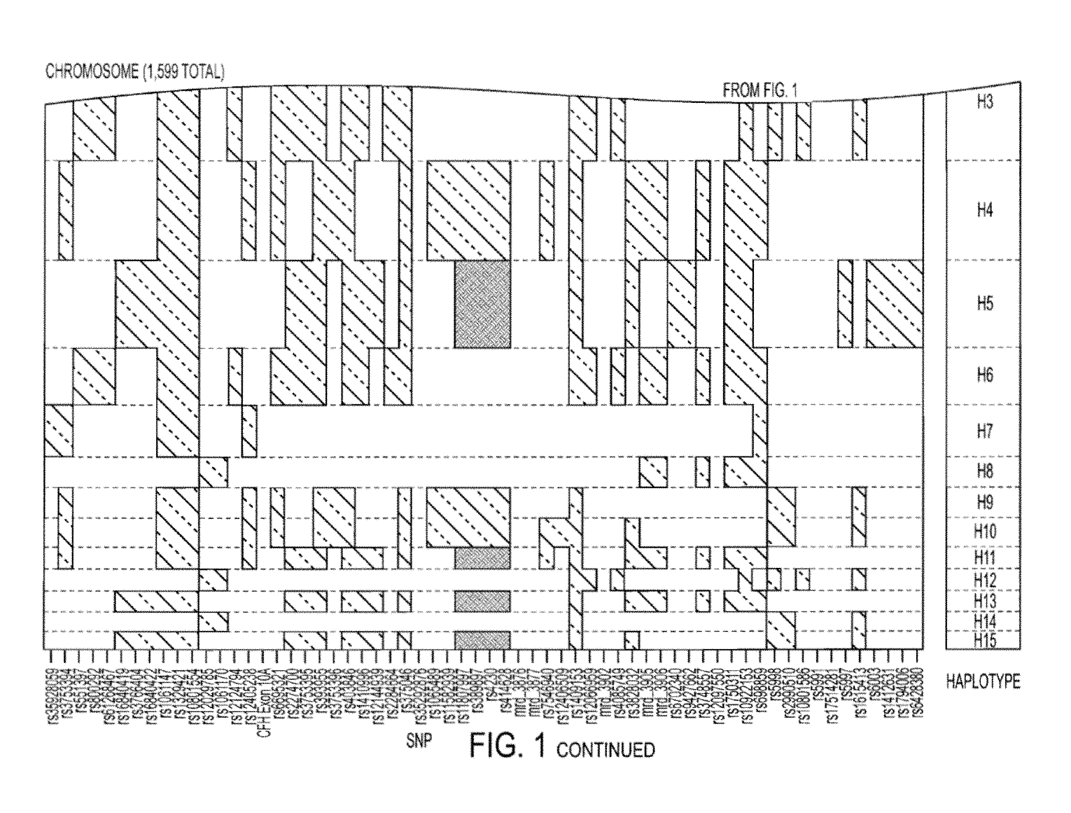

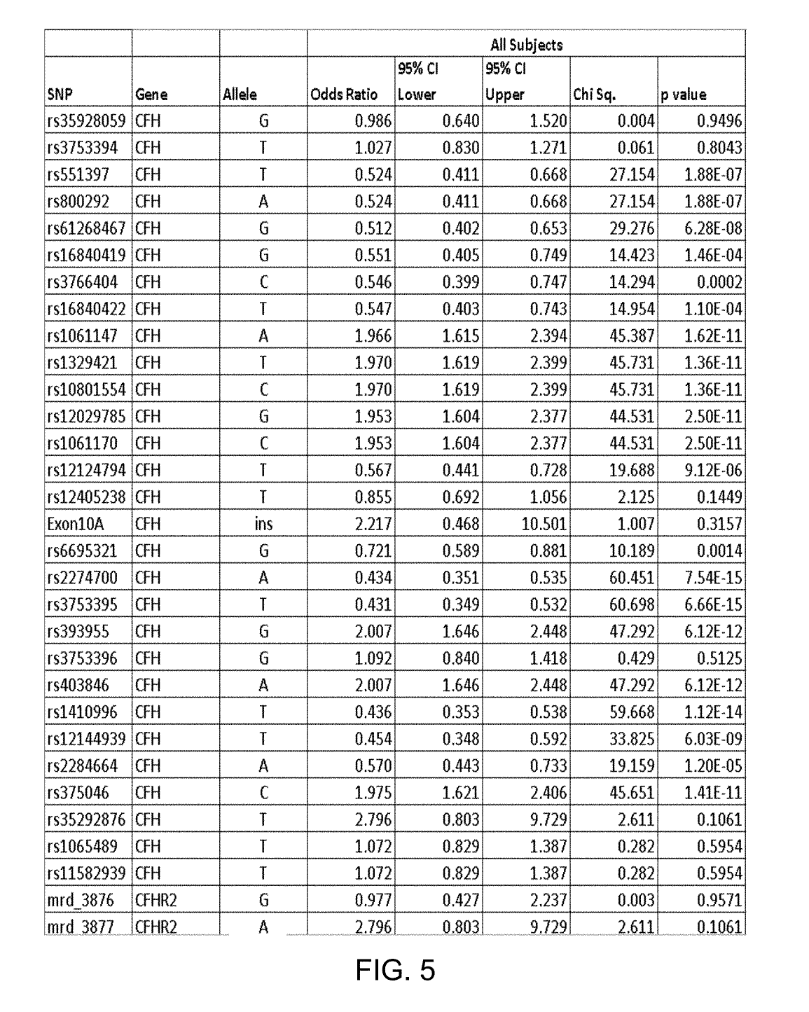

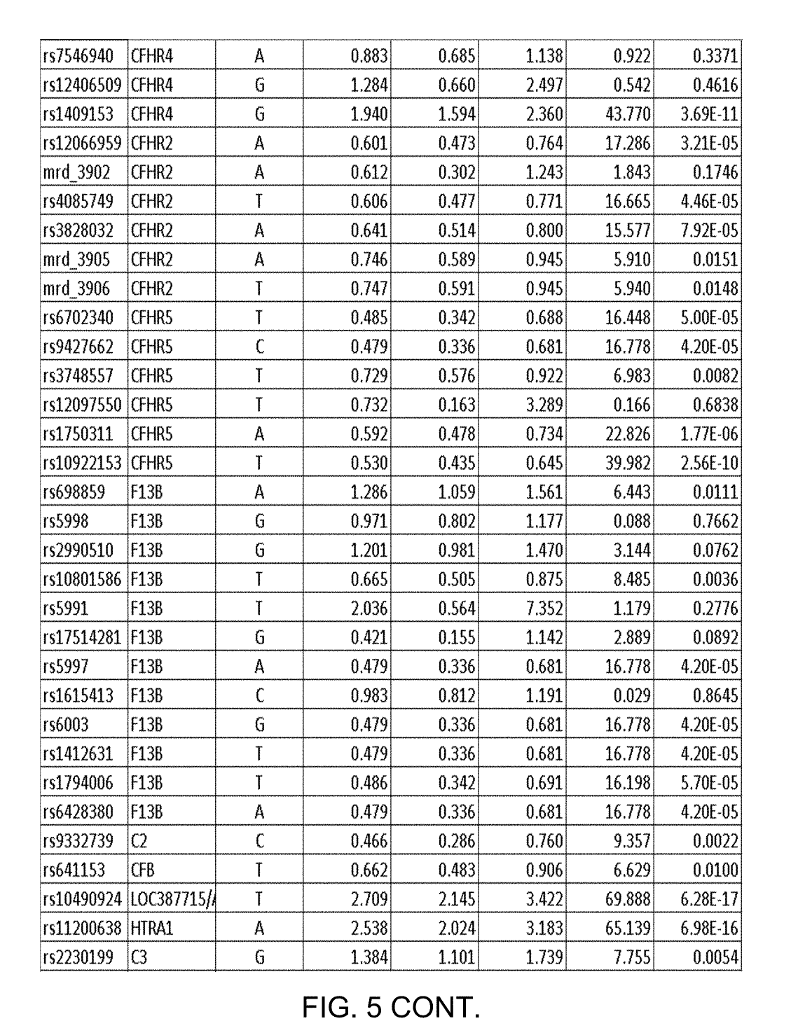

DNA from each subject was screened for 41 to 63 of the 63 SNPs employed (FIG. 2 and FIG. 29). Of the SNPs genotyped by ParAllele using MIP genotyping, five were unreported in the literature (FIG. 27) at the time of their discovery. These were identified using mismatch repair detection (MRD) to amplify a variant-enriched pool of amplified genomic DNA. This pool of amplified DNA was then sequenced to identify rare alleles. The rare mrd_3876 and mrd_3877 SNPs were located within the promoter region of CFHR4. The minor allele of mrd_3876 was present in haplotype(s) that skewed toward risk for AMD, and the minor allele of mrd_3877 occurred in haplotype(s) that skewed significantly with AMD risk. The minor allele of mrd_3902, located within exon 2 of CFHR2, results in a Cys72Tyr coding mutation and occurred in haplotypes that appeared to skew toward protection for AMD. The minor alleles of SNPs mrd_3905 and mrd_3906 were more common (minor allele frequencies of approximately 20%) and were located within the promoter region of CFHR5; they did not appear to skew with AMD risk or protection (see FIGS. 1 and 5).

One additional novel SNP was identified within the CFH-to-F13B locus at the time these assays were conducted. This variant is located within CFH exon 10A—the last exon of the truncated form of complement factor H (FIG. 2). This SNP was identified via SSCP and from sequencing is thought to be a one or two base-pair insertion into the 3′ untranslated region of exon 10A. The minor allele, the 1-2 bp deletion, was present in only 1.2% of chromosomes.

2.11 Results: Deletion Chromosome Identification—TaqMan qPCR Copy Number Variation Assay

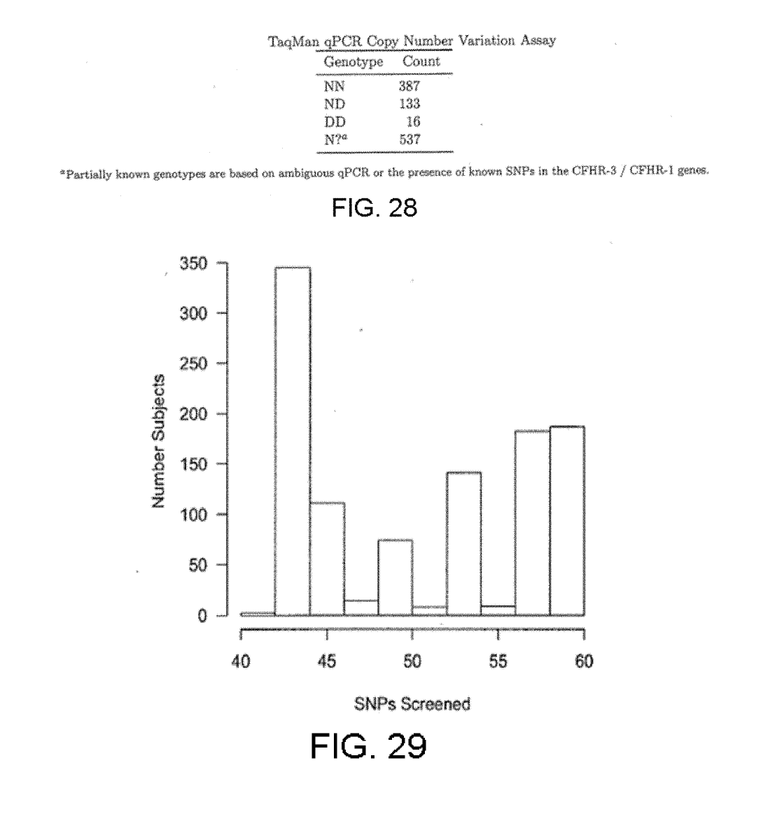

The results of the deletion assay are shown in FIG. 28. Homozygous deletion samples were verified by SSCP analysis. Of those samples that gave definitive copy number values, 28% had a deletion of one or both copies of CFHR3 and CFHR1. 15% of individual chromosomes did not contain these genes. After including imputed deletion status, 19% of individual chromosomes did not contain the deletion (absence of CFHR3 and CFHR1).

Experiments were also conducted to determine the reliability of this assay. As an example, one experiment produced 192 replicates for each of two DNA samples—one homozygous non-deletion sample and a related heterozygous deletion sample. Each qPCR assay was perfomed using total genomic DNA between 100 ng and 0.6 ng. Based on these data, for a given amount of genomic DNA, an assay using heterozygous deletion DNA was predicted to have a Ct value 0.645±0.002 cycles greater than an identical assay utilizing homozygous normal DNA. The expected distance from the line Ct,deletion=Ct,RNaseP for an individual sample was 0.16 cycles. Thus, the expected power for correctly classifying a heterozygous deletion of the CFHR3/CFHR1 genes using five to six qPCR replicates was 83%. This is similar to the 75% call rate shown in (FIG. 28). Thus, to obtain a power of 95% with a 5% type-I error rate, nine replicates were run on each DNA sample.

2.12 Results: Deletion Prediction

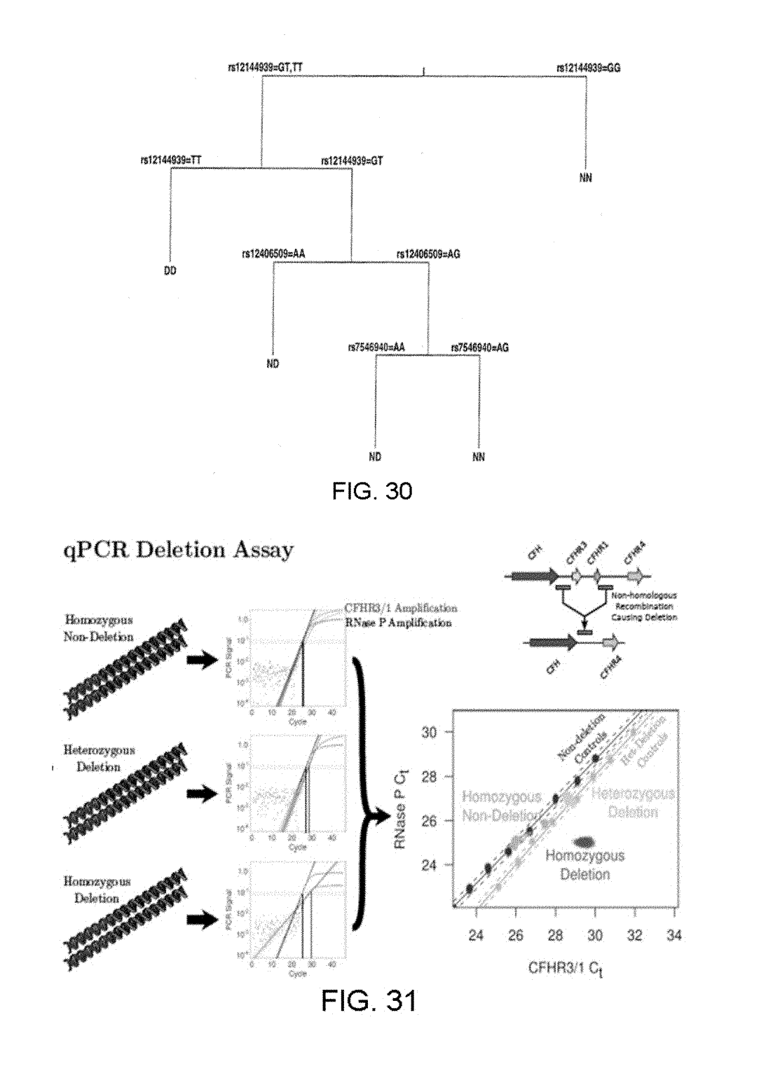

Based upon measured (not imputed) genotypes, a decision tree (FIG. 30) was made to predict deletion status from three SNPs. All homozygous deletion individuals had a TT genotype at SNP rs12144939 in CFH, although 17 heterozygous deletion individuals had genotype GG at this location. Thus, it was determined that deletion status in Caucasians can be predicted reliably by a ‘T’ at rs12144939 located within intron 15. The deletion status of the CFHR3/CFHR1 genes could be predicted with 97% specificity and 96% sensitivity based on the genotype of this single SNP. Fourteen percent of chromosomes in the discovery cohort contained this deletion. The inclusion of rs12406509 and rs7546940 (mrd_3878) slightly increased the sensitivity and specificity of the deletion prediction.

2.13 Results: Identification and Validation of 63 SNP-Based CFH-to-F13B Haplotypes

63 SNP-based haplotypes (FIG. 32) spanning the extended CFH-to-F13B locus and including the CFHR3/CFHR1 deletion were determined. Nineteen major haplotypes (>1% frequencies) were identified within the approximately 260 kb CFH-to-F13B locus based on haplotype construction using PHASE v.2.1.1 (FIG. 32). Haplotype fidelity was confirmed in large, multi-generation Utah Genetic Reference Project families (Annu. Rev. Genomics Hum. Genet. 9:347-358 2008) (FIG. 33). Haplotypes H1 and H2, the two most prevalent haplotypes (approximately 47% of the chromosomes in this cohort), contained the vast majority of the CFH Y402H variant. Five of the 19 haplotypes (H3, H8, H10, H14, and H19) contained the vast majority of all chromosomes with CFHR3/CFHR1 deletions. The CFH I62V protective variant was contained primarily in haplotypes H4, H9, and H12. Eleven SNPs (arrows in FIG. 32) were sufficient to infer the nineteen haplotypes.

2.15 Results: Association of 63 SNP-Based CFH-to-F13B Haplotypes with Disease

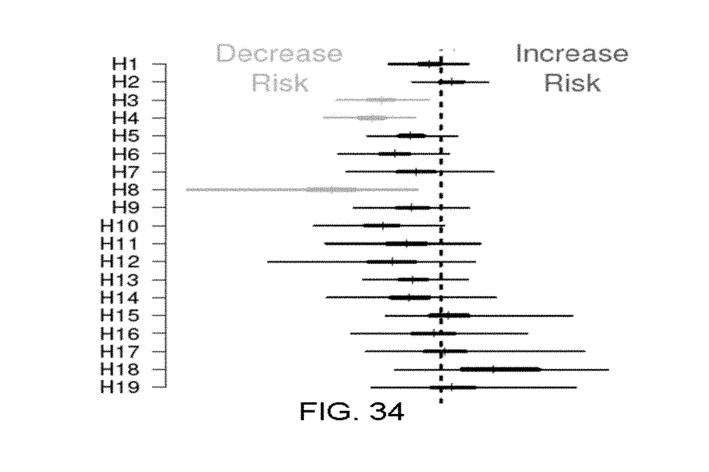

Preliminary assessment of AMD disease association showed significant associations of three of the nineteen major haplotypes with different AMD risk phenotypes (as a binary and quantitative trait) (p<0.01), after computing bootstrap confidence intervals and controlling for age, sex, and smoking status. After controlling for age, gender, and smoking status, haplotypes H3, H4, and H8 were associated with significant decreased risk of developing AMD both as binary (p<0.01) and as a quantitative trait (p<0.05) (FIG. 34).

### Example 3: Refined Assessment of Extended Haplotypes Spanning the Chromosome 1q32 CFH-to-F13B Locus

3.1 Methods: Study Cohorts

The same 1,073 subject cohort (1,073 Cohort′, ‘GSH Cohort’ or ParAllele Cohort′) described in Example 2 was employed in studies to refine the understanding of extended CFH-to-F13B haplotypes and their association with AMD and other diseases. In addition, the highly discordant sib-pair cohort (657 subjects from 284 families), a combined AMD case-control cohort comprised of subjects ascertained in Utah (837 subjects), Iowa (1,555 subjects), Australia Melbourne (1,704 subjects) (this collective cohort is referred to as the ‘Combined Cohort’) and the UGRP cohort (604 subjects) were employed.

3.2 Methods: Genotyping

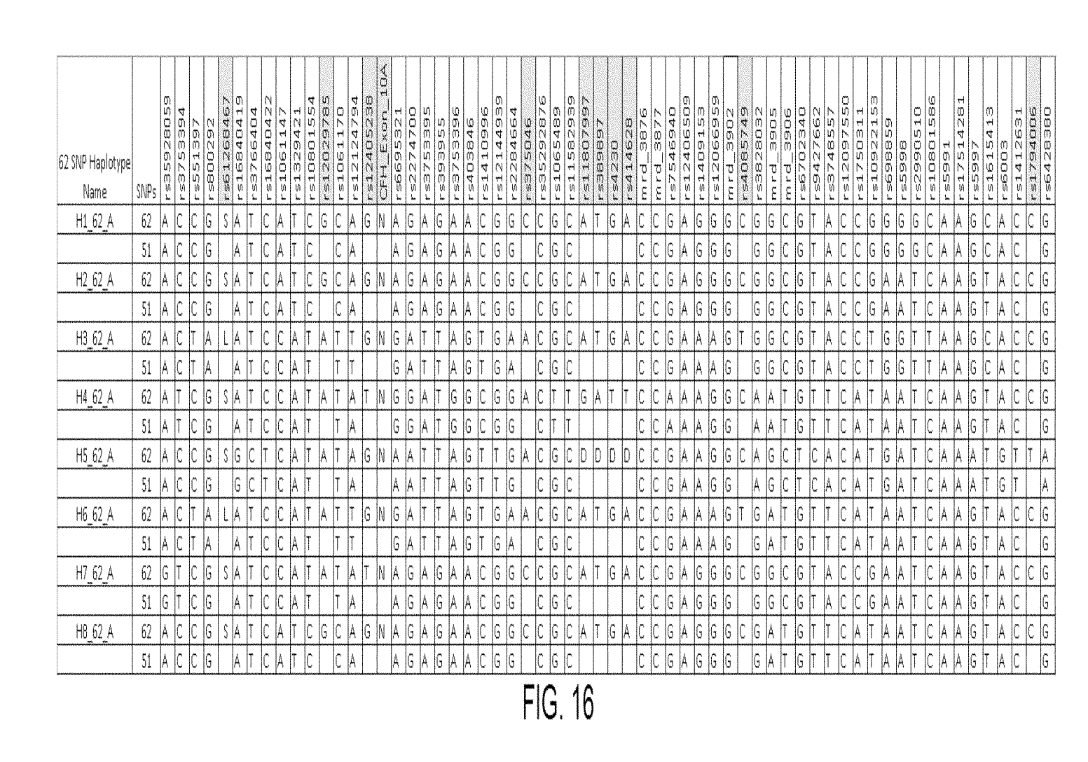

62 SNPs of the 63 SNPs employed in Example 2 were employed in the refined assessment. It was determined that one of the SNPs used to generate the 63 SNP haplotypes, rs432366, was not polymorphic in the location for which it was listed in the NCBI and other databases. Thus, this SNP was removed from all future analyses. Haplotype determinations and disease associations were reassessed based on the removal of rs432366. Genotype call rates exceeding 99% were obtained for 51 of the 62 variants and less than 99% for the remaining SNPs; missing genotypes were imputed using PHASE 2.1.1. Genotyping on all other cohorts was performed using either pre-designed or custom ordered Taqman assays (ABI) and, as such, limited to 51 SNPs (see bolded SNPs in FIG. 2 and FIG. 16).

3.3 Methods: Generation of 62 SNP-Based and 51 SNP-Based Haplotypes

PHASE 2.1.1 was employed to determine the SNP-based haplotypes present in the 1,073 patient cohort. Chromosomal positions used were from the NCBI db SNP build 129 using the reference human genome (build 18). Following genotype phasing, the multi-allelic deletion status was expanded back into four separate tri-allelic SNPs (major, minor, and deleted).

In addition, 51 SNP-based haplotypes were generated for the highly discordant sib-pair cohort, the AMD case-control cohorts ascertained in Utah, Iowa, Australia (Combined Cohort′) and the UGRP cohort. Of the 62 SNPs employed for the 62 SNP-based haplotype determinations, the four SNPs lying within the CFHR3/CFHR1 deletion region in the 62 SNP-based set (rs11807997, rs389897, rs4230 and r514628) were not used (instead, the deletion tagging SNP rs12144939 was used as a proxy) for generating the 51 SNP-based haplotypes. In addition, Taqman assays could not be designed to genotype 7 of the remaining 58 SNPs, so these were also not used for generating the 51 SNP-based haplotypes (see FIGS. 15 and 16 for SNPs employed to generate 51 SNP-based CFH-to-F13B haplotypes).

3.4 Methods: Validation of 51 SNP-Based Haplotypes

To validate haplotypes and their frequencies, reference control phased haplotypes were obtained from the members of the Utah Genetic Reference Project (UGRP) families (Annu. Rev. Genomics Hum. Genet. 9:347-358, 2008). TaqMan SNP genotyping assays were used to obtain genotypes for the same 51 additional SNP markers previously genotyped in the GSH cohort (see above). PedCheck program was used to detect Mendelian inconsistencies and misinherited genotype calls were removed. Phased haplotypes were constructed based solely on the Mendelian inheritance model in each of the three-generation families. None of the haplotype inference programs were used. Haplotype frequencies were calculated on 136 unrelated UGRP individuals, who were mostly first generation grandparents, using Haploview program's phased haplotype input mode, Haps Format (Bioinformatics 21:263-265, 2005).

3.5 Methods: Disease Association Assessment Using Single Markers and 62 SNP-Based and 51 SNP-Based Haplotypes

For determination of unrelated case/control disease (AMD) associations using 62 SNP-based haplotypes, only subjects 60 years or older in the 1,073 Cohort were used. Controls were defined as subjects with AMD grades 0, while cases consisted of all subjects with grades 1B-4C (as described in Example 1). Using these criteria, 927 samples from the GSH Cohort (the ‘927 GSH Cohort’) were employed for analysis of disease association. Frequencies of extended haplotypes in the GSH 927 cohort used for disease association were nearly identical to those imputed for the entire 1,073 GSH Cohort (FIG. 4). AMD disease association with the 62 SNP-based haplotypes was assessed using the Vassar 2×2 contingency table for Chi-2 based on allele frequencies.

51 SNPs were also genotyped in the Extremely Discordant Sib-pair Cohort (657 subjects from 284 families). Haploview (v4.2) was used to determine descriptive statistics using normal subjects (only one normal sibling per family for the Family Cohort), i.e. deviation from Hardy Weinberg Equilibrium (HWE) and the minor allele in the population. Tests for association of the minor allele with AMD were performed assuming an additive genetic model, and all AMD subtypes were compared to normals in the entire cohort, and then in males only and separately females only. For the Family Cohort, tests for association in the entire cohort were performed using Conditional Logistic Regression (CLR). In the analysis of males and females only, one subject of each gender was chosen per family (choosing the subject with the most severe disease), and tested for association with all AMD subtypes using logistic regression. Logistic regression was also used to assess 51 SNP-based haplotypes derived from the ‘927 GSH Cohort’. All analyses were performed in SAS (v9.1) and/or Haploview. Meta-Analysis was performed using Comprehensive Meta-Analysis 2.0.

3.6 Results: 62 SNP-Based CFH-to-F13B Haplotypes Generated from the 1,073 Cohort

To generate haplotypes encompassing the CFH-to-F13B locus, 1,073 unrelated Caucasians of Northwestern European decent, were genotyped for 62 SNPs using a combination of assays, with missing genotypes imputed using PHASE2.1.1, as described above. Haplotypes for all samples were then determined using PHASE 2.1.1.

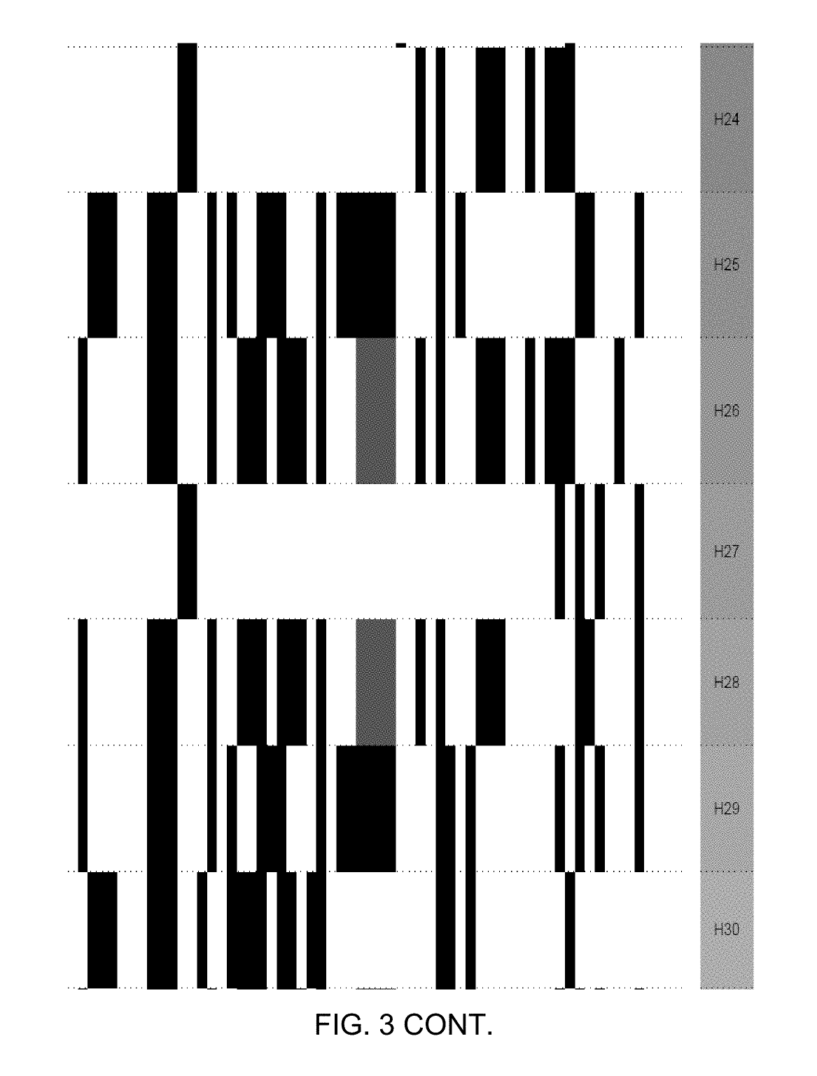

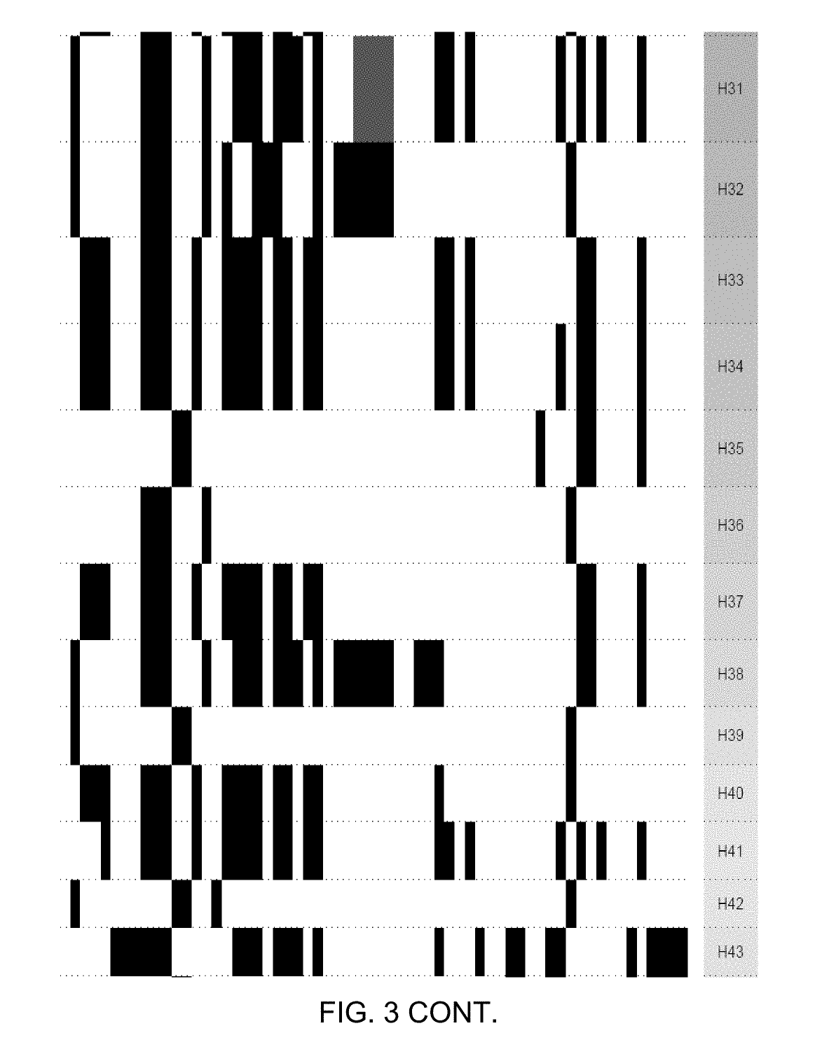

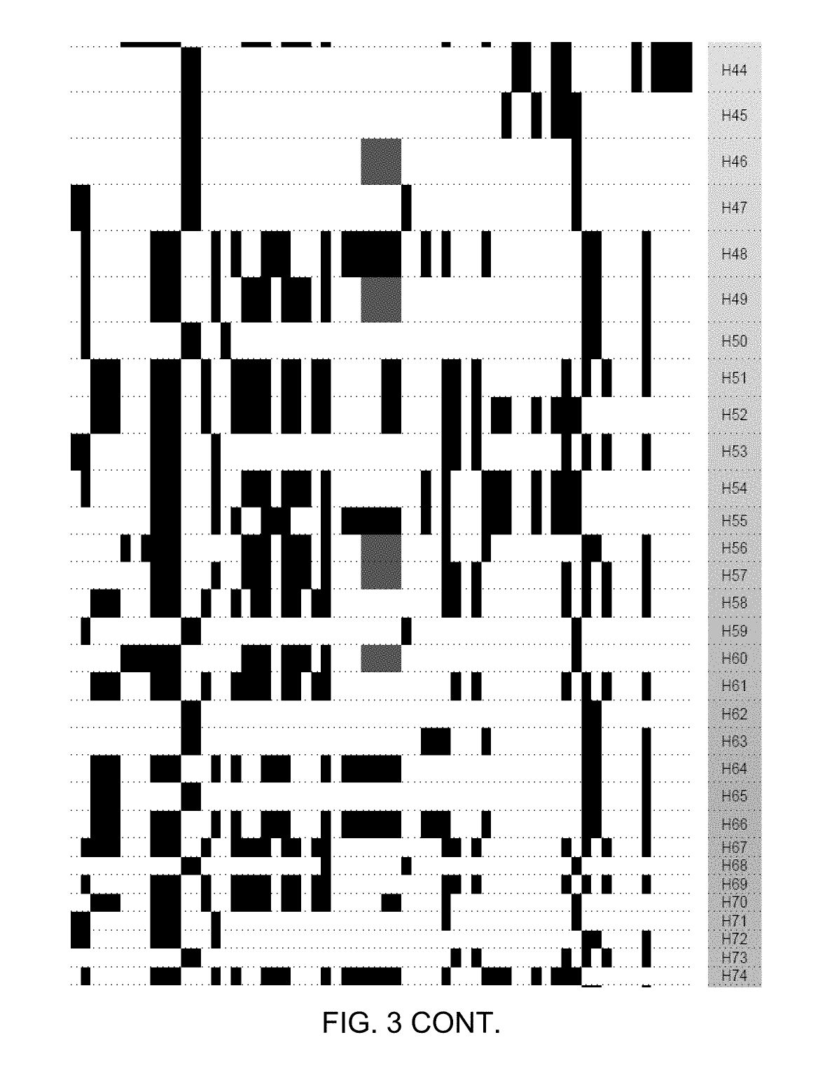

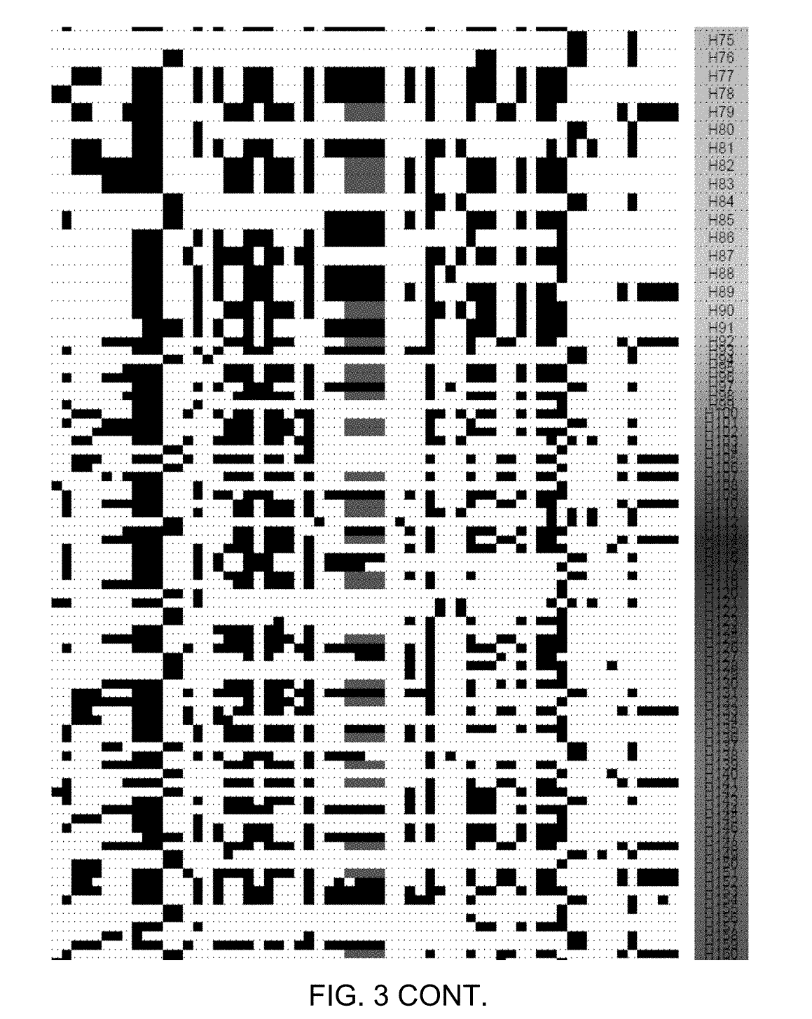

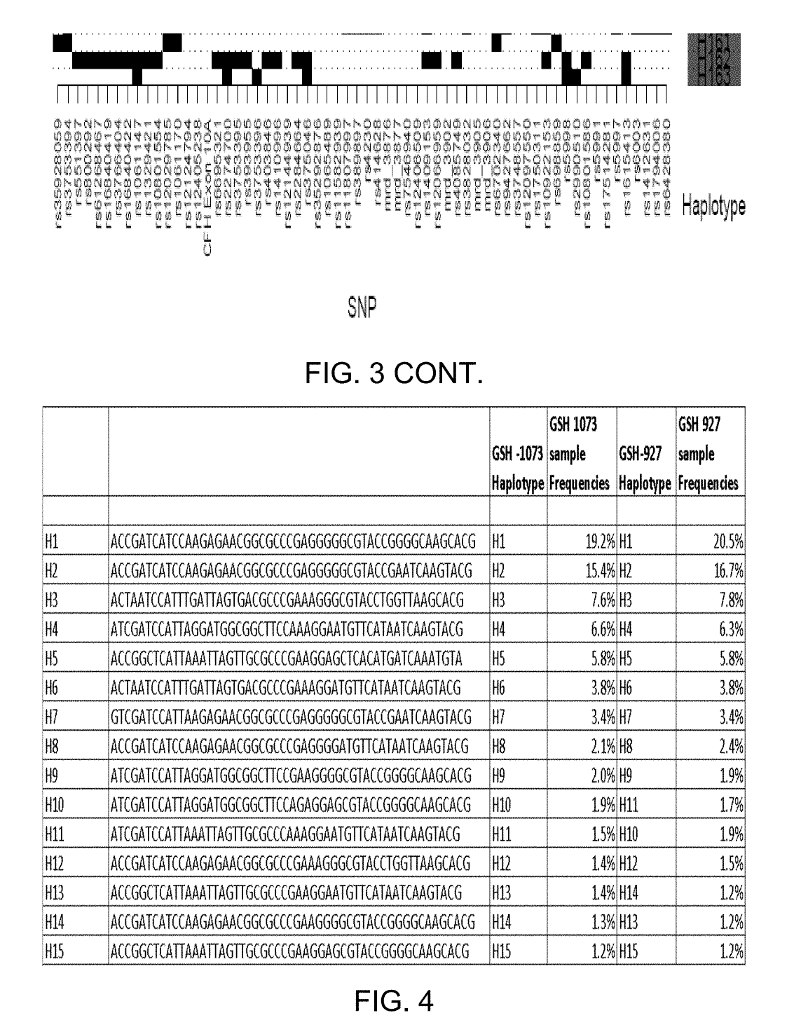

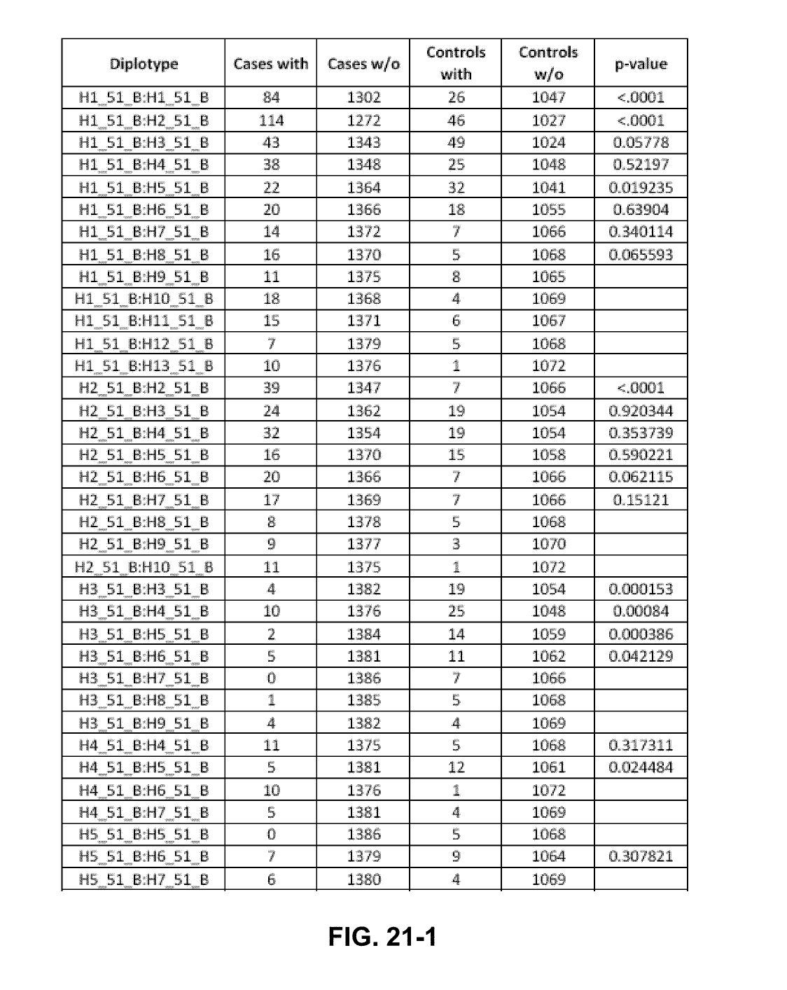

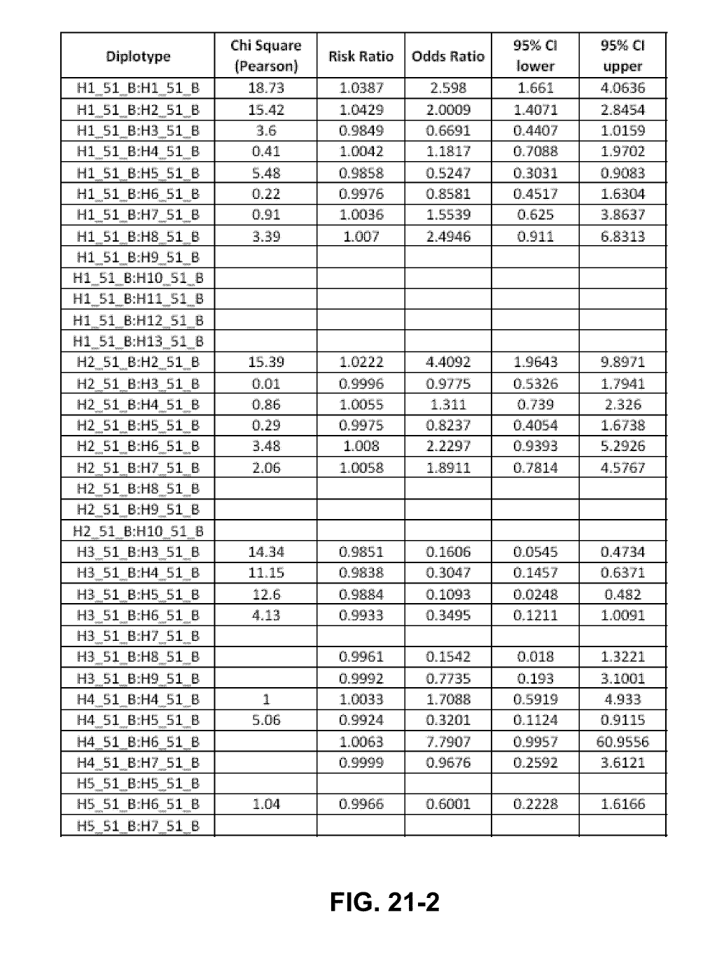

This analysis resulted in detection of 15 major haplotypes (H1_62_A through H15_62_A; FIG. 1 and FIG. 17) present at frequencies greater than 1% and 148 minor haplotypes, each present at less than 1% frequency (H16_62_A through H163_62_A; FIG. 3 and FIG. 17). In total, major haplotypes accounted for 1,599 total chromosomes, while the minor haplotypes contained 547 chromosomes, or 25.5% of the chromosomes in the cohort. A haplotype tree for the 62_A cohort and the hierarchical relationships between the 163_62 SNP-based haplotypes is shown in FIG. 21 of application Ser. No. 14/114,751 (published as US 2014/0357732) which is incorporated herein by reference. (H1_62_A through H163_62_A). The 163 haplotypes were clustered based on the number of similar loci into this tree using the R statistical package hclust function using hierarchical clustering analysis. Similar haplotypes are the most related (closer together) and dissimilar haplotypes are further apart in this ‘tree’, shown in FIG. 21 of application Ser. No. 14/114,751. Importantly, three major clusters containing risk, protective and neutral haplotypes, respectively, were observed. The neutral haplotypes clustered at the left, or top, portion of the tree. Specifically, the neutral haplotype cluster spanned the region from H126_62_A to H163_62_A. The risk haplotypes clustered in the middle portion of the tree. Specifically, the risk haplotype cluster spanned the region from H46_62_A to H73_62_A. The protective haplotypes clustered at the right, or bottom, portion of the tree. Specifically, the protective haplotype cluster spanned the region from H152_62_A to H98_62_A.

The two most frequent of the 15 major haplotypes, H1_62_A (19.2% of total haplotypes) and H2_62_A (15.4% of total haplotypes) both contained the risk allele “C” for rs1061170 (402H). H1_62_A and H2_62_A were identical from the region proximal to CFH through CFHR2, based on the variants examined within this region; only in the F13B gene (beginning with rs698859 in the 3′ UTR of F13B), do SNPs distinguish the H1_62_A and H2_62_A haplotypes (FIG. 1). Other F13B SNPs that distinguish H1_62_A and H2_62_A include rs5998, rs2990510 and rs1615413. Three other major haplotypes contained the CFH 402H risk allele (H8, H12, H14). Two major haplotypes (H3_62_A and H6_62_A) contained the protective minor allele “A” for CFH rs800292. Additionally, 4 major haplotypes contained the CFHR3/CFHR1 deletion (H5_62_A, H11_62_A, H13_62_A, and H15_62_A; FIG. 1). Four SNPs in near perfect LD (rs11807997, rs389897, rs4230, and rs414628) were located in the CFHR3/CFHR1 deletion region; they are shown as tri-allelic SNPs in FIG. 1 (major allele shown in white, minor allele shown in line-filled boxes, and deleted CFHR3/CFHR1 shown in darkly shaded boxes).

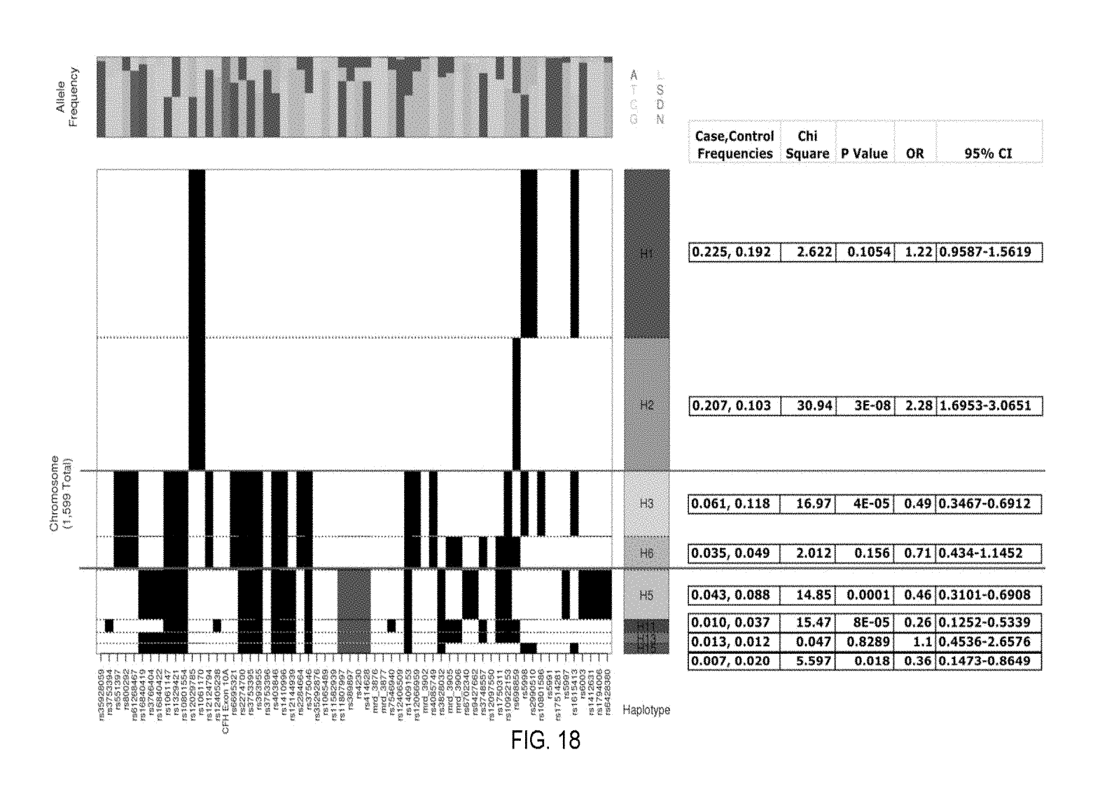

Extensive assessment of the 1,073 cohort (see Example 2), as well as families from the UGRP cohort, resulted in the identification of rs12144939 as a reliable surrogate “tag” for the CFHR3/CFHR1 deletion in >98% of Caucasian individuals, with the minor allele “T” accurately identifying an accompanying deletion. Surrogate proxies for this SNP that were in complete LD include rs6677604, rs16840522 and rs2019727. Major haplotypes containing the deletion of CFHR3/CFHR1 (H5_62_A, H11_62_A, H13_62_A and H15_62_A; FIG. 1 and FIG. 18), with the exception of H13_62_A, are highly protective against the development of AMD in this cohort (FIG. 18).

Importantly, all major haplotypes containing either the protective allele at rs800292 (H5_62_A, H11_62_A, H13_62_A, and H15_62_A) or the CFHR3/CFHR1 deletion (H5_62_A, H11_62_A, H13_62_A, and H15_62_A; include rs11807997, rs389897, rs4230 and rs414628 and are tagged by rs12144939 or surrogate SNPs, as listed above) also contain the minor alleles of the rs2274700, rs3753395, rs393955, rs403846, and rs1410996 variants (FIG. 1 and FIG. 18). The variant rs1409153 is also in near complete LD with all major haplotypes associated with protection. Any of these SNPs, and any other SNP in near LD with these SNPs, singly or in combination, can serve as surrogates for the major protective haplotypes H3_62_A, H6_62_A, H5_62_A, H11_62_A, H13_62_A, and H15_62_A, as well as some of the minor protective haplotypes. Other SNPs within this region of DNA that are in near complete LD (and can serve as surrogates) were determined by generating an LD plot using all of the SNPs in HapMap lying within the CFH-to-CFHR4 region. This was done using the CEU cohort (CEU, Utah residents with Northern and Western European Ancestry from the CEPH Collection, is one of the 11 populations in HapMap Phase 3) genotypes. In this population, 7 SNPs were identified to be in complete or near complete LD with rs1410996, the most significantly associated variant in this “block.” They were (in order of 5′-to-3′ direction) rs10737680, rs7535263 (LD=0.96), rs3753395, rs10922106, rs395998, rs1329428 and rs7540032. There are multiple other SNPs in this 22 kb region that are in various levels of LD with rs1410996.

3.7 Results: 51 SNP-Based CFH-to-F13B Haplotypes in the 1,073 (927 Subset) Cohort

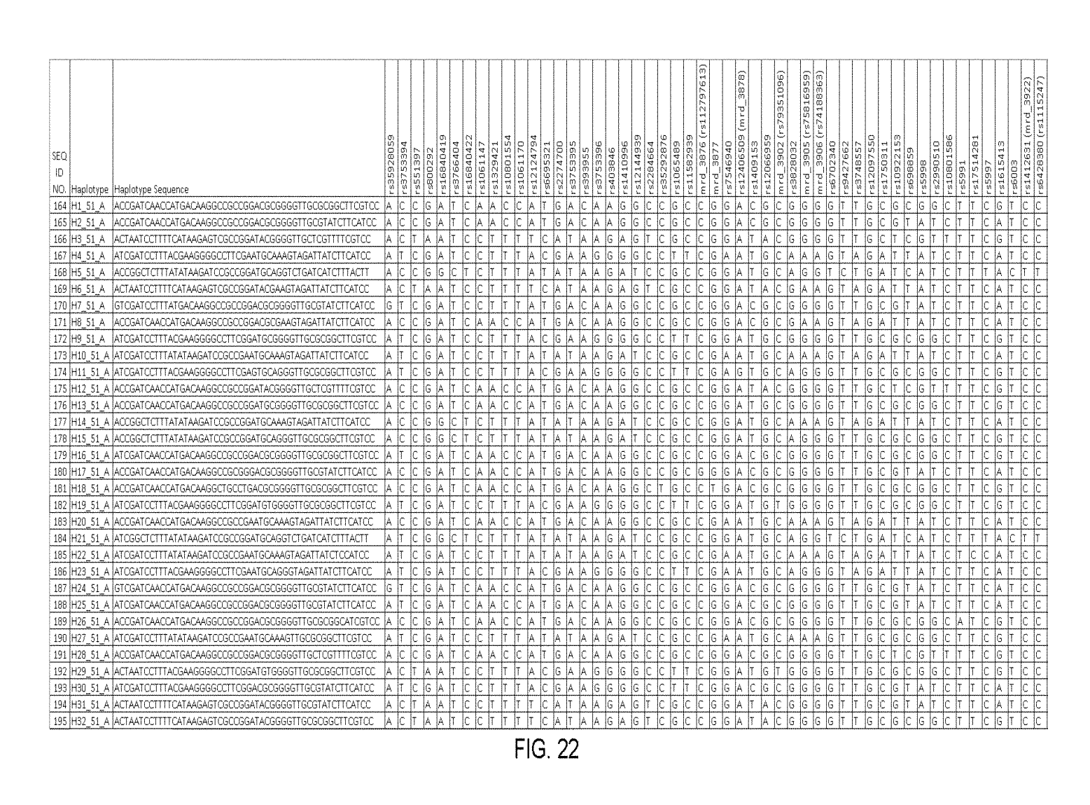

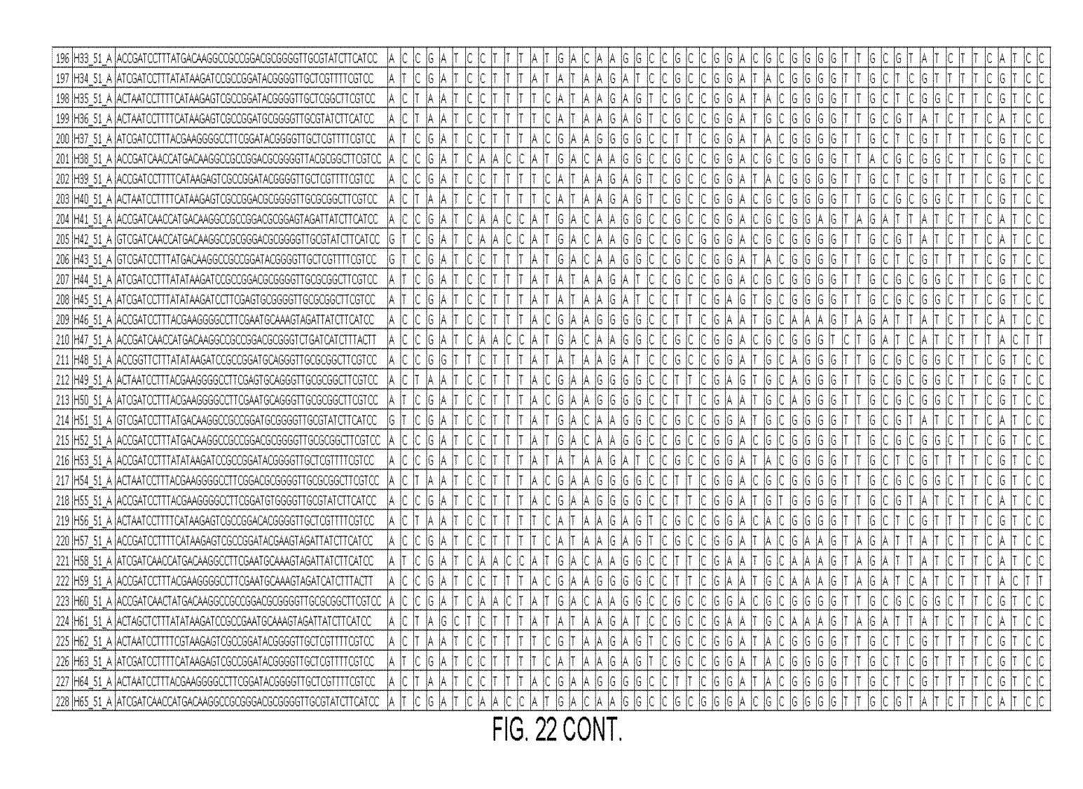

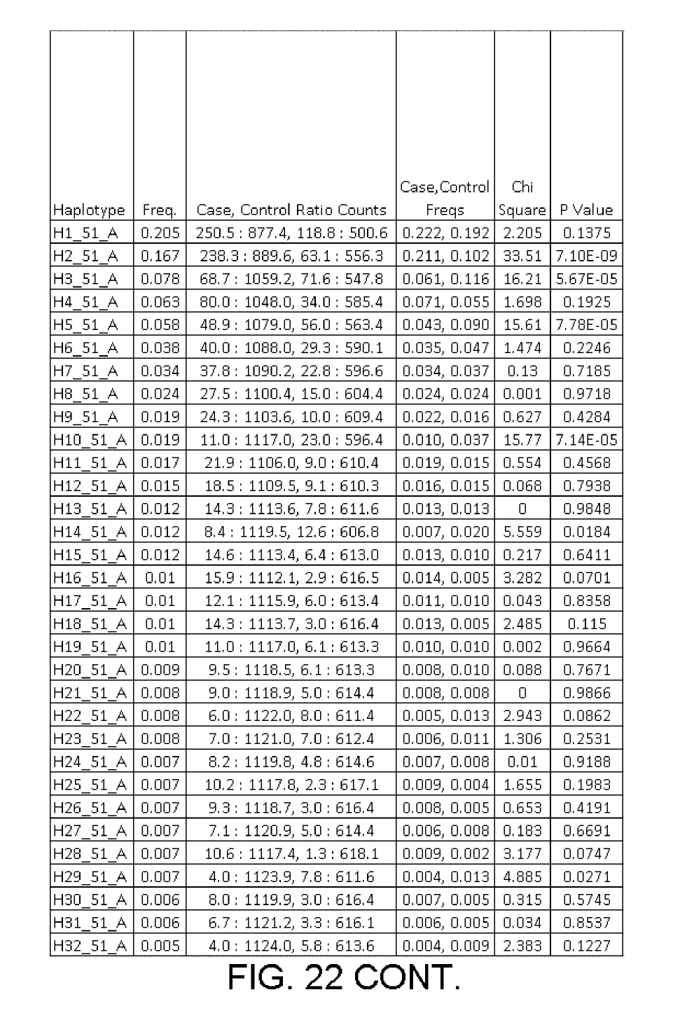

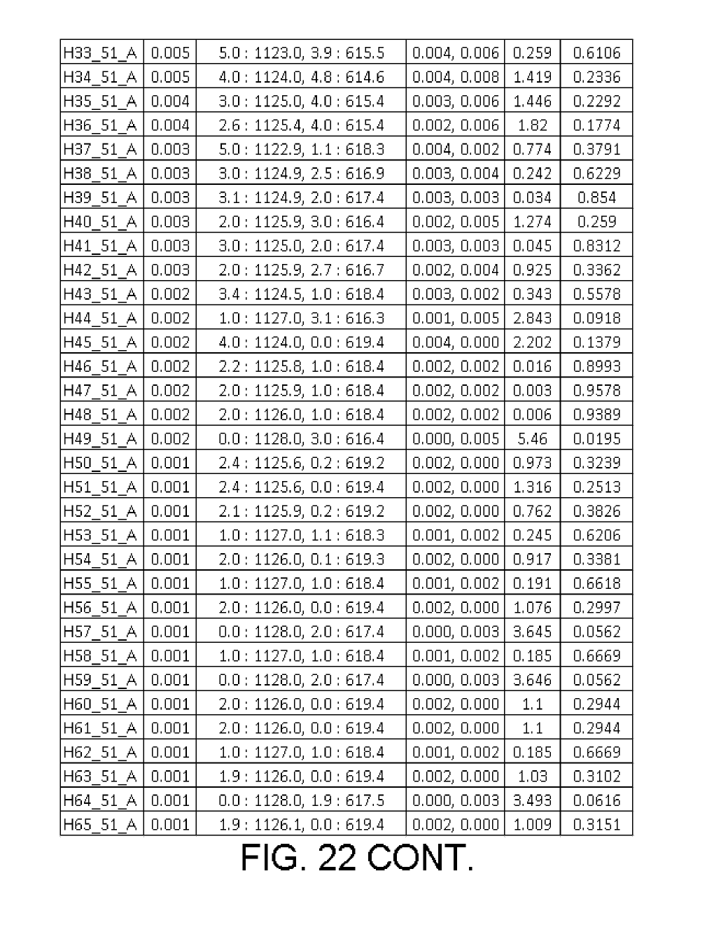

51 SNP-based haplotypes were also generated using the 1,073 (927 Subset) Cohort (FIG. 22). Converting from 62 to 51 SNPs caused some of the 62 SNP-based haplotypes to collapse into the 51 SNP-based haploytpes (FIG. 20). 17 major haplotypes occurred at frequencies greater than 1% in this cohort.

3.8 Results: Validation of 62 SNP-Based and 51 SNP-Based Haplotypes Using the UGRP Cohort

Because haplotypes were imputed, independent verification was sought that the haplotype structures and frequencies determined were real and representative of a Caucasian population. 62 SNP-based haplotypes were validated as described in see Example 2.

Using 136 unrelated individuals from the CEPH/UGRP reference cohort, non-imputed haplotypes were generated using 51 SNPs. Given the small size of the UGRP unrelated cohort, some differences in haplotype frequency between the GSH Cohort and the UGRP cohort was expected. However, when compared to the haplotypes shown in FIG. 1, these differences were minor and haplotype frequencies were nearly similar (FIG. 33), indicating that these haplotype frequencies were representative of aged Caucasian populations in general (FIG. 10).

3.9 Results: Single SNP AMD Disease Associations in the 1,073 Cohort (927 Subset) Using 62 SNPs

Of the 1,073 samples in our original cohort, 927 were 60 years or older at diagnosis and had AMD (Grade 1B-4C) or did not have AMD and served as controls (Grade 0). A case/control analysis of risk was performed using logistic regression for single marker associations (FIG. 5). Results showed patterns of association. rs1061170 was significantly associated with risk (p=2.5e-11, OR=1.953, 95% CI 1.604-2.377). The minor alleles of rs1410996, rs2274700, and rs3753395 [and other surrogates tagging the block of DNA containing variants that associate with the majority of protective haplotypes (chr1:196679455-196701284; Hg19)], were even more strongly associated than rs1061170, but with protection from developing, rather than risk of developing AMD (p=1.12e-14, OR=0.436, 95% CI 0.353-0.538). Variants in the distal region of this locus including CFHR5 and F13B were also significantly associated with AMD, albeit at much lower levels (FIG. 5).

3.10 Results: 62 SNP-Based Haplotypes Associated with AMD Risk: 1,073 Cohort (927 Subset)

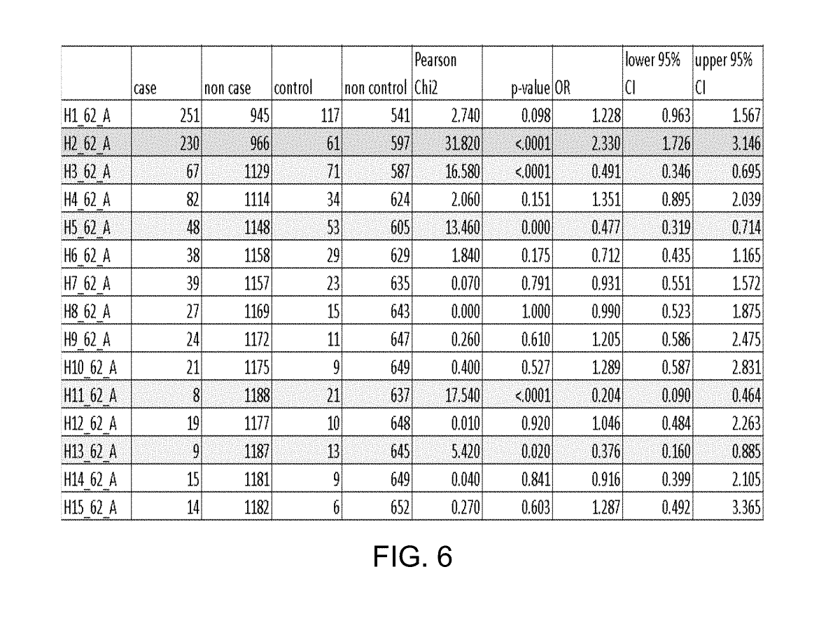

Disease associations were analyzed for the entire extended haplotypes using Haploview 4.2 (FIG. 6) (Barrett J C, Bioinformatics, 2005 Jan. 15).

Of the 15 major haplotypes present at a frequency greater than 1%, H2_62_A exhibited significant risk for AMD (FIG. 6 and FIG. 18; p<0.0001; note that values vary slightly between FIGS. 6 & 18 due to clinical reclassification of a few patients between the time these two analyses were conducted). H1_62_A, which also contains the 402H risk allele trended slightly toward risk for AMD (p=0.098) in this cohort, but was less significant than H1_62_A. Whereas four distal SNPs from the 62 SNP panel distinguish H1_62_A from H2_62_A, only one of these SNPs significantly associated with AMD in single marker assessment (rs698859, p=0.0111) (FIG. 5). This indicated that additional features/elements in the haplotypes tagged by these SNPs are responsible either for risk in H2_62_A or lack of risk in H1_62_A. Given multiple haplotypes contain the risk allele at rs1061170 (H1_62_A, H2_62_A, H8_62_A, H12_62_A, and H14_62_A) and yet only one of these haplotypes associated significantly with risk in this cohort, there is likely a risk factor present in the H2_62_A haplotype that is lacking in all other non-H2_62_A 402H haploytpes. In fact, previous studies using less extended haplotypes that failed to include the F13B SNPs have attributed a significant risk of developing AMD to all patients containing the Y402H risk allele (42.3% of major haplotypes or 49% of all haplotypes in the GSH cohort), when in fact risk only associated with H2_62_A, or 16.5% of the cohort described herein. Thus, the haplotypes described herein represent a significant increase in the sensitivity of defining risk at the CFH locus.

Furthermore, the vast majority of AMD risk association, although not all, occurred in major haplotypes tagged by CFH 402H (H2_62_A, H8_62_A, H12_62_A, H14_62_A and H16_62_A; FIG. 6 and FIG. 18) in this cohort. CFH 402H, however, was not always associated with risk for AMD; H2_62_A was the most strongly associated haplotype with AMD risk, whereas major haplotypes H1_62_A, H8_62_A, H12_62_A, and H14_62_A were not significantly associated in this cohort. The minor haplotype H16_62_A trended toward risk in this cohort.

3.11 Results: 62 SNP-Based Haplotypes Associated with AMD Protection: 1,073 Cohort (927 Subset)

In addition to risk, several major CFH-to-F13B haplotypes showed protection from AMD relative to the cohort as a whole. H3_62_A (includes the rs800292 A allele) was significantly protective (FIG. 6). Just as H1_62_A and H2_62_A differed in their risk association, so to did H3_62_A (protection) with the other major haplotype containing the rs800292 A Allele, H6_62_A (neutral). Of the haplotypes containing CFHR3/CFHR1 deletions, H5_62_A and H11_62_A showed significant protection, while H13_62_A showed marginal protection and H15_62_A was neutral. H3_62_A and H6_62_A are identical until mrd_3905/rs75816959, which is in CFHR5. Similarly the CFHR3/CFHR1 deletion containing haplotypes H5_62_A, H13_62_A, and H15_62_A are identical until mrd_3905. Thus, placing these haplotypes in the context of extended haplotypes that included CFHR5 and F13B resulted in significantly refined association with AMD (FIGS. 6 & 18).

3.12 Results: 62 SNP-Based Haplotypes Associated with Neutrality: GSH Cohort

Several major CFH-to-F13B haplotypes, including H4_62_A, H7_62_A, H10_62_A, H12_62_A, and H14_62_A, were neutral with respect to the development of AMD relative to the cohort as a whole. The minor allele of rs3753396 tagged many of these neutral haplotypes (FIG. 1, FIG. 3, FIG. 6, and FIG. 18). Other SNPs in LD with rs3753396 at a r2 value of 90 or greater and that can be employed as surrogates for rs3753396 include (listed proximal to distal): rs10489456, rs70620, rs742855, rs11799380, rs1065489, rs11582939, rs385390, rs421820, rs426736, rs370953 and rs371075.

3.13 Results: Gender Associations in 62 SNP-Based Haplotypes: 1,073 Cohort (927 Subset)

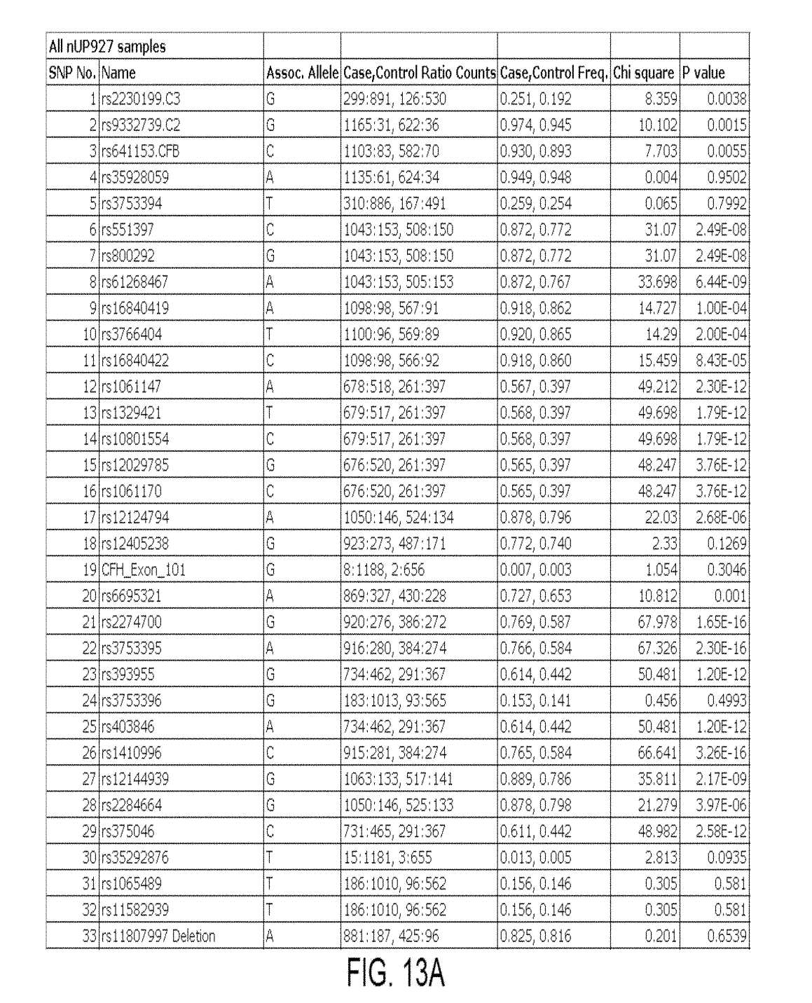

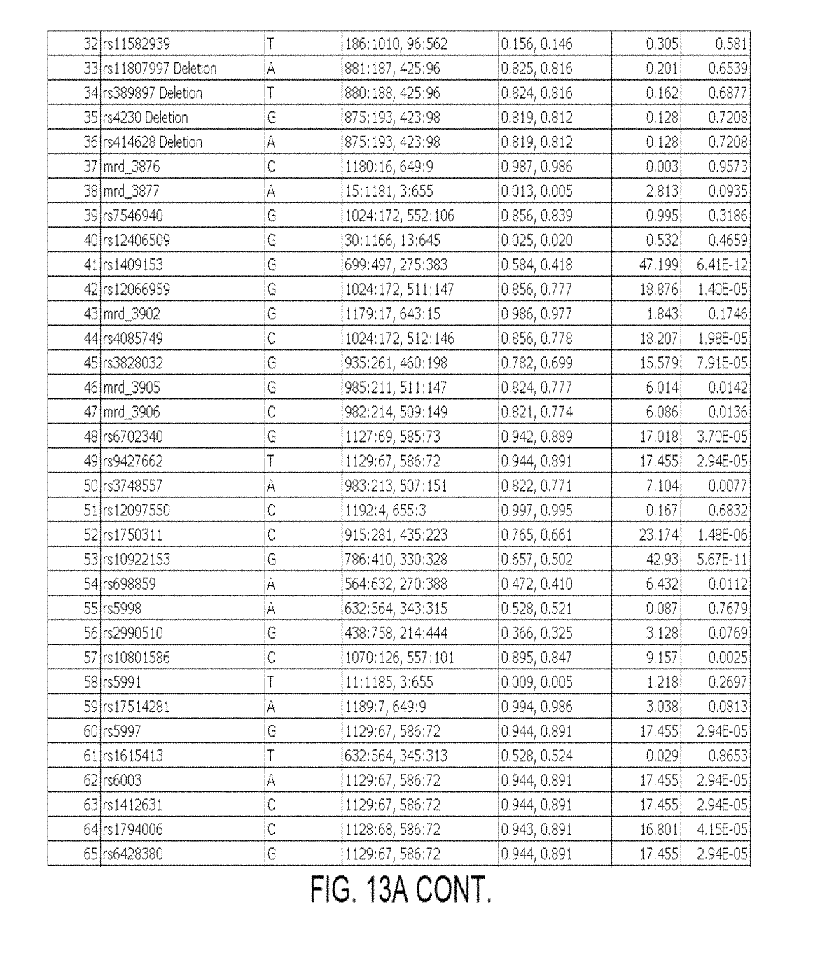

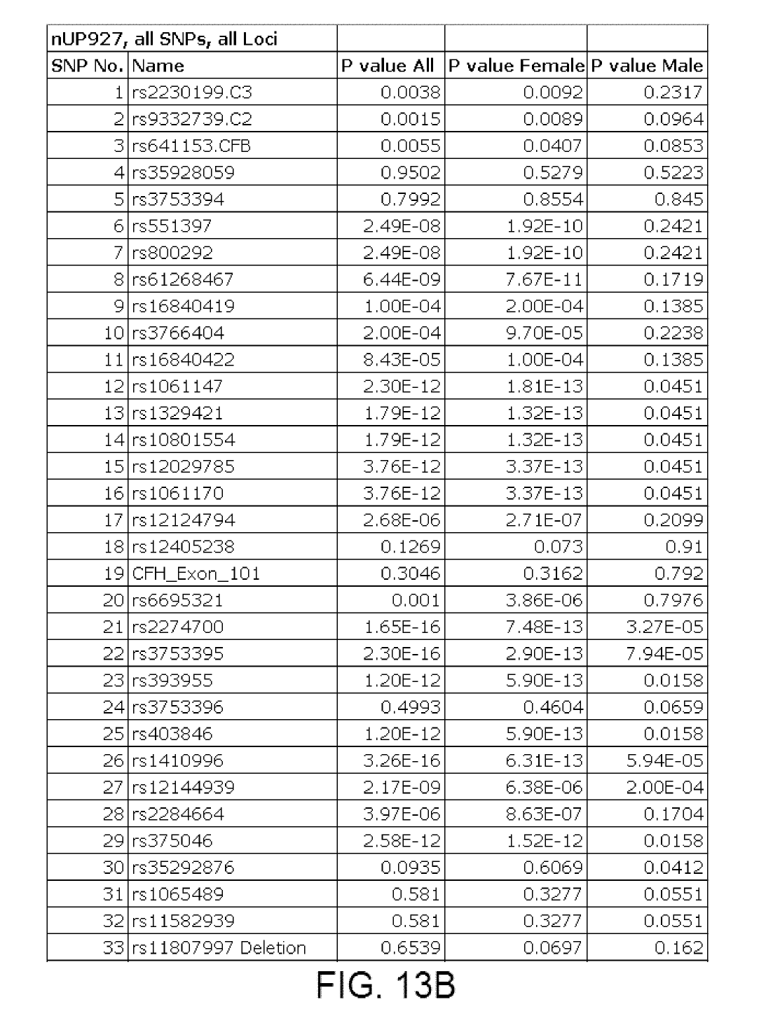

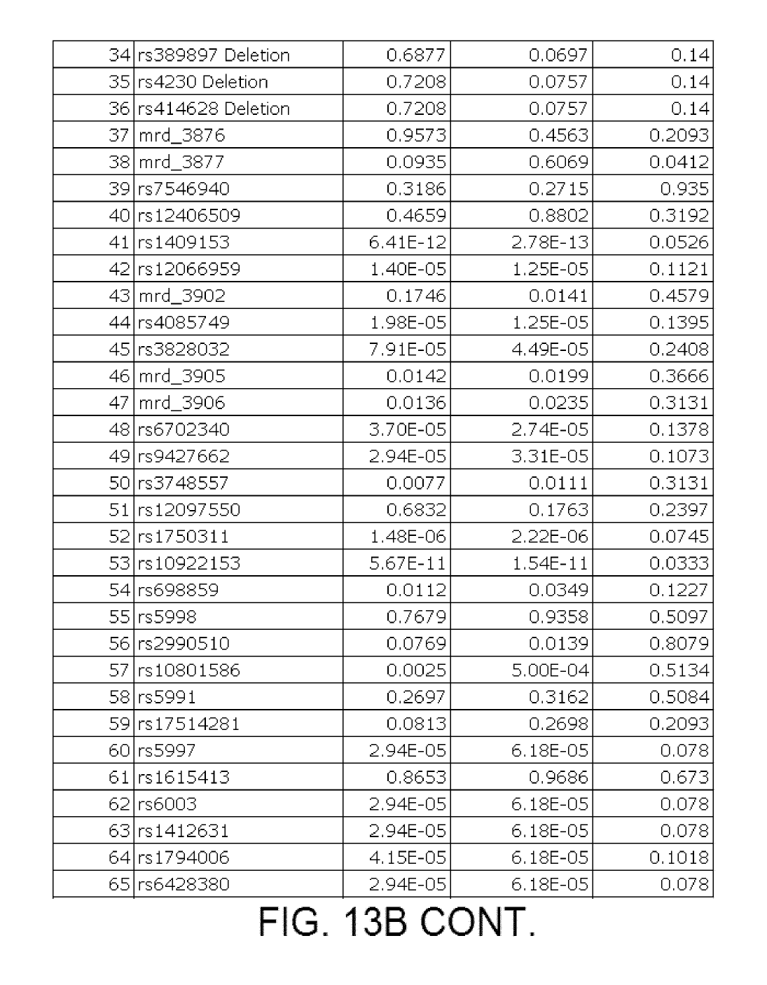

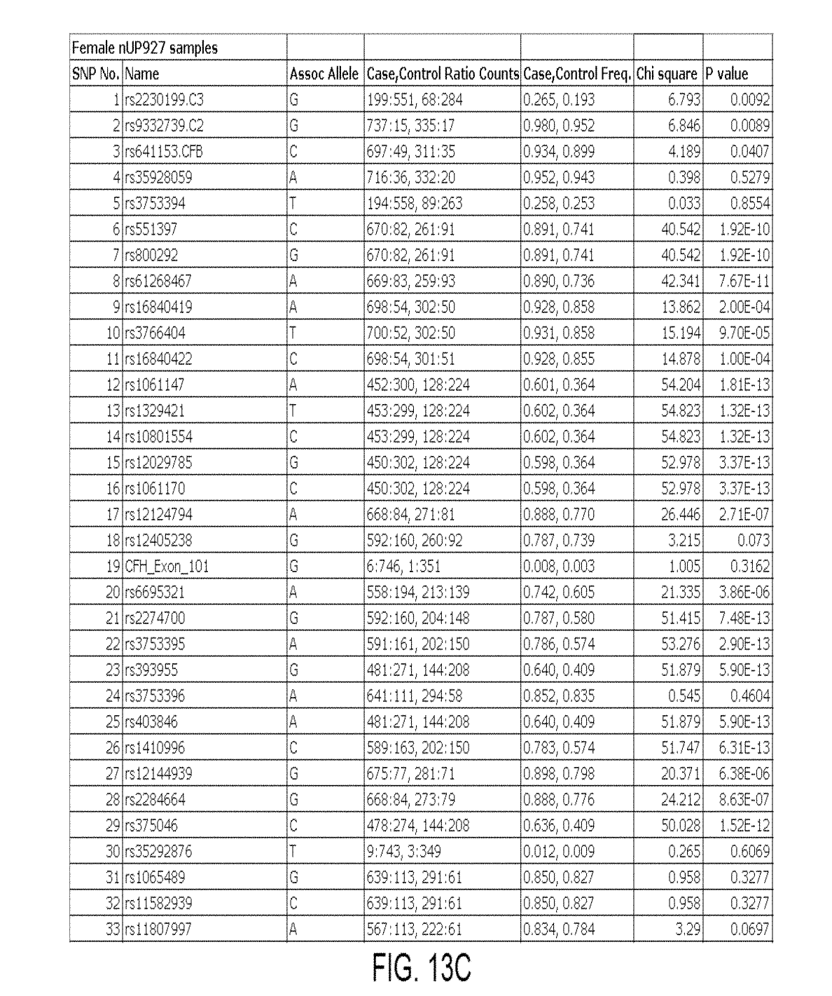

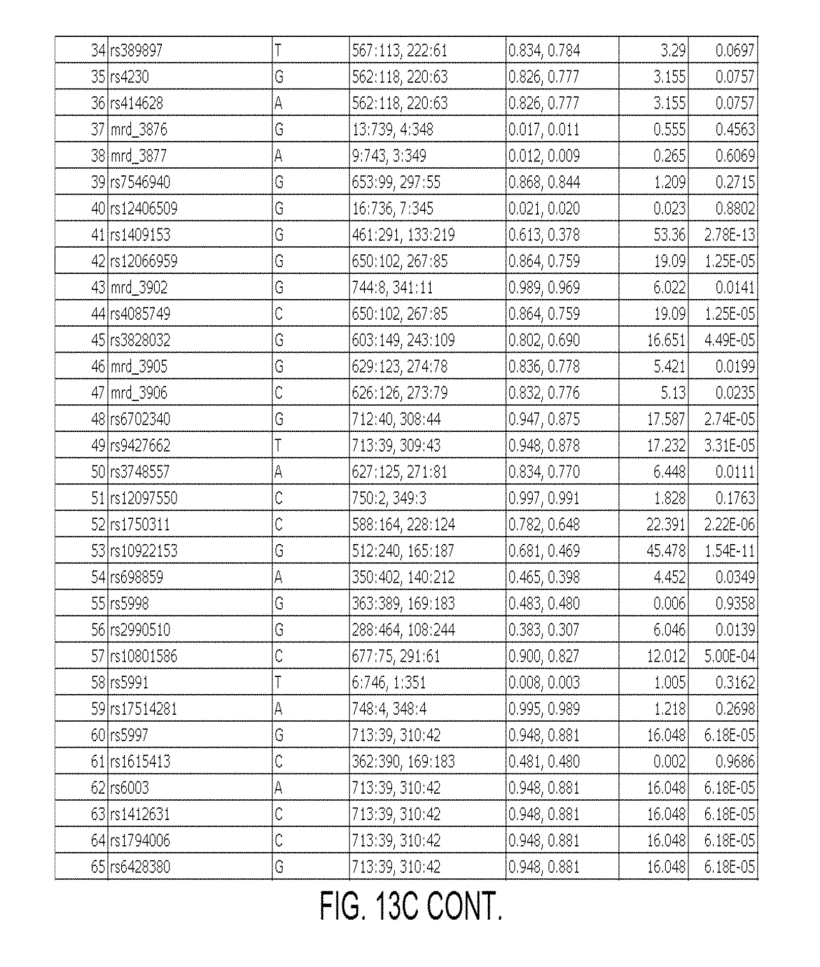

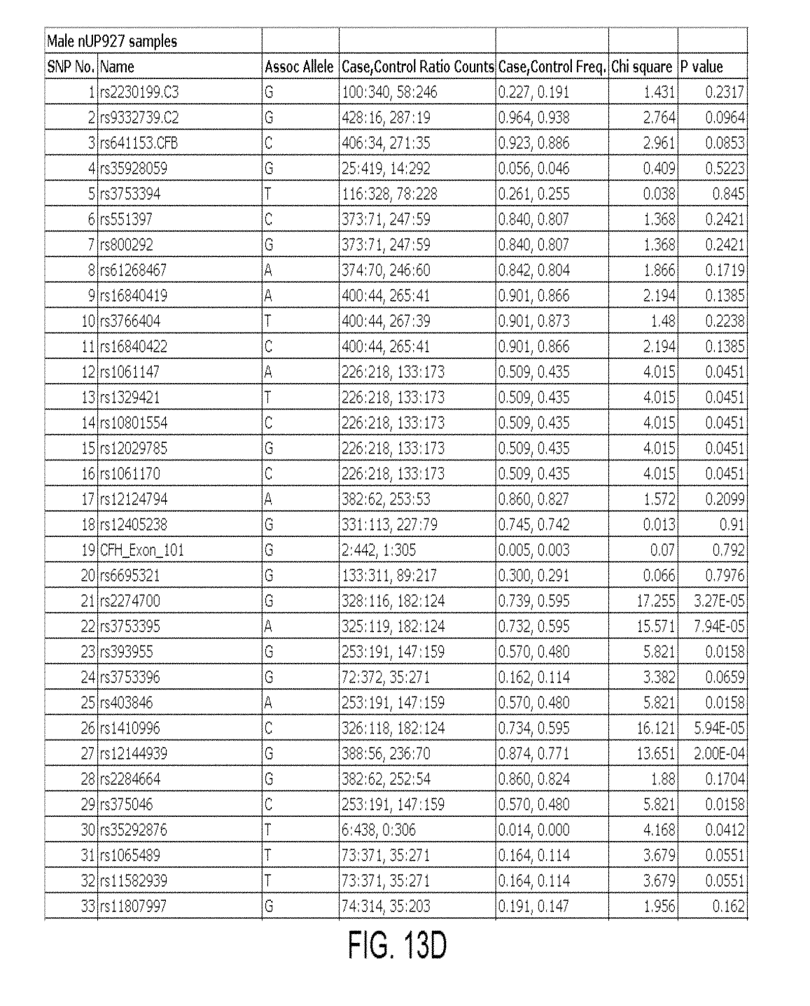

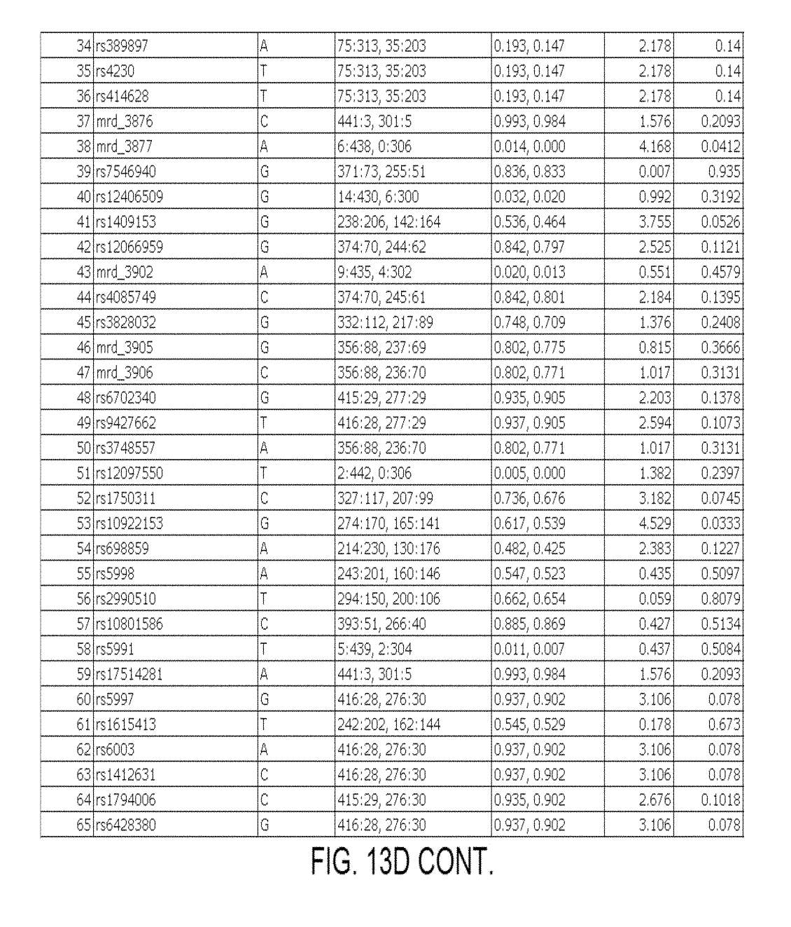

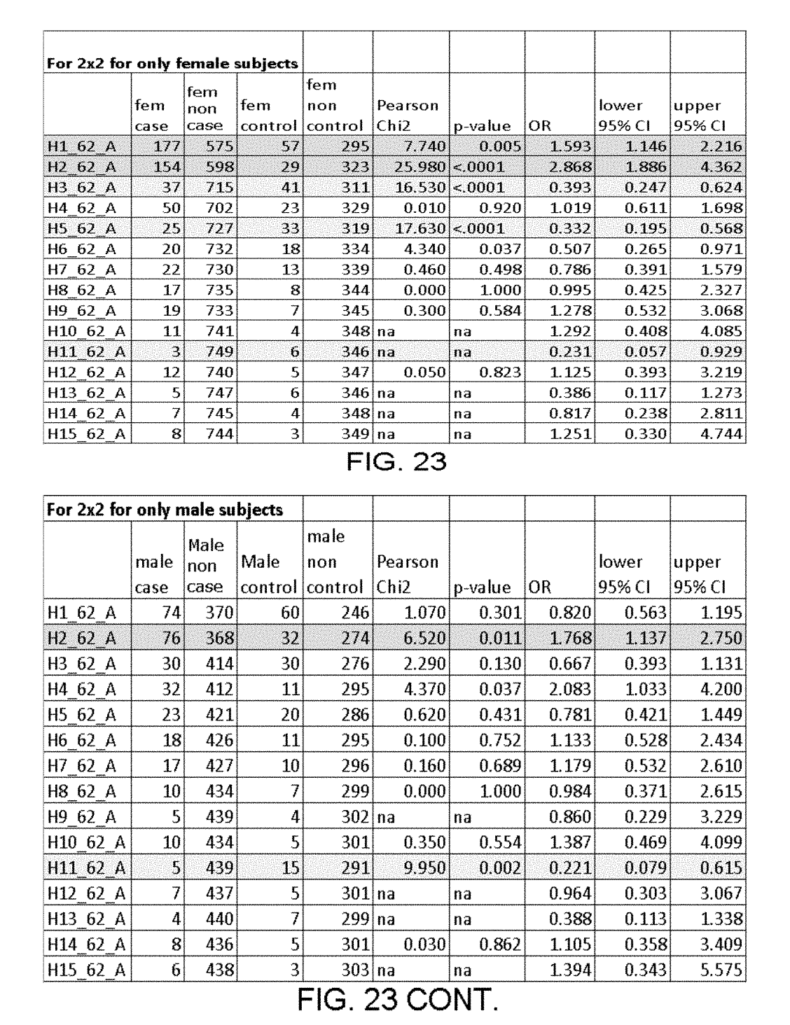

62 Single marker (SNPs) and haplotypes were examined for potential association with gender in the GSH case-control cohort. Associations were assessed using the entire cohort, the male sub-cohort only, and the female sub-cohort only (FIG. 13 and FIG. 23). The female cohort showed very similar levels of significance in the association of the 62 individual SNPs to AMD when compared to the '927 GSH Cohort (FIG. 13). Male associations differed from the female associations. For example, association of rs800292, rs1061170 and rs1329421 was not observed in the male sub-cohort, a difference of approximately 11-orders of magnitude relative to females. Males did show lower levels of significant association with rs1410996, rs3753395 and rs393955, although also at levels up to 11-orders of magnitude less than was seen in the female cohort (FIG. 13). The rs800292 A allele, previously associated with protection or reduced risk for AMD, also showed decreased association in the male sub-cohort. This data indicated that by controlling for sex, previous studies may have missed this stratification because of masking.

While the loss of association with AMD in males was significant throughout the CFH gene, one block of significant association remained, albeit at reduced levels. rs1410996 showed a p-value of 5.94e-5 (compared to 3.26e-16 in the full cohort and 6.31e-13 in females) (FIG. 13). This SNP was found to be in close to 100% LD with several proxy SNPs (r52274700, rs3753395, and r510737680). When looking at the female sub-cohort, rs1410996 had a similar strength of association to AMD as does rs1061170. It was the association of rs1410996 in males, an association lacking at rs1061170, which accounted for the higher significance of rs1410996 (and its proxy SNPs rs2274700, rs3753395, and r510737680). By analyzing males and females independently, a basis was identified for the disparity between risk at rs1061170 and rs1410996.

Studies suggested the male/female stratification seen at CFH was not a result of AMD disease in general, but of a specific mechanism of disease associated with CFH. Analysis of SNPs in C3 and C2/CFB showed much the same trend as with CFH: weaker but significant risk was found in both the full 927 cohort and the female-only cohort, while no risk was found in the male-only cohort. Thus, risk for AMD in males was much less associated with the complement system as a whole than in females.

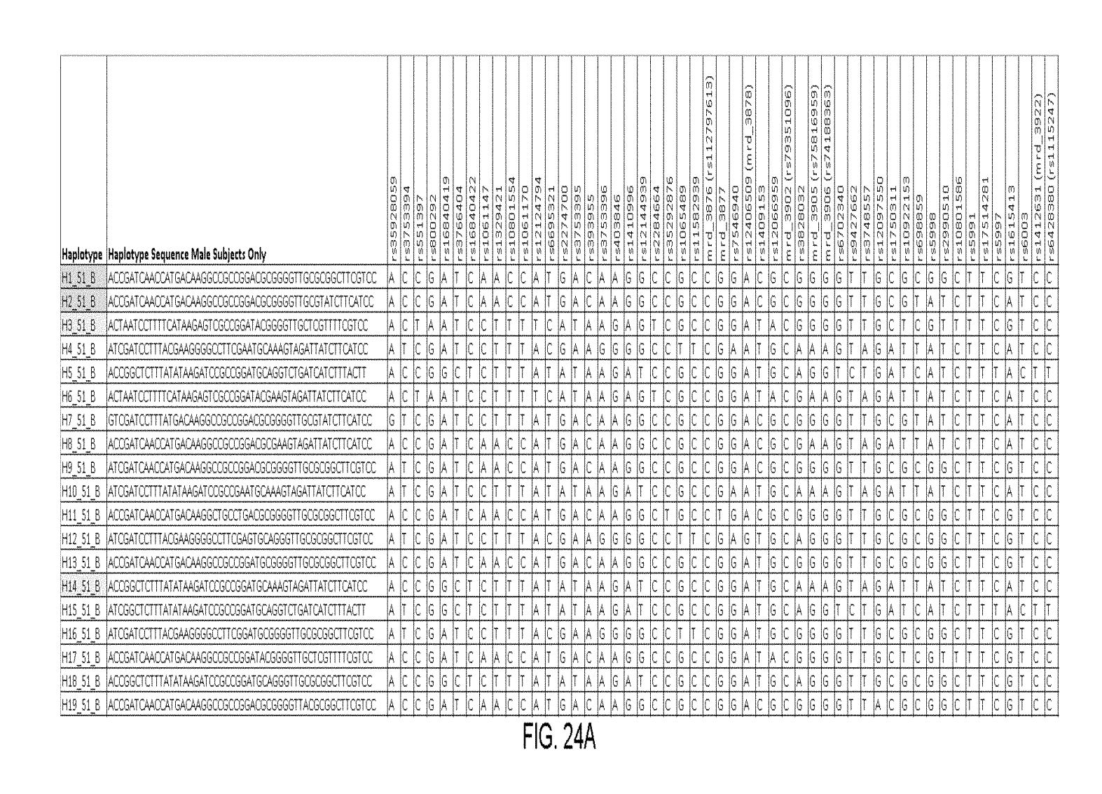

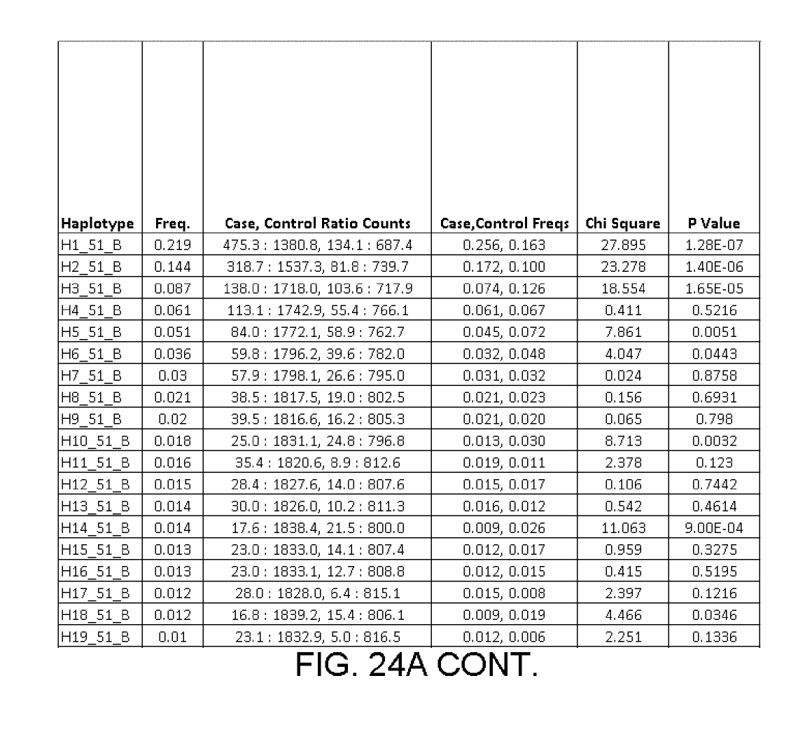

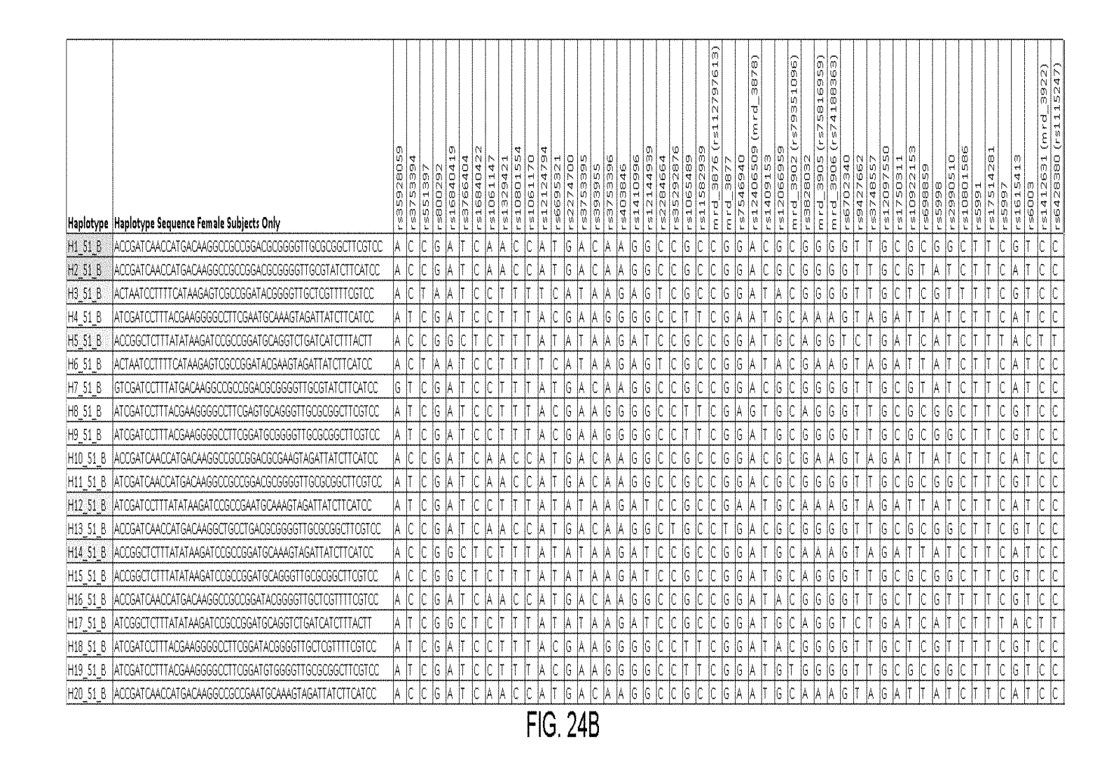

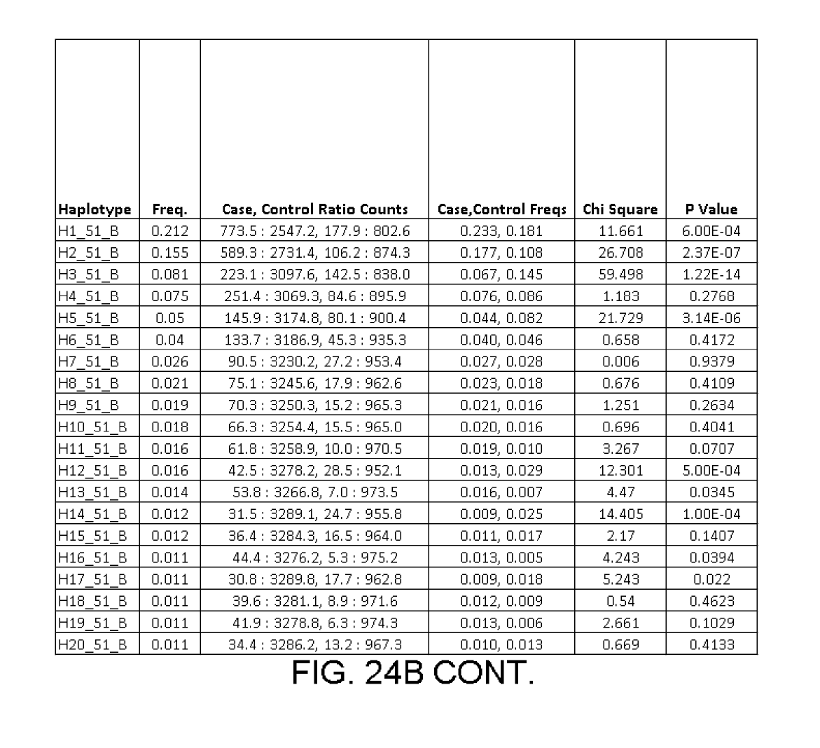

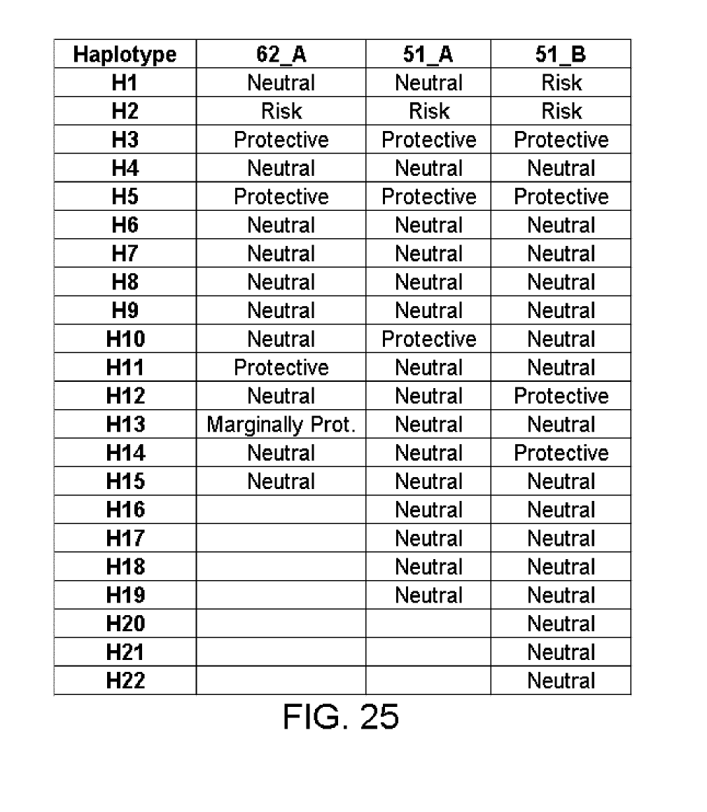

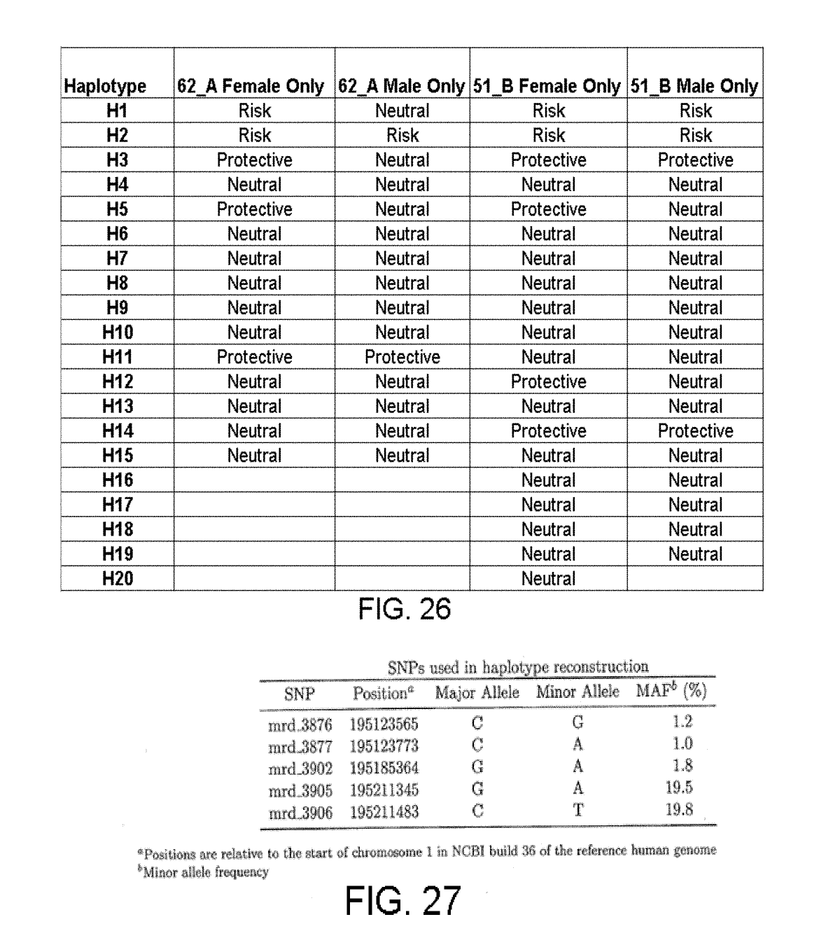

Haplotype disease associations were also examined independently in males and females. H2_62_A was barely significant in males but strongly significant in females (FIG. 23). H2_51_B was more significant in males than H2_62_A, but still less significant than in females (FIG. 24). H1_62_A was significant in females, not at all in males. Whereas, H1_51_B was significant in both males and females; however, it was again more significant in females (FIG. 24). H11_62_A was significant in both males and females, but was more significant in males. H2_62_A, H3_62_A, H5_62_A, and H11_62_A were all significantly associated with AMD in all of the subjects (FIG. 6) and also in the females (FIG. 23). Females were slightly more significantly associated than the all-subjects in H1_62_A and H6_62_A, and less significantly associated in H13_62_A, but these differences were fairly minor.

3.14 Results: 51 SNP-Based Haplotypes Associated with AMD: 1,073 Cohort (927 Subset)

The association of haplotypes in the GSH 927 Cohort was also assessed using a 51 SNP-based haplotype analysis (FIG. 22). The 51 SNPs included are depicted in FIG. 22. The association of risk and protective haplotypes was similar to those observed in the 62 SNP-based haplotype analysis (FIG. 6 and FIG. 18; also see FIG. 20 and FIG. 22).

3.15 Results: 51 SNP-Based Haplotypes and their Association with AMD and AMD Phenotypes: ‘Combined Cohort’ and Extremely Discordant Sib-Pair Cohort

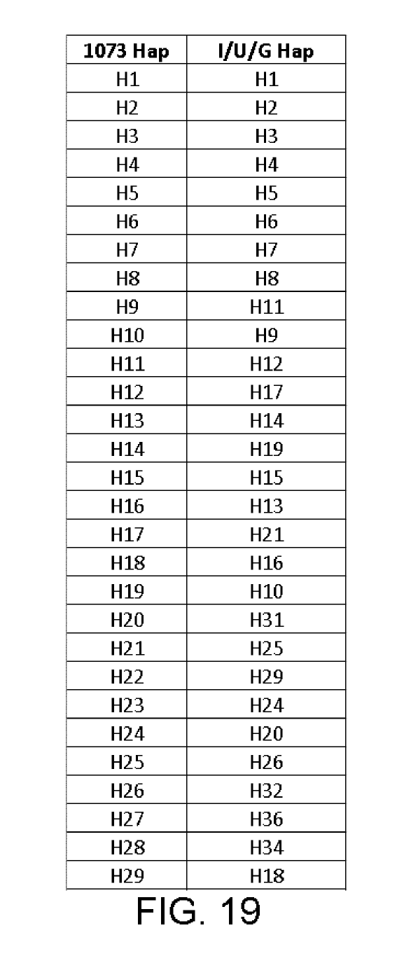

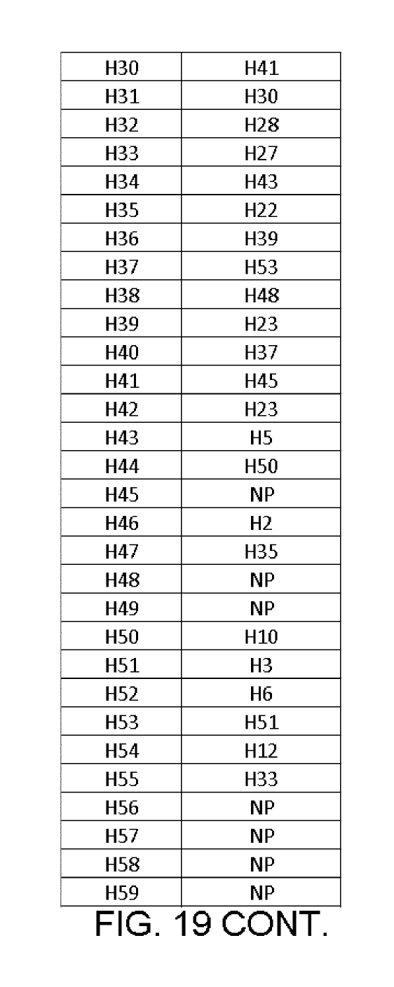

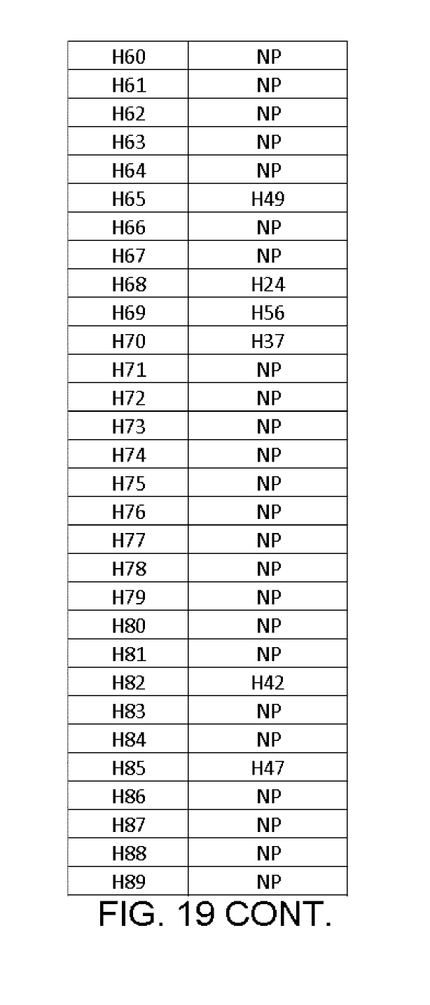

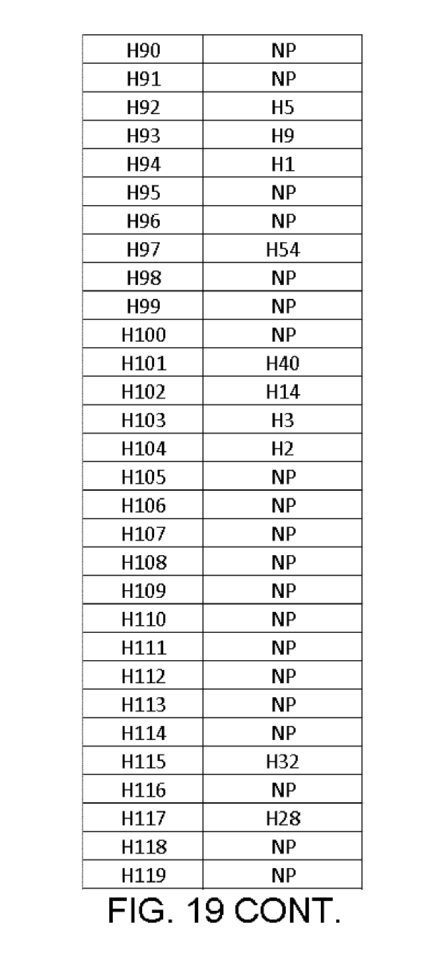

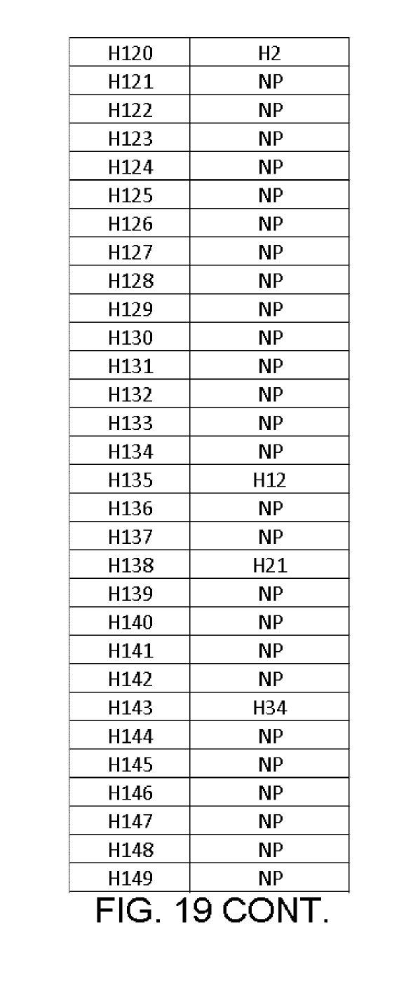

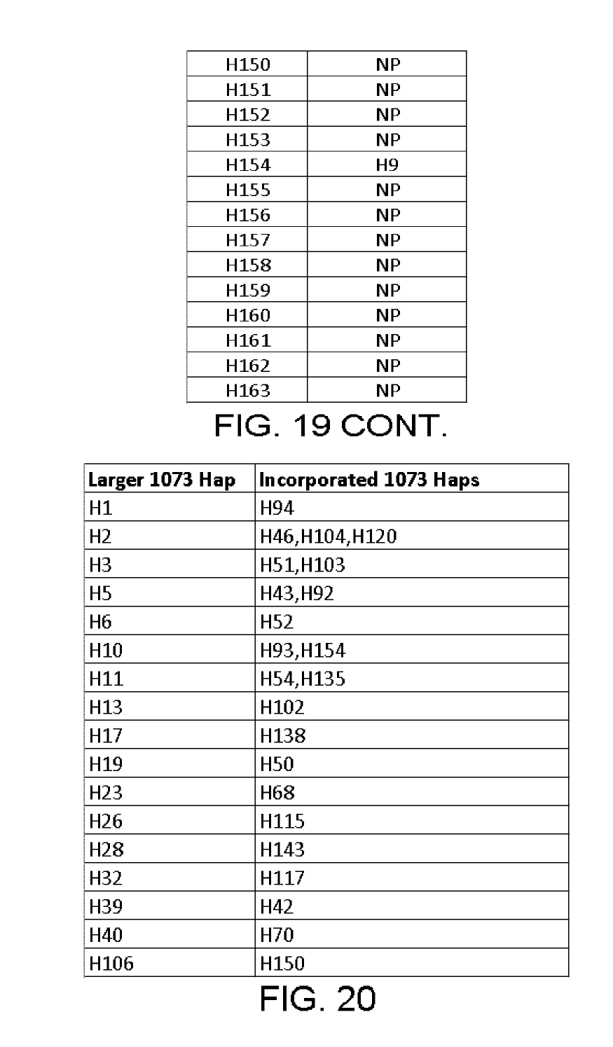

A large case-control cohort comprised of individuals with and without AMD from the Iowa, Utah, and Melbourne cohorts (the ‘Combined Cohort’) was employed to assess 51 SNP-based CFH-to-F13B haplotypes with AMD (controls versus all AMD) risk, protection, and neutrality. Fifty-six haplotypes were generated from the Combined Cohort, 18 of them occurred at frequencies above 1% (FIG. 15). The relationships between the major 51 SNP-based and 62 SNP-based haplotypes are shown in FIG. 16 and the relationship between 51 SNP-based haplotypes in the 1,073 and Combined Cohorts is depicted in FIG. 19. Associations similar to those of 62 SNP-based (Hx_62_A) and 51 SNP-based assessments in the GSH 927 Cohort (Hx_51_A) were observed (FIG. 15). H1_51_B, however, became more significant in this larger cohort.

Several minor CFH-to-F13B haplotypes showed risk and protection from AMD relative to the cohort as a whole. For example, H24_62_A and H28_62_A trended toward risk and H30_62_A (p=0.007) and H40_62_A trended toward protection (FIG. 15).

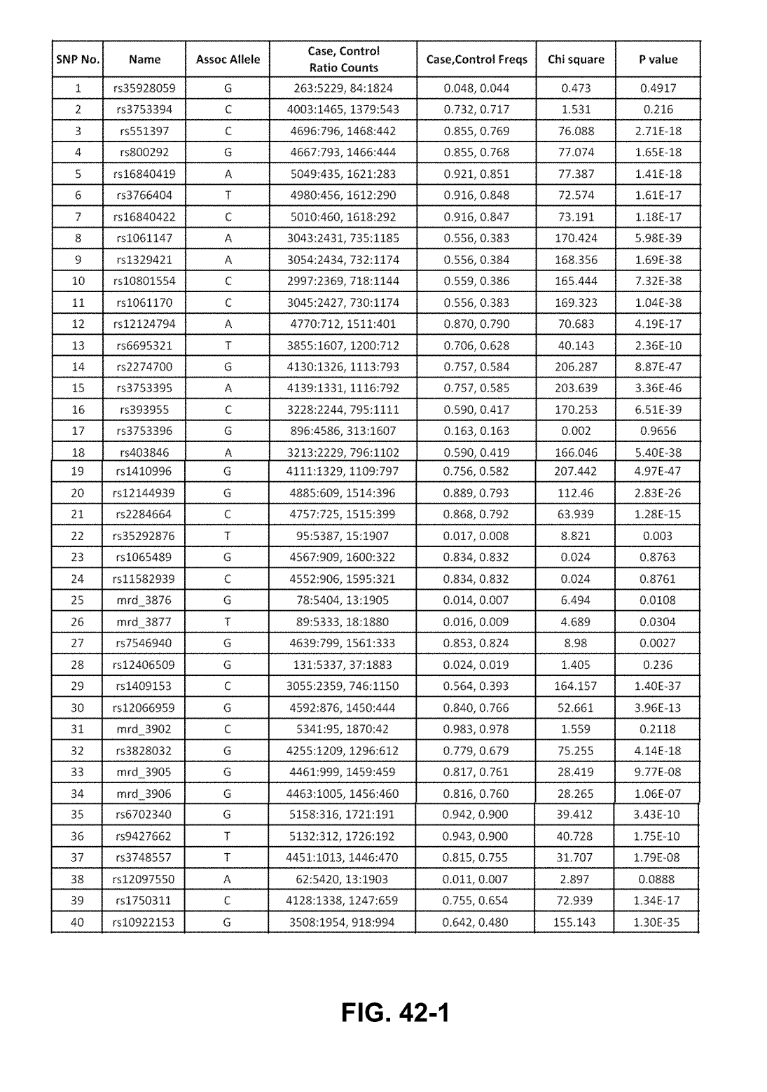

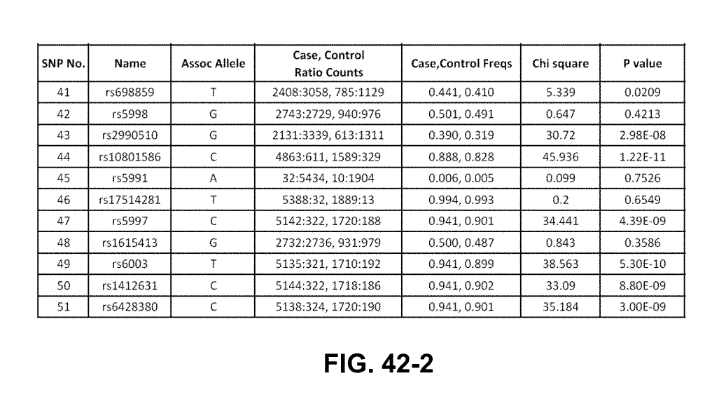

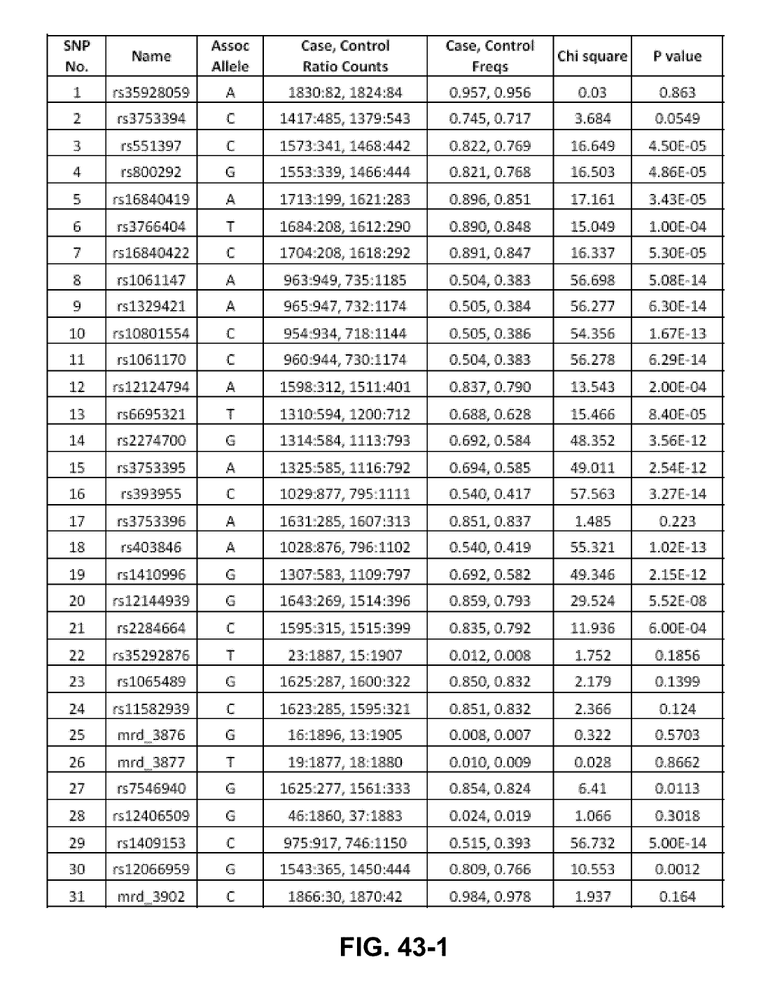

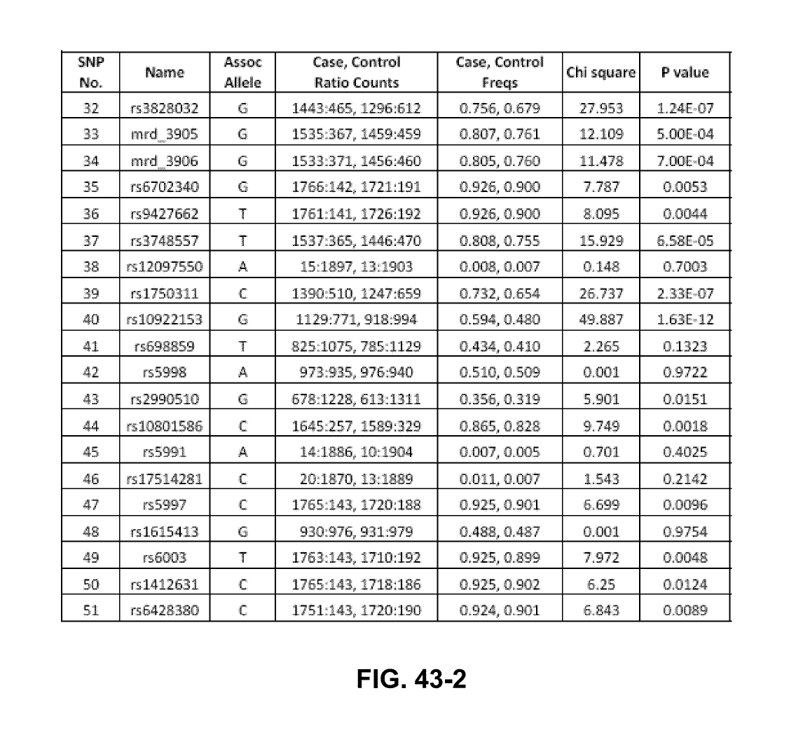

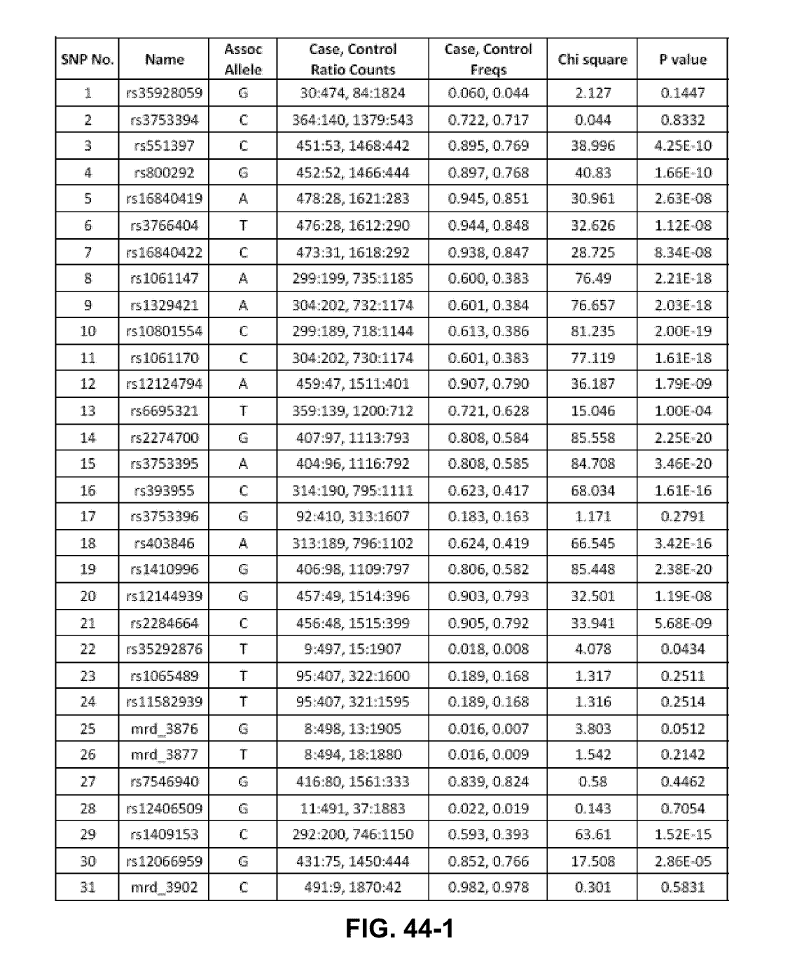

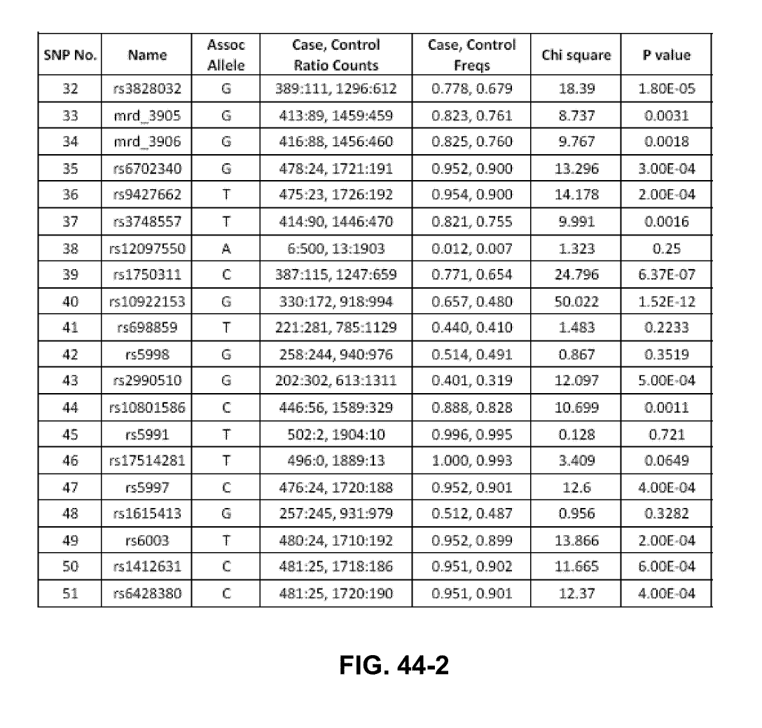

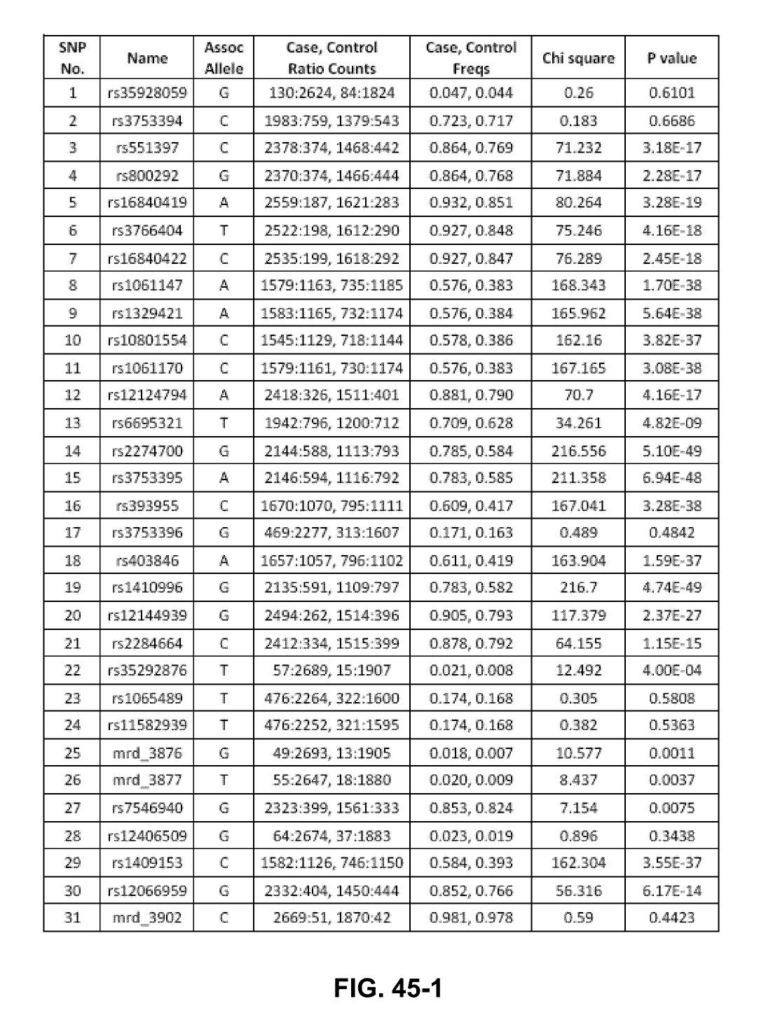

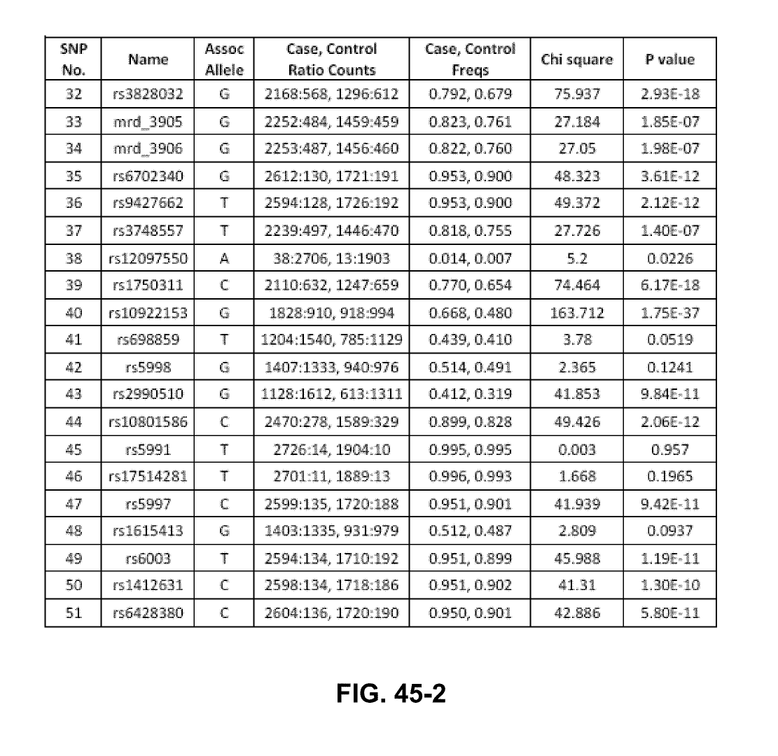

The ‘Combined Cohort’ was also employed to assess the association of single SNP associations of the 51 SNP panel and 51 SNP-based CFH-to-F13B haplotypes with the various phenotypes/stages of AMD, including comparisons of controls (stage 0) to all AMD (stages 1B-4C), early stage AMD (stages 1B-3), and the late stages of geographic atrophy (stage 4A) and CNV (stage 4B). Single marker associations in this cohort (FIG. 42) did not differ from those of the 1,073 and highly discordant sib-pair cohorts when controls were compared to all AMD. Moreover, single marker associations with risk, protection and neutrality did not vary significantly when controls were compared to all AMD stages (FIG. 42), early stage only (FIG. 43), geographic atrophy only (FIG. 44), or CNV only (FIG. 45).

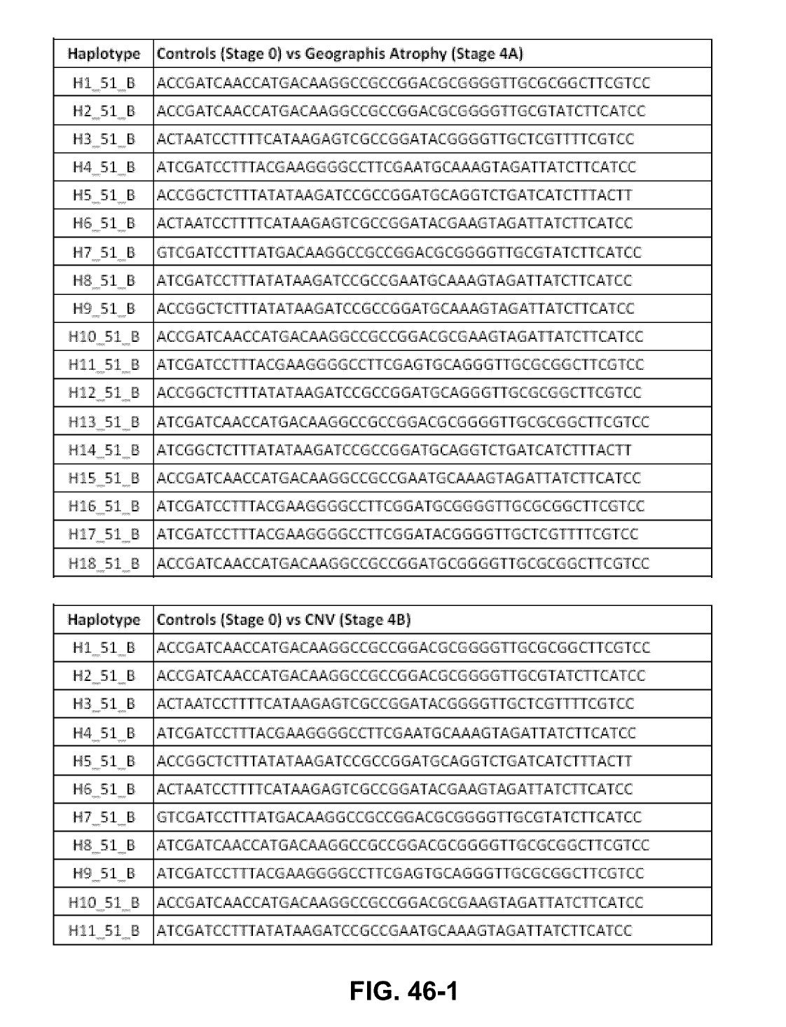

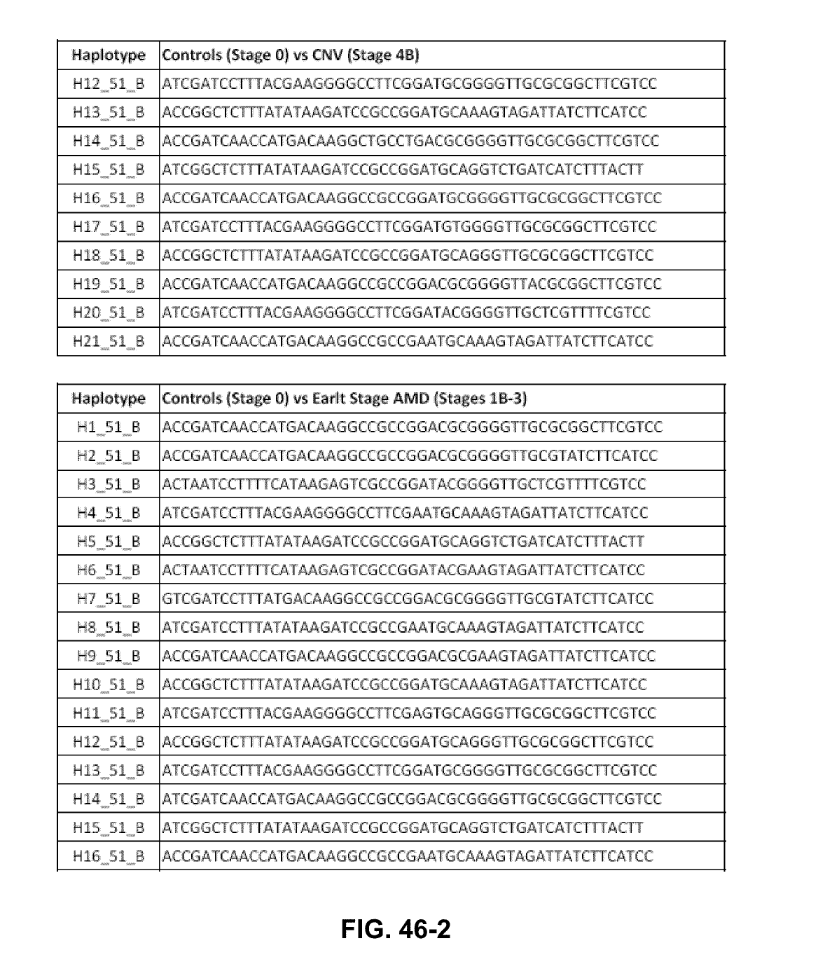

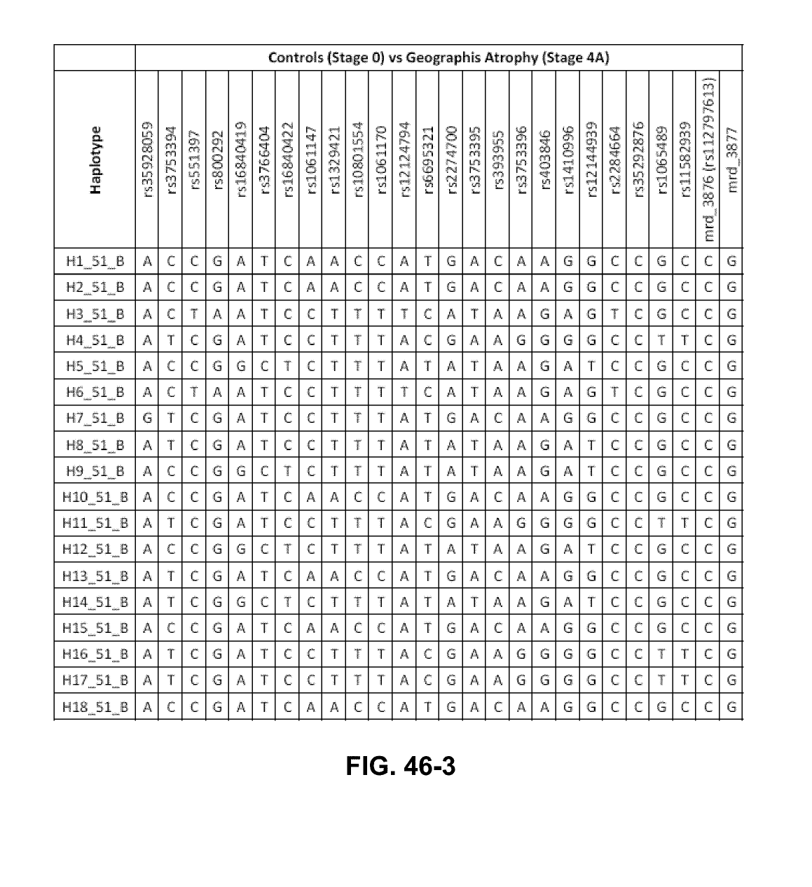

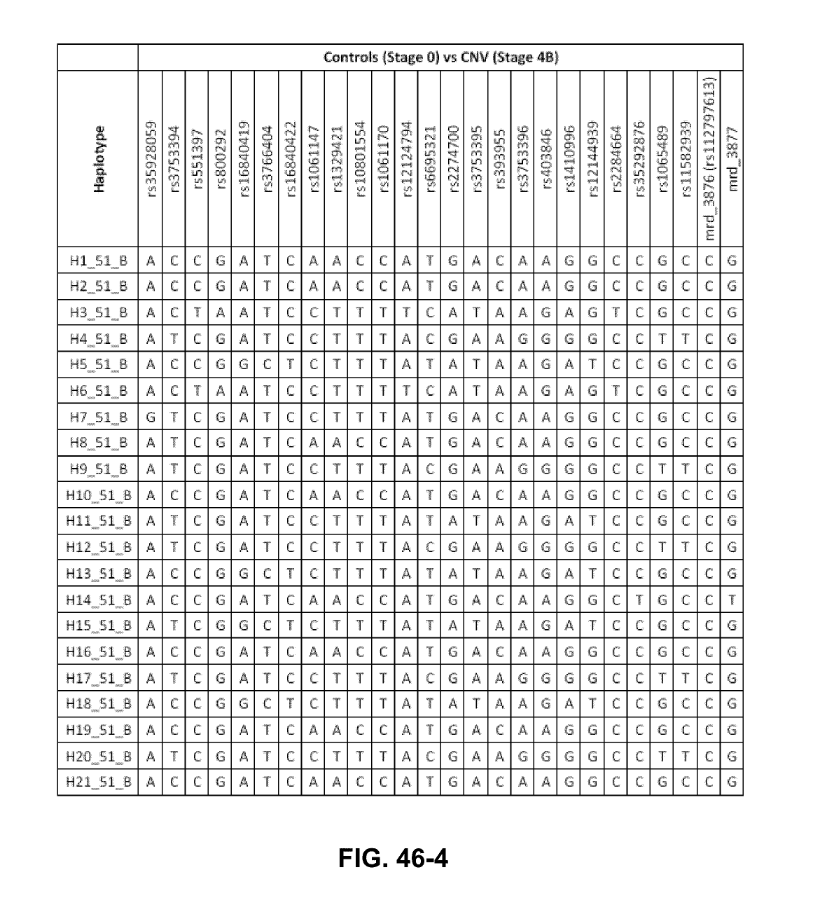

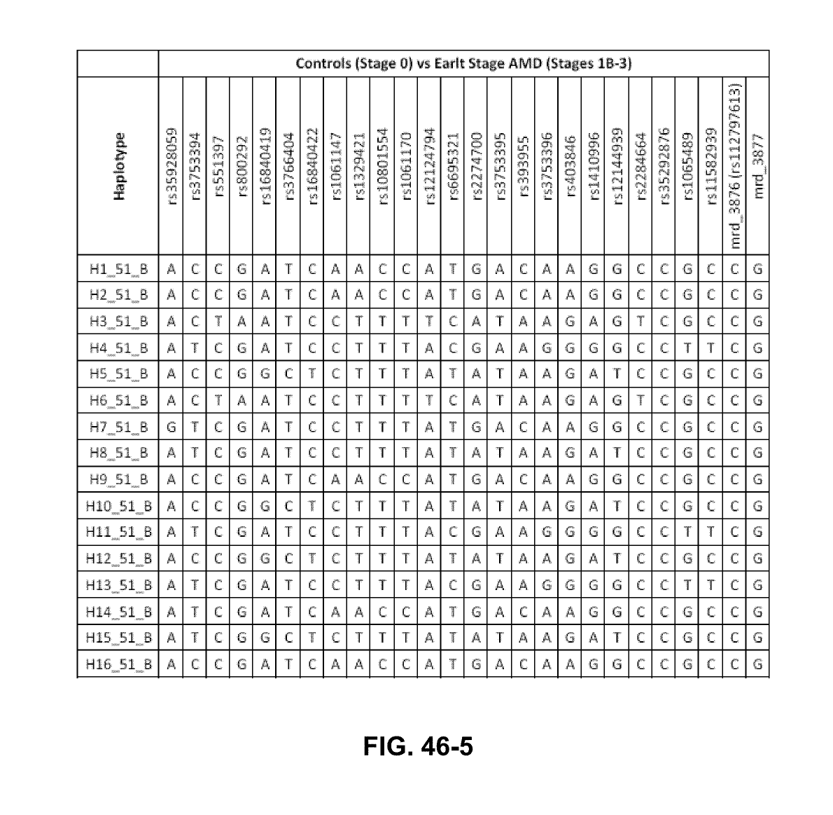

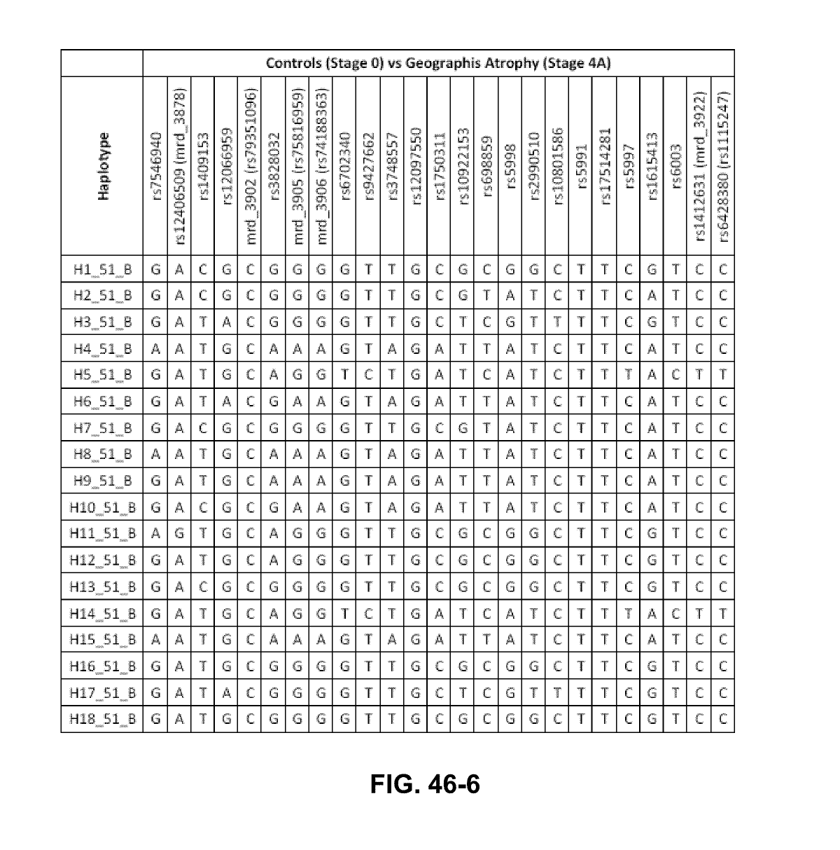

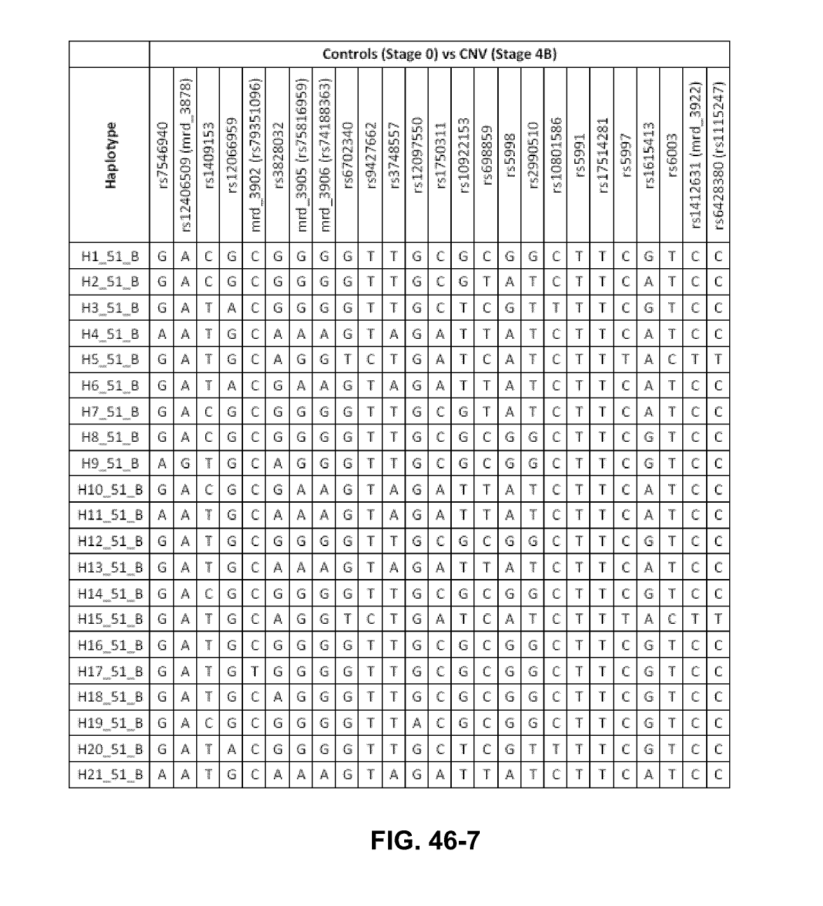

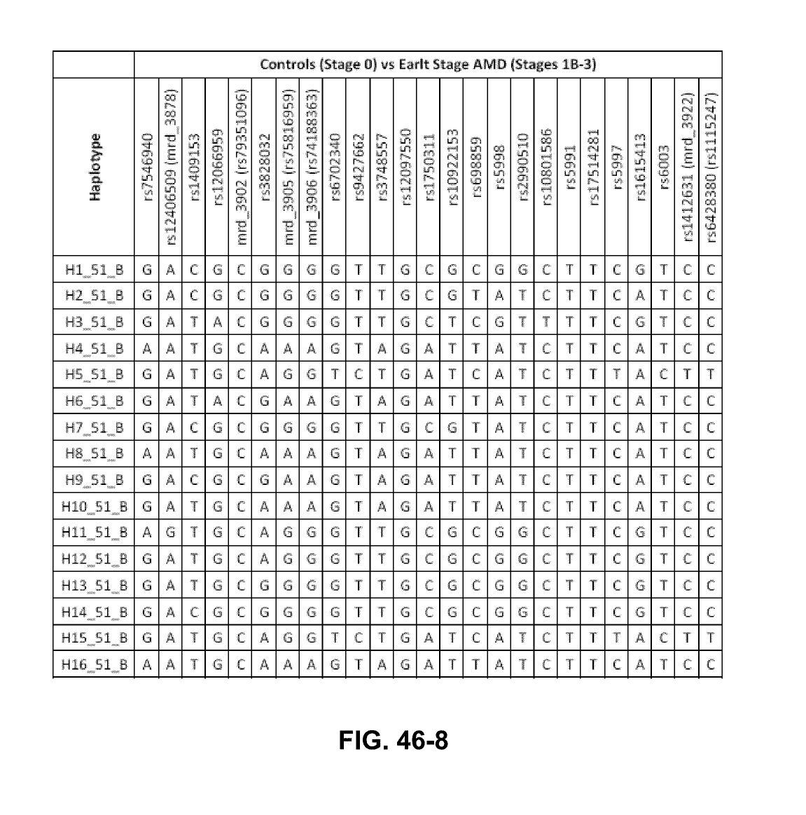

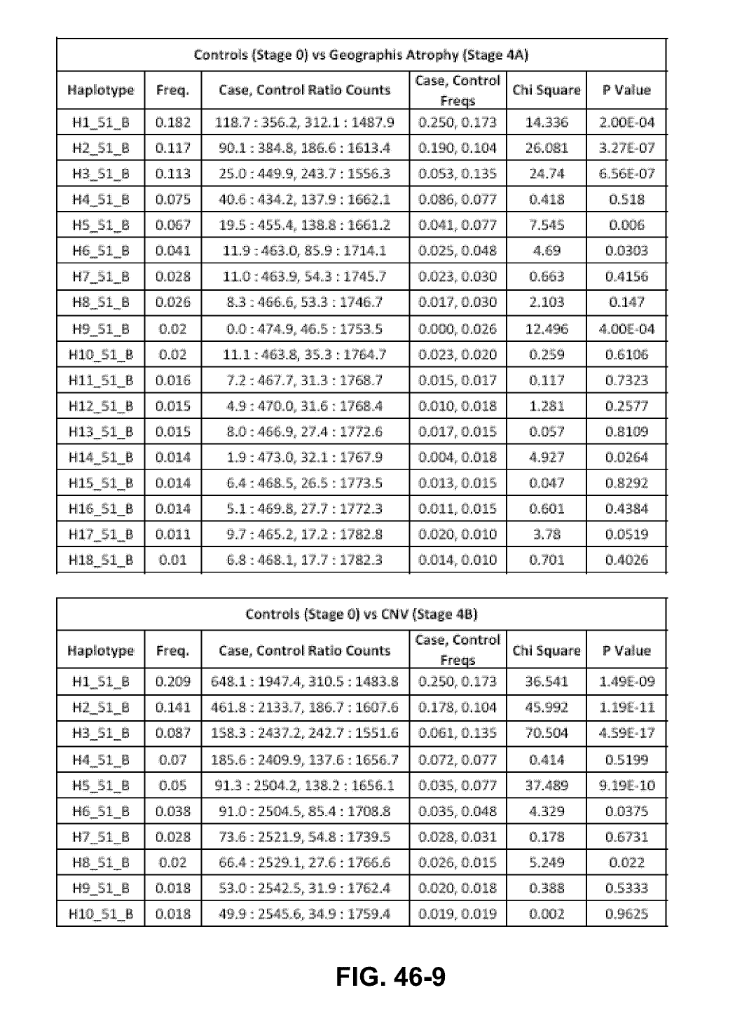

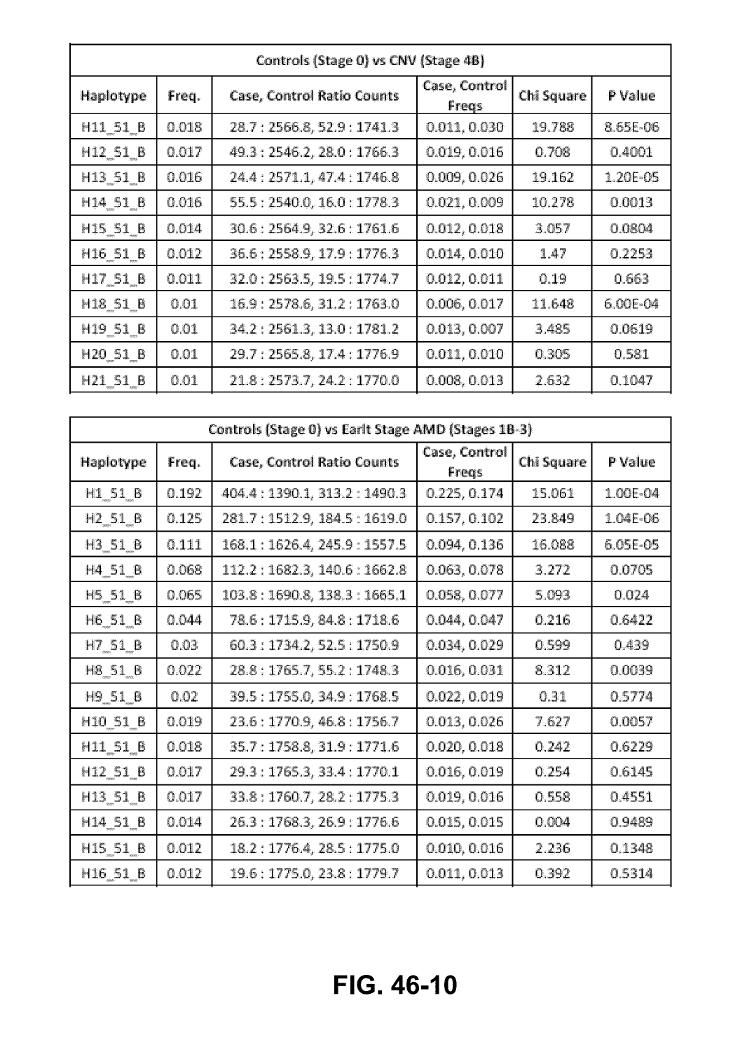

Haplotype associations with risk, protection and neutrality did not vary significantly between AMD phenotypes in the Combined Cohort (nor were they significantly different from analyses of the 1,073 cohort; FIG. 20 and FIG. 22) when controls were compared to early stage only, to geographic atrophy only, or to CNV only (FIG. 46). Note that the haplotype order (based on frequencies) was not identical in the three analyses shown in FIG. 46.

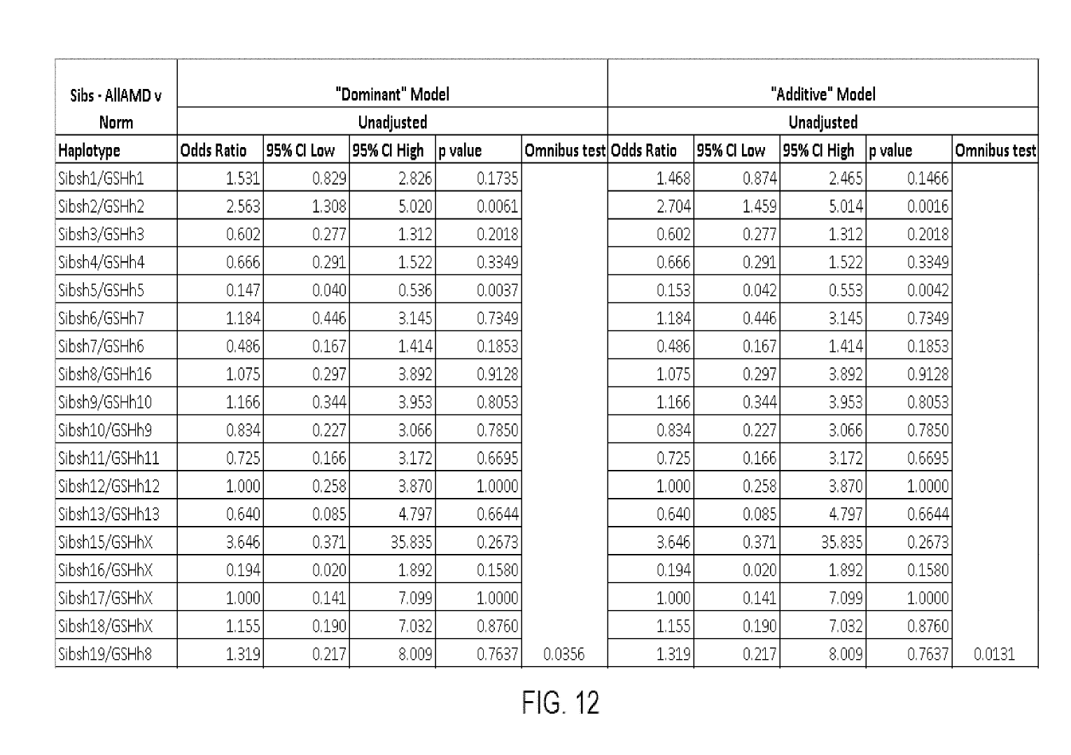

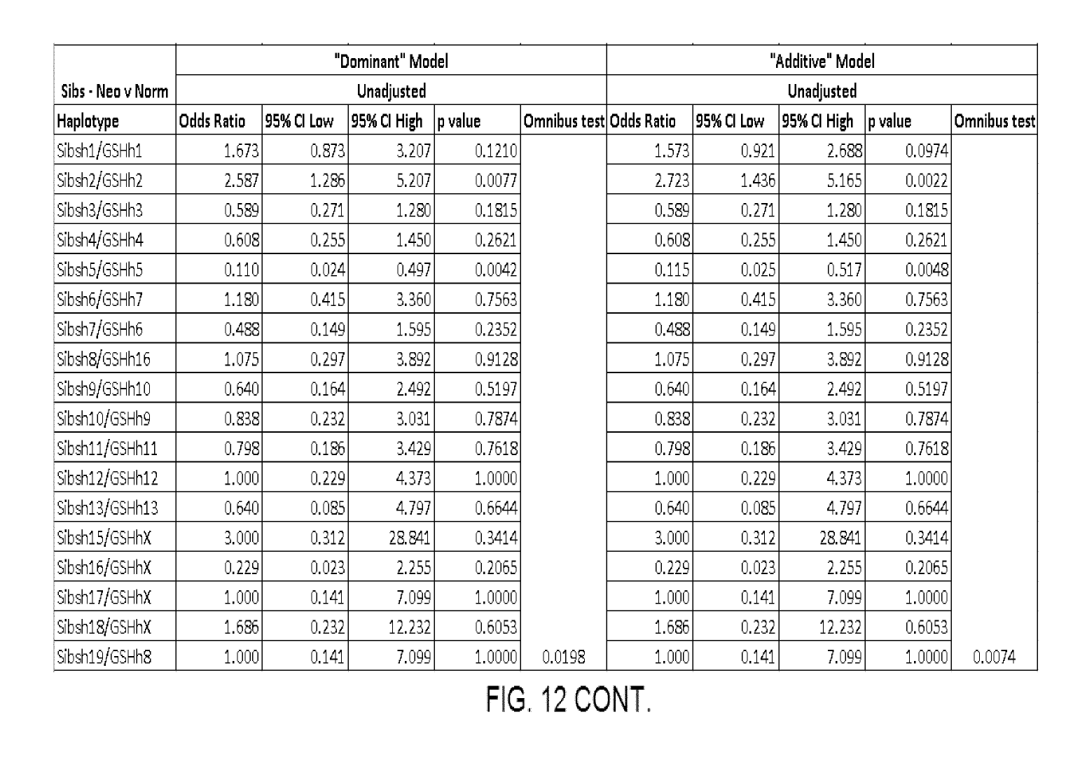

The differential association of H1_51_B and H2_51_B with AMD risk was verified using a highly discordant sib-pair cohort. 51 SNP-based haplotypes were generated for this cohort. Using logistic regression, a pattern was seen in which H1_51_A (as described in the GSH cohort) was not significantly associated with risk. The equivalent to H2_51_A, namely H2_51_B, wa highly associated with risk and wa the only haplotype that associates with risk (FIG. 12). Of the remaining haplotypes, only H5_51_B (containing the CFHR3/CFHR1 deletion) associated with AMD: in this case as a protective haplotype (FIG. 12). Thus, the difference in risk association between H1_51_A and H2_51_A was been replicated in a second independent cohort.

3.16 Results 51 SNP-based Diplotype Associations with AMD Risk, Protection and Neutrality: Combined Cohort

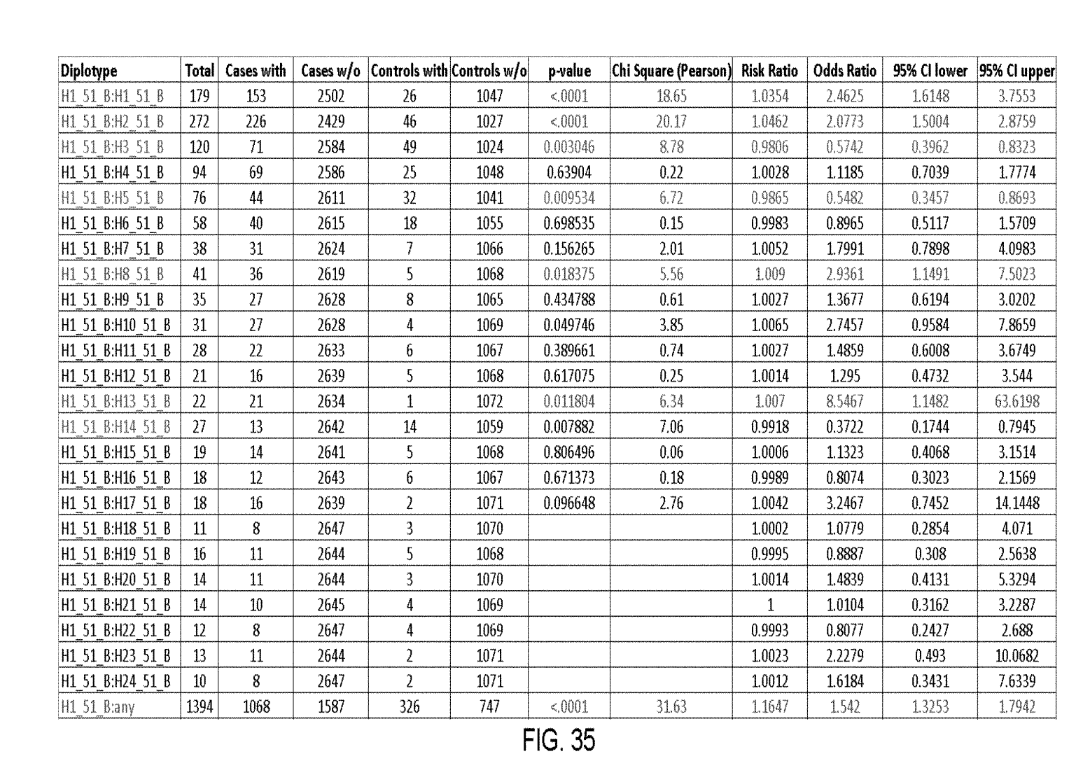

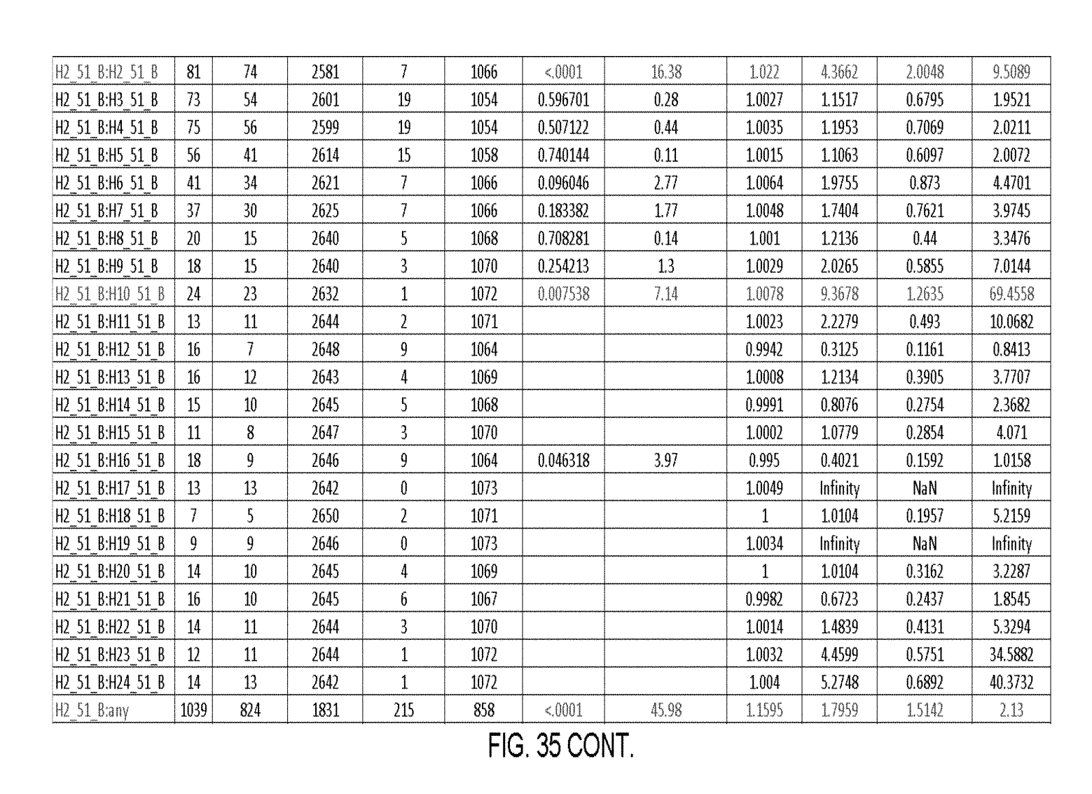

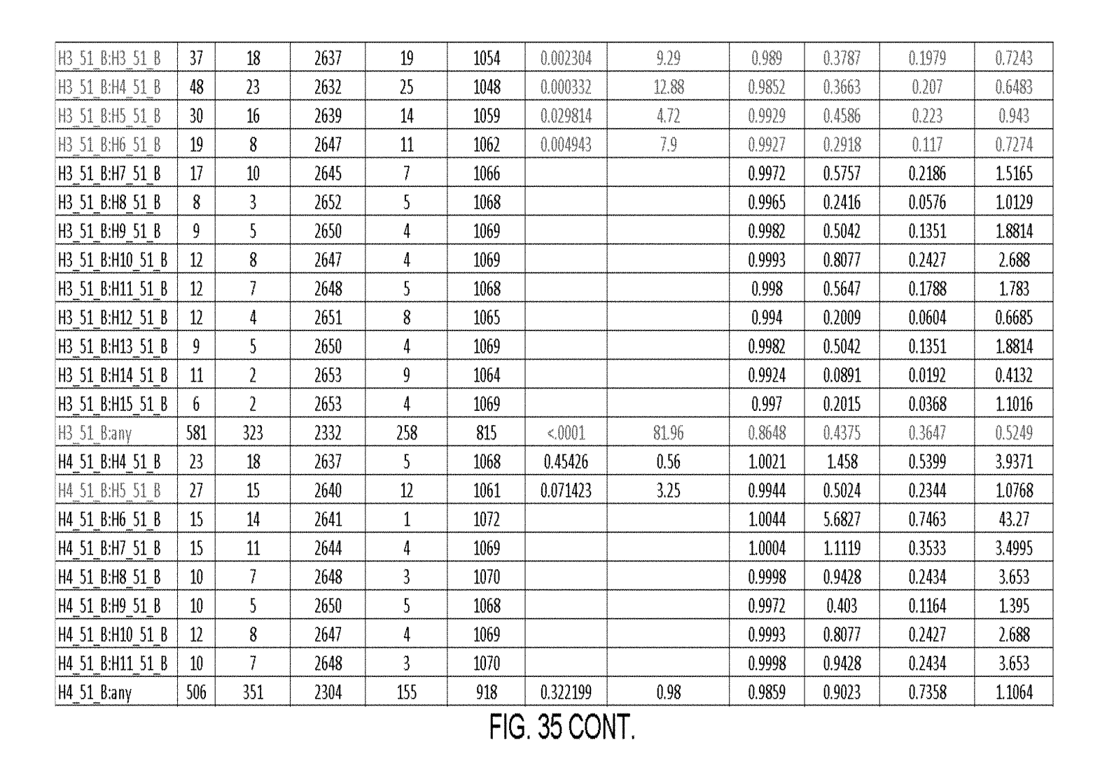

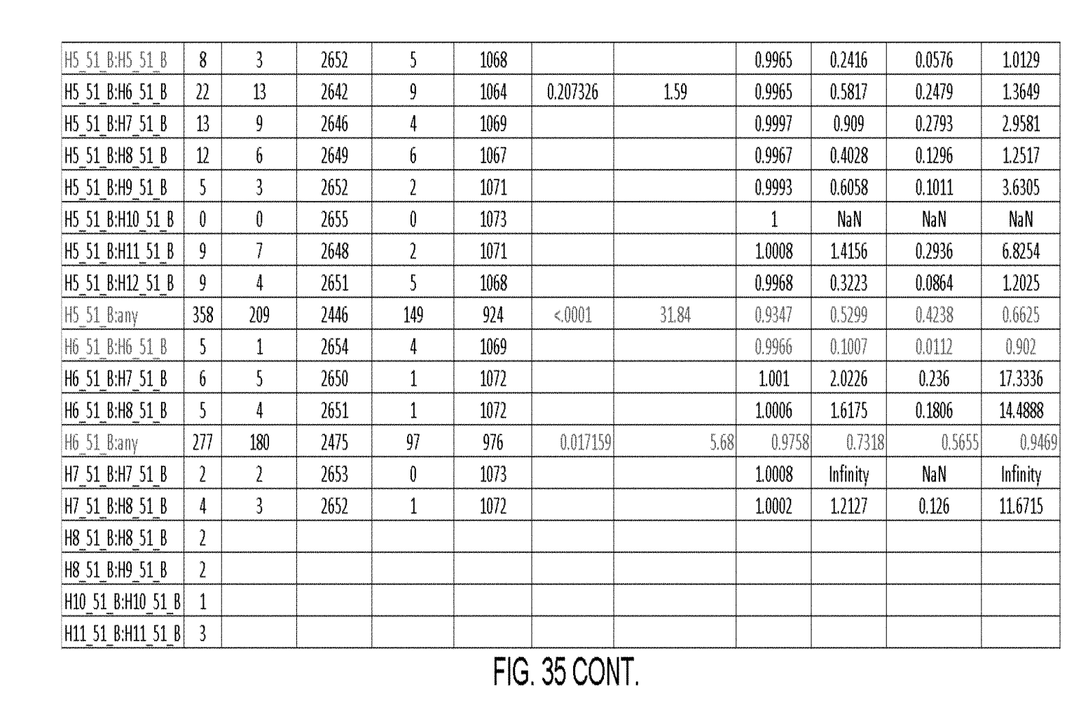

51 SNP-based diplotypes were phased and determined in the Combined Cohort using BEAGLE version 3.3.2 (S R Browning and B L Browning, 2007, Rapid and accurate haplotype phasing and missing data inference for whole genome association studies using localized haplotype clustering. Am J Hum Genet 81:1084-1097). Association with AMD risk, protection, and neutrality was assessed in the entire cohort (FIG. 35), in males only (FIG. 36) and in females only (FIG. 37). Combinations of all diplotype combinations for which there was appropriate statistical power are shown in FIG. 35; statistical power was sufficient to assess associations through haplotype 7.

A single H1 haplotype when combined with another H1 haplotype, or a H2, H8 or H13 haplotype was associated with increased risk for developing AMD whereas combination of H1 haplotype with a H3 or H5 haplotype was associated with protection. Only when a single H2 haplotype occurred with another H2 haplotype, a H1 haplotype or a H10 haplotype was there an association with risk. No combinations showing association with protection were observed, suggesting that the H2 haplotype is more strongly associated with risk than is H1. No risk association was observed with any combinations of a single H3, H4, H5, H6 or H7 haplotype in combination with any other haplotype. However, many of these combinations were protective, as depicted in FIG. 35. The overall results for the male and female only groups were similar to those of the whole cohort, with some minor differences noted in males only, perhaps due to loss of statistical power (FIGS. 36 and 37).

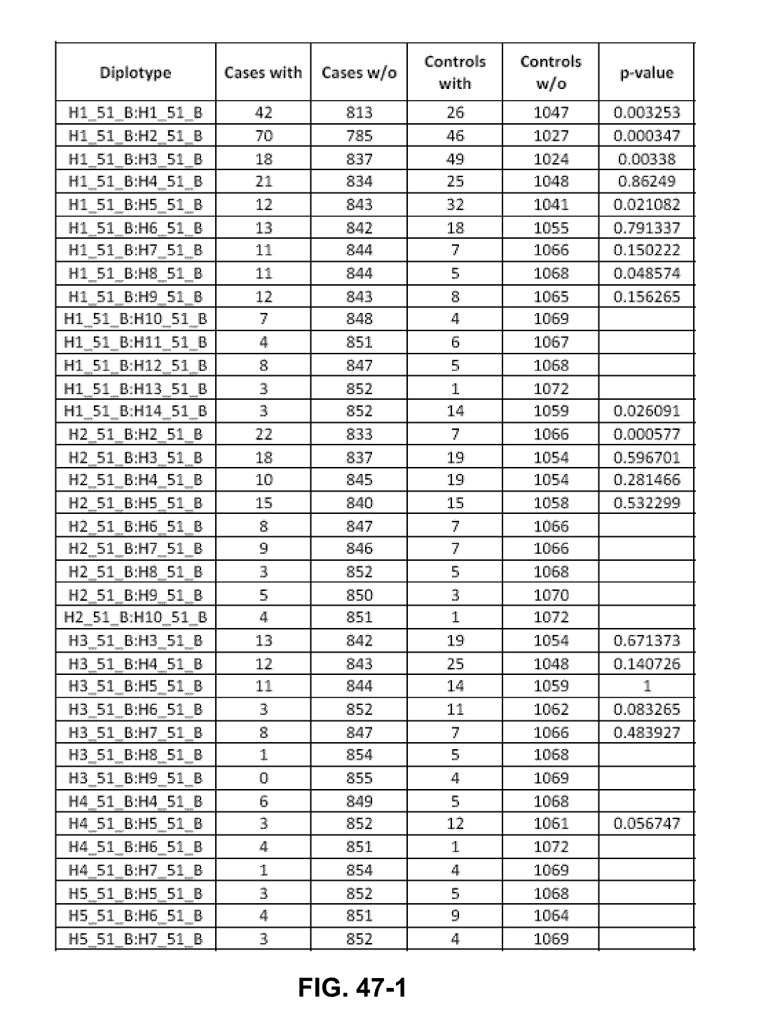

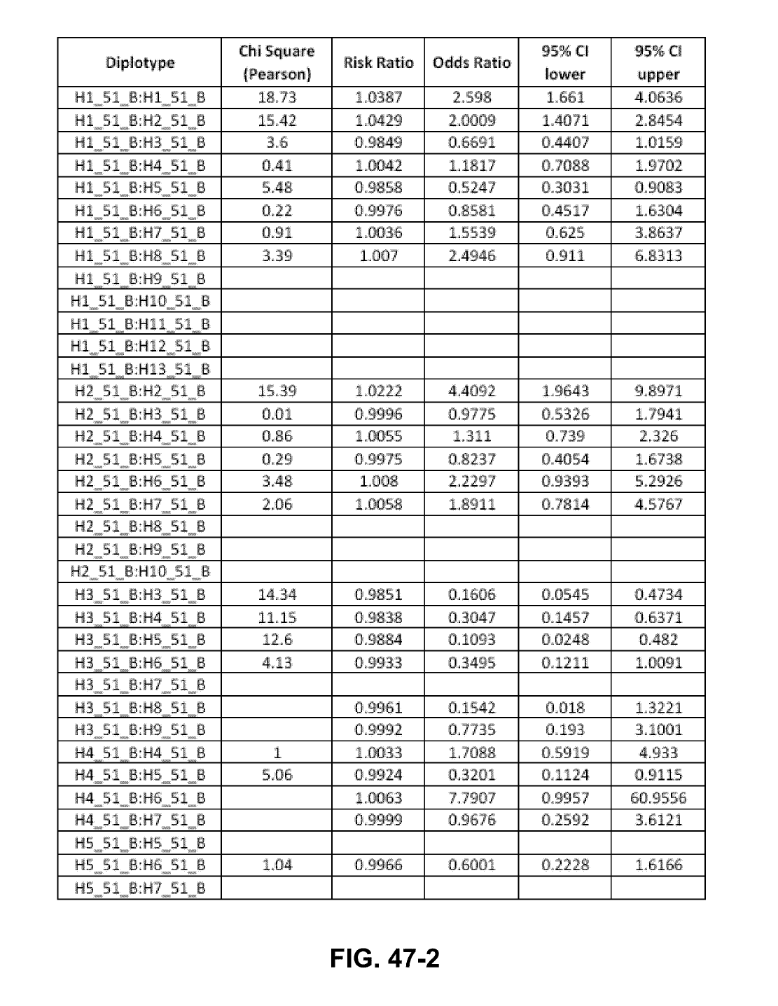

The ‘Combined Cohort’ was also employed to assess the association of 51 SNP-based CFH-to-F13B diplotypes with the various phenotypes/stages of AMD, including comparisons of controls (stage 0) to all AMD (stages 1B-4C), to early stage AMD (stages 1B-3), and to CNV only (stage 4B). Diplotype associations with risk, protection and neutrality did not vary significantly between the following groups: 1) controls compared to all AMD stages (FIG. 35); and 2) controls compared to CNV only (FIGS. 21-1 and 21-2). Nor did these associations differ from those in other cohorts. However, early when controls were compared to early stage AMD only (FIG. 47), the protective effect of all H3 combinations was greatly diminished, suggesting that H3 diplotype combinations may not be protective for phenotypes (drusen) associated with early AMD development or with specific types of drusen associated with early AMD development.

3.17 Results: Haplotype Tagging SNPs

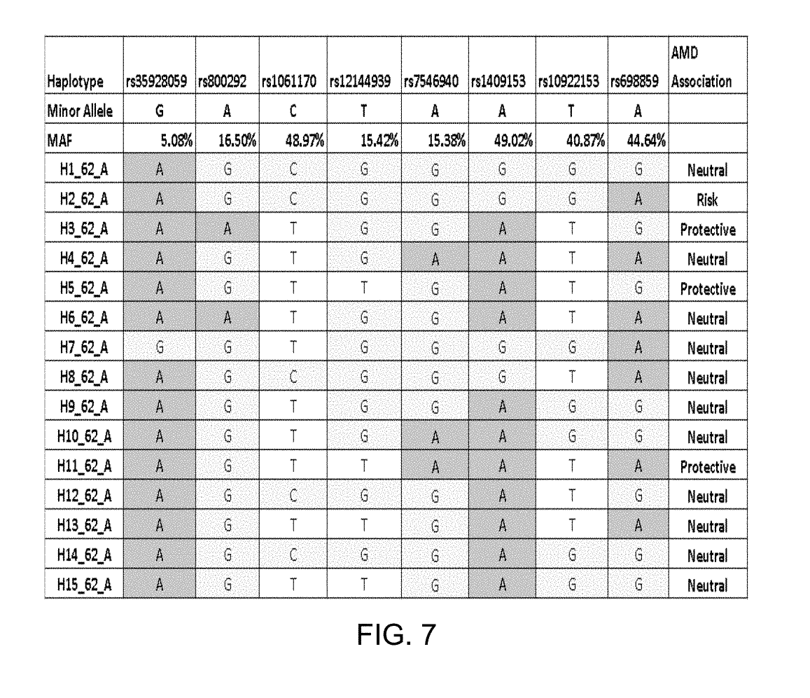

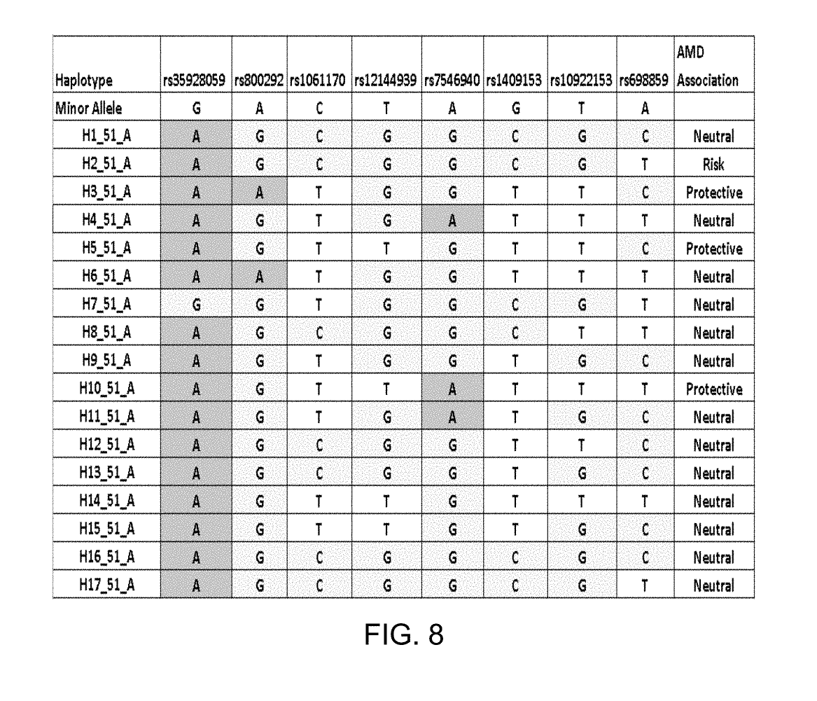

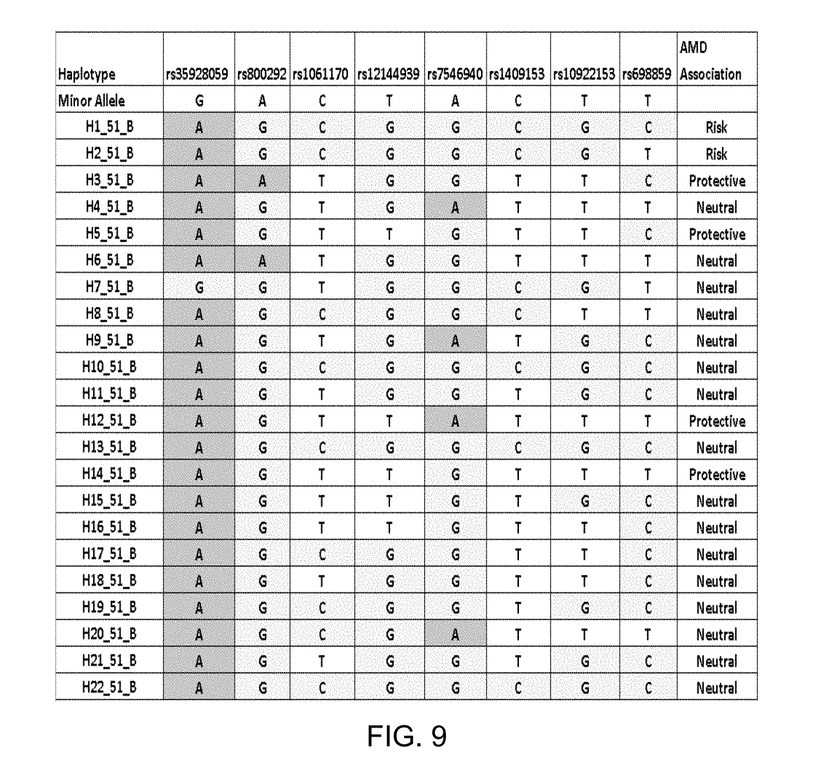

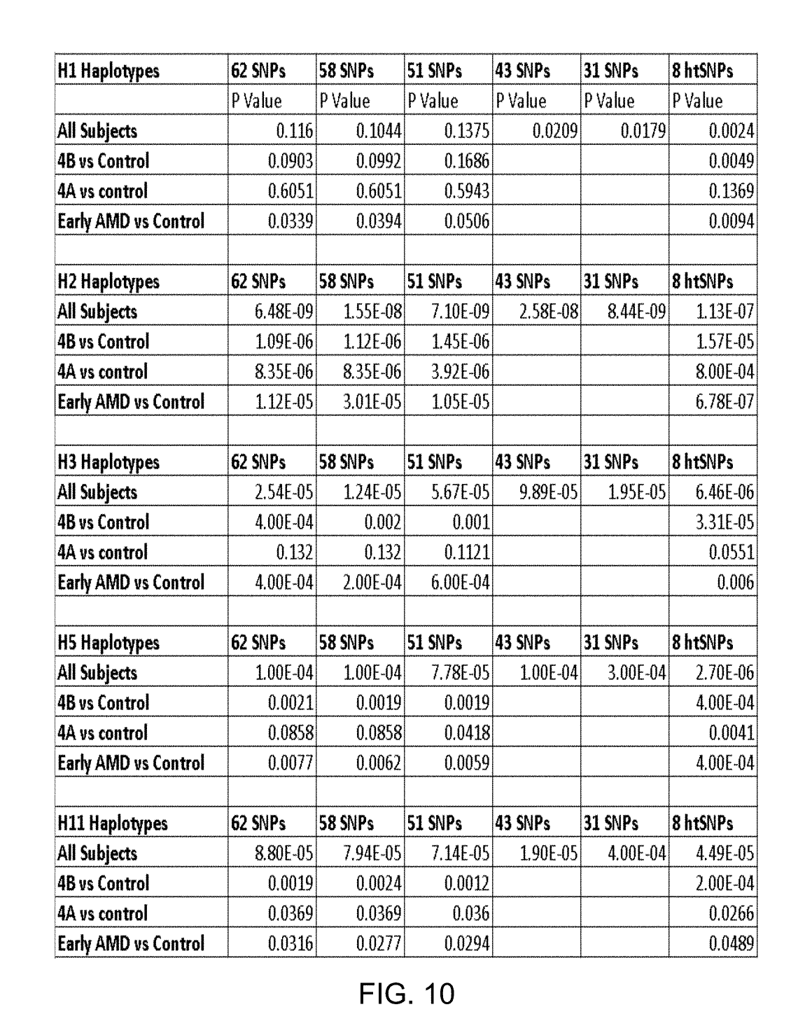

Each of the major 62 SNP-based haplotypes and each of the major 51 SNP-based haplotypes can be uniquely identified using a combination of eight haplotype tagging SNPs (htSNPs; FIG. 7; note that rs800292 and rs35928059 are optional for tagging the 62 and 51 SNP-based haplotypes). Case/control analysis was repeated on the GSH 1,073 Cohort using only the eight htSNPs to define haplotypes using Haploview 4.2. Risk association with H1 differed by several orders of magnitude and was statistically significant (FIG. 10; 8 htSNPs H1 p-value=0.0024; 62 SNPs H1 p-value=0.116; note that 62 SNP-based significance values are slightly different in FIG. 10 as compared to FIG. 6 because Haploview cannot handle the tri-allelic deletion SNPs) compared to the 62 SNP-based analysis. In order to show this effect more clearly, the 927 GSH cohort was reanalyzed in Haploview using progressively fewer SNPs in the following numbers: 1) all CFH locus SNPs (62); 2) all SNPs minus the four deletion-associated SNPs (58); 3) all SNPs for which Taqman assays were developed (51); 4) all SNPs for which MAF >5% frequency (43); 6) all SNPs with highly significant associations to AMD plus rs698859 (31); and 7) only the htSNPs (8) (FIG. 10). As fewer SNPs were used to generate haplotypes, more minor haplotypes were recast as major haplotypes and H1 became significantly associated with risk for AMD. All other significant haplotypes showed only small changes in their association to AMD that did not affect the interpretation of risk.

3.18 Results: Extension of 51 SNP-Based CFH-to-F13B Haplotypes to Genome Level Haplotypes; Association of HapMap Haplotypes with AMD

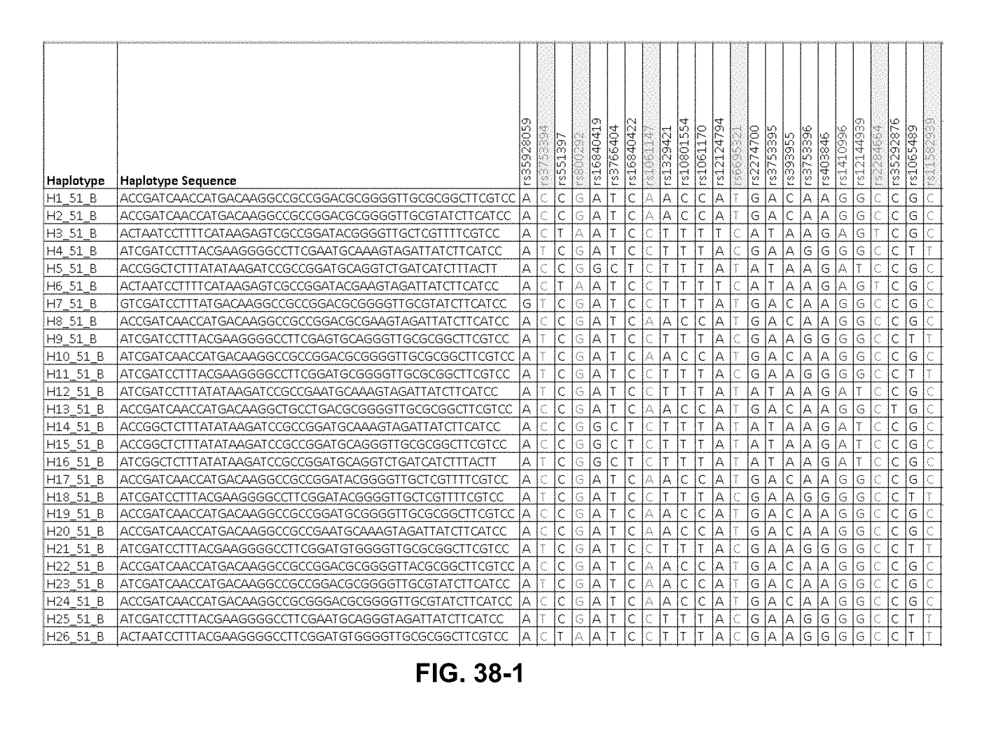

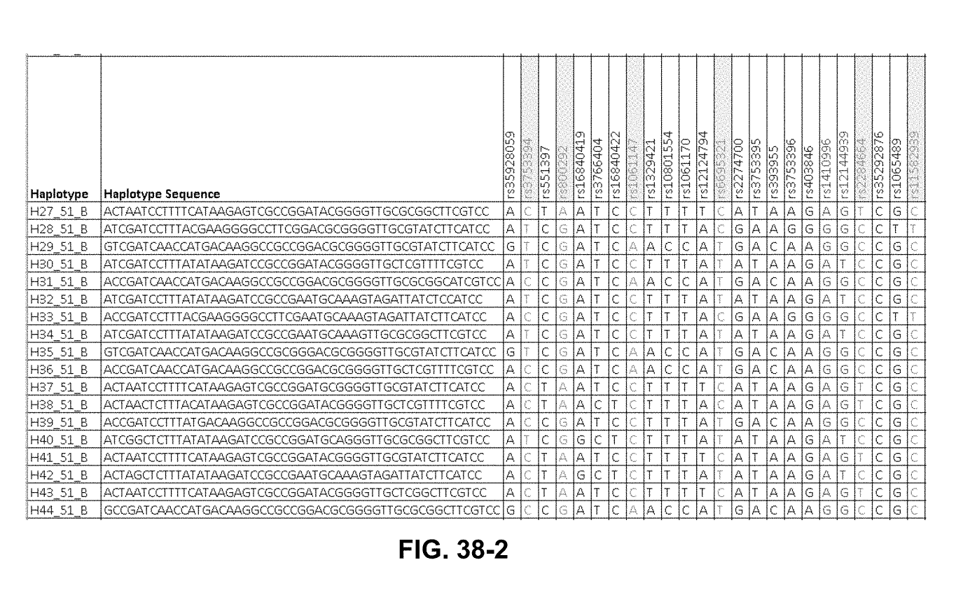

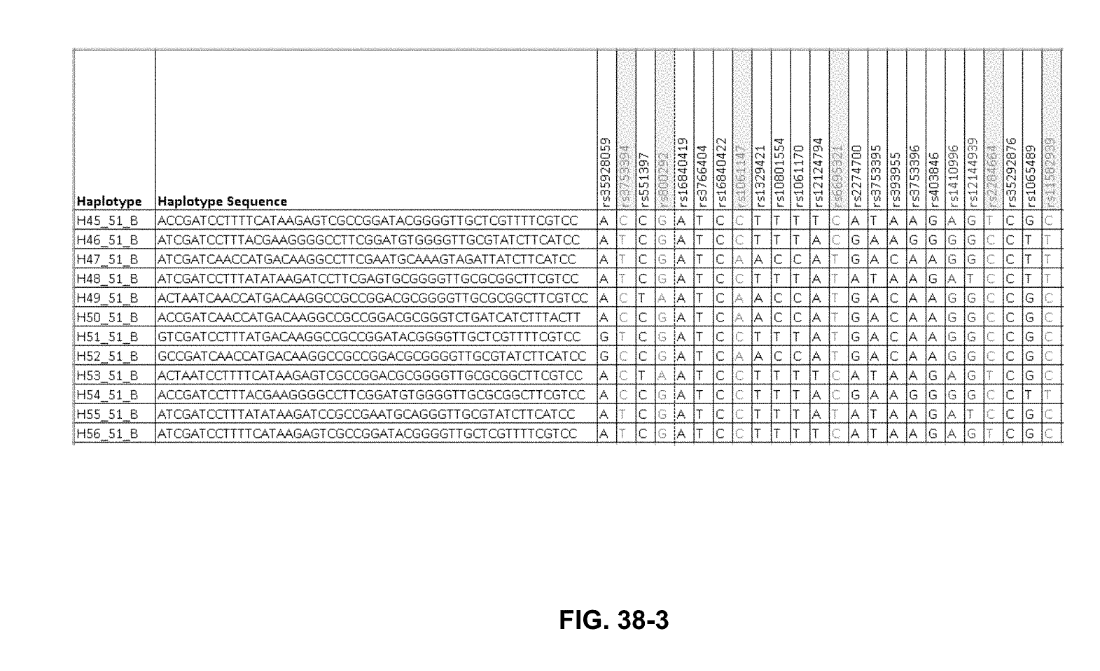

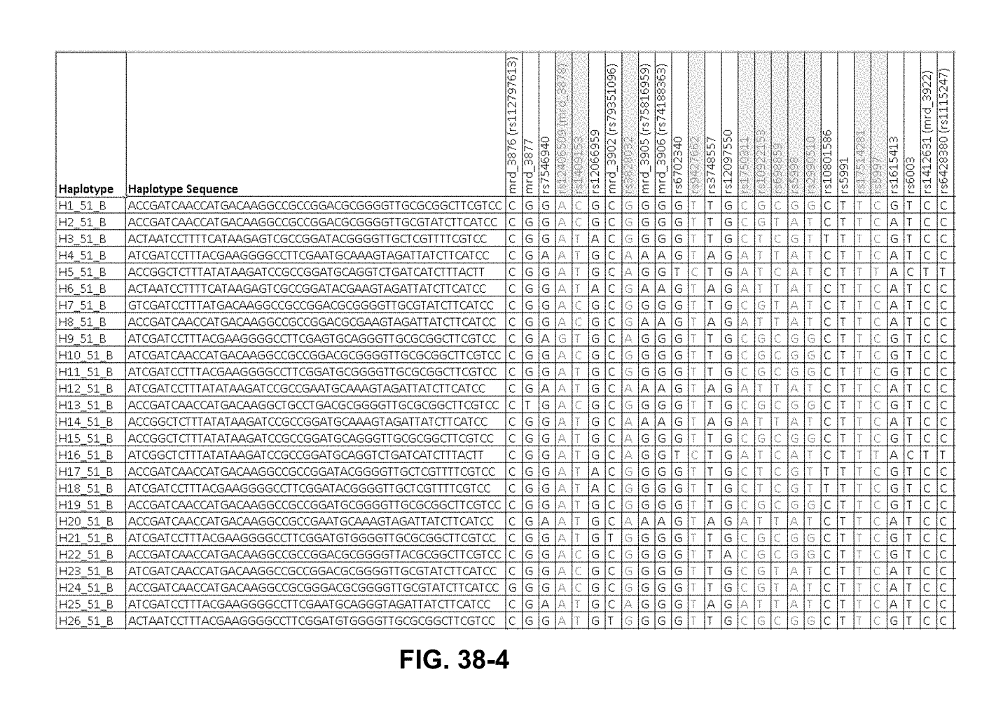

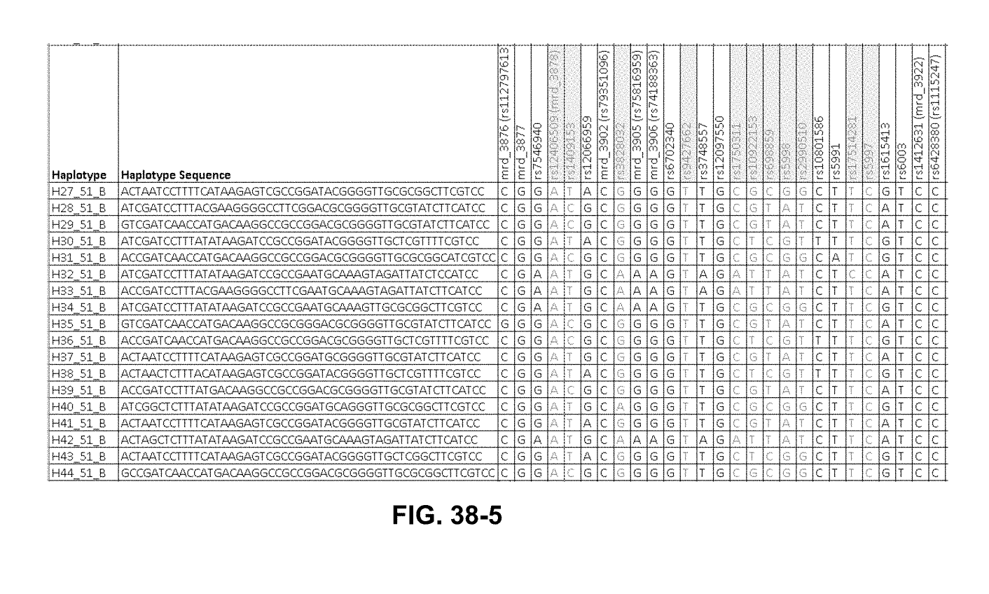

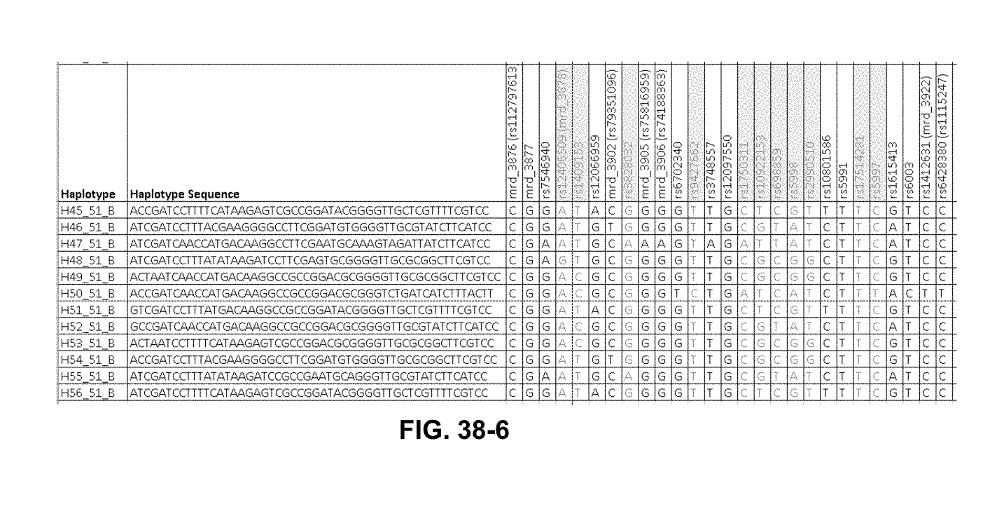

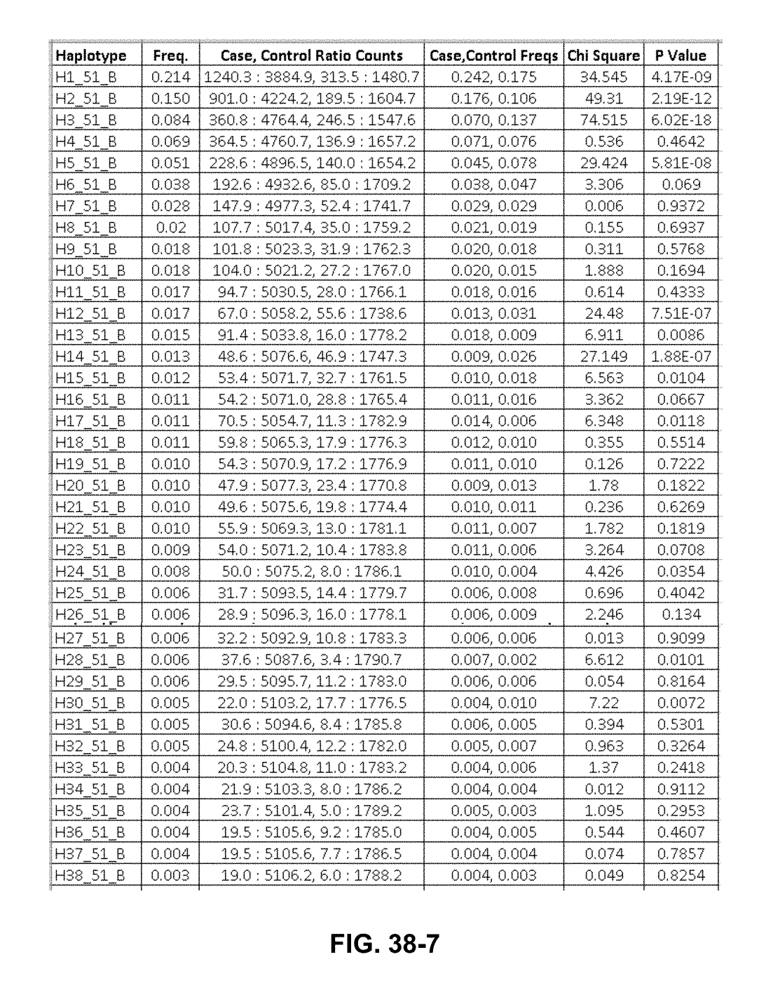

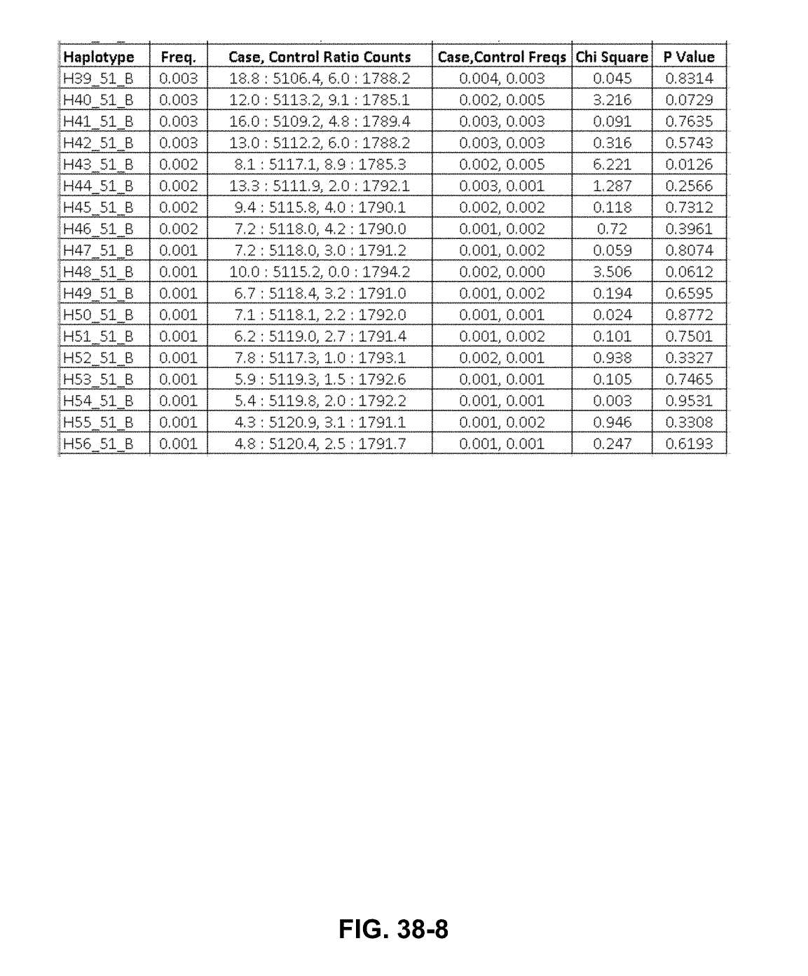

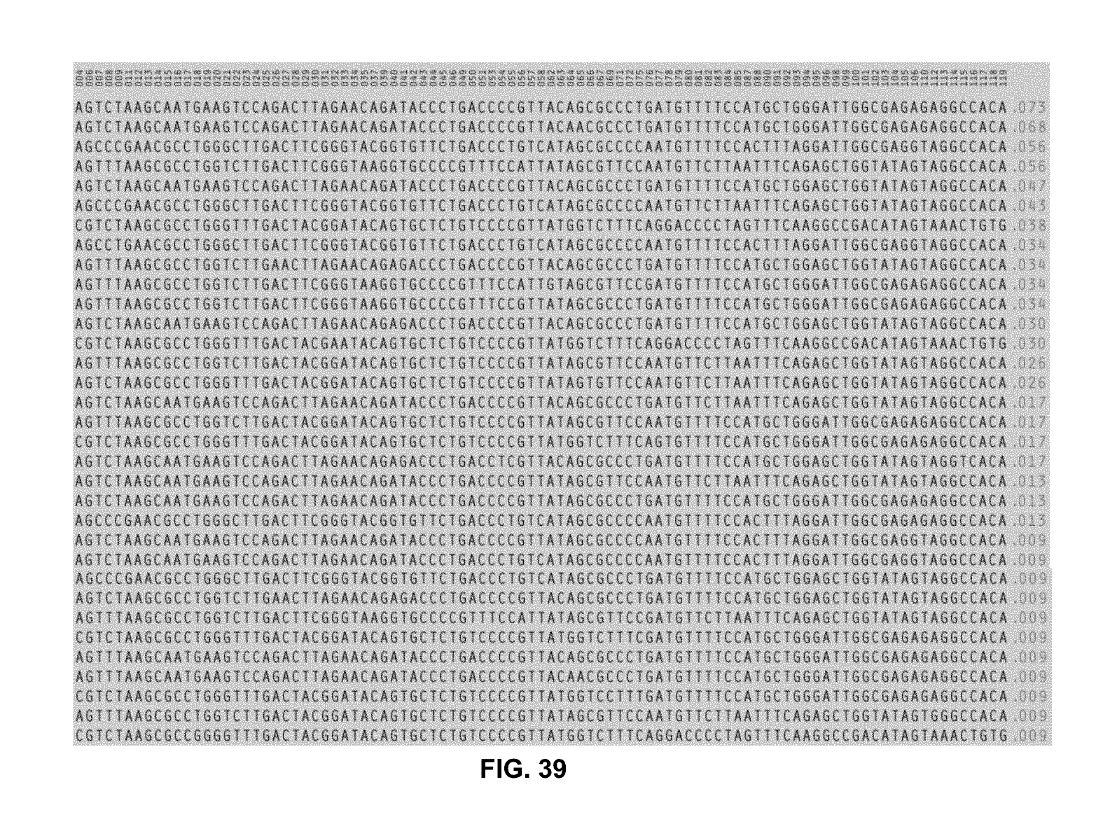

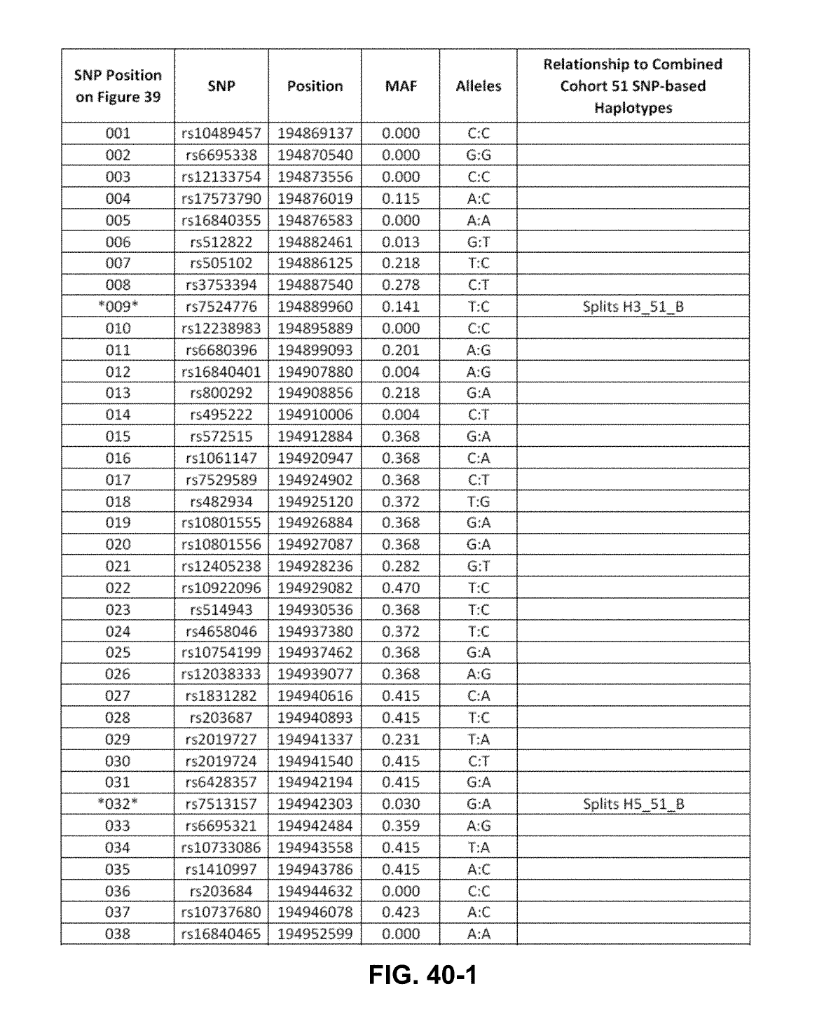

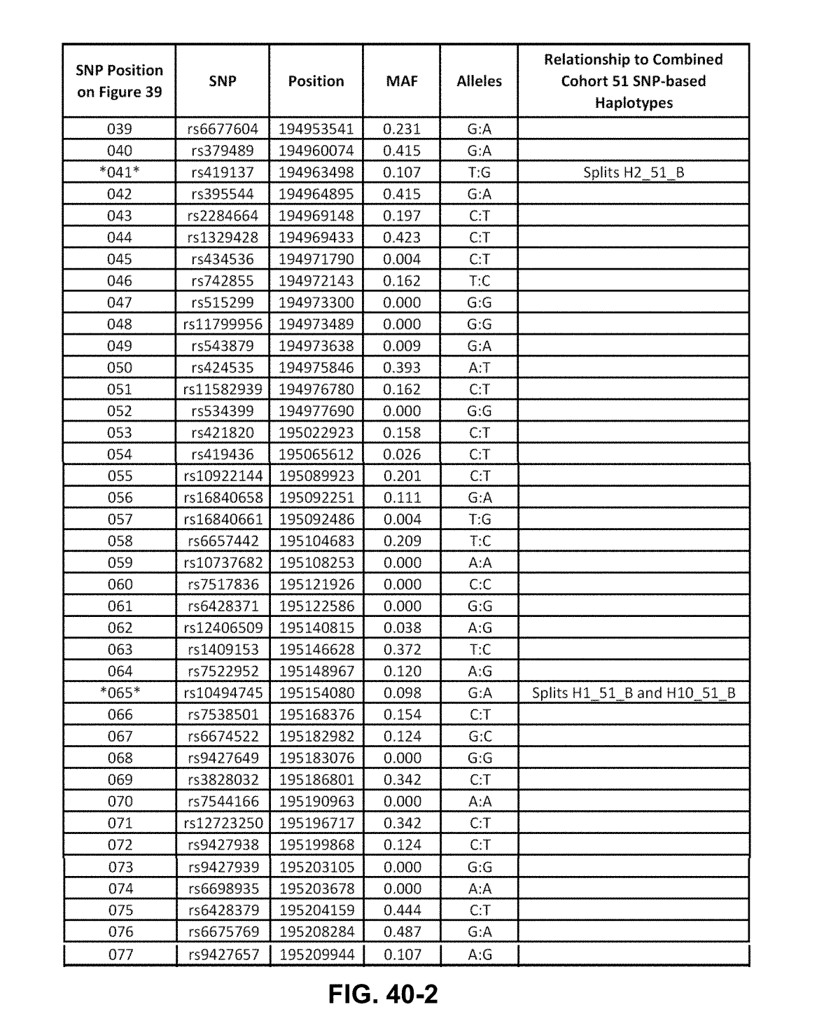

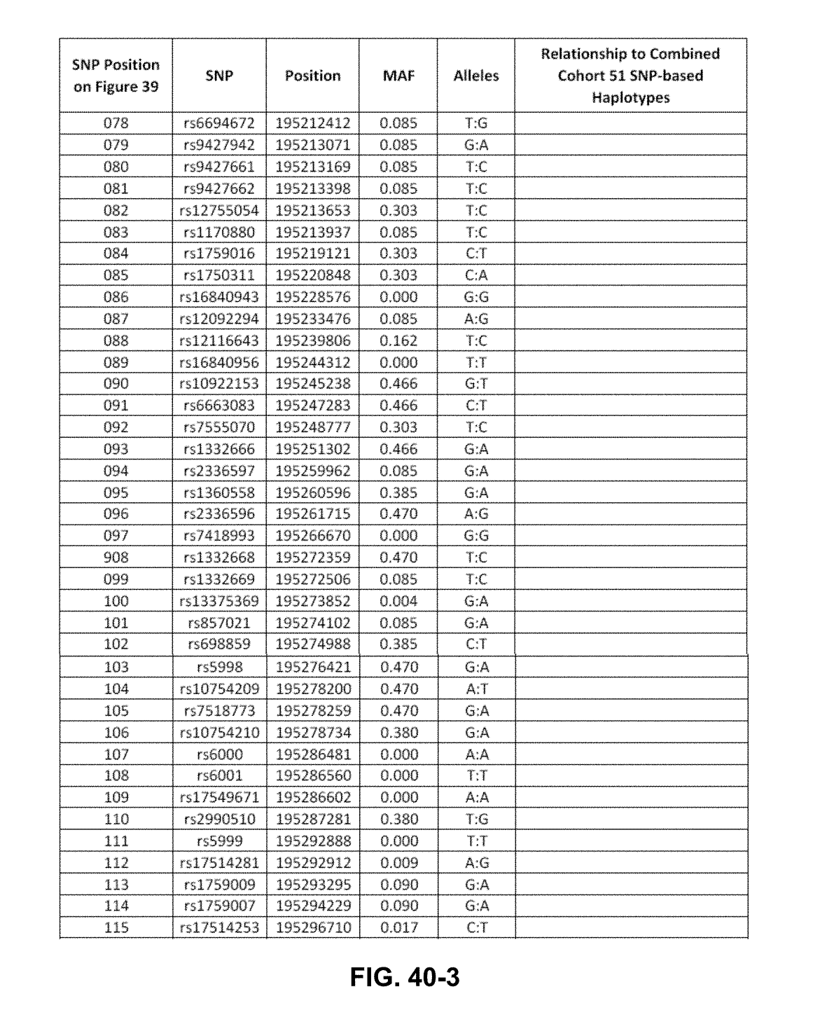

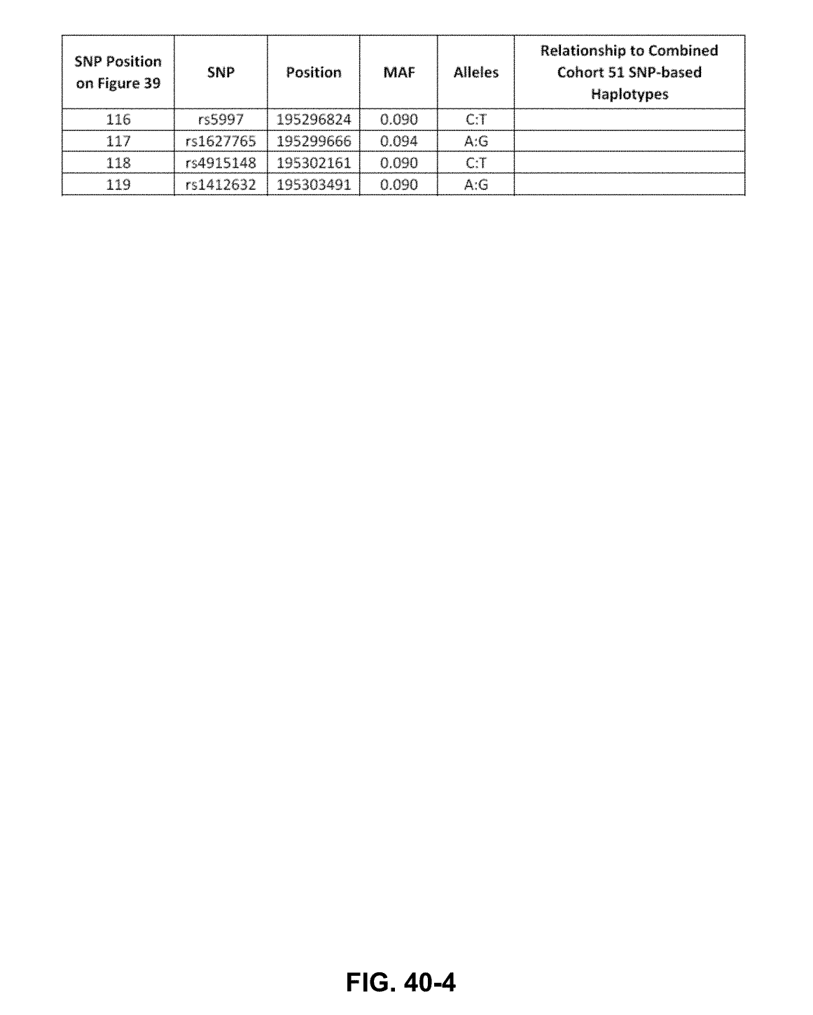

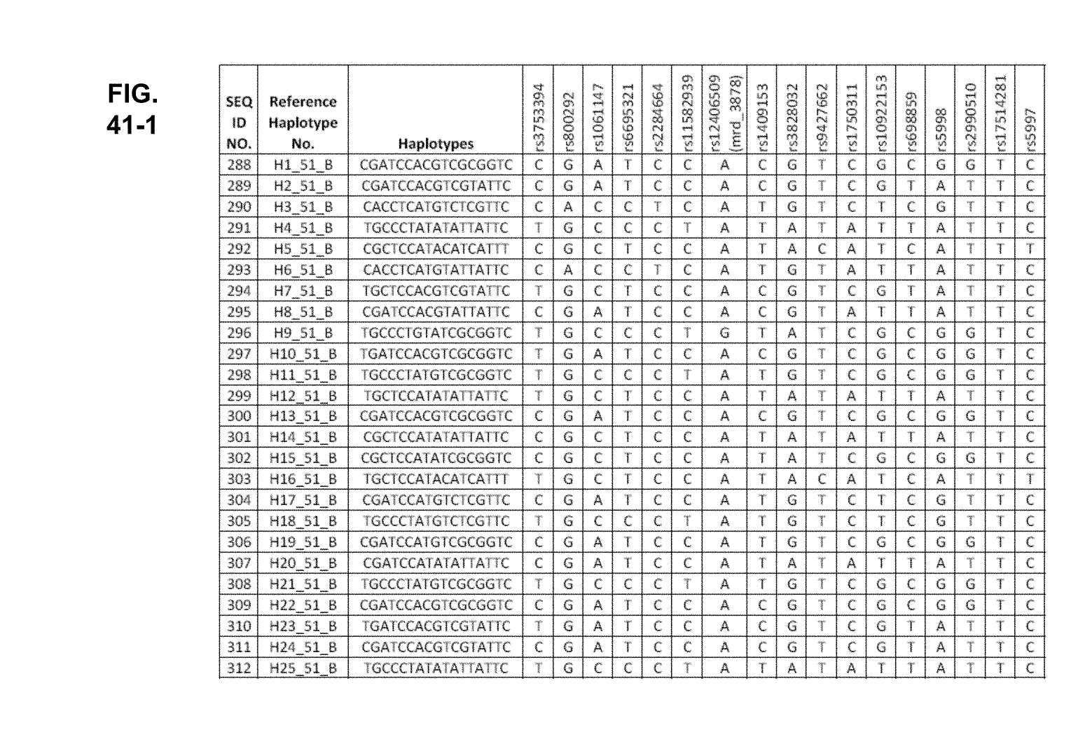

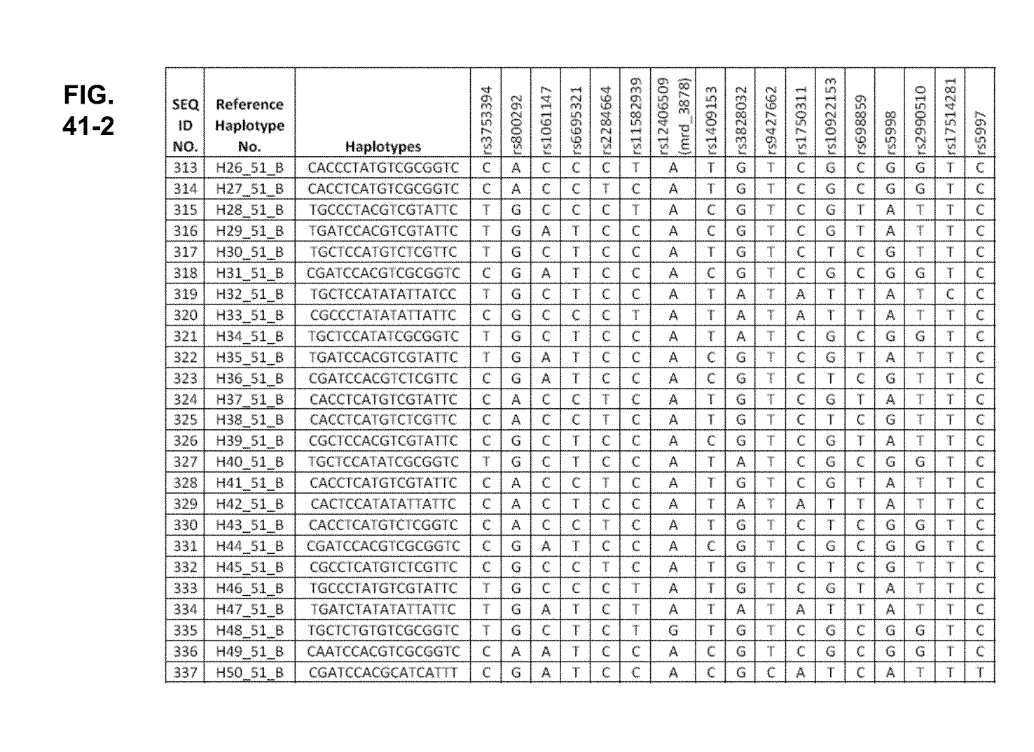

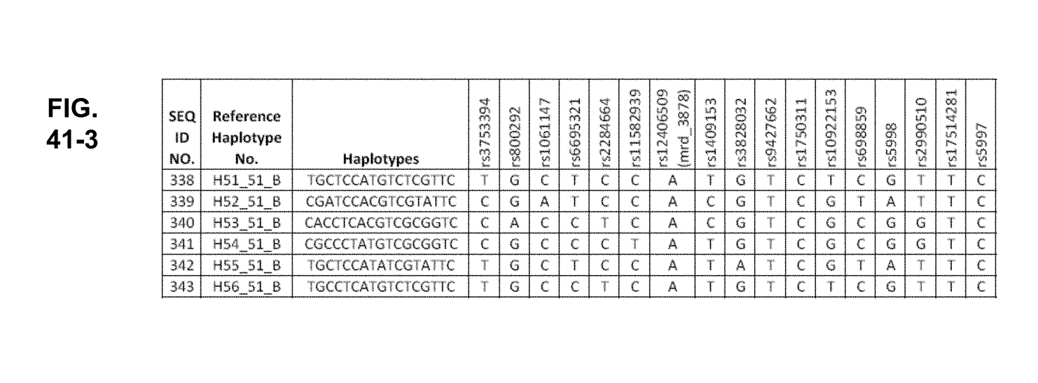

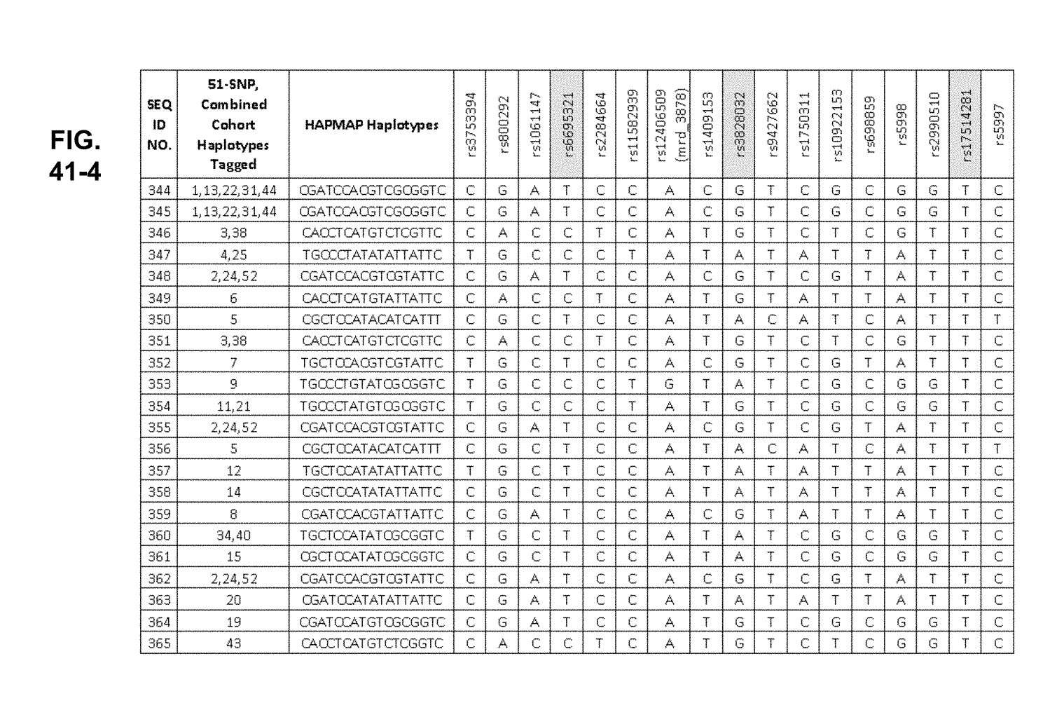

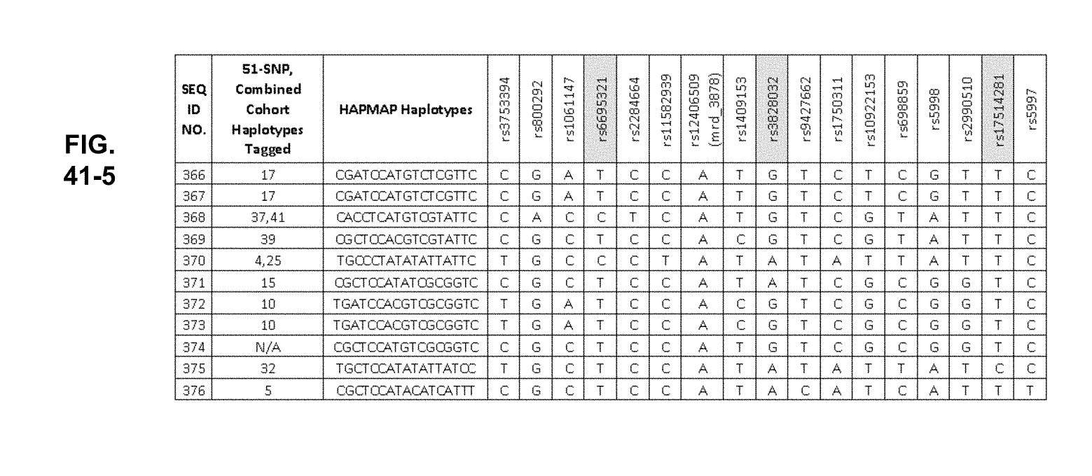

51 SNP-based haplotypes were compared to haplotypes generated using HapMap data in order to further refine haplotype association with AMD. The International HapMap Project database (http://hapmap.ncbi.nlm.nih.gov/) contains over 500 SNPs in the region encompassing the CFH-to-F13B locus (Hg18 chr1: 194869137-195303491). Genotypes from 180 Caucasian individuals (referred to as the CEU population) were downloaded for each of these 500 plus SNPs. Phased genotype data from the CEU set was downloaded, including the most informative SNPs (i.e. those that segregate in the CEU population) in this region of interest. Using Haploview (www.broadinstitute.org), haplotypes were constructed for the CEU dataset and their frequencies calculated (FIGS. 39 and 40). There is substantial overlap between the AMD case/control (combined cohort′) 51 SNP-based haplotypes and the HapMap haplotypes (FIGS. 38, 39 and 40). These overlapping SNPs were used as ‘tagging’ SNPs to match case/control haplotypes to their equivalent HapMap CEU haplotypes (FIGS. 38 and 41). AMD association with haplotype was previously established in the 51 SNP-based case/control (combined′) cohort (FIG. 22); thus association could be extended to the equivalent HapMap haplotypes. In some instances, 51 SNP-based haplotypes overlapped with multiple HapMap haplotypes and vice versa due to the presence of HapMap SNPs that were not included in the 51 SNP-based haplotypes. Thus, in cases where HapMap haplotypes overlapped with multiple equivalent case/control haplotypes, key SNPs were identified that further refined the discriminative value of our major 51 SNP-based haplotypes. As an example, the ‘combined cohort’ haplotype H1 (FIGS. 39 and 41) matched the two most frequent of the major HapMap generated haplotypes (FIGS. 39 and 41). The only SNP that differentiated these two HapMap haplotypes was rs10494745. Other similar informative SNPs discovered in this analysis that will refine assessment of disease association are indicated with asterisks and their relationship to the Combined Cohort 51 SNP-based haplotypes are described in FIG. 40. They are rs7524776, rs7513157, rs419137, and rs10494745.

3.19 Results: Extended Haplotype Blocks

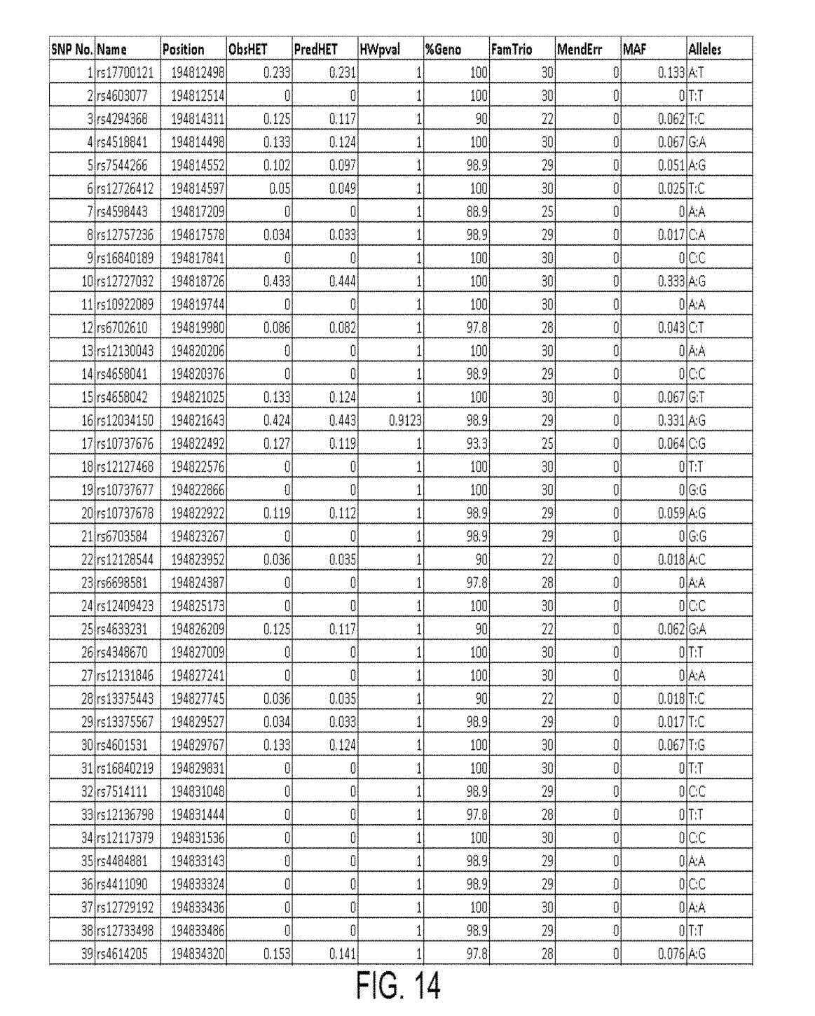

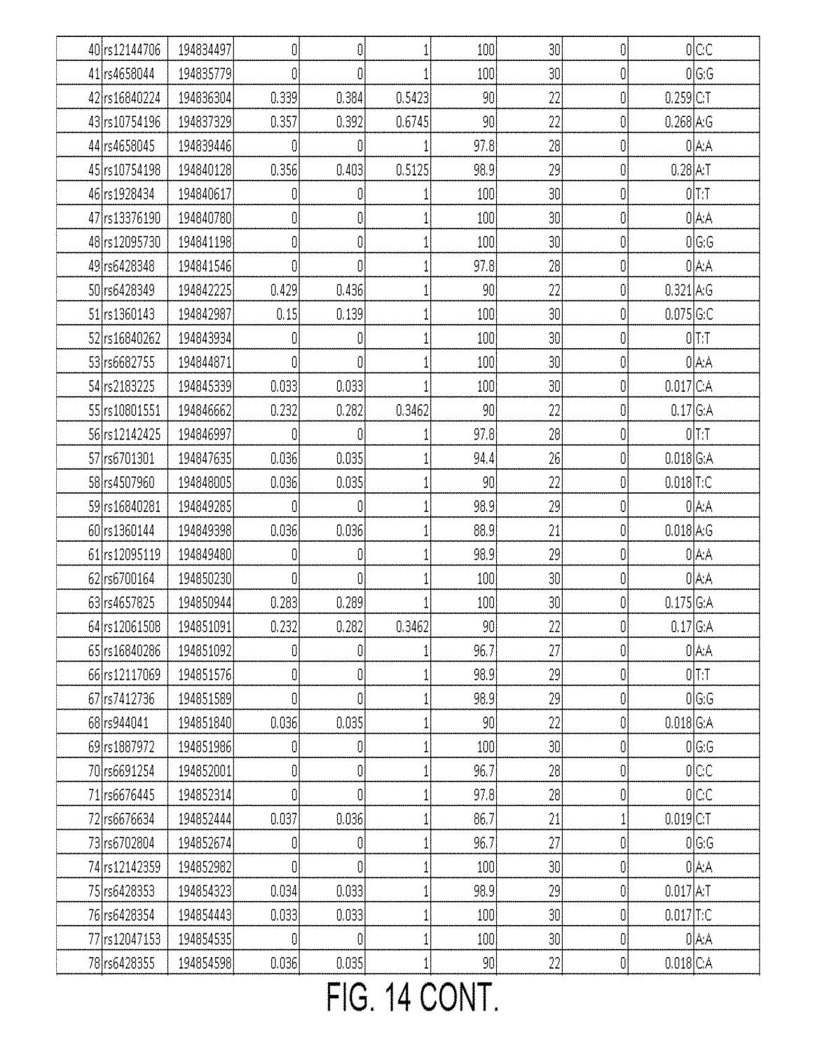

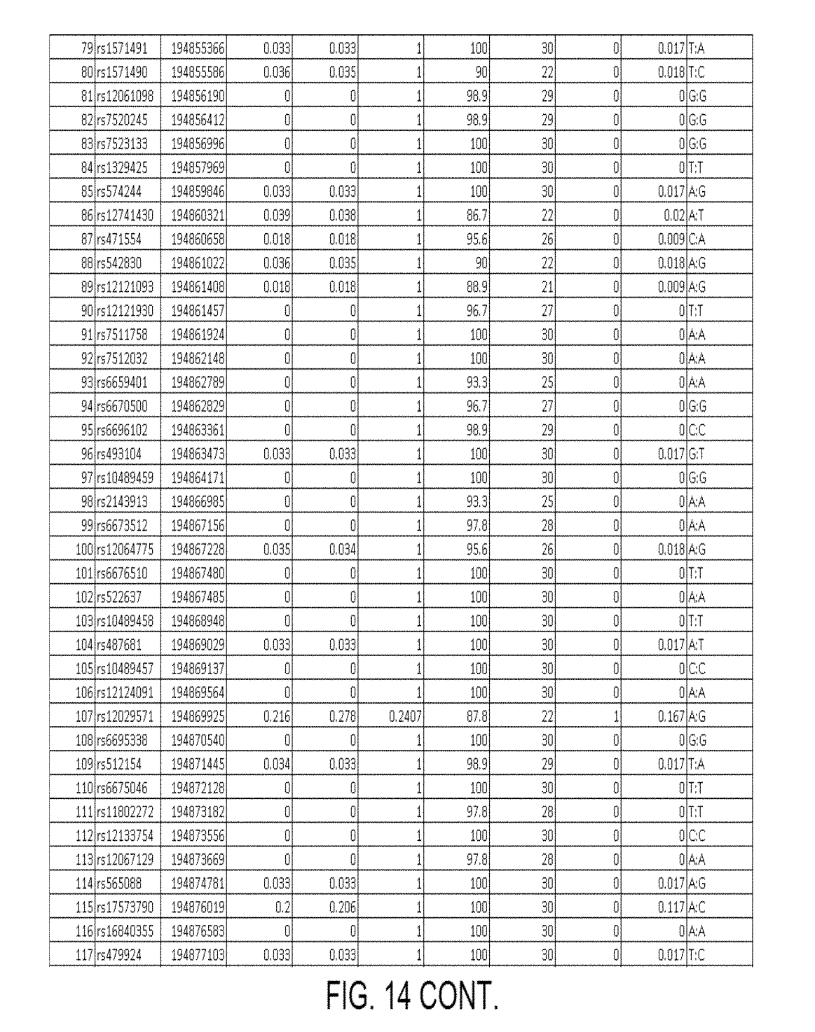

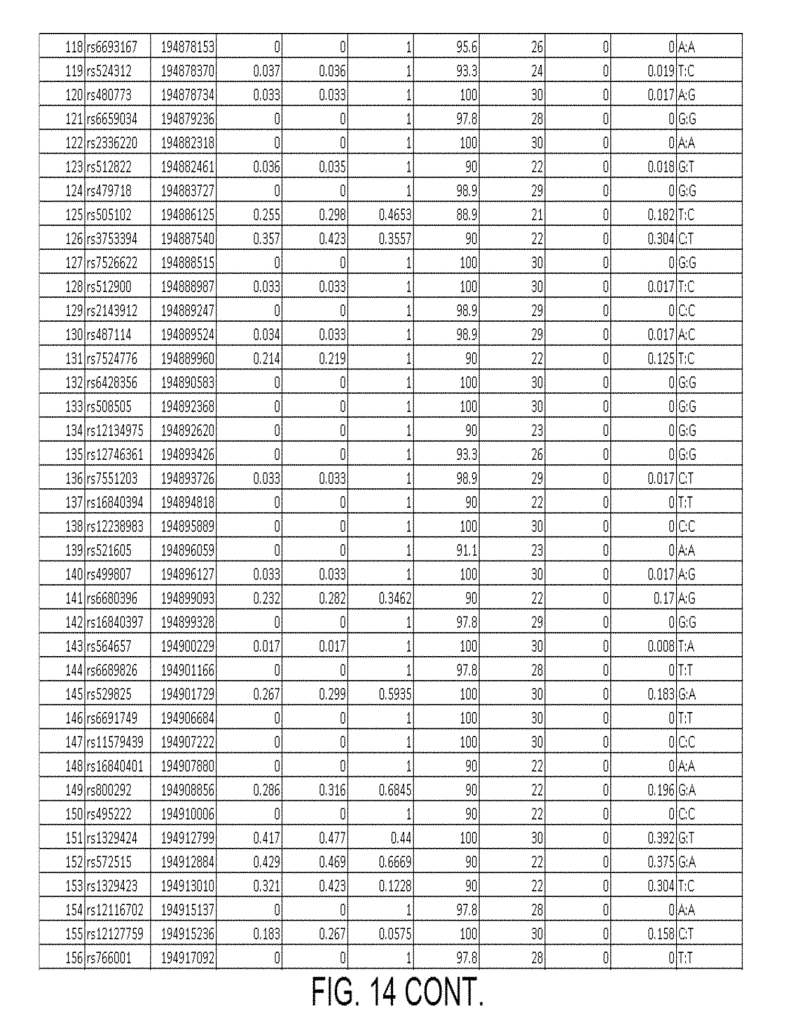

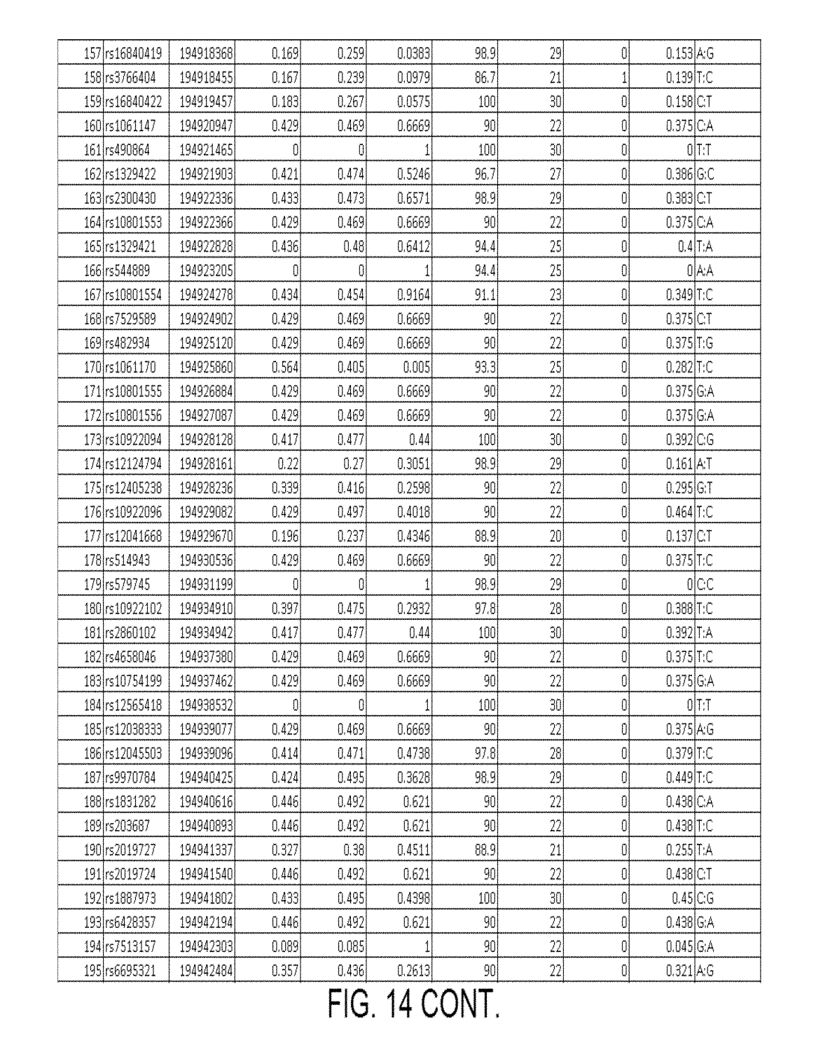

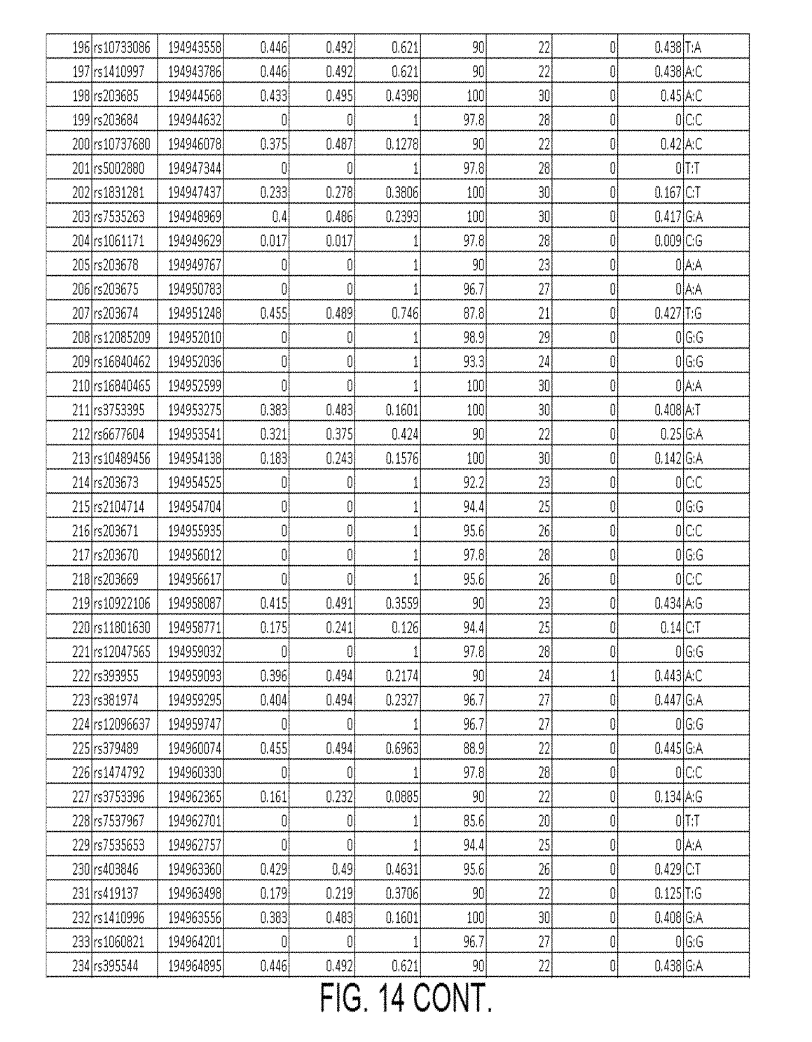

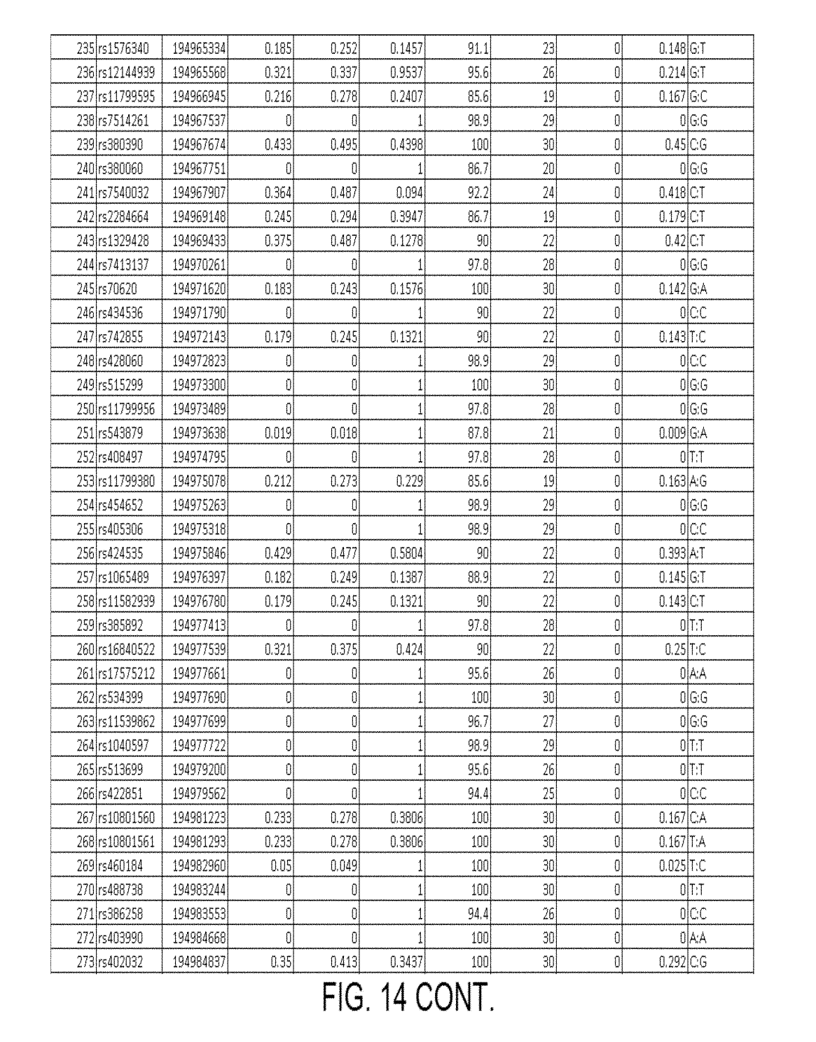

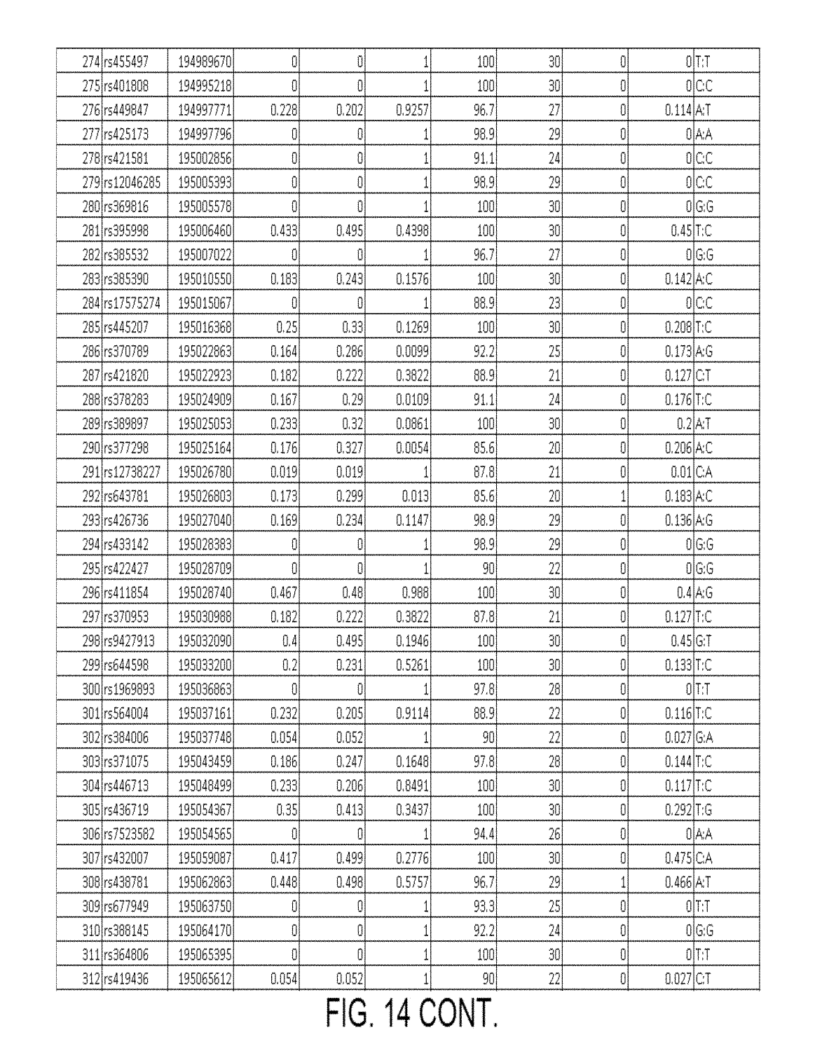

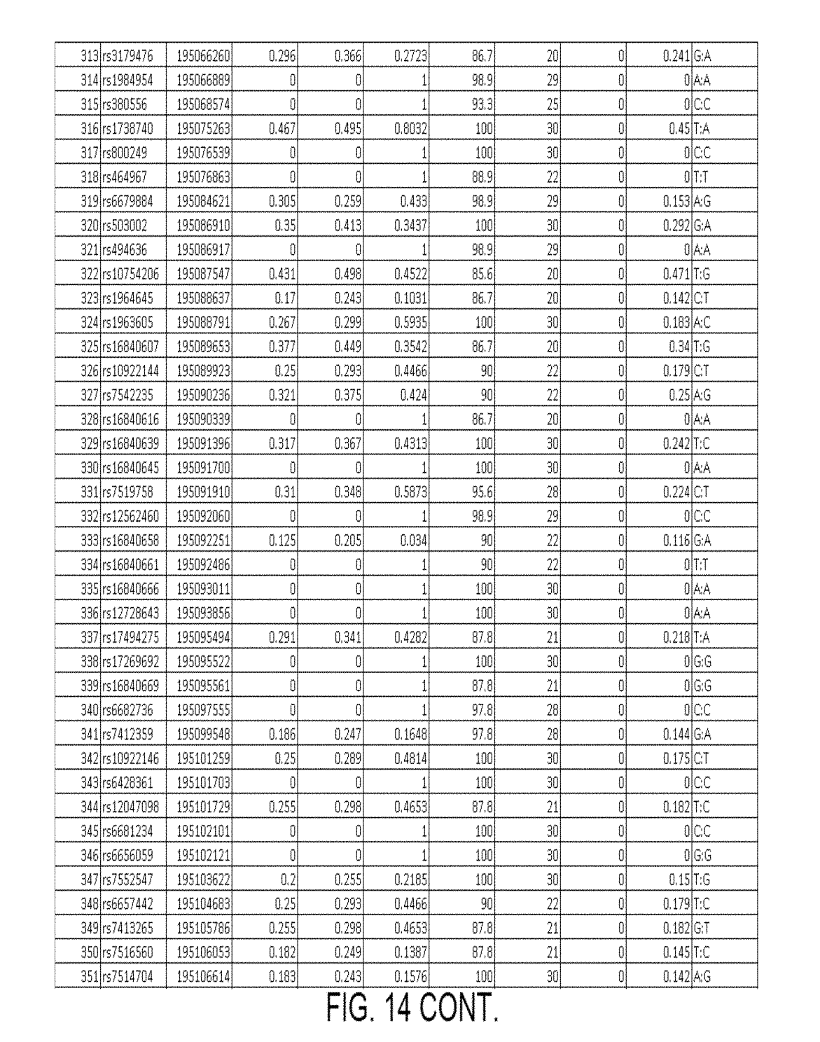

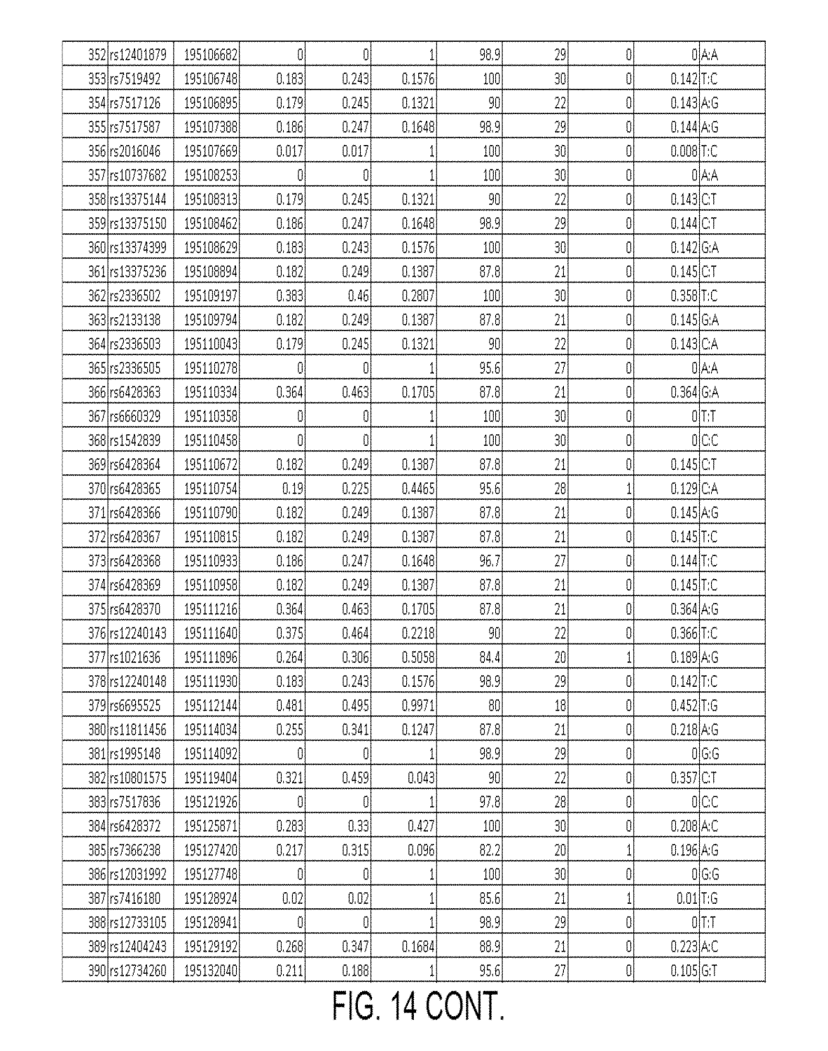

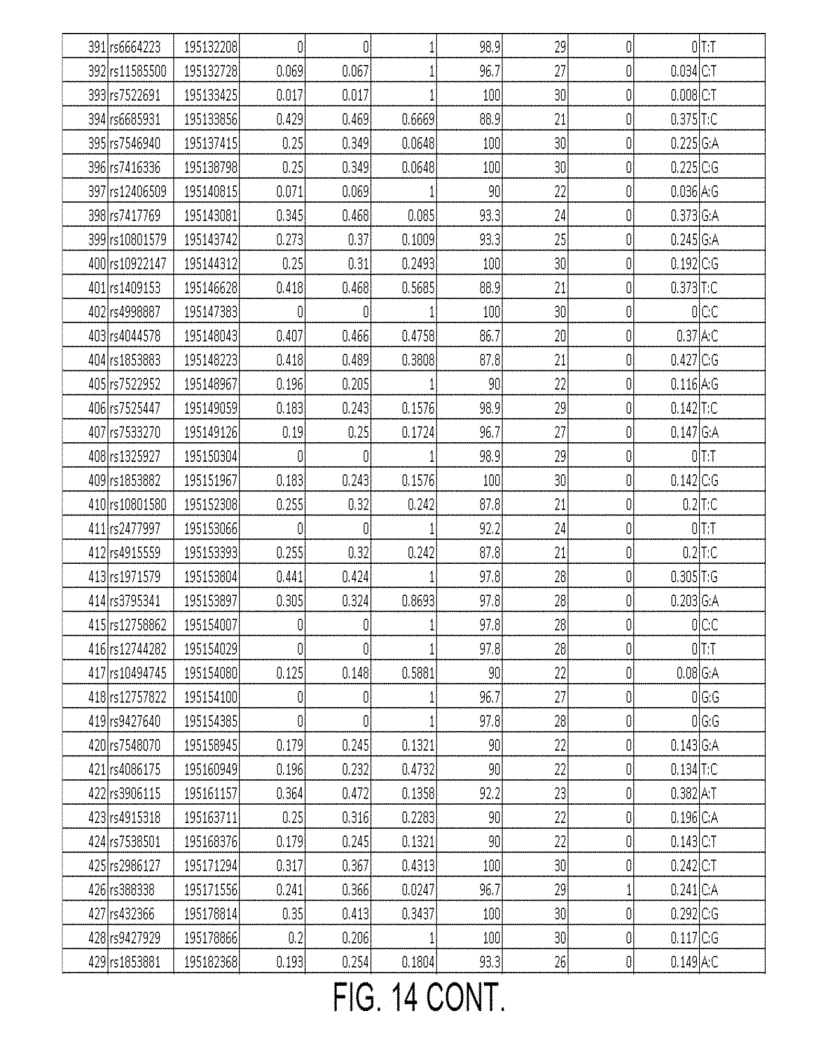

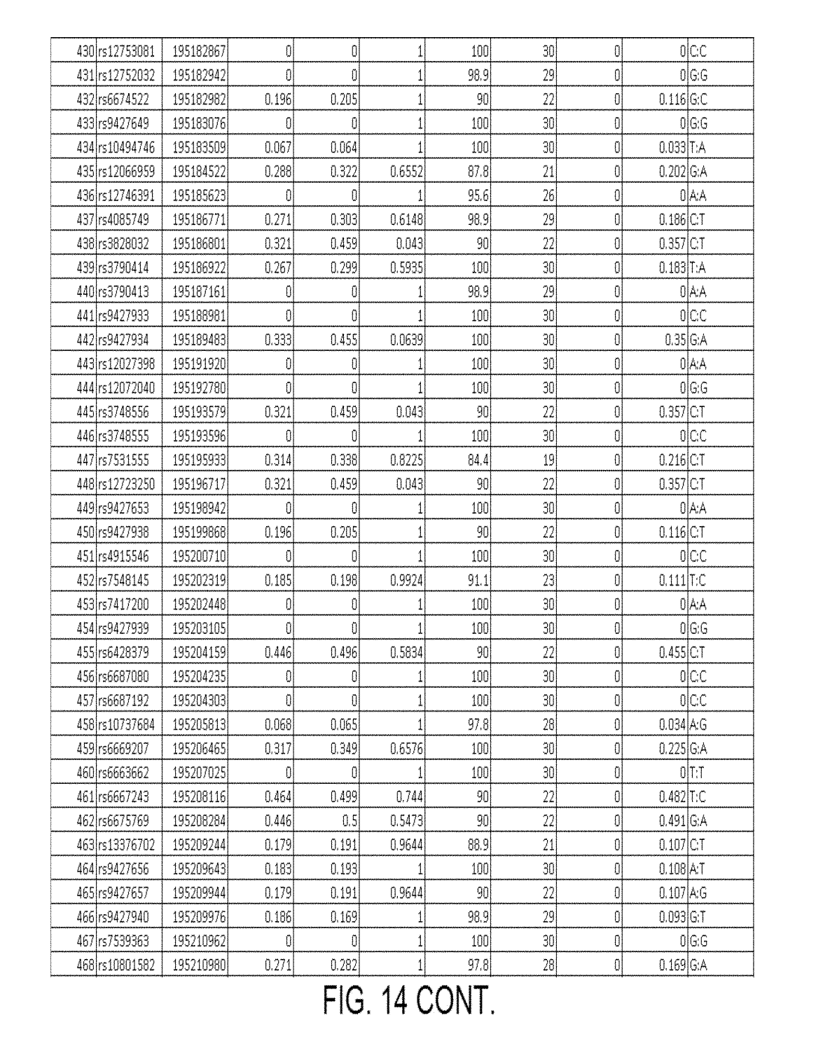

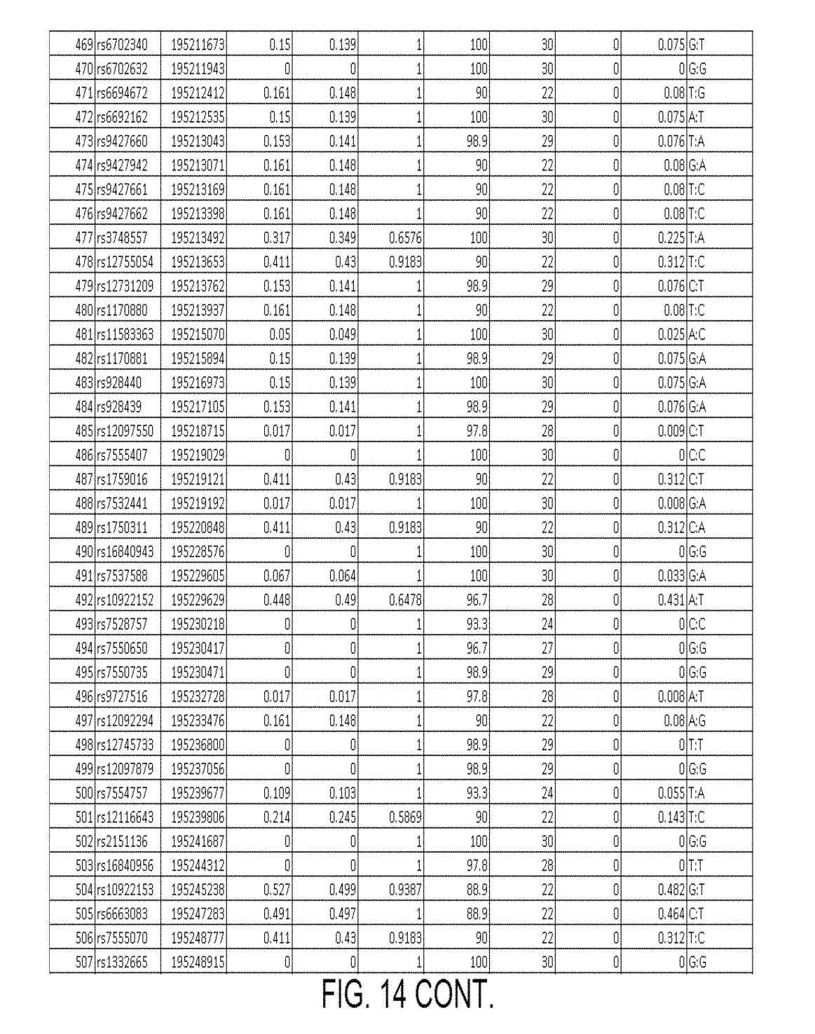

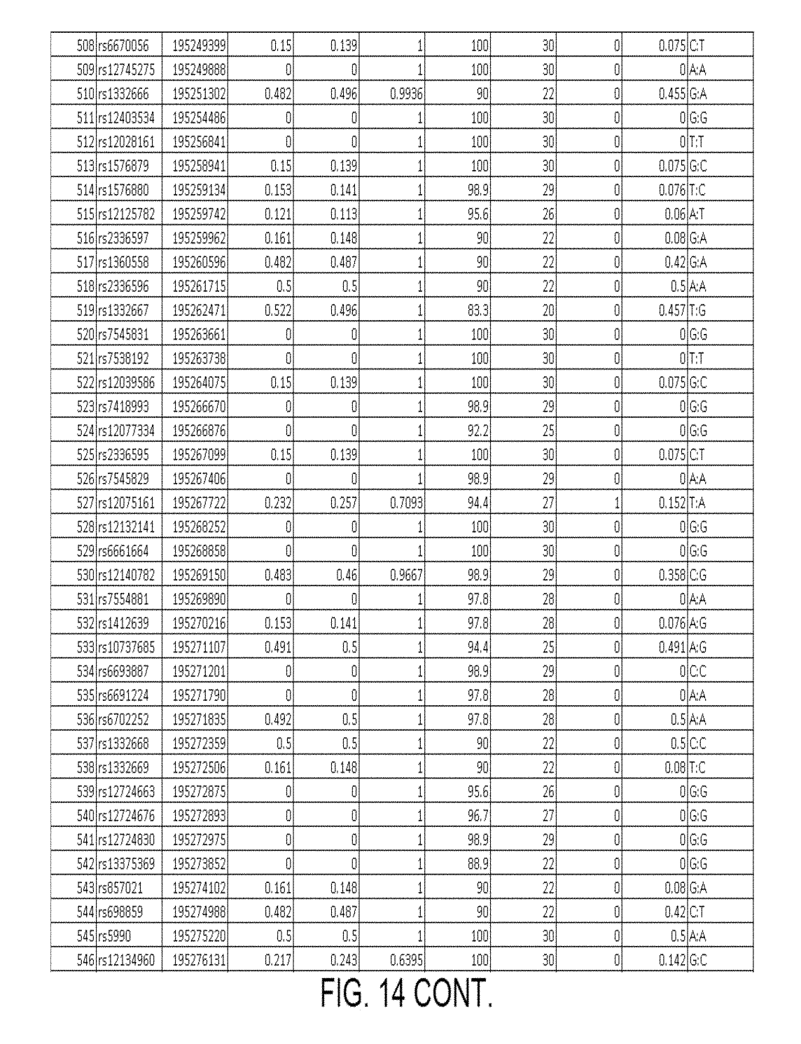

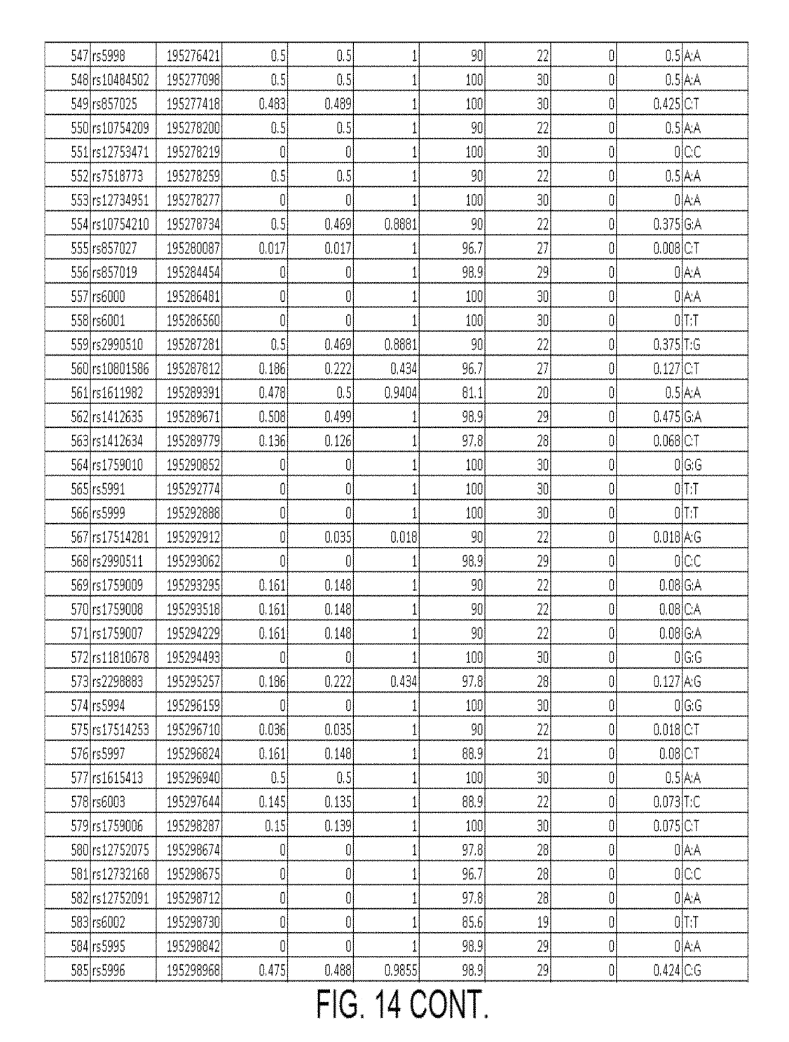

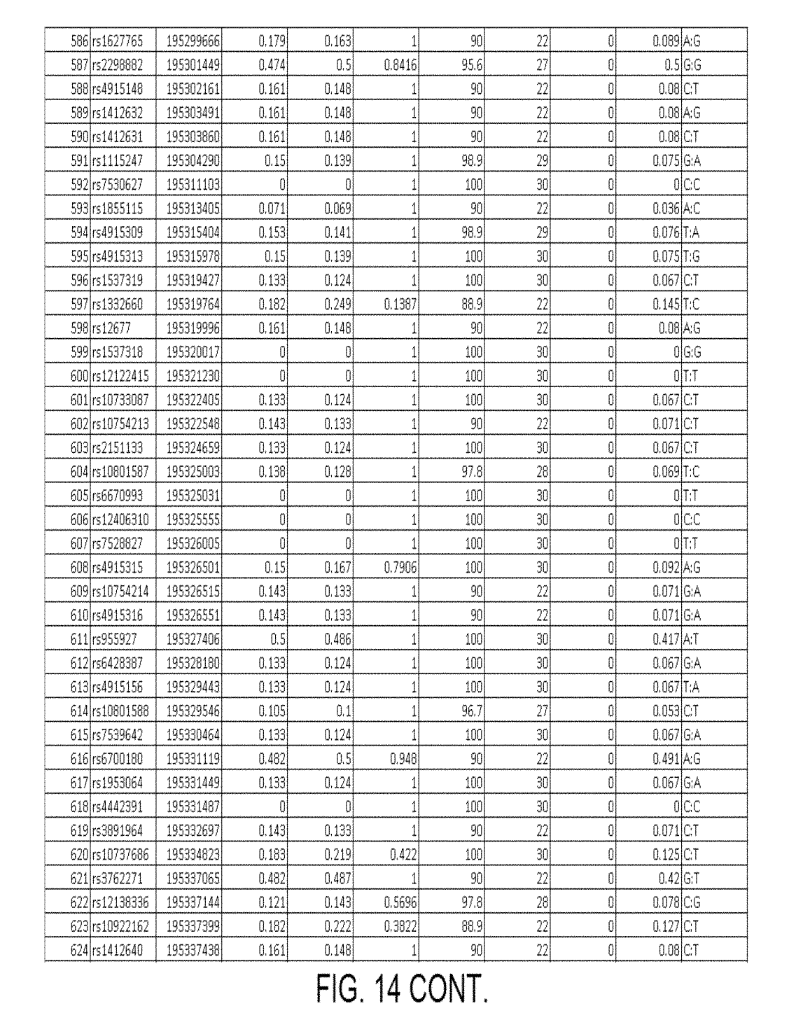

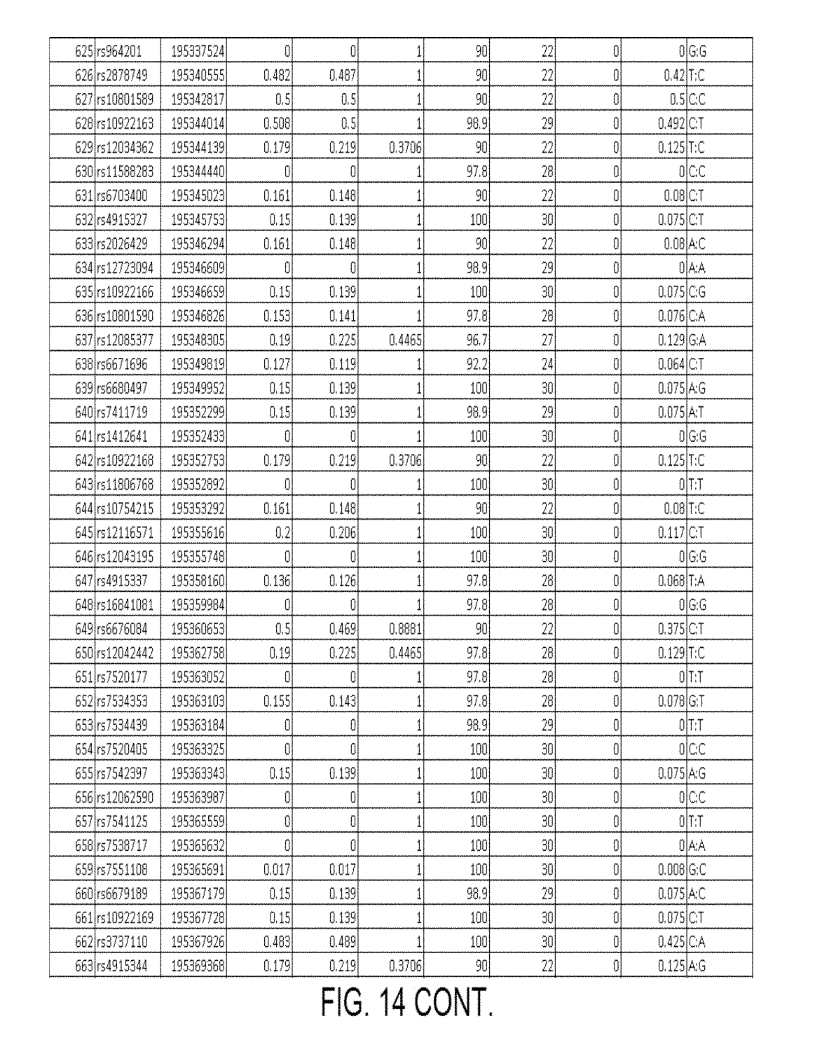

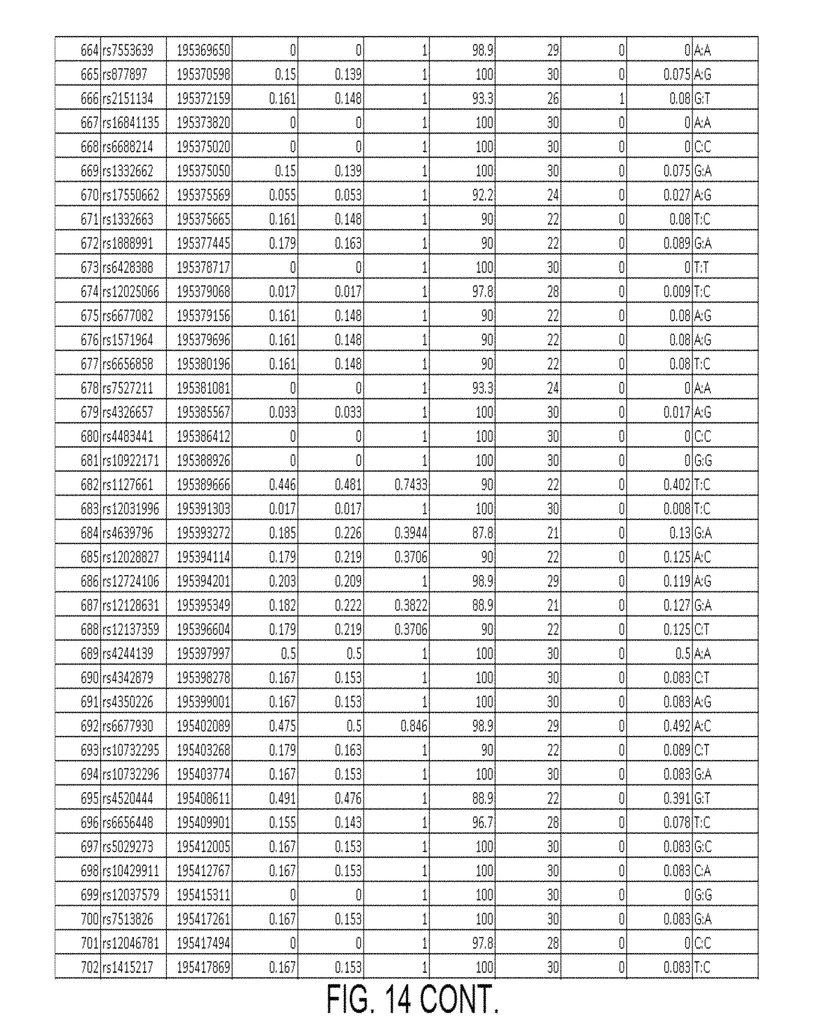

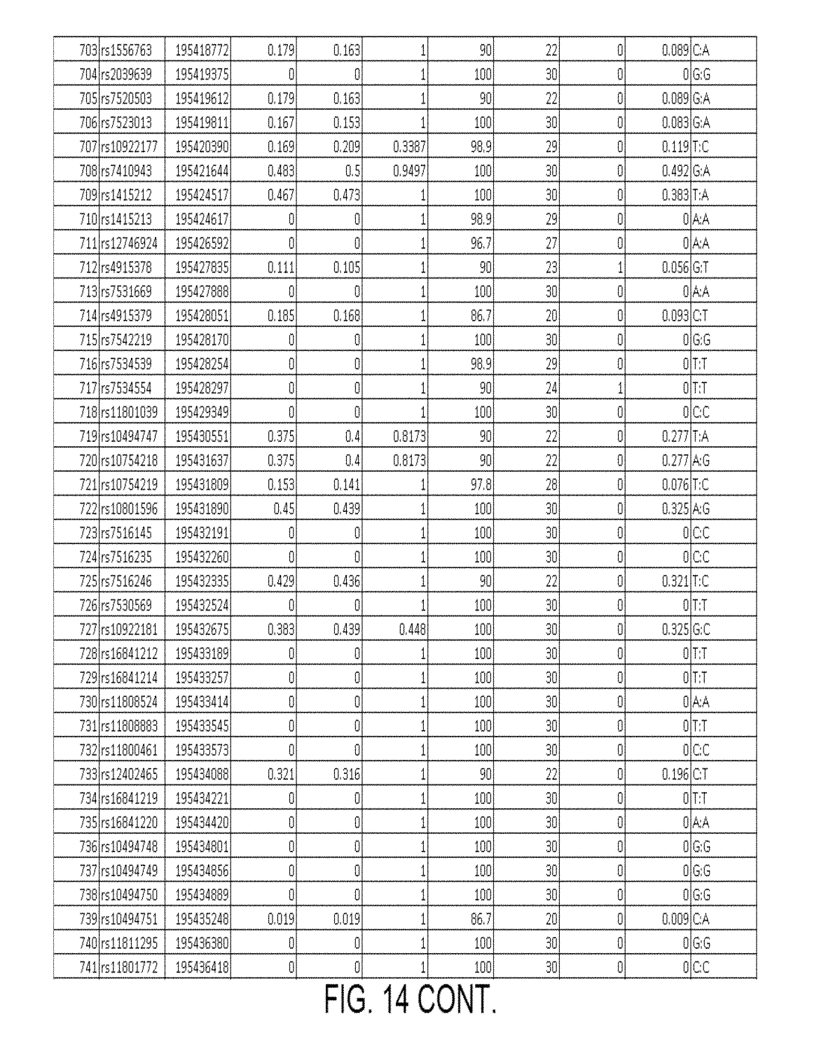

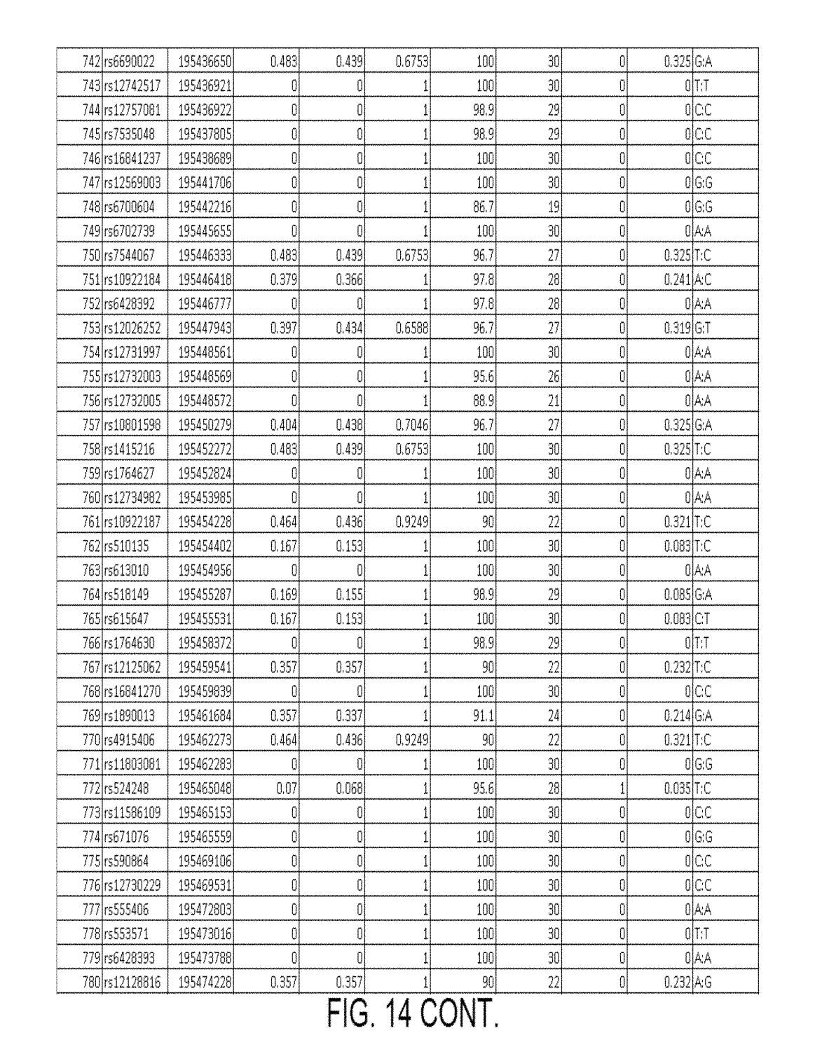

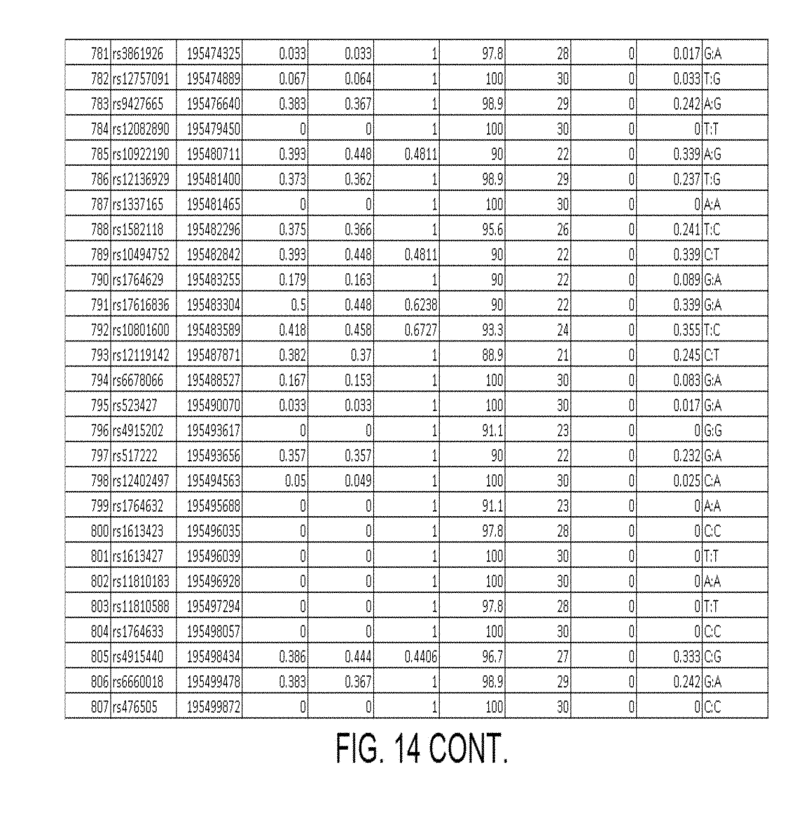

It was determined whether there were SNPs proximal and distal to the boundaries defined by the 62 SNP-based haplotypes that are in LD with the defined major haplotype blocks. All Hapmap SNPs for Caucasians lying between Chr1:194,814,311 and 195,431,890 were downloaded with subject information and analyzed in Haploview (FIG. 14). Default (Gabriel et al.) blocks encompassed the entire region. The second block, defined by rs10801551 (194,848,662) through rs800292 (194,908,856) encompassed rs35928059, the most 5′ SNP employed in the 62-SNP set. This suggests that the rs35928059 is in LD with SNPs as far proximal as rs10801551. Similarly, at the distal 3′ end of our locus, rs6428380, the most 3′ SNP in the 62_A SNP set, lies between rs4915148 (195,302,161) and rs1537319 (195,319,427), suggesting that the distal SNPs within this block are likely in LD with rs1537319.

3.20 Results: Characterization of CFHR3/CFHR1 Deletion Boundaries

Quantitative PCR (qPCR) was performed on multiple CEPH individuals representing each deletion tagging SNP genotype. Three qPCR primer sets were designed to amplify both duplicated regions equally, and two additional probes were designed in the sequence between the segmental duplications. All experiments were performed using QuantiFast SYBR RT-PCR Master Mix (Qiagen) and the MyiQ Single Color Real-Time PCR Detection System (BioRad). Non-deletion homozygotes showed the presence of both segmental duplications, as well as normal amplification of probes between the duplications. Heterozygotes showed intermediate values for probes within the duplications and a one-copy deletion in the region between the duplications. Individuals homozygous for the deletion tagging allele showed the presence of only one segmental duplication region and no amplification of probes located between the duplicated regions. Thus this qPCR assay can be used to reliably identify individuals as homozygous R3/R1 deletion, heterozygous R3/R1 deletion, or homozygous R3/R1 non-deletion.

3.21 Results: Development of Alternative CFHR3/CFHR1 Deletion Status Assays and Confirmation of Rs12144939 as a CFHR3/CFHR1 Deletion Tagging SNP

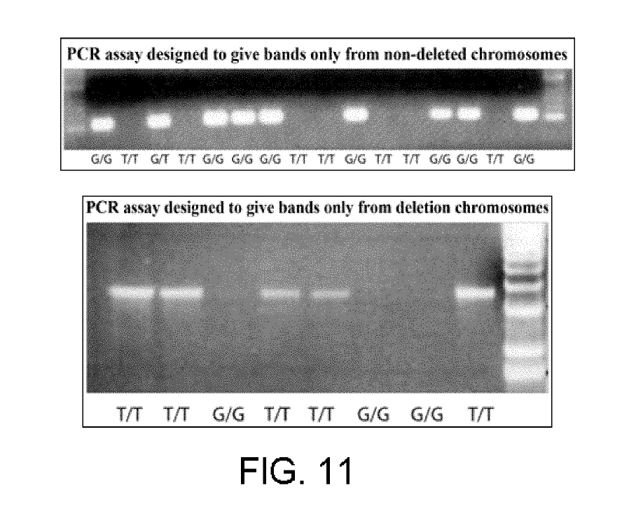

Two additional PCR assays were developed to confirm presence of deletion or presence on non-deletion chromosomes (FIG. 11). These assays were not designed to distinguish heterozygotes, but to determine status of homozygous deletion or non-deletion samples. Using the sequence variant signatures found in deletion homozygotes, primers were designed to generate a 3.7 kb PCR product only when the deletion is present, either in heterozygous or homozygous form. Reactions were performed using AccuPrime High Fidelity polymerase, buffers, and protocols (Invitrogen). Samples from twelve unrelated CEPH individuals and five African individuals were amplified and run on a 1% agarose gel. All homozygous deletion and heterozygous individuals produced a 3.7 kb band in the assay designed to amplify across the deletion, while none of the homozygote non-deletion individuals amplified. In addition, primers were designed and used to amplify a small (200 bp) PCR product only when the deletion is not present. Reactions were performed on eighty-eight CEPH individuals. All amplified well except for deletion homozygotes, which failed without exception. Thus these two assays can be used to unambiguously identify individuals homozygous either for the CFHR3-R1 deletion or for non-deletion status and in combination can determine heterozygotes.

3.21 Confirmation of the Deletion Tagging SNP

A predesigned Taqman CNV assay was run on all 604 CEPH individuals in order to confirm deletion status. This assay was located on chr1:195011298-195011397, just proximal to the CFHR3 gene. Assays were run on a 7900HT Fast Real-Time PCR System (Applied Biosystems) and analyzed using CopyCaller software (Applied Biosystems). Copy number calls were compared against rs12144939 (deletion tagging SNP) genotypes. In all cases where a clear call was made by CopyCaller software, copy number calls were consistent with what would be predicted by rs12144939, with the exception of one family in which six individuals were heterozygous for the tagging SNP but were called homozygous non-deletion in the CNV analysis. This confirmed that in Caucasians, rs12144939 tagged the deletion with near 100% reliability. Thus, CFHR1/R3 deletion status can now be obtained using the three assays described above. Analysis of the 1000-genome data indicates that the following SNPs accurately tag rs12144939, and therefore the CFHR3/CFHR1 deletion: rs6689009, rs35253683, rs731557, and rs60642321.

It will be apparent to those skilled in the art that various modifications and variations can be made in the present invention without departing from the scope or spirit of the invention. Other aspects of the invention will be apparent to those skilled in the art from consideration of the specification and practice of the invention described herein. It is intended that the specification and examples be considered as exemplary only, with a true scope and spirit of the invention being indicated by the following claims.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### E. REFERENCES

1. Abrera-Abeleda M A, Nishimura C, Smith J L, Sethi S, McRae J L, Murphy B F, Silvestri G, Skerka C, Jozsi M, Zipfel P F, Hageman G S, Smith R J. Variations in the complement regulatory genes factor H (CFH) and factor H related 5 (CFHR5) are associated with membranoproliferative glomerulonephritis type II (dense deposit disease). J Med Genet. 2006 July; 43(7):582-9.
2. Allikmets R, Bergen A A, Dean M, Guymer R H, Hageman G S, Klaver C C, Stefansson K, Weber B H; International Age-related Macular Degeneration Genetics Consortium. Geographic atrophy in age-related macular degeneration and TLR3. N Engl J Med. 2009 May 21; 360(21):2252.
3. Baird P N, Islam F M, Richardson A J, Cain M, Hunt N, Guymer R. Analysis of the Y402H variant of the complement factor H gene in age-related macular degeneration. Invest Ophthalmol Vis Sci. 2006 October; 47(10):4194-4198.
4. Breiman, Friedman, Olshen, and Stone. Classification and Regression Trees. Wadsworth, 1984.
5. S. R. Browning. Multilocus association mapping using variable-length Markov chains. Am J Hum Genet, 78:903-913, 2006.
6. DeAngelis M M, Ji F, Adams S, Morrison M A, Harring A J, Sweeney M O, Capone A Jr, Miller J W, Dryj a TP, Ott J, Kim I K. Alleles in the HtrA serine peptidase 1 gene alter the risk of neovascular age-related macular degeneration. Ophthalmology. 2008 July; 115(7):1209-1215.e7.
7. W. S. De Jong, D. M. De Jong, and M. Bodis. A fluorogenic 50 nuclease (taqman) assay to assess dosage of a marker tightly linked to red skin color in autotetraploid potato. Theor Appl Genet, 107:1384-1390, 2003.
8. H. Fakhrai-Rad, J. Zheng, T. D. Willis, K. Wong, K. Suyenaga, M. Moorhead, J. Eberle, Y. R. Thorstenson, T. Jones, R. W. Davis, E. Namsaraev, and M. Faham. Snp discovery in pooled samples with mismatch repair detection. Genome Res, 14:1404-1412, 2004.
9. Gold B, Merriam J E, Zernant J, Hancox L S, Taiber A J, Gehrs K, Cramer K, Neel J, Bergeron J, Barile G R, Smith R T; AMD Genetics Clinical Study Group, Hageman G S, Dean M, Allikmets R. Variation in factor B (BF) and complement component 2 (C2) genes is associated with age-related macular degeneration. Nat Genet. 2006 April; 38(4):458-62.
10. Gotoh N, Yamada R, Hiratani H, Renault V, Kuroiwa S, Monet M, et al. No association between complement factor H gene polymorphism and exudative age-related macular degeneration in Japanese. Hum Genet. 2006 August; 120(1):139-43.
11. Hageman G S, Gehrs K, Lejnine S, Bansal A T, Deangelis M M, Guymer R H, Baird P N, Allikmets R, Deciu C, Oeth P, Perlee L T. Clinical validation of a genetic model to estimate the risk of developing choroidal neovascular age-related macular degeneration. Hum Genomics. 2011 July; 5(5):420-40.
12. Hageman G S, Anderson D H, Johnson L V, Hancox L S, Taiber A J, Hardisty L I, Hageman J L, Stockman H A, Borchardt J D, Gehrs K M, Smith R J, Silvestri G, Russell S R, Klaver C C, Barbazetto I, Chang S, Yannuzzi L A, Barile G R, Merriam J C, Smith R T, Olsh A K, Bergeron J, Zernant J, Merriam J E, Gold B, Dean M, Allikmets R. A common haplotype in the complement regulatory gene factor H (HF1/CFH) predisposes individuals to age-related macular degeneration. Proc Natl Acad Sci USA. 2005 May 17; 102(20):7227-7232.
13. Hageman G S, Hancox L S, Taiber A J, Gehrs K M, Anderson D H, Johnson L V, Radeke M J, Kavanagh D, Richards A, Atkinson J, Men S, Bergeron J, Zernant J, Merriam J, Gold B, Allikmets R, Dean M; AMD Clinical Study Group. Extended haplotypes in the complement factor H (CFH) and CFH-related (CFHR) family of genes protect against age-related macular degeneration: characterization, ethnic distribution and evolutionary implications. Ann Med. 2006; 38(8):592-604.
14. P. Hardenbol, J. Banr, M. Jain, M. Nilsson, E. A. Namsaraev, G. A. Karlin-Neumann, H. Fakhrai-Rad, M. Ronaghi, T. D. Willis, U. Landegren, and R. W. Davis. Multiplexed genotyping with sequence-tagged molecular inversion probes. Nat Biotechnol, 21:673-678, 2003.
15. Lau L I, Chen S J, Cheng C Y, Yen M Y, Lee F L, Lin M W, et al. Association of the Y402H polymorphism in complement factor H gene and neovascular age-related macular degeneration in Chinese patients. Invest Ophthalmol Vis Sci. 2006 August; 47(8):3242-6.
16. Lim J H, Wickremasinghe S S, Xie J, Chauhan D S, Baird P N, Robman L D, Hageman G S, Guymer R H. Delay to treatment and visual outcomes in patients treated with anti-vascular endothelial growth factor for age-related macular degeneration. Am J Ophthalmol. 2012 April; 153(4):678-686.
17. R Development Core Team. R: A Language and Environment for Statistical Computing. R Foundation for Statistical Computing, Vienna, Austria, 2006, ISBN 3-900051-07-0.
18. Robman L, Baird P N, Dimitrov P N, Richardson A J, Guymer R H. C-reactive protein levels and complement factor H polymorphism interaction in age-related macular degeneration and its progression. Ophthalmol. 2010 October; 117(10):1982-1988.
19. M. N. Stephens and P. Donnelly. A comparison of bayesian methods for haplotype reconstruction. Am J Hum Genet, 73:1162-1169, 2003.
20. M. N. Stephens, N. J. Smith, and P. Donnelly. A new statistical method for haplotype reconstruction from population data. Am J Hum Genet, 68:978-989, 2001.
21. Wickremasinghe S S, Xie J, Lim J, Chauhan D S, Robman L, Richardson A J, Hageman G, Baird P N, Guymer R. Variants in the APOE gene are associated with improved outcome after anti-VEGF treatment for neovascular AMD. Invest Ophthalmol Vis Sci. 2011 Jun. 8; 52(7):4072-9.
22. Zhang H, Morrison M A, Dewan A, Adams S, Andreoli M, Huynh N, Regan M, Brown A, Miller J W, Kim I K, Hoh J, Deangelis M M. The NEI NCBI dbGAP database: genotypes and haplotypes that may specifically predispose to risk of neovascular age-related macular degeneration. BMC Med Genet. 2008 Jun. 9; 9:51.

1. A method for determining a Caucasian subject's susceptibility to having or developing age-related macular degeneration comprising determining in the Caucasian subject the identity of one or more haplotypes, wherein the one or more haplotypes are H1_62_A, H2_62_A, H3_62_A, H4_62_A, H5_62_A, H6_62_A, H7_62_A, H8_62_A, H9_62_A, H10_62_A, H11_62_A, H12_62_A, H13_62_A, H14_62_A, H15_62_A, or a complement thereof, and wherein the presence of one or more of the haplotypes indicates the subject's susceptibility for having or developing age-related macular degeneration.

2. The method of claim 1, wherein the subject's haplotype is determined from a sample obtained from the subject.

3. The method of claim 1, wherein the subject's haplotype is determined by amplifying or sequencing a nucleic acid sample obtained from the subject.

4. The method of claim 1, wherein H2_62_A, or a complement thereof, is indicative of the subject's increased risk for having or developing age-related macular degeneration.

5. The method of claim 4, further comprising administering a therapeutic composition to the subject.

6. The method of claim 1, wherein H3_62_A, H5_62_A, H11_62_A, or a complement thereof, is indicative of the subject's decreased risk for having or developing age-related macular degeneration.

7. The method of claim 1, wherein the subject is female.

8. The method of claim 7, wherein H1_62_A, H2_62_A, or a complement thereof, is indicative of the subject's increased risk for having or developing age-related macular degeneration

9. The method of claim 7, wherein H3_62_A, H5_62_A, H11_62_A, or a complement thereof, is indicative of the subject's decreased risk for having or developing age-related macular degeneration

10. The method of claim 1, wherein the subject is male.

11. The method of claim 10, wherein H2_62_A, or a complement thereof, is indicative of the subject's increased risk for having or developing age-related macular degeneration

12. The method of claim 10, wherein H11_62_A, or a complement thereof, is indicative of the subject's decreased risk for having or developing age-related macular degeneration.

13. A method for determining a Caucasian subject's susceptibility to having or developing age-related macular degeneration comprising determining in the Caucasian subject the identity of one or more haplotypes, wherein the one or more haplotypes are H1_51_A, H2_51_A, H3_51_A, H4_51_A, H5_51_A, H6_51_A, H7_51_A, H8_51_A, H9_51_A, H10_51_A, H11_51_A, H12_51_A, H13_51_A, H14_51_A, H15_51_A, H16_51_A, H17_51_A, or a complement thereof, and wherein the presence of one or more of the haplotypes indicates the subject's susceptibility for having or developing age-related macular degeneration.

14. The method of claim 13, wherein the subject's haplotype is determined from a sample obtained from the subject.

15. The method of claim 13, wherein the subject's haplotype is determined by amplifying or sequencing a nucleic acid sample obtained from the subject.

16. The method of claim 13, wherein H2_51_A, or a complement thereof, is indicative of the subject's increased risk for having or developing age-related macular degeneration.

17. The method of claim 16, further comprising administering a therapeutic composition to the subject.

18. The method of claim 13, wherein H3_51_A, H5_51_A, H10_51_A, or a complement thereof, is indicative of the subject's decreased risk for having or developing age-related macular degeneration.

19. A method for determining a Caucasian subject's susceptibility to having or developing age-related macular degeneration comprising determining in the Caucasian subject the identity of one or more haplotypes, wherein the one or more haplotypes are H1_51_B, H2_51_B, H3_51_B, H4_51_B, H5_51_B, H6_51_B, H7_51_B, H8_51_B, H9_51_B, H10_51_B, H11_51_B, H12_51_B, H13_51_B, H14_51_B, H15_51_B, H16_51_B, H17_51_B, H18_51_B, H19_51_B, H20_51_B, H21_51_B, H22_51_B, or a complement thereof, and wherein the presence of one or more of the haplotypes indicates the subject's susceptibility for having or developing age-related macular degeneration.

20. The method of claim 19, wherein the subject's hap lo type is determined from a sample obtained from the subject.

21-240. (canceled)


SEQUENCE LISTING (376 sequences)

SEQ ID NO: 1 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgagggcggcgtaccggggcaagcaccg

SEQ ID NO: 2 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgagggcggcgtaccgaatcaagtaccg

SEQ ID NO: 3 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgngattagtgaacgcatgaccgaaagtggcgtacctggttaagcaccg

SEQ ID NO: 4 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnggatggcggacttgattccaaaggcaatgttcataatcaagtaccg

SEQ ID NO: 5 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgngctcatatagnaattagttgacgcnnnnccgaaggcagctcacatgatcaaatgtta

SEQ ID NO: 6 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgngattagtgaacgcatgaccgaaagtgatgttcataatcaagtaccg

SEQ ID NO: 7 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
gtcgnatccatatatnagagaacggccgcatgaccgagggcggcgtaccgaatcaagtaccg

SEQ ID NO: 8 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgagggcgatgttcataatcaagtaccg

SEQ ID NO: 9 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnggatggcggacttgattccgaaggcggcgtaccggggcaagcaccg

SEQ ID NO: 10 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnggatggcggacttgattccagaggcagcgtaccggggcaagcaccg

SEQ ID NO: 11 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
atcgnatccatatatnaattagttgacgcnnnnccaaaggcaatgttcataatcaagtaccg

SEQ ID NO: 12 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgaaagtggcgtacctggttaagcaccg

SEQ ID NO: 13 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgngctcatatagnaattagttgacgcnnnnccgaaggcaatgttcataatcaagtaccg

SEQ ID NO: 14 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgaaggcggcgtaccggggcaagcaccg

SEQ ID NO: 15 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgngctcatatagnaattagttgacgcnnnnccgaaggcagcgtaccggggcaagcaccg

SEQ ID NO: 16 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggctgcatgacagagggcggcgtaccggggcaagcaccg

SEQ ID NO: 17 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnggatggcggacttgattccgaagacggcgtaccggggcaagcaccg

SEQ ID NO: 18 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
atcgngctcatatagnaattagttgacgcnnnnccgaaggcagctcacatgatcaaatgtta

SEQ ID NO: 19 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatcatcgcagnagagaacggccgcatgaccgagggcggcgtaccggggcaagcaccg

SEQ ID NO: 20 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgagggcggcgtaccggggctagcaccg

SEQ ID NO: 21 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnggatggcggacttgattccaaaggcagcgttcataatcaagtaccg

SEQ ID NO: 22 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
gtcgnatcatcgcagnagagaacggccgcatgaccgagggcggcgtaccgaatcaagtaccg

SEQ ID NO: 23 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgagcgagggcggcgtaccgaatcaagtaccg

SEQ ID NO: 24 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccaaaggcaatgttcataatcaagtaccg

SEQ ID NO: 25 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatatatnggatggcggacttgattccgaagacggcgtaccggggcaagcaccg

SEQ ID NO: 26 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
atcgnatccatatatnaattagttgacgcnnnnccaaaggcaatgttcataatcaggtaccg

SEQ ID NO: 27 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgagggcggcgtacctggttaagcaccg

SEQ ID NO: 28 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
atcgnatccatatatnaattagttgacgcnnnnccaaaggcaatgtaccggggcaagcaccg

SEQ ID NO: 29 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnggatggcggacttgattccgaaagtggcgtacctggttaagcaccg

SEQ ID NO: 30 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgngattagtgaacgcatgaccgaaagtggcgtaccgaatcaagtaccg

SEQ ID NO: 31 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
atcgnatccatatatnaattagttgacgcnnnnccgaaagtggcgtacctggttaagcaccg

SEQ ID NO: 32 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnggatggcggacttgattccgagggcggcgtaccgaatcaagtaccg

SEQ ID NO: 33 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgngattagtgaacgcatgaccgaaagtggcgtaccggggcaagcaccg

SEQ ID NO: 34 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgngattagtgaacgcatgaccgaaagtggcgtacctgggcaagcaccg

SEQ ID NO: 35 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgagggcggcgtatcggggcaagcaccg

SEQ ID NO: 36 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatccatatatnagagaacggccgcatgaccgagggcggcgtaccgaatcaagtaccg

SEQ ID NO: 37 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgngattagtgaacgcatgaccgagggcggcgtaccggggcaagcaccg

SEQ ID NO: 38 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnaattagttgacttgattccagaggcggcgtaccggggcaagcaccg

SEQ ID NO: 39 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatcatcgcagnagagaacggccgcatgaccgagggcggcgtaccgaatcaagtaccg

SEQ ID NO: 40 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgngattagtgaacgcatgaccgaaggcggcgtaccgaatcaagtaccg

SEQ ID NO: 41 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatccatattgngattagtgaacgcatgaccgaaagtggcgtacctggttaagcaccg

SEQ ID NO: 42 - DNA, 62 bp, Artificial Sequence; identical to SEQ ID NO: 39
  Description of Artificial Sequence; Note = Synthetic Construct
  misc_feature (5)..(5): n is a, c, g, or t
  misc_feature (16)..(16): n is a, c, g, or t

SEQ ID NO: 43 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgngctcatatagnaattagttgacgcatgaccgaaggcagctcacatgatcaaatgtta

SEQ ID NO: 44 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgagggcggctcacatgatcaaatgtta

SEQ ID NO: 45 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgagggcggtgttcataatcaagtaccg

SEQ ID NO: 46 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgnatcatcgcagnagagaacggccgcnnnnccgagggcggcgtaccgaatcaagtaccg

SEQ ID NO: 47 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
gtcgnatcatcgcagnagagaacggccgcatgagcgagggcggcgtaccgaatcaagtaccg

SEQ ID NO: 48 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnggatggcggacttgattccaaaggcagcgtaccggggcaagcaccg

SEQ ID NO: 49 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
atcgnatccatatatnaattagttgacgcnnnnccgagggcggcgtaccggggcaagcaccg

SEQ ID NO: 50 - DNA, 62 bp, Artificial Sequence; identical to SEQ ID NO: 19
  Description of Artificial Sequence; Note = Synthetic Construct
  misc_feature (5)..(5): n is a, c, g, or t
  misc_feature (16)..(16): n is a, c, g, or t

SEQ ID NO: 51 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgngattagtgaacgcatttccgaaagtggcgtacctggttaagcaccg

SEQ ID NO: 52 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgngattagtgaacgcatttccgaaagtgatgttcataatcaagtaccg

SEQ ID NO: 53 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
gtcgnatccatatatnagagaacggccgcatgaccgaaagtggcgtacctggttaagcaccg

SEQ ID NO: 54 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnaattagttgacgcatgaccaaaggcaatgttcataatcaagtaccg

SEQ ID NO: 55 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatccatatatnggatggcggacttgattccaaaggcaatgttcataatcaagtaccg

SEQ ID NO: 56 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgngttcatatagnaattagttgacgcnnnnccgaaggcagcgtaccggggcaagcaccg

SEQ ID NO: 57 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgnatccatatatnaattagttgacgcnnnnccgaaagtggcgtacctggttaagcaccg

SEQ ID NO: 58 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgnggttagtgaacgcatgaccgaaagtggcgtacctggttaagcaccg

SEQ ID NO: 59 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatcatcgcagnagagaacggccgcatgagcgagggcggcgtaccgaatcaagtaccg

SEQ ID NO: 60 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgngctcatatagnaattagttgacgcnnnnccgagggcggcgtaccgaatcaagtaccg

SEQ ID NO: 61 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgngattagtgaacgcatgaccgagagtggcgtacctggttaagcaccg

SEQ ID NO: 62 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgagggcggcgtaccggggcaagtaccg

SEQ ID NO: 63 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccagaggcagcgtaccggggcaagcaccg

SEQ ID NO: 64 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatatatnggatggcggacttgattccgagggcggcgtaccggggcaagcaccg

SEQ ID NO: 65 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatcatcgcagnagagaacggccgcatgaccgagggcggcgtaccggggcaagcaccg

SEQ ID NO: 66 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatatatnggatggcggacttgattccagaggcagcgtaccggggcaagcaccg

SEQ ID NO: 67 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
attanatccatattgngattagtgaacgcatgaccgaaagtggcgtacctggttaagcaccg

SEQ ID NO: 68 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggacgcatgagcgagggcggcgtaccgaatcaagtaccg

SEQ ID NO: 69 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatattgngattagtgaacgcatgaccgaaagtggcgtacctggttaagcaccg

SEQ ID NO: 70 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgngattagtgaacgcatttccgaaggcggcgtaccgaatcaagtaccg

SEQ ID NO: 71 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
gtcgnatccatatatnagagaacggccgcatgaccgaaggcggcgtaccgaatcaagtaccg

SEQ ID NO: 72 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
gtcgnatccatatatnagagaacggccgcatgaccgagggcggcgtaccggggcaagcaccg

SEQ ID NO: 73 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgagagtggcgtacctggttaagcaccg

SEQ ID NO: 74 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnggatggcggacttgattccgaaggcaatgttcataatcaagtaccg

SEQ ID NO: 75 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcatagnagagaacggccgcatgaccgagggcggcgtaccggggcaagcaccg

SEQ ID NO: 76 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgagggcgatgtaccggggcaagcaccg

SEQ ID NO: 77 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatatatnggatggcggacttgattccaaaggcaatgttcataatcaagtaccg

SEQ ID NO: 78 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
gtcgnatccatatatnggatggcggacttgattccaaaggcaatgttcataatcaagtaccg

SEQ ID NO: 79 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
actanattcatatagnaattagttgacgcnnnnccgaaggcagctcacatgatcaaatgtta

SEQ ID NO: 80 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatccatatatnagagaacggccgcatgaccgagggcggcgtaccggggcaagcaccg

SEQ ID NO: 81 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatatatnggatggcggacttgattccgaaagtggcgtacctggttaagcaccg

SEQ ID NO: 82 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
actangctcatatagnaattagttgacgcnnnnccaaaggcaatgttcataatcaagtaccg

SEQ ID NO: 83 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgngctcatatagnaattagttgacgcnnnnccaaaggcaatgttcataatcaagtaccg

SEQ ID NO: 84 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgaaagtggcgtaccggggcaagcaccg

SEQ ID NO: 85 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatcatcgcagnagagaacggccttgattccaaaggcaatgttcataatcaagtaccg

SEQ ID NO: 86 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatccatatatnggatggcggacttgattccgagggcagcgtaccgaatcaagtaccg

SEQ ID NO: 87 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatccatattgngattagtgaacgcatgaccgaaagtgatgttcataatcaagtaccg

SEQ ID NO: 88 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatccatatatnggatggcggacttgattccgaagacggcgtaccgaatcaagtaccg

SEQ ID NO: 89 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatccatatatnggatggcggacttgattccaaaggcaatgttcatgatcaaatgtta

SEQ ID NO: 90 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgnatccatatatnaattagttgacgcnnnnccgaaagtgatgttcataatcaagtaccg

SEQ ID NO: 91 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcaatgcatnggatagcggccttgattccgaaggcaatgttcataatcaagtaccg

SEQ ID NO: 92 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgngctcatatagnaattagttgccgcnnnnccgaaggcagctcacatgatcaaatgtta

SEQ ID NO: 93 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnggatggcggccttgattccagaggcagcgtaccggggcaagcaccg

SEQ ID NO: 94 - DNA, 62 bp, Artificial Sequence; identical to SEQ ID NO: 1
  Description of Artificial Sequence; Note = Synthetic Construct
  misc_feature (5)..(5): n is a, c, g, or t
  misc_feature (16)..(16): n is a, c, g, or t

SEQ ID NO: 95 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgnattcatatagnaattagttgacgcnnnnccgaaggcaatgttcataatcaagtaccg

SEQ ID NO: 96 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
atcgngctcatatagnaattagttgacgcnnnnccgaaggcaatgttcataatcaagtaccg

SEQ ID NO: 97 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatccatatatnggatggcggacttgattccgaagacggcgtaccggggcaagcaccg

SEQ ID NO: 98 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgnattcatatagnagttggttgacgcnnnnccgaaggcaatgttcataatcaagtaccg

SEQ ID NO: 99 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnagagaacggccgcatgaccgagggcggcgtaccgaatcaagtaccg

SEQ ID NO: 100 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgnggttagtgaacgcatgaccgaaagtgatgttcataatcaagtaccg

SEQ ID NO: 101 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
atcgngctcatatagnaattagttgacgcnnnnccgaaggcagcgtaccggggcaagcaccg

SEQ ID NO: 102 - DNA, 62 bp, Artificial Sequence; identical to SEQ ID NO: 13
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t

SEQ ID NO: 103 - DNA, 62 bp, Artificial Sequence; identical to SEQ ID NO: 3
  Description of Artificial Sequence; Note = Synthetic Construct
  misc_feature (5)..(5): n is a, c, g, or t
  misc_feature (16)..(16): n is a, c, g, or t

SEQ ID NO: 104 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggacgcatgaccgagggcggcgtaccgaatcaagtaccg

SEQ ID NO: 105 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgngattagtgaacgcatgaccgaaggcagctcacatgatcaaatgtta

SEQ ID NO: 106 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatcatcgcagnagagaacggccgcatgaccgagggcggcgtaccgaatcaagtaccg

SEQ ID NO: 107 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgngttcatatagnaattagttgacgcnnnnccgaaggcagctcacatgatcaaatgtta

SEQ ID NO: 108 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
gccgnatccatatatnagagaacggccgcatgaccgagggcggcgtaccgaatcaagtaccg

SEQ ID NO: 109 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatngaatggcggacttgattccaaaggcaatgttcataatcaagtaccg

SEQ ID NO: 110 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
atcgngctcatatagnagttagttgacgcnnnnccgaaggcagctcacatgatcaaatgtta

SEQ ID NO: 111 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
atcgnatccatatatnagttagttgacgcnnnnccgaaagtggcgtacctggttaagcaccg

SEQ ID NO: 112 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggctgcatgacagagggcggcgtacctggttaagcaccg

SEQ ID NO: 113 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnattcatgcagnagttagttgccgcgattccaaaggcaatgttcataatcaagtaccg

SEQ ID NO: 114 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgngctcatatagnagttagttgacgcnnnnccgaaggcagctcacatgatcaaatgtta

SEQ ID NO: 115 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnaattagttgacgcatgaccaaaggcaatgttcataatcaggtaccg

SEQ ID NO: 116 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnggatggcggacttgattccgagggcggcgtaccggggcaagcaccg

SEQ ID NO: 117 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnggatggcggacttgagaccgagggcggcgtaccgaatcaagtaccg

SEQ ID NO: 118 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
atcgnatccatatatnaattagttgacgcnnnnccaaaggcagcgtaccggggcaagcaccg

SEQ ID NO: 119 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgngctcatatagnagttagttgacgcnnnnccgaaggcggcgtaccgaatcaagtaccg

SEQ ID NO: 120 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcatnagagaacggccgcatgaccgagggcggcgtaccgaatcaagtaccg

SEQ ID NO: 121 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
gtcgnatccatatatnagagaacggccgcatgaccgagagtggcgtacctggttaagcaccg

SEQ ID NO: 122 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgagagtggcgtaccgaatcaagtaccg

SEQ ID NO: 123 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaatggccgcatgaccaaaggcagcgttcataatcaagtaccg

SEQ ID NO: 124 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatccatatagngattagtggacgcatgaccgaaggcggcgtaccgaatcaagtaccg

SEQ ID NO: 125 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgngctcatatagnaattagttgacgcnnnnccgaaggcagctcacataatcaagtaccg

SEQ ID NO: 126 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatattgngattagtgaacttgattccaaaggcaatgttcataatcaagtaccg

SEQ ID NO: 127 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagatagcggacgcaattccaaaggcggcttacatgatcaagtaccg

SEQ ID NO: 128 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccaaaggcaatgttcataatcaggtaccg

SEQ ID NO: 129 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccaaaggcggcgtaccgaatcaagtaccg

SEQ ID NO: 130 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgngctcatatagnaattagttgacgcnnnnccgaaggcagctttcataatcaagtaccg

SEQ ID NO: 131 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgngattagtgaacttgattccagaggcagcgtaccggggcaagcaccg

SEQ ID NO: 132 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
actangctcatatagnagatagttgacgcnnnnccgaaggcggcgtaccgaatcaagtaccg

SEQ ID NO: 133 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgnggttagtgaacgcatgaccgaaggcagctcacatgatcaaatgtta

SEQ ID NO: 134 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgngaagaacggccgcatgaccgagggcggcgtaccgaatcaagtaccg

SEQ ID NO: 135 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnaattagttgacgcgattccaaaggcaatgttcataatcaagtaccg

SEQ ID NO: 136 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
atcgnatccatatatnaattagttgacgcnnnnccaaaggcggcgtaccgaatcaagtaccg

SEQ ID NO: 137 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgngccatcgcagnagagaacggccgcatgaccgagggcggcgtaccggggcaagcaccg

SEQ ID NO: 138 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnggatggcggacttgagaccgaagacggcgtaccggggcaagcaccg

SEQ ID NO: 139 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgngctcatatagnaattagttgacgcnnnnccgaaggcagctcacatgatcaagtaccg

SEQ ID NO: 140 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgagggcggcgtaccgaatcaggtaccg

SEQ ID NO: 141 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
atcgnattcatatagnaattagttgacgcnnnnccgaaggcagctcacatgatcaaatgtta

SEQ ID NO: 142 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
gtcgnatcatcgcagnagagaacggccgcatgaccaaaggcaatgttcataatcaagtaccg

SEQ ID NO: 143 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnaattagttgacgcatgaccaaaggcaatgtaccggggcaagcaccg

SEQ ID NO: 144 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgngctcatatagnaattggcggacttgattccaaaggcaatgttcataatcaagtaccg

SEQ ID NO: 145 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatatatnagagaacggccgcatgaccgagggcggcgtaccgaatcaagtaccg

SEQ ID NO: 146 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnggatggcggacgcatgaccaaaggcaatgttcataatcaagtaccg

SEQ ID NO: 147 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcaatgcatnggatggcggacttgattccaaaggcaatgttcataatcaagtaccg

SEQ ID NO: 148 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
accgngctcatatagnaattagttgacgcnnnnccgagggcagctcacatgatcaaatgtta

SEQ ID NO: 149 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnaaagaacggccgcatgaccgagggcggcgtaccggggctagcaccg

SEQ ID NO: 150 - DNA, 62 bp, Artificial Sequence; identical to SEQ ID NO: 106
  Description of Artificial Sequence; Note = Synthetic Construct
  misc_feature (5)..(5): n is a, c, g, or t
  misc_feature (16)..(16): n is a, c, g, or t

SEQ ID NO: 151 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
actanatccatattgngattagttgacgcnnnnccgaaggcagctcacatgatcaaatgtta

SEQ ID NO: 152 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatatatnggatagcggacgtaattccgaaggcagctcacatgatcaaatgtta

SEQ ID NO: 153 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatatatnggatggcggacttgattccaaaagcgatgttcataatcaagtaccg

SEQ ID NO: 154 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
atcgnatccatatatnggatggcggacttgattccagaggcagcgtaccggggcaagcactg

SEQ ID NO: 155 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgagggcggcgtaccgaatcaagcaccg

SEQ ID NO: 156 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcatcgcagnagagaacggccgcatgaccgagggcagcgtaccgaatcaagtaccg

SEQ ID NO: 157 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatccatatatnagagaacggccgcatgaccgagggcgatgttcataatcaagtaccg

SEQ ID NO: 158 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgngccctcatagnagagaacggacgcatgaccgagggcggcgtaccggggcaagcaccg

SEQ ID NO: 159 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actanatccatattgngattagtgaacttgattccgagggcggcgtaccggggcaagcaccg

SEQ ID NO: 160 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
  misc_feature (30)..(33): n is a, c, g, or t
atcgnatcatcgcagnagagaacggccgcnnnnccgaaggcagctcacatgatcaaatgtta

SEQ ID NO: 161 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
gtcgnatcatcgcagnagagaacggccgcatgaccgagggcggcttaccgaatcaagtaccg

SEQ ID NO: 162 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
actangctcatatagngattagtgaacgcatgaccgaaagtggcgtacctggttaagcaccg

SEQ ID NO: 163 - DNA, 62 bp, Artificial Sequence
  Description of Artificial Sequence; Note = Synthetic Construct
  misc_feature (5)..(5): n is a, c, g, or t
  misc_feature (16)..(16): n is a, c, g, or t
accgnatcctcatagnaaaggacggacgcatgaccgagggcggcgtaccggggcaagcaccg

SEQ ID NO: 164 - DNA, 51 bp, Artificial Sequence
  Description of Artificial Sequence; Note = Synthetic Construct
accgatcaaccatgacaaggccgccggacgcggggttgcgcggcttcgtcc

SEQ ID NO: 165 - DNA, 51 bp, Artificial Sequence
  Description of Artificial Sequence; Note = Synthetic Construct
accgatcaaccatgacaaggccgccggacgcggggttgcgtatcttcatcc

SEQ ID NO: 166 - DNA, 51 bp, Artificial Sequence
  Description of Artificial Sequence; Note = Synthetic Construct
actaatccttttcataagagtcgccggatacggggttgctcgttttcgtcc

SEQ ID NO: 167 - DNA, 51 bp, Artificial Sequence
  Description of Artificial Sequence; Note = Synthetic Construct
atcgatcctttacgaaggggccttcgaatgcaaagtagattatcttcatcc

SEQ ID NO: 168 - DNA, 51 bp, Artificial Sequence
  Description of Artificial Sequence; Note = Synthetic Construct
accggctctttatataagatccgccggatgcaggtctgatcatctttactt

SEQ ID NO: 169 - DNA, 51 bp, Artificial Sequence
  Description of Artificial Sequence; Note = Synthetic Construct
actaatccttttcataagagtcgccggatacgaagtagattatcttcatcc

SEQ ID NO: 170 - DNA, 51 bp, Artificial Sequence
  Description of Artificial Sequence; Note = Synthetic Construct
gtcgatcctttatgacaaggccgccggacgcggggttgcgtatcttcatcc

SEQ ID NO: 171 - DNA, 51 bp, Artificial Sequence
  Description of Artificial Sequence; Note = Synthetic Construct
accgatcaaccatgacaaggccgccggacgcgaagtagattatcttcatcc

SEQ ID NO: 172 - DNA, 51 bp, Artificial Sequence
  Description of Artificial Sequence; Note = Synthetic Construct
atcgatcctttacgaaggggccttcggatgcggggttgcgcggcttcgtcc

SEQ ID NO: 173 - DNA, 51 bp, Artificial Sequence
  Description of Artificial Sequence; Note = Synthetic Construct
atcgatcctttatataagatccgccgaatgcaaagtagattatcttcatcc

SEQ ID NO: 174 - DNA, 51 bp, Artificial Sequence
  Description of Artificial Sequence; Note = Synthetic Construct
atcgatcctttacgaaggggccttcgagtgcagggttgcgcggcttcgtcc

SEQ ID NO: 175 - DNA, 51 bp, Artificial Sequence
  Description of Artificial Sequence; Note = Synthetic Construct
accgatcaaccatgacaaggccgccggatacggggttgctcgttttcgtcc

SEQ ID NO: 176 - DNA, 51 bp, Artificial Sequence
  Description of Artificial Sequence; Note = Synthetic Construct
accgatcaaccatgacaaggccgccggatgcggggttgcgcggcttcgtcc

SEQ ID NO: 177 - DNA, 51 bp, Artificial Sequence
  Description of Artificial Sequence; Note = Synthetic Construct
accggctctttatataagatccgccggatgcaaagtagattatcttcatcc

SEQ ID NO: 178 - DNA, 51 bp, Artificial Sequence
  Description of Artificial Sequence; Note = Synthetic Construct
accggctctttatataagatccgccggatgcagggttgcgcggcttcgtcc

SEQ ID NO: 179 - DNA, 51 bp, Artificial Sequence
  Description of Artificial Sequence; Note = Synthetic Construct
atcgatcaaccatgacaaggccgccggacgcggggttgcgcggcttcgtcc

SEQ ID NO: 180 - DNA, 51 bp, Artificial Sequence
  Description of Artificial Sequence; Note = Synthetic Construct
accgatcaaccatgacaaggccgcgggacgcggggttgcgtatcttcatcc

SEQ ID NO: 181 - DNA, 51 bp, Artificial Sequence
  Description of Artificial Sequence; Note = Synthetic Construct
accgatcaaccatgacaaggctgcctgacgcggggttgcgcggcttcgtcc

SEQ ID NO: 182 - DNA, 51 bp, Artificial Sequence
  Description of Artificial Sequence; Note = Synthetic Construct
atcgatcctttacgaaggggccttcggatgtggggttgcgcggcttcgtcc

SEQ ID NO: 183 - DNA, 51 bp, Artificial Sequence
  Description of Artificial Sequence; Note = Synthetic Construct
accgatcaaccatgacaaggccgccgaatgcaaagtagattatcttcatcc

SEQ ID NO: 184 - DNA, 51 bp, Artificial Sequence
  Description of Artificial Sequence; Note = Synthetic Construct
atcggctctttatataagatccgccggatgcaggtctgatcatctttactt

SEQ ID NO: 185 - DNA, 51 bp, Artificial Sequence
  Description of Artificial Sequence; Note = Synthetic Construct
atcgatcctttatataagatccgccgaatgcaaagtagattatctccatcc

SEQ ID NO: 186 - DNA, 51 bp, Artificial Sequence
  Description of Artificial Sequence; Note = Synthetic Construct
atcgatcctttacgaaggggccttcgaatgcagggtagattatcttcatcc

SEQ ID NO: 187 - DNA, 51 bp, Artificial Sequence
  Description of Artificial Sequence; Note = Synthetic Construct
gtcgatcaaccatgacaaggccgccggacgcggggttgcgtatcttcatcc

SEQ ID NO: 188 - DNA, 51 bp, Artificial Sequence
  Description of Artificial Sequence; Note = Synthetic Construct
atcgatcaaccatgacaaggccgccggacgcggggttgcgtatcttcatcc

SEQ ID NO: 189 - DNA, 51 bp, Artificial Sequence
  Description of Artificial Sequence; Note = Synthetic Construct
accgatcaaccatgacaaggccgccggacgcggggttgcgcggcatcgtcc

SEQ ID NO: 190 - DNA, 51 bp, Artificial Sequence
  Description of Artificial Sequence; Note = Synthetic Construct
atcgatcctttatataagatccgccgaatgcaaagttgcgcggcttcgtcc

SEQ ID NO: 191 - DNA, 51 bp, Artificial Sequence
  Description of Artificial Sequence; Note = Synthetic Construct
accgatcaaccatgacaaggccgccggacgcggggttgctcgttttcgtcc

SEQ ID NO: 192 - DNA, 51 bp, Artificial Sequence
  Description of Artificial Sequence; Note = Synthetic Construct
actaatcctttacgaaggggccttcggatgtggggttgcgcggcttcgtcc

SEQ ID NO: 193 - DNA, 51 bp, Artificial Sequence
  Description of Artificial Sequence; Note = Synthetic Construct
atcgatcctttacgaaggggccttcggacgcggggttgcgtatcttcatcc

SEQ ID NO: 194 - DNA, 51 bp, Artificial Sequence
  Description of Artificial Sequence; Note = Synthetic Construct
actaatccttttcataagagtcgccggatacggggttgcgtatcttcatcc

SEQ ID NO: 195 - DNA, 51 bp, Artificial Sequence
  Description of Artificial Sequence; Note = Synthetic Construct
actaatccttttcataagagtcgccggatacggggttgcgcggcttcgtcc

SEQ ID NO: 196 - DNA, 51 bp, Artificial Sequence
  Description of Artificial Sequence; Note = Synthetic Construct
accgatcctttatgacaaggccgccggacgcggggttgcgtatcttcatcc

SEQ ID NO: 197 - DNA, 51 bp, Artificial Sequence
  Description of Artificial Sequence; Note = Synthetic Construct
atcgatcctttatataagatccgccggatacggggttgctcgttttcgtcc

SEQ ID NO: 198 - DNA, 51 bp, Artificial Sequence
  Description of Artificial Sequence; Note = Synthetic Construct
actaatccttttcataagagtcgccggatacggggttgctcggcttcgtcc

SEQ ID NO: 199 - DNA, 51 bp, Artificial Sequence
  Description of Artificial Sequence; Note = Synthetic Construct
actaatccttttcataagagtcgccggatgcggggttgcgtatcttcatcc

SEQ ID NO: 200 - DNA, 51 bp, Artificial Sequence
  Description of Artificial Sequence; Note = Synthetic Construct
atcgatcctttacgaaggggccttcggatacggggttgctcgttttcgtcc

SEQ ID NO: 201 - DNA, 51 bp, Artificial Sequence
  Description of Artificial Sequence; Note = Synthetic Construct
accgatcaaccatgacaaggccgccggacgcggggttacgcggcttcgtcc

SEQ ID NO: 202 - DNA, 51 bp, Artificial Sequence
  Description of Artificial Sequence; Note = Synthetic Construct
accgatccttttcataagagtcgccggatacggggttgctcgttttcgtcc

SEQ ID NO: 203 - DNA, 51 bp, Artificial Sequence
  Description of Artificial Sequence; Note = Synthetic Construct
actaatccttttcataagagtcgccggacgcggggttgcgcggcttcgtcc

SEQ ID NO: 204 - DNA, 51 bp, Artificial Sequence
  Description of Artificial Sequence; Note = Synthetic Construct
accgatcaaccatgacaaggccgccggacgcggagtagattatcttcatcc

SEQ ID NO: 205 - DNA, 51 bp, Artificial Sequence
  Description of Artificial Sequence; Note = Synthetic Construct
gtcgatcaaccatgacaaggccgcgggacgcggggttgcgtatcttcatcc

SEQ ID NO: 206 - DNA, 51 bp, Artificial Sequence
  Description of Artificial Sequence; Note = Synthetic Construct
gtcgatcctttatgacaaggccgccggatacggggttgctcgttttcgtcc

SEQ ID NO: 207 - DNA, 51 bp, Artificial Sequence
  Description of Artificial Sequence; Note = Synthetic Construct
atcgatcctttatataagatccgccggacgcggggttgcgcggcttcgtcc

SEQ ID NO: 208 - DNA, 51 bp, Artificial Sequence
  Description of Artificial Sequence; Note = Synthetic Construct
atcgatcctttatataagatccttcgagtgcggggttgcgcggcttcgtcc

SEQ ID NO: 209 - DNA, 51 bp, Artificial Sequence
  Description of Artificial Sequence; Note = Synthetic Construct
accgatcctttacgaaggggccttcgaatgcaaagtagattatcttcatcc

SEQ ID NO: 210 - DNA, 51 bp, Artificial Sequence
  Description of Artificial Sequence; Note = Synthetic Construct
accgatcaaccatgacaaggccgccggacgcgggtctgatcatctttactt

SEQ ID NO: 211 - DNA, 51 bp, Artificial Sequence
  Description of Artificial Sequence; Note = Synthetic Construct
accggttctttatataagatccgccggatgcagggttgcgcggcttcgtcc

SEQ ID NO: 212 - DNA, 51 bp, Artificial Sequence
  Description of Artificial Sequence; Note = Synthetic Construct
actaatcctttacgaaggggccttcgagtgcagggttgcgcggcttcgtcc

SEQ ID NO: 213 - DNA, 51 bp, Artificial Sequence
  Description of Artificial Sequence; Note = Synthetic Construct
atcgatcctttacgaaggggccttcgaatgcagggttgcgcggcttcgtcc

SEQ ID NO: 214 - DNA, 51 bp, Artificial Sequence
  Description of Artificial Sequence; Note = Synthetic Construct
gtcgatcctttatgacaaggccgccggatgcggggttgcgtatcttcatcc

SEQ ID NO: 215 - DNA, 51 bp, Artificial Sequence
  Description of Artificial Sequence; Note = Synthetic Construct
accgatcctttatgacaaggccgccggacgcggggttgcgcggcttcgtcc

SEQ ID NO: 216 - DNA, 51 bp, Artificial Sequence
  Description of Artificial Sequence; Note = Synthetic Construct
accgatcctttatataagatccgccggatacggggttgctcgttttcgtcc

SEQ ID NO: 217 - DNA, 51 bp, Artificial Sequence
  Description of Artificial Sequence; Note = Synthetic Construct
actaatcctttacgaaggggccttcggacgcggggttgcgcggcttcgtcc

SEQ ID NO: 218 - DNA, 51 bp, Artificial Sequence
  Description of Artificial Sequence; Note = Synthetic Construct
accgatcctttacgaaggggccttcggatgtggggttgcgtatcttcatcc

SEQ ID NO: 219 - DNA, 51 bp, Artificial Sequence
  Description of Artificial Sequence; Note = Synthetic Construct
actaatccttttcataagagtcgccggacacggggttgctcgttttcgtcc

SEQ ID NO: 220 - DNA, 51 bp, Artificial Sequence
  Description of Artificial Sequence; Note = Synthetic Construct
accgatccttttcataagagtcgccggatacgaagtagattatcttcatcc

SEQ ID NO: 221 - DNA, 51 bp, Artificial Sequence
  Description of Artificial Sequence; Note = Synthetic Construct
atcgatcaaccatgacaaggccttcgaatgcaaagtagattatcttcatcc

SEQ ID NO: 222 - DNA, 51 bp, Artificial Sequence
  Description of Artificial Sequence; Note = Synthetic Construct
accgatcctttacgaaggggccttcgaatgcaaagtagatcatctttactt

SEQ ID NO: 223 - DNA, 51 bp, Artificial Sequence
  Description of Artificial Sequence; Note = Synthetic Construct
accgatcaactatgacaaggccgccggacgcggggttgcgcggcttcgtcc

SEQ ID NO: 224 - DNA, 51 bp, Artificial Sequence
  Description of Artificial Sequence; Note = Synthetic Construct
actagctctttatataagatccgccgaatgcaaagtagattatcttcatcc

SEQ ID NO: 225 - DNA, 51 bp, Artificial Sequence
  Description of Artificial Sequence; Note = Synthetic Construct
actaatccttttcgtaagagtcgccggatacggggttgctcgttttcgtcc

SEQ ID NO: 226 - DNA, 51 bp, Artificial Sequence
  Description of Artificial Sequence; Note = Synthetic Construct
atcgatccttttcataagagtcgccggatacggggttgctcgttttcgtcc

SEQ ID NO: 227 - DNA, 51 bp, Artificial Sequence
  Description of Artificial Sequence; Note = Synthetic Construct
actaatcctttacgaaggggccttcggatacggggttgctcgttttcgtcc

SEQ ID NO: 228 - DNA, 51 bp, Artificial Sequence
  Description of Artificial Sequence; Note = Synthetic Construct
atcgatcaaccatgacaaggccgcgggacgcggggttgcgtatcttcatcc

SEQ ID NO: 229 - DNA, 51 bp, Artificial Sequence; identical to SEQ ID NO: 164
  Description of Artificial Sequence; Note = Synthetic Construct

SEQ ID NO: 230 - DNA, 51 bp, Artificial Sequence; identical to SEQ ID NO: 165
  Description of Artificial Sequence; Note = Synthetic Construct

SEQ ID NO: 231 - DNA, 51 bp, Artificial Sequence; identical to SEQ ID NO: 166
  Description of Artificial Sequence; Note = Synthetic Construct

SEQ ID NO: 232 - DNA, 51 bp, Artificial Sequence; identical to SEQ ID NO: 167
  Description of Artificial Sequence; Note = Synthetic Construct

SEQ ID NO: 233 - DNA, 51 bp, Artificial Sequence; identical to SEQ ID NO: 168
  Description of Artificial Sequence; Note = Synthetic Construct

SEQ ID NO: 234 - DNA, 51 bp, Artificial Sequence; identical to SEQ ID NO: 169
  Description of Artificial Sequence; Note = Synthetic Construct

SEQ ID NO: 235 - DNA, 51 bp, Artificial Sequence; identical to SEQ ID NO: 170
  Description of Artificial Sequence; Note = Synthetic Construct

SEQ ID NO: 236 - DNA, 51 bp, Artificial Sequence; identical to SEQ ID NO: 171
  Description of Artificial Sequence; Note = Synthetic Construct

SEQ ID NO: 237 - DNA, 51 bp, Artificial Sequence; identical to SEQ ID NO: 174
  Description of Artificial Sequence; Note = Synthetic Construct

SEQ ID NO: 238 - DNA, 51 bp, Artificial Sequence; identical to SEQ ID NO: 179
  Description of Artificial Sequence; Note = Synthetic Construct

SEQ ID NO: 239 - DNA, 51 bp, Artificial Sequence; identical to SEQ ID NO: 172
  Description of Artificial Sequence; Note = Synthetic Construct

SEQ ID NO: 240 - DNA, 51 bp, Artificial Sequence; identical to SEQ ID NO: 173
  Description of Artificial Sequence; Note = Synthetic Construct

SEQ ID NO: 241 - DNA, 51 bp, Artificial Sequence; identical to SEQ ID NO: 181
  Description of Artificial Sequence; Note = Synthetic Construct

SEQ ID NO: 242 - DNA, 51 bp, Artificial Sequence; identical to SEQ ID NO: 177
  Description of Artificial Sequence; Note = Synthetic Construct

SEQ ID NO: 243 - DNA, 51 bp, Artificial Sequence; identical to SEQ ID NO: 178
  Description of Artificial Sequence; Note = Synthetic Construct

SEQ ID NO: 244 - DNA, 51 bp, Artificial Sequence; identical to SEQ ID NO: 184
  Description of Artificial Sequence; Note = Synthetic Construct

SEQ ID NO: 245 - DNA, 51 bp, Artificial Sequence; identical to SEQ ID NO: 175
  Description of Artificial Sequence; Note = Synthetic Construct

SEQ ID NO: 246 - DNA, 51 bp, Artificial Sequence; identical to SEQ ID NO: 200
  Description of Artificial Sequence; Note = Synthetic Construct

SEQ ID NO: 247 - DNA, 51 bp, Artificial Sequence; identical to SEQ ID NO: 176
  Description of Artificial Sequence; Note = Synthetic Construct

SEQ ID NO: 248 - DNA, 51 bp, Artificial Sequence; identical to SEQ ID NO: 183
  Description of Artificial Sequence; Note = Synthetic Construct

SEQ ID NO: 249 - DNA, 51 bp, Artificial Sequence; identical to SEQ ID NO: 182
  Description of Artificial Sequence; Note = Synthetic Construct

SEQ ID NO: 250 - DNA, 51 bp, Artificial Sequence; identical to SEQ ID NO: 201
  Description of Artificial Sequence; Note = Synthetic Construct

SEQ ID NO: 251 - DNA, 51 bp, Artificial Sequence; identical to SEQ ID NO: 188
  Description of Artificial Sequence; Note = Synthetic Construct

SEQ ID NO: 252 - DNA, 51 bp, Artificial Sequence; identical to SEQ ID NO: 180
  Description of Artificial Sequence; Note = Synthetic Construct

SEQ ID NO: 253 - DNA, 51 bp, Artificial Sequence; identical to SEQ ID NO: 186
  Description of Artificial Sequence; Note = Synthetic Construct

SEQ ID NO: 254 - DNA, 51 bp, Artificial Sequence; identical to SEQ ID NO: 192
  Description of Artificial Sequence; Note = Synthetic Construct

SEQ ID NO: 255 - DNA, 51 bp, Artificial Sequence; identical to SEQ ID NO: 195
  Description of Artificial Sequence; Note = Synthetic Construct

SEQ ID NO: 256 - DNA, 51 bp, Artificial Sequence; identical to SEQ ID NO: 193
  Description of Artificial Sequence; Note = Synthetic Construct

SEQ ID NO: 257 - DNA, 51 bp, Artificial Sequence; identical to SEQ ID NO: 187
  Description of Artificial Sequence; Note = Synthetic Construct

SEQ ID NO: 258 - DNA, 51 bp, Artificial Sequence; identical to SEQ ID NO: 197
  Description of Artificial Sequence; Note = Synthetic Construct

SEQ ID NO: 259 - DNA, 51 bp, Artificial Sequence; identical to SEQ ID NO: 189
  Description of Artificial Sequence; Note = Synthetic Construct

SEQ ID NO: 260 - DNA, 51 bp, Artificial Sequence; identical to SEQ ID NO: 185
  Description of Artificial Sequence; Note = Synthetic Construct

SEQ ID NO: 261 - DNA, 51 bp, Artificial Sequence; identical to SEQ ID NO: 209
  Description of Artificial Sequence; Note = Synthetic Construct

SEQ ID NO: 262 - DNA, 51 bp, Artificial Sequence; identical to SEQ ID NO: 190
  Description of Artificial Sequence; Note = Synthetic Construct

SEQ ID NO: 263 - DNA, 51 bp, Artificial Sequence; identical to SEQ ID NO: 205
  Description of Artificial Sequence; Note = Synthetic Construct

SEQ ID NO: 264 - DNA, 51 bp, Artificial Sequence; identical to SEQ ID NO: 191
  Description of Artificial Sequence; Note = Synthetic Construct

SEQ ID NO: 265 - DNA, 51 bp, Artificial Sequence; identical to SEQ ID NO: 199
  Description of Artificial Sequence; Note = Synthetic Construct

SEQ ID NO: 266 - DNA, 51 bp, Artificial Sequence
  Description of Artificial Sequence; Note = Synthetic Construct
actaactctttacataagagtcgccggatacggggttgctcgttttcgtcc

SEQ ID NO: 267 - DNA, 51 bp, Artificial Sequence; identical to SEQ ID NO: 196
  Description of Artificial Sequence; Note = Synthetic Construct

SEQ ID NO: 268 - DNA, 51 bp, Artificial Sequence
  Description of Artificial Sequence; Note = Synthetic Construct
atcggctctttatataagatccgccggatgcagggttgcgcggcttcgtcc

SEQ ID NO: 269 - DNA, 51 bp, Artificial Sequence; identical to SEQ ID NO: 194
  Description of Artificial Sequence; Note = Synthetic Construct

SEQ ID NO: 270 - DNA, 51 bp, Artificial Sequence; identical to SEQ ID NO: 224
  Description of Artificial Sequence; Note = Synthetic Construct

SEQ ID NO: 271 - DNA, 51 bp, Artificial Sequence; identical to SEQ ID NO: 198
  Description of Artificial Sequence; Note = Synthetic Construct

SEQ ID NO: 272 - DNA, 51 bp, Artificial Sequence
  Description of Artificial Sequence; Note = Synthetic Construct
gccgatcaaccatgacaaggccgccggacgcggggttgcgcggcttcgtcc

SEQ ID NO: 273 - DNA, 51 bp, Artificial Sequence; identical to SEQ ID NO: 202
  Description of Artificial Sequence; Note = Synthetic Construct

SEQ ID NO: 274 - DNA, 51 bp, Artificial Sequence
  Description of Artificial Sequence; Note = Synthetic Construct
atcgatcctttacgaaggggccttcggatgtggggttgcgtatcttcatcc

SEQ ID NO: 275 - DNA, 51 bp, Artificial Sequence; identical to SEQ ID NO: 221
  Description of Artificial Sequence; Note = Synthetic Construct

SEQ ID NO: 276 - DNA, 51 bp, Artificial Sequence; identical to SEQ ID NO: 208
  Description of Artificial Sequence; Note = Synthetic Construct

SEQ ID NO: 277 - DNA, 51 bp, Artificial Sequence
  Description of Artificial Sequence; Note = Synthetic Construct
actaatcaaccatgacaaggccgccggacgcggggttgcgcggcttcgtcc

SEQ ID NO: 278 - DNA, 51 bp, Artificial Sequence; identical to SEQ ID NO: 210
  Description of Artificial Sequence; Note = Synthetic Construct

SEQ ID NO: 279 - DNA, 51 bp, Artificial Sequence; identical to SEQ ID NO: 206
  Description of Artificial Sequence; Note = Synthetic Construct

SEQ ID NO: 280 - DNA, 51 bp, Artificial Sequence
  Description of Artificial Sequence; Note = Synthetic Construct
gccgatcaaccatgacaaggccgccggacgcggggttgcgtatcttcatcc

SEQ ID NO: 281 - DNA, 51 bp, Artificial Sequence; identical to SEQ ID NO: 203
  Description of Artificial Sequence; Note = Synthetic Construct

SEQ ID NO: 282 - DNA, 51 bp, Artificial Sequence
  Description of Artificial Sequence; Note = Synthetic Construct
accgatcctttacgaaggggccttcggatgtggggttgcgcggcttcgtcc

SEQ ID NO: 283 - DNA, 51 bp, Artificial Sequence
  Description of Artificial Sequence; Note = Synthetic Construct
atcgatcctttatataagatccgccgaatgcagggttgcgtatcttcatcc

SEQ ID NO: 284 - DNA, 51 bp, Artificial Sequence; identical to SEQ ID NO: 226
  Description of Artificial Sequence; Note = Synthetic Construct

SEQ ID NO: 285 - DNA, 22 bp, Artificial Sequence
  Description of Artificial Sequence; Note = Synthetic Construct
tctgctcctgctcttcttttcc

SEQ ID NO: 286 - DNA, 25 bp, Artificial Sequence
  Description of Artificial Sequence; Note = Synthetic Construct
ctctatgcttgctacaagagacaca

SEQ ID NO: 287 - DNA, 16 bp, Artificial Sequence
  Description of Artificial Sequence; Note = Synthetic Construct
aacagcatggaatatc

SEQ ID NO: 288 - DNA, 17 bp, Artificial Sequence
  Description of Artificial Sequence; Note = Synthetic Construct
cgatccacgtcgcggtc

SEQ ID NO: 289 - DNA, 17 bp, Artificial Sequence
  Description of Artificial Sequence; Note = Synthetic Construct
cgatccacgtcgtattc

SEQ ID NO: 290 - DNA, 17 bp, Artificial Sequence
  Description of Artificial Sequence; Note = Synthetic Construct
cacctcatgtctcgttc

SEQ ID NO: 291 - DNA, 17 bp, Artificial Sequence
  Description of Artificial Sequence; Note = Synthetic Construct
tgccctatatattattc

SEQ ID NO: 292 - DNA, 17 bp, Artificial Sequence
  Description of Artificial Sequence; Note = Synthetic Construct
cgctccatacatcattt

SEQ ID NO: 293 - DNA, 17 bp, Artificial Sequence
  Description of Artificial Sequence; Note = Synthetic Construct
cacctcatgtattattc

SEQ ID NO: 294 - DNA, 17 bp, Artificial Sequence
  Description of Artificial Sequence; Note = Synthetic Construct
tgctccacgtcgtattc

SEQ ID NO: 295 - DNA, 17 bp, Artificial Sequence
  Description of Artificial Sequence; Note = Synthetic Construct
cgatccacgtattattc

SEQ ID NO: 296 - DNA, 17 bp, Artificial Sequence
  Description of Artificial Sequence; Note = Synthetic Construct
tgccctgtatcgcggtc

SEQ ID NO: 297 - DNA, 17 bp, Artificial Sequence
  Description of Artificial Sequence; Note = Synthetic Construct
tgatccacgtcgcggtc

SEQ ID NO: 298 - DNA, 17 bp, Artificial Sequence
  Description of Artificial Sequence; Note = Synthetic Construct
tgccctatgtcgcggtc

SEQ ID NO: 299 - DNA, 17 bp, Artificial Sequence
  Description of Artificial Sequence; Note = Synthetic Construct
tgctccatatattattc

SEQ ID NO: 300 - DNA, 17 bp, Artificial Sequence
  Description of Artificial Sequence; Note = Synthetic Construct
cgatccacgtcgcggtc

SEQ ID NO: 301 - DNA, 17 bp, Artificial Sequence
  Description of Artificial Sequence; Note = Synthetic Construct
cgctccatatattattc

SEQ ID NO: 302 - DNA, 17 bp, Artificial Sequence
  Description of Artificial Sequence; Note = Synthetic Construct
cgctccatatcgcggtc

SEQ ID NO: 303 - DNA, 17 bp, Artificial Sequence
  Description of Artificial Sequence; Note = Synthetic Construct
tgctccatacatcattt

SEQ ID NO: 304 - DNA, 17 bp, Artificial Sequence
  Description of Artificial Sequence; Note = Synthetic Construct
cgatccatgtctcgttc

SEQ ID NO: 305 - DNA, 17 bp, Artificial Sequence
  Description of Artificial Sequence; Note = Synthetic Construct
tgccctatgtctcgttc

SEQ ID NO: 306 - DNA, 17 bp, Artificial Sequence
  Description of Artificial Sequence; Note = Synthetic Construct
cgatccatgtcgcggtc

SEQ ID NO: 307 - DNA, 17 bp, Artificial Sequence
  Description of Artificial Sequence; Note = Synthetic Construct
cgatccatatattattc

SEQ ID NO: 308 - DNA, 17 bp, Artificial Sequence
  Description of Artificial Sequence; Note = Synthetic Construct
tgccctatgtcgcggtc

SEQ ID NO: 309 - DNA, 17 bp, Artificial Sequence
  Description of Artificial Sequence; Note = Synthetic Construct
cgatccacgtcgcggtc

SEQ ID NO: 310 - DNA, 17 bp, Artificial Sequence
  Description of Artificial Sequence; Note = Synthetic Construct
tgatccacgtcgtattc

SEQ ID NO: 311 - DNA, 17 bp, Artificial Sequence
  Description of Artificial Sequence; Note = Synthetic Construct
cgatccacgtcgtattc

SEQ ID NO: 312 - DNA, 17 bp, Artificial Sequence
  Description of Artificial Sequence; Note = Synthetic Construct
tgccctatatattattc

SEQ ID NO: 313 - DNA, 17 bp, Artificial Sequence
  Description of Artificial Sequence; Note = Synthetic Construct
caccctatgtcgcggtc

SEQ ID NO: 314 - DNA, 17 bp, Artificial Sequence
  Description of Artificial Sequence; Note = Synthetic Construct
cacctcatgtcgcggtc

SEQ ID NO: 315 - DNA, 17 bp, Artificial Sequence
  Description of Artificial Sequence; Note = Synthetic Construct
tgccctacgtcgtattc

SEQ ID NO: 316 - DNA, 17 bp, Artificial Sequence
  Description of Artificial Sequence; Note = Synthetic Construct
tgatccacgtcgtattc

SEQ ID NO: 317 - DNA, 17 bp, Artificial Sequence
  Description of Artificial Sequence; Note = Synthetic Construct
tgctccatgtctcgttc

SEQ ID NO: 318 - DNA, 17 bp, Artificial Sequence
  Description of Artificial Sequence; Note = Synthetic Construct
cgatccacgtcgcggtc

SEQ ID NO: 319 - DNA, 17 bp, Artificial Sequence
  Description of Artificial Sequence; Note = Synthetic Construct
tgctccatatattatcc

SEQ ID NO: 320 - DNA, 17 bp, Artificial Sequence
  Description of Artificial Sequence; Note = Synthetic Construct
cgccctatatattattc

SEQ ID NO: 321 - DNA, 17 bp, Artificial Sequence
  Description of Artificial Sequence; Note = Synthetic Construct
tgctccatatcgcggtc

SEQ ID NO: 322 - DNA, 17 bp, Artificial Sequence
  Description of Artificial Sequence; Note = Synthetic Construct
tgatccacgtcgtattc

SEQ ID NO: 323 - DNA, 17 bp, Artificial Sequence
  Description of Artificial Sequence; Note = Synthetic Construct
cgatccacgtctcgttc

SEQ ID NO: 324 - DNA, 17 bp, Artificial Sequence
  Description of Artificial Sequence; Note = Synthetic Construct
cacctcatgtcgtattc

SEQ ID NO: 325 - DNA, 17 bp, Artificial Sequence
  Description of Artificial Sequence; Note = Synthetic Construct
cacctcatgtctcgttc

SEQ ID NO: 326 - DNA, 17 bp, Artificial Sequence
  Description of Artificial Sequence; Note = Synthetic Construct
cgctccacgtcgtattc

SEQ ID NO: 327 - DNA, 17 bp, Artificial Sequence
  Description of Artificial Sequence; Note = Synthetic Construct
tgctccatatcgcggtc

SEQ ID NO: 328 - DNA, 17 bp, Artificial Sequence
  Description of Artificial Sequence; Note = Synthetic Construct
cacctcatgtcgtattc

SEQ ID NO: 329 - DNA, 17 bp, Artificial Sequence
  Description of Artificial Sequence; Note = Synthetic Construct
cactccatatattattc

SEQ ID NO: 330 - DNA, 17 bp, Artificial Sequence
  Description of Artificial Sequence; Note = Synthetic Construct
cacctcatgtctcggtc

SEQ ID NO: 331 - DNA, 17 bp, Artificial Sequence
  Description of Artificial Sequence; Note = Synthetic Construct
cgatccacgtcgcggtc

SEQ ID NO: 332 - DNA, 17 bp, Artificial Sequence
  Description of Artificial Sequence; Note = Synthetic Construct
cgcctcatgtctcgttc

SEQ ID NO: 333 - DNA, 17 bp, Artificial Sequence
  Description of Artificial Sequence; Note = Synthetic Construct
tgccctatgtcgtattc

SEQ ID NO: 334 - DNA, 17 bp, Artificial Sequence
  Description of Artificial Sequence; Note = Synthetic Construct
tgatctatatattattc

SEQ ID NO: 335 - DNA, 17 bp, Artificial Sequence
  Description of Artificial Sequence; Note = Synthetic Construct
tgctctgtgtcgcggtc

SEQ ID NO: 336 - DNA, 17 bp, Artificial Sequence
  Description of Artificial Sequence; Note = Synthetic Construct
caatccacgtcgcggtc

SEQ ID NO: 337 - DNA, 17 bp, Artificial Sequence
  Description of Artificial Sequence; Note = Synthetic Construct
cgatccacgcatcattt

SEQ ID NO: 338 - DNA, 17 bp, Artificial Sequence
  Description of Artificial Sequence; Note = Synthetic Construct
tgctccatgtctcgttc

SEQ ID NO: 339 - DNA, 17 bp, Artificial Sequence
  Description of Artificial Sequence; Note = Synthetic Construct
cgatccacgtcgtattc

SEQ ID NO: 340 - DNA, 17 bp, Artificial Sequence
  Description of Artificial Sequence; Note = Synthetic Construct
cacctcacgtcgcggtc

SEQ ID NO: 341 - DNA, 17 bp, Artificial Sequence
  Description of Artificial Sequence; Note = Synthetic Construct
cgccctatgtcgcggtc

SEQ ID NO: 342 - DNA, 17 bp, Artificial Sequence
  Description of Artificial Sequence; Note = Synthetic Construct
tgctccatatcgtattc

SEQ ID NO: 343 - DNA, 17 bp, Artificial Sequence
  Description of Artificial Sequence; Note = Synthetic Construct
tgcctcatgtctcgttc

SEQ ID NO: 344 - DNA, 17 bp, Artificial Sequence
  Description of Artificial Sequence; Note = Synthetic Construct
cgatccacgtcgcggtc

SEQ ID NO: 345 - DNA, 17 bp, Artificial Sequence
  Description of Artificial Sequence; Note = Synthetic Construct
cgatccacgtcgcggtc

SEQ ID NO: 346 - DNA, 17 bp, Artificial Sequence
  Description of Artificial Sequence; Note = Synthetic Construct
cacctcatgtctcgttc

SEQ ID NO: 347 - DNA, 17 bp, Artificial Sequence
  Description of Artificial Sequence; Note = Synthetic Construct
tgccctatatattattc

SEQ ID NO: 348 - DNA, 17 bp, Artificial Sequence
  Description of Artificial Sequence; Note = Synthetic Construct
cgatccacgtcgtattc

SEQ ID NO: 349 - DNA, 17 bp, Artificial Sequence
  Description of Artificial Sequence; Note = Synthetic Construct
cacctcatgtattattc

SEQ ID NO: 350 - DNA, 17 bp, Artificial Sequence
  Description of Artificial Sequence; Note = Synthetic Construct
cgctccatacatcattt

SEQ ID NO: 351 - DNA, 17 bp, Artificial Sequence
  Description of Artificial Sequence; Note = Synthetic Construct
cacctcatgtctcgttc

SEQ ID NO: 352 - DNA, 17 bp, Artificial Sequence
  Description of Artificial Sequence; Note = Synthetic Construct
tgctccacgtcgtattc

SEQ ID NO: 353 - DNA, 17 bp, Artificial Sequence
  Description of Artificial Sequence; Note = Synthetic Construct
tgccctgtatcgcggtc

SEQ ID NO: 354 - DNA, 17 bp, Artificial Sequence
  Description of Artificial Sequence; Note = Synthetic Construct
tgccctatgtcgcggtc

SEQ ID NO: 355 - DNA, 17 bp, Artificial Sequence
  Description of Artificial Sequence; Note = Synthetic Construct
cgatccacgtcgtattc

SEQ ID NO: 356 - DNA, 17 bp, Artificial Sequence
  Description of Artificial Sequence; Note = Synthetic Construct
cgctccatacatcattt

SEQ ID NO: 357 - DNA, 17 bp, Artificial Sequence
  Description of Artificial Sequence; Note = Synthetic Construct
tgctccatatattattc

SEQ ID NO: 358 - DNA, 17 bp, Artificial Sequence
  Description of Artificial Sequence; Note = Synthetic Construct
cgctccatatattattc

SEQ ID NO: 359 - DNA, 17 bp, Artificial Sequence
  Description of Artificial Sequence; Note = Synthetic Construct
cgatccacgtattattc

SEQ ID NO: 360 - DNA, 17 bp, Artificial Sequence
  Description of Artificial Sequence; Note = Synthetic Construct
tgctccatatcgcggtc

SEQ ID NO: 361 - DNA, 17 bp, Artificial Sequence
  Description of Artificial Sequence; Note = Synthetic Construct
cgctccatatcgcggtc

SEQ ID NO: 362 - DNA, 17 bp, Artificial Sequence
  Description of Artificial Sequence; Note = Synthetic Construct
cgatccacgtcgtattc

SEQ ID NO: 363 - DNA, 17 bp, Artificial Sequence
  Description of Artificial Sequence; Note = Synthetic Construct
cgatccatatattattc

SEQ ID NO: 364 - DNA, 17 bp, Artificial Sequence
  Description of Artificial Sequence; Note = Synthetic Construct
cgatccatgtcgcggtc

SEQ ID NO: 365 - DNA, 17 bp, Artificial Sequence
  Description of Artificial Sequence; Note = Synthetic Construct
cacctcatgtctcggtc

SEQ ID NO: 366 - DNA, 17 bp, Artificial Sequence
  Description of Artificial Sequence; Note = Synthetic Construct
cgatccatgtctcgttc

SEQ ID NO: 367 - DNA, 17 bp, Artificial Sequence
  Description of Artificial Sequence; Note = Synthetic Construct
cgatccatgtctcgttc

SEQ ID NO: 368 - DNA, 17 bp, Artificial Sequence
  Description of Artificial Sequence; Note = Synthetic Construct
cacctcatgtcgtattc

SEQ ID NO: 369 - DNA, 17 bp, Artificial Sequence
  Description of Artificial Sequence; Note = Synthetic Construct
cgctccacgtcgtattc

SEQ ID NO: 370 - DNA, 17 bp, Artificial Sequence
  Description of Artificial Sequence; Note = Synthetic Construct
tgccctatatattattc

SEQ ID NO: 371 - DNA, 17 bp, Artificial Sequence
  Description of Artificial Sequence; Note = Synthetic Construct
cgctccatatcgcggtc

SEQ ID NO: 372 - DNA, 17 bp, Artificial Sequence
  Description of Artificial Sequence; Note = Synthetic Construct
tgatccacgtcgcggtc

SEQ ID NO: 373 - DNA, 17 bp, Artificial Sequence
  Description of Artificial Sequence; Note = Synthetic Construct
tgatccacgtcgcggtc

SEQ ID NO: 374 - DNA, 17 bp, Artificial Sequence
  Description of Artificial Sequence; Note = Synthetic Construct
cgctccatgtcgcggtc

SEQ ID NO: 375 - DNA, 17 bp, Artificial Sequence
  Description of Artificial Sequence; Note = Synthetic Construct
tgctccatatattatcc

SEQ ID NO: 376 - DNA, 17 bp, Artificial Sequence
  Description of Artificial Sequence; Note = Synthetic Construct
cgctccatacatcattt